(19) United States
(12) Patent Application Publication
(10) Pub. No.: US 2022/0307043 A1
(43) Pub. Date: Sep. 29, 2022

(54) GENETICALLY MODIFIED PLANTS AND METHODS OF MAKING THE SAME

(71) Applicant: EMPYREAN NEUROSCIENCE, INC., New York, NY (US)

(72) Inventors: Thomas HENLEY, New York, NY (US); Modassir CHOUDHRY, New York, NY (US); Jose FERNANDEZ-GOMEZ, New York, NY (US)

(21) Appl. No.: 17/711,258

(22) Filed: Apr. 1, 2022

Related U.S. Application Data
(63) Continuation of application No. PCT/US2020/053871, filed on Oct. 1, 2020.
(60) Provisional application No. 62/909,094, filed on Oct. 1, 2019.

(51) Int. Cl. C12N 15/82; A61K 31/352

(52) U.S. Cl. CPC C12N 15/8243; A61K 31/352

(57) ABSTRACT

Provided herein are methods for modulating the cannabinoid biosynthesis pathway in plants. Also provided are cannabinoid compositions comprising rare cannabinoids, new cannabinoids, and variant cannabinoids generated by the provided methods.

### CROSS REFERENCE

This application is a continuation of International Application No. PCT/US2020/053871, filed Oct. 1, 2020, which claims the benefit of U.S. Provisional Patent Application No. 62/909,094, filed Oct. 1, 2019, which is entirely incorporated herein by reference.

### SEQUENCE LISTING

The instant application contains a Sequence Listing which has been submitted electronically in ASCII format and is hereby incorporated by reference in its entirety. Said ASCII copy, created on Nov. 12, 2020, is named 199827-706301_SL.txt and is 112,582 bytes in size.

### BACKGROUND

Naturally occurring components in cannabis may impact the efficacy of therapy and any potential side effects. Accordingly, cannabis plants having a modified therapeutic component(s) profile may be useful in the production of cannabis and/or may also be useful in the production of genetically modified cannabis providing a desired drug profile.

### SUMMARY

In one aspect, provided herein are transgenic plants that comprises at least one genetic modification, wherein said genetic modification results in an increased level of a compound of:

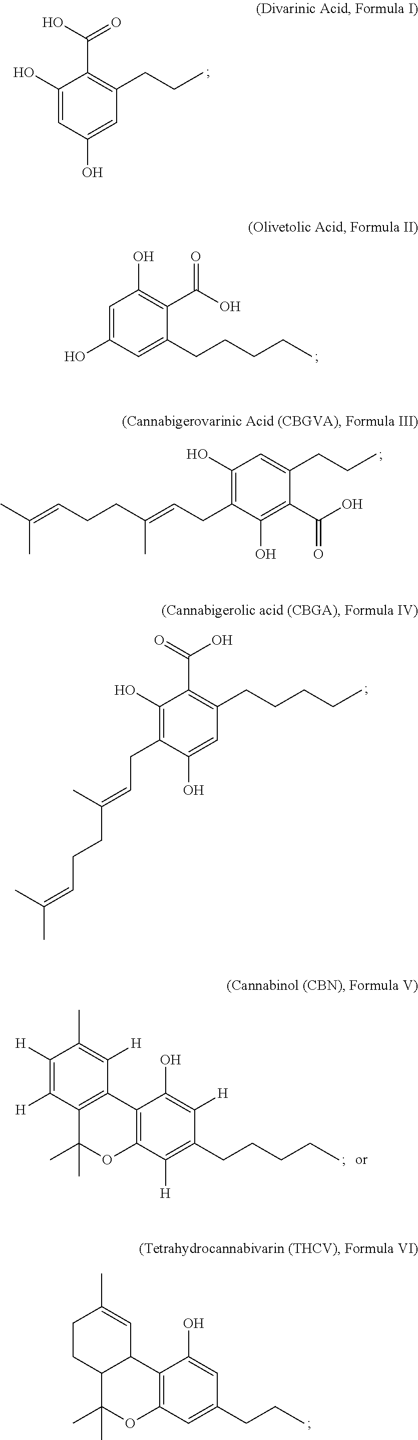

or a derivative or analog thereof, compared to a level of said compound in a comparable plant lacking said genetic modification.

In another aspect, provided herein are transgenic plants that comprises at least one genetic modification, wherein said genetic modification results in an increased level of a compound of:

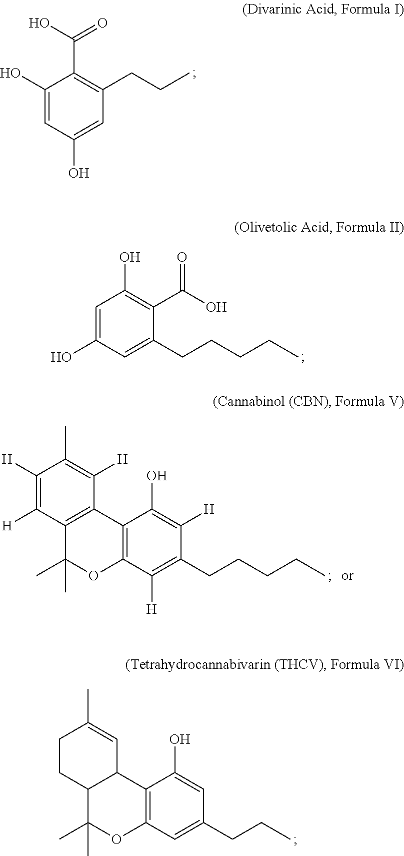 [SMILES: CCCc1cc(O)cc(O)c1C(=O)O ; CCCCCc1cc(O)cc(O)c1C(=O)O ; CCCCCc1cc(O)c2c(c1)OC(C)(C)c1ccc(C)cc1-2 ; CCCc1cc(O)c2c(c1)OC(C)(C)C1CCC(C)=CC21]

or a derivative or analog thereof, compared to a level of said compound in a comparable plant lacking said genetic modification.

In some embodiments, said at least one genetic modification is in a promoter or enhancer sequence of a gene encoding a protein.

In some embodiments, said gene encodes a polyketide cyclase or a polyketide synthase.

In some embodiments, said polyketide cyclase is olivetolic acid cyclase.

In some embodiments, said polyketide synthase is olivetolic acid synthase.

In some embodiments, said at least one genetic modification increases expression of said protein compared to a comparable plant lacking said genetic modification.

In some embodiments, said at least one genetic modification increases activity of said promoter or enhancer.

In some embodiments, said at least one genetic modification results in an increased level of a compound of Formula II, compared to a level of said compound in a comparable plant lacking said genetic modification.

In some embodiments, said at least one genetic modification results in an increased level of olivetolic acid compared to a comparable plant lacking said genetic modification.

In some embodiments, said transgenic plant comprises at least two genetic modifications, wherein each genetic modification is in a promoter or enhancer sequence of a gene encoding a protein.

In some embodiments, said transgenic plant comprises a genetic modification in a promoter or enhancer of a sequence of a gene encoding a polyketide cyclase and a genetic modification in a promoter or enhancer of a sequence of a gene encoding a polyketide synthase.

In some embodiments, said polyketide cyclase is olivetolic acid cyclase.

In some embodiments, said polyketide synthase is olivetolic acid synthase.

In some embodiments, said at least two genetic modifications increases expression of said olivetolic acid cyclase compared to a comparable plant lacking said at least genetic modification.

In some embodiments, said at least two genetic modifications increases expression of said olivetolic acid synthase compared to a comparable plant lacking said at least genetic modification.

In some embodiments, said at least two genetic modifications increases expression of said olivetolic acid synthase and olivetolic acid synthase compared to a comparable plant lacking said at least genetic modification.

In some embodiments, said at least two genetic modifications results in an increased level of a compound of Formula II, compared to a level of said compound in a comparable plant lacking said genetic modification.

In some embodiments, said at least two genetic modifications results in an increased level of olivetolic acid compared to a comparable plant lacking said at least two genetic modification.

In some embodiments, said at least two genetic modifications increases activity of said promoters or enhancers.

In some embodiments, said gene encodes Geranyl-pyrophosphate—olivetolic acid geranyltransferase (GOT).

In some embodiments, said at least one genetic modification increases expression of Geranyl-pyrophosphate—olivetolic acid geranyltransferase (GOT) protein.

In some embodiments, said at least one genetic modification results in an increased level of a compound of Formula IV, compared to a level of said compound in a comparable plant lacking said genetic modification.

In some embodiments, said at least one genetic modification results in an increased level of cannabigerolic acid (CBGA), compared to a comparable plant lacking said genetic modification.

In some embodiments, said at least one genetic modification increases activity of said promoter or enhancer.

In some embodiments, said at least one genetic modification is in a gene sequence that encodes a protein.

In some embodiments, said at least one genetic modification disrupts expression of said protein.

In some embodiments, said at least one genetic modification decreases expression of said protein compared to a comparable plant lacking said genetic modification.

In some embodiments, said at least one genetic modification decreases expression of said protein by at least 30%, 40%, 50%, 60%, 70%, 80%, 90%, 100%, compared to a comparable plant lacking said genetic modification.

In some embodiments, said at least one genetic modification is in a gene sequence that encodes a tetrahydrocannabinolic acid synthase, a cannabidiolic acid synthase, or a cannabichromenic acid synthase.

In some embodiments, said transgenic plant comprises at least two genetic modifications each in a gene sequence that encodes a protein.

In some embodiments, said transgenic plant comprises at least two genetic modifications each in a different gene sequence that encode different proteins.

In some embodiments, said at least two genetic modifications disrupts expression of said proteins.

In some embodiments, said at least two genetic modifications decrease expression of said proteins compared to a comparable plant lacking said at least two genetic modifications.

In some embodiments, said at least two genetic modifications decrease expression of said proteins by at least 30%, 40%, 50%, 60%, 70%, 80%, 90%, 100%, compared to a comparable plant lacking said at least two genetic modifications.

In some embodiments, said at least two genetic modifications are in a gene sequence that encodes a tetrahydrocannabinolic acid synthase, a cannabidiolic acid synthase, or a cannabichromenic acid synthase.

In some embodiments, said transgenic plant that comprises a genetic modification in a promoter or enhancer sequence of a gene encoding a polyketide cyclase or a polyketide synthase, wherein said genetic modification in said promoter or enhancer increases expression of said polyketide cyclase or polyketide synthase, compared to a comparable plant lacking said genetic modificationin said promoter or enhancer sequence; a genetic disruption in a gene sequence that encodes a Tetrahydrocannabinolic acid synthase, a cannabidiolic acid synthase, or a cannabichromenic acid synthase, wherein said genetic disruption decreases expression of said tetrahydrocannabinolic acid synthase, cannabidiolic acid synthase, or cannabichromenic acid synthase; compared to a comparable plant lacking said genetic disruption in said gene sequence that encodes a Tetrahydrocannabinolic acid synthase, a cannabidiolic acid synthase, or a cannabichromenic acid synthase.

In some embodiments, said genetic modification in said promoter or enhancer increases expression of said polyketide cyclase or polyketide synthase by at least 5%, 10%, 20%, 30%, 40%, 50%, 60%, 70%, 80%, 90%, 100%, compared to a comparable plant lacking said genetic modification in said promoter or enhancer sequence.

In some embodiments, said genetic modification in said promoter or enhancer increases expression of said polyketide cyclase or polyketide synthase by at least 2 fold, 5 fold, 10 fold, 100 fold, 500 fold, 1000 fold, or 10000 fold compared to a comparable plant lacking said genetic modification in said promoter or enhancer sequence.

In some embodiments, said genetic disruption in said gene sequence that encodes a tetrahydrocannabinolic acid synthase, a cannabidiolic acid synthase, or a cannabichromenic acid synthase decreases expression of said tetrahydrocannabinolic acid synthase, cannabidiolic acid synthase, or cannabichromenic acid synthase by at least 5%, 10%, 20%, 30%, 40%, 50%, 60%, 70%, 80%, 90%, 100%, compared to a comparable plant lacking said genetic disruption in said gene sequence that encodes a tetrahydrocannabinolic acid synthase, a cannabidiolic acid synthase, or a cannabichromenic acid synthase.

In some embodiments, said genetic disruption in said gene sequence that encodes a tetrahydrocannabinolic acid synthase, a cannabidiolic acid synthase, or a cannabichromenic acid synthase decreases expression of said tetrahydrocannabinolic acid synthase, cannabidiolic acid synthase, or cannabichromenic acid synthase by at least 2 fold, 5 fold, 10 fold, 100 fold, 500 fold, 1000 fold, or 10000 fold, compared to a comparable plant lacking said genetic disruption in said gene sequence that encodes a tetrahydrocannabinolic acid synthase, a cannabidiolic acid synthase, or a cannabichromenic acid synthase.

In some embodiments, said wherein said at least one genetic modification results in an increased level of a compound of Formula II, compared to a level of said compound in a comparable plant lacking said genetic modification.

In some embodiments, said wherein said at least one genetic modification results in an increased level of a compound of Formula IV, compared to a level of said compound in a comparable plant lacking said genetic modification.

In some embodiments, said gene encodes tetrahydrocannabinolic acid synthase.

In some embodiments, said at least one genetic modification increases expression of said tetrahydrocannabinolic acid synthase compared to a comparable plant lacking said at least one genetic modification.

In some embodiments, said at least one genetic modification increases activity of said promoter or enhancer.

In some embodiments, said at least one genetic modification results in an increased level of a compound of Formula V, compared to a level of said compound in a comparable plant lacking said genetic modification.

In some embodiments, said at least one genetic modification results in an increased level of cannabinol compared to a comparable plant lacking said genetic modification.

In some embodiments, said at least one genetic modification is in a gene sequence that encodes a cannabidiolic acid synthase or a cannabichromenic acid synthase.

In some embodiments, said at least one genetic modification disrupts expression of said protein.

In some embodiments, said at least one genetic modification decreases expression of said protein compared to a comparable plant lacking said genetic modification.

In some embodiments, said at least one genetic modification decreases expression of said protein by at least 30%, 40%, 50%, 60%, 70%, 80%, 90%, 100%, compared to a comparable plant lacking said genetic modification.

In some embodiments, said at least one genetic modification decreases expression of said protein by at least 2 fold, 5 fold, 10 fold, 100 fold, 500 fold, 1000 fold, or 10000 fold, compared to a comparable plant lacking said genetic modification.

In some embodiments, said transgenic plant comprises a genetic modification in a promoter or enhancer sequence of a gene encoding a THC synthase, wherein said genetic modification in said promoter or enhancer increases expression of said THC synthase, compared to a comparable plant lacking said genetic modification in said promoter or enhancer sequence; and a genetic disruption in a gene sequence that encodes a cannabidiolic acid synthase or a cannabichromenic acid synthase, wherein said genetic disruption decreases expression of said cannabidiolic acid synthase or said cannabichromenic acid synthase; compared to a comparable plant lacking said genetic disruption in said gene sequence that encodes a cannabidiolic acid synthase or a cannabichromenic acid synthase.

In some embodiments, said genetic modificationin said promoter or enhancer increases expression of said THC synthase by at least 5%, 10%, 20%, 30%, 40%, 50%, 60%, 70%, 80%, 90%, 100%, compared to a comparable plant lacking said genetic modification in said promoter or enhancer sequence.

In some embodiments, said genetic modification in said promoter or enhancer increases expression of said THC synthase by at least 2 fold, 5 fold, 10 fold, 100 fold, 500 fold, 1000 fold, or 10000 fold compared to a comparable plant lacking said genetic modification in said promoter or enhancer sequence.

In some embodiments, said genetic disruption in said gene sequence that encodes a cannabidiolic acid synthase or a cannabichromenic acid synthase decreases expression of said cannabidiolic acid synthase or said cannabichromenic acid synthase by at least 5%, 10%, 20%, 30%, 40%, 50%, 60%, 70%, 80%, 90%, 100%, compared to a comparable plant lacking said genetic disruption in said gene sequence that encodes a cannabidiolic acid synthase or a cannabichromenic acid synthase.

In some embodiments, said genetic disruption in said gene sequence that encodes a cannabidiolic acid synthase or a cannabichromenic acid synthase decreases expression of said cannabidiolic acid synthase or said cannabichromenic acid synthase by at least 2 fold, 5 fold, 10 fold, 100 fold, 500 fold, 1000 fold, or 10000 fold, compared to a comparable plant lacking said genetic disruption in said gene sequence that encodes a cannabidiolic acid synthase or a cannabichromenic acid synthase.

In some embodiments, said wherein said at least one genetic modification results in an increased level of a compound of Formula V, compared to a level of said compound in a comparable plant lacking said genetic modification.

In some embodiments, said wherein said at least one genetic modification results in an increased level of a cannabinoil (CBN), compared to a level of said compound in a comparable plant lacking said genetic modification.

In some embodiments, said gene encodes THC synthase, olivetolate geranyltransferase (GOT), geranyl pyrophosphate synthase (GPPS), polyketide synthase, or divarinic acid cyclase.

In some embodiments, said gene encodes THC synthase.

In some embodiments, said gene encodes olivetolate geranyltransferase (GOT).

In some embodiments, said gene encodes geranyl pyrophosphate synthase (GPPS).

In some embodiments, said gene encodes polyketide synthase.

In some embodiments, said gene encodes divarinic acid cyclase.

In some embodiments, said at least one genetic modification increases expression of said protein compared to a comparable plant lacking said genetic modification.

In some embodiments, said at least one genetic modification increases activity of said promoter or enhancer.

In some embodiments, said at least one genetic modification results in an increased level of a compound of Formula I, compared to a level of said compound in a comparable plant lacking said genetic modification.

In some embodiments, said at least one genetic modification results in an increased level of a compound of Formula II, compared to a level of said compound in a comparable plant lacking said genetic modification.

In some embodiments, said at least one genetic modification results in an increased level of a compound of Formula III, compared to a level of said compound in a comparable plant lacking said genetic modification.

In some embodiments, said at least one genetic modification results in an increased level of a compound of Formula VI, compared to a level of said compound in a comparable plant lacking said genetic modification.

In some embodiments, said at least one genetic modification results in an increased level of a compound of Formula I, Formula II, Formula III, and Formula VI, compared to a level of said compound in a comparable plant lacking said genetic modification.

In some embodiments, said at least one genetic modification results in an increased level of a compound of Formula III and Formula VI, compared to a level of said compound in a comparable plant lacking said genetic modification.

In some embodiments, said at least one genetic modification results in an increased level of tetrahydrocannabivarinic Acid (THCVA) compared to a comparable plant lacking said genetic modification.

In some embodiments, said at least one genetic modification results in an increased level of cannabigerovarinic acid (CBGVA) compared to a comparable plant lacking said genetic modification.

In some embodiments, said transgenic plant comprises genetic disruption in a promoter or enhancer sequence of at least one, two, three, four, or five different genes, wherein said genes encode for olivetolate geranyltransferase (GOT), geranyl pyrophosphate synthase (GPPS), polyketide synthase, or divarinic acid cyclase.

In some embodiments, said transgenic plant comprises a genetic modification in a promoter or enhancer of a gene that encodes for olivetolate geranyltransferase (GOT), geranyl pyrophosphate synthase (GPPS), polyketide synthase, and divarinic acid cyclase.

In some embodiments, said genetic modifications increase expression of olivetolate geranyltransferase (GOT), geranyl pyrophosphate synthase (GPPS), polyketide synthase, and divarinic acid cyclase.

In some embodiments, said at least one genetic modification is in a gene sequence that encodes a cannabidiolic acid synthase or a cannabichromenic acid synthase.

In some embodiments, said at least one genetic modification disrupts expression of said protein.

In some embodiments, said at least one genetic modification decreases expression of said protein compared to a comparable plant lacking said genetic modification.

In some embodiments, said at least one genetic modification decreases expression of said protein by at least 30%, 40%, 50%, 60%, 70%, 80%, 90%, 100%, compared to a comparable plant lacking said genetic modification.

In some embodiments, said at least one genetic modification decreases expression of said protein by at least 2 fold, 5 fold, 10 fold, 100 fold, 500 fold, 1000 fold, or 10000 fold, compared to a comparable plant lacking said genetic modification.

In some embodiments, said at least one genetic modification results in an increased level of a compound of Formula I, compared to a level of said compound in a comparable plant lacking said genetic modification.

In some embodiments, said at least one genetic modification results in an increased level of a compound of Formula II, compared to a level of said compound in a comparable plant lacking said genetic modification.

In some embodiments, said at least one genetic modification results in an increased level of a compound of Formula III, compared to a level of said compound in a comparable plant lacking said genetic modification.

In some embodiments, said at least one genetic modification results in an increased level of a compound of Formula VI, compared to a level of said compound in a comparable plant lacking said genetic modification.

In some embodiments, said at least one genetic modification results in an increased level of a compound of Formula I, Formula II, Formula III, and Formula VI, compared to a level of said compound in a comparable plant lacking said genetic modification.

In some embodiments, said at least one genetic modification results in an increased level of a compound of Formula III and Formula VI, compared to a level of said compound in a comparable plant lacking said genetic modification.

In some embodiments, said at least one genetic modification results in an increased level of tetrahydrocannabivarinic Acid (THCVA) compared to a comparable plant lacking said genetic modification.

In some embodiments, said at least one genetic modification results in an increased level of cannabigerovarinic acid (CBGVA) compared to a comparable plant lacking said genetic modification.

In some embodiments, said transgenic plant comprises a genetic modification in a promoter or enhancer sequence of a gene encoding THC synthase, olivetolate geranyltransferase (GOT), geranyl pyrophosphate synthase (GPPS), polyketide synthase, or divarinic acid cyclase, wherein said genetic modification in said promoter or enhancer increases expression of said THC synthase, olivetolate geranyltransferase (GOT), geranyl pyrophosphate synthase (GPPS), polyketide synthase, or divarinic acid cyclase, compared to a comparable plant lacking said genetic modification in said promoter or enhancer sequence; and a genetic disruption in a gene sequence that encodes a cannabidiolic acid synthase or a cannabichromenic acid synthase, wherein said genetic disruption decreases expression of said cannabidiolic acid synthase or said cannabichromenic acid synthase; compared to a comparable plant lacking said genetic disruption in said gene sequence that encodes a cannabidiolic acid synthase or a cannabichromenic acid synthase.

In some embodiments, said transgenic plant comprises a genetic modification in a promoter or enhancer sequence of at least one, two, three, four, or five different genes, wherein said genes encode for THC synthase, olivetolate geranyltransferase (GOT), geranyl pyrophosphate synthase (GPPS), polyketide synthase, or divarinic acid cyclase, wherein said genetic modification in said promoter or enhancer increases expression of said THC synthase, olivetolate geranyltransferase (GOT), geranyl pyrophosphate synthase (GPPS), polyketide synthase, or divarinic acid cyclase, compared to a comparable plant lacking said genetic modificationin said promoter or enhancer sequence; and a genetic disruption in at least one or two gene sequences that encode a cannabidiolic acid synthase or a cannabichromenic acid synthase, wherein said genetic disruption decreases expression of said cannabidiolic acid synthase or said cannabichromenic acid synthase; compared to a comparable plant lacking said genetic disruption in said gene sequence that encodes a cannabidiolic acid synthase or a cannabichromenic acid synthase.

In some embodiments, said genetic modification in said promoter or enhancer increases expression of said THC synthase, olivetolate geranyltransferase (GOT), geranyl pyrophosphate synthase (GPPS), polyketide synthase, or divarinic acid cyclase by at least 5%, 10%, 20%, 30%, 40%, 50%, 60%, 70%, 80%, 90%, 100%, compared to a comparable plant lacking said genetic modification in said promoter or enhancer sequence.

In some embodiments, said genetic modification in said promoter or enhancer increases expression of said THC synthase, olivetolate geranyltransferase (GOT), geranyl pyrophosphate synthase (GPPS), polyketide synthase, or divarinic acid cyclase by at least 2 fold, 5 fold, 10 fold, 100 fold, 500 fold, 1000 fold, or 10000 fold compared to a comparable plant lacking said genetic modification in said promoter or enhancer sequence.

In some embodiments, said genetic disruption in said gene sequence that encodes a cannabidiolic acid synthase or a cannabichromenic acid synthase decreases expression of said cannabidiolic acid synthase or said cannabichromenic acid synthase by at least 5%, 10%, 20%, 30%, 40%, 50%, 60%, 70%, 80%, 90%, 100%, compared to a comparable plant lacking said genetic disruption in said gene sequence that encodes a cannabidiolic acid synthase or a cannabichromenic acid synthase.

In some embodiments, said genetic disruption in said gene sequence that encodes a cannabidiolic acid synthase or a cannabichromenic acid synthase decreases expression of said cannabidiolic acid synthase or said cannabichromenic acid synthase by at least 2 fold, 5 fold, 10 fold, 100 fold, 500 fold, 1000 fold, or 10000 fold, compared to a comparable plant lacking said genetic disruption in said gene sequence that encodes a cannabidiolic acid synthase or a cannabichromenic acid synthase.

In some embodiments, said wherein said at least one genetic modification results in an increased level of a compound of Formula II, compared to a level of said compound in a comparable plant lacking said genetic modification.

In some embodiments, said wherein said at least one genetic modification results in an increased level of a compound of Formula IV, compared to a level of said compound in a comparable plant lacking said genetic modification.

In some embodiments, said at least one genetic modification results in an increased level of a compound of Formula I, compared to a level of said compound in a comparable plant lacking said genetic modification.

In some embodiments, said at least one genetic modification results in an increased level of a compound of Formula II, compared to a level of said compound in a comparable plant lacking said genetic modification.

In some embodiments, said at least one genetic modification results in an increased level of a compound of Formula III, compared to a level of said compound in a comparable plant lacking said genetic modification.

In some embodiments, said at least one genetic modification results in an increased level of a compound of Formula VI, compared to a level of said compound in a comparable plant lacking said genetic modification.

In some embodiments, said at least one genetic modification results in an increased level of a compound of Formula I, Formula II, Formula III, and Formula VI, compared to a level of said compound in a comparable plant lacking said genetic modification.

In some embodiments, said at least one genetic modification results in an increased level of a compound of Formula III and Formula VI, compared to a level of said compound in a comparable plant lacking said genetic modification.

In some embodiments, said at least one genetic modification results in an increased level of tetrahydrocannabivarinic Acid (THCVA) compared to a comparable plant lacking said genetic modification.

In some embodiments, said at least one genetic modification results in an increased level of cannabigerovarinic acid (CBGVA) compared to a comparable plant lacking said genetic modification.

In some embodiments, said transgenic plant further comprises an increased amount of cannabigerol (CBG), derivative or analog thereof, compared to an amount of the same compound in a comparable control plant without said genetic modification.

The transgenic plant of claim 1 or 108, wherein said genetic modification comprises a genetic disruption that results in an increased expression of Formula II, or a derivative or analog thereof

In some embodiments, said first group of genes comprises olivetolic acid cyclase (OAC) and olivetolic acid synthase (OLS).

In some embodiments, said genetic modification comprises a disruption of gene encoding prenyl-transferase, wherein said disruption results in an increased amount of prenyl-transferase compared to an amount of the same compound comparable control plant without said disruption.

In some embodiments, said prenyl-transferase is olivetolic acid geranyltransferase (GOT).

In some embodiments, said disruption is in a promoter region of said genes.

In some embodiments, said genetic modification comprises a disruption of a second of group of genes encoding CBCA synthase, CBDA synthase, and THCA synthase.

In some embodiments, said disruption results in a decreased amount of CBCA synthase, CBDA synthase, THCA synthase, derivatives or analogs thereof compared to an amount of the same compound of a comparable control plant without said disruption.

In some embodiments, said disruption is in a coding region of said genes.

In some embodiments, said transgenic plant comprises 10% more Formula IV measured by dry weight as compared to a comparable control plant without said modification.

In some embodiments, said transgenic plant comprises 25% more Formula IV measured by dry weight as compared to a comparable control plant without said modification.

In some embodiments, said transgenic plant comprises 35% more Formula IV measured by dry weight as compared to a comparable control plant without said modification.

In some embodiments, said transgenic plant comprises 50% more Formula IV measured by dry weight as compared to a comparable control plant without said modification.

In some embodiments, said transgenic plant comprises 10% less cannabichromenic acid (CBCA) measured by dry weight as compared to a comparable control plant without said modification.

In some embodiments, said transgenic plant comprises 10% less cannabidiolic acid (CBDA) measured by dry weight as compared to a comparable control plant without said modification.

In some embodiments, said transgenic plant comprises 10% less tetrahydrocannabinolic acid (THCA) measured by dry weight as compared to a comparable control plant without said modification.

In some embodiments, said transgenic plant comprises an increased amount of cannabinol (CBN), derivative or analog thereof compared to an amount of the same compound in a comparable control plant without said genetic modification.

In some embodiments, said genetic modification comprises a disruption of gene encoding THCA synthase.

In some embodiments, said disruption results in an increased amount of THCA synthase compared to an amount of the same compound in a comparable control plant without said disruption.

In some embodiments, said disruption is in a promoter region of said gene.

In some embodiments, said genetic modification comprises a disruption of genes encoding CBDA synthase and CBCA synthase respectively.

In some embodiments, said disruption results in decreased amount of CBDA synthase and CBCA synthase compared to a comparable control plant without said disruption.

In some embodiments, said disruption is in a coding region of said genes.

In some embodiments, said genetic modification comprises a disruption of a third group of genes, wherein said disruption results in increased UV absorption of said transgenic plant compared to a comparable control without said disruption.

In some embodiments, said transgenic plant comprises 10% more THC measured by dry weight as compared to a comparable control plant without said genetic modification.

In some embodiments, said transgenic plant comprises 25% more THC measured by dry weight as compared to a comparable control plant without said genetic modification.

In some embodiments, said transgenic plant comprises 35% more THC measured by dry weight as compared to a comparable control plant without said genetic modification.

In some embodiments, said transgenic plant comprises 50% more THC measured by dry weight as compared to a comparable control plant without said genetic modification.

In some embodiments, said transgenic plant comprises 10% less CBCA measured by dry weight as compared to a comparable control plant without said genetic modification.

In some embodiments, said transgenic plant comprises 10% less CBDA measured by dry weight as compared to a comparable control plant without said genetic modification.

In some embodiments, said transgenic plant comprises an increased amount of tetrahydrocannabivarin (THCV), derivative or analog thereof compared to an amount of the same compound in a comparable control plant without said genetic modification.

In some embodiments, said genetic modification comprises a disruption of a first of group of genes, wherein said disruption results in an increased amount of Formula I, derivative or analog thereof

In some embodiments, said genetic modification comprises a disruption of a second group of genes, wherein said disruption results in a decreased amount of

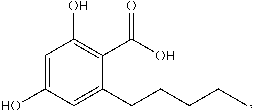 [SMILES: CCCCCc1cc(O)cc(O)c1C(=O)O]

derivative or analog thereof.

In some embodiments, said second group of genes comprises OAC and OLS.

In some embodiments, said disruption is in a coding region of said genes.

In some embodiments, said genetic modification comprises a disruption of a THCA synthase.

In some embodiments, said disruption results in an increased amount of THCA synthase, derivative or analog thereof, compared to an amount of the same compound in a comparable control plant without said disruption.

In some embodiments, said disruption is in a promoter region of said genes.

In some embodiments, said genetic modification comprises a disruption of a third group of genes encoding CBCA synthase and CBDA synthase respectively.

In some embodiments, said disruption results in a decreased amount of CBCA synthase and CBDA synthase, derivatives or analogs thereof

In some embodiments, said disruption is in a coding region of said genes.

In some embodiments, said transgenic plant comprises 10% more tetrahydrocannabivarin (THCV) measure by dry weight as compared to a comparable control plant without said modification.

In some embodiments, said transgenic plant comprises 25% more tetrahydrocannabivarin (THCV) measure by dry weight as compared to a comparable control plant without said modification.

In some embodiments, said transgenic plant comprises 35% more tetrahydrocannabivarin (THCV) measure by dry weight as compared to a comparable control plant without said modification.

In some embodiments, said transgenic plant comprises 50% more tetrahydrocannabivarin (THCV) measure by dry weight as compared to a comparable control plant without said modification.

In some embodiments, said transgenic plant comprises 10% less cannabichromevarin (CBCV) measure by dry weight as compared to a comparable control plant without said modification.

In some embodiments, said transgenic plant comprises 10% less cannabidivarin (CBDV) measure by dry weight as compared to a comparable control plant without said modification.

In some embodiments, said transgenic plant comprises a genetic modification, wherein said genetic modification results in an increased amount of cannabigerol (CBG), derivative or analog thereof, compared to an amount of the same compound in a comparable control plant without said genetic modification.

In some embodiments, said genetic modification comprises a disruption of a first group of genes, wherein said disruption results in an increased amount of

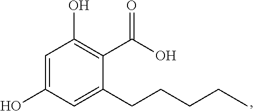 [SMILES: CCCCCc1cc(O)cc(O)c1C(=O)O]

derivative or analog thereof.

In some embodiments, said first group of genes comprises olivetolic acid cyclase (OAC) and olivetolic acid synthase (OLS).

In some embodiments, said genetic modification comprises a disruption of gene encoding prenyl-transferase, wherein said disruption results in an increased amount of prenyl-transferase compared to a comparable control plant without said disruption.

In some embodiments, said prenyl-transferase is olivetolic acid geranyltransferase (GOT).

In some embodiments, said disruption is in a promoter region of said genes.

In some embodiments, said genetic modification comprises a disruption of a second of group of genes encoding CBCA synthase, CBDA synthase, and THCA synthase.

In some embodiments, said disruption results in a decreased amount of CBCA synthase, CBDA synthase, THCA synthase, derivatives or analogs thereof, compared to an amount of the same compound in a comparable control plant without said disruption.

In some embodiments, said disruption is in a coding region of said genes.

In some embodiments, said transgenic plant comprises 10% more Formula IV measured by dry weight as compared to a comparable control plant without said modification.

In some embodiments, said transgenic plant comprises 25% more Formula IV measured by dry weight as compared to a comparable control plant without said modification.

In some embodiments, said transgenic plant comprises 35% more Formula IV measured by dry weight as compared to a comparable control plant without said modification.

In some embodiments, said transgenic plant comprises 50% more Formula IV measured by dry weight as compared to a comparable control plant without said modification.

In some embodiments, said transgenic plant comprises 10% less cannabichromenic acid (CBCA) measured by dry weight as compared to a comparable control plant without said modification.

In some embodiments, said transgenic plant comprises 10% less cannabidiolic acid (CBDA) measured by dry weight as compared to a comparable control plant without said modification.

In some embodiments, said transgenic plant comprises 10% less tetrahydrocannabinolic acid (THCA) measured by dry weight as compared to a comparable control plant without said modification.

In one aspect, provided herein are genetically modified cells comprising a genetic modification, wherein said genetic modification results in an increased amount of

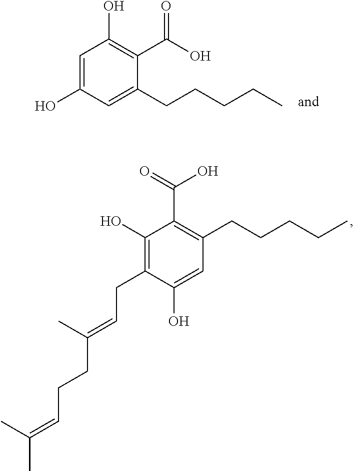

derivatives or analogs thereof, wherein said genetic modification does not result in a change of amount of

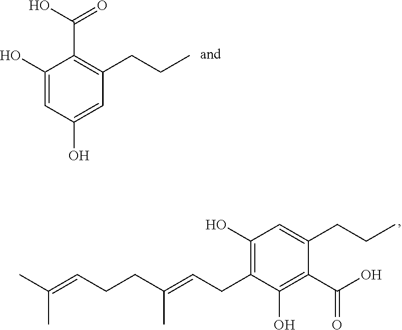 [SMILES: CCCc1cc(O)cc(O)c1C(=O)O ; CCCc1cc(O)c(C/C=C(\C)CCC=C(C)C)c(O)c1C(=O)O]

derivatives or analogs thereof, compared to an amount of the same compound in a comparable control cell without said genetic modification.

In some embodiments, said genetic modification further results in an increased amount of cannabigerol (CBG), derivative or analog thereof, compared to an amount of the same compound in a comparable control cell without said genetic modification.

In some embodiments, said genetic modification results in an increased amount of cannabinol (CBN), derivative or analog thereof, compared to an amount of the same compound in a comparable control cell without said genetic modification.

In some embodiments, said genetic modification results in an increased amount of tetrahydrocannabivarin (THCV), derivative or analog thereof, compared to an amount of the same compound in a comparable control cell without said genetic modification.

In some embodiments, said genetic modification comprises a disruption of gene encoding geranyl pyrophosphate synthase (GPPS), resulting in increased amount of geranyl pyrophosphate (GPP).

In some embodiments, genetic modification comprises a disruption of gene encoding polyketide synthase (PKS), resulting in increased amount of either Formula I or Formula II or both.

In some embodiments, the genetically modified cell is a plant cell, an algae cell, a agrobacterium cell, a E.coli cell, a yeast cell, an animal cell, or an insect cell.

In some embodiments, said genetically modified cell is a plant cell.

In some embodiments, said genetically modified cell is a cannabis plant cell.

In some embodiments, said genetically modified cell is a callus cell, a protoplast, an embryonic cell, a leaf cell, a seed cell, a stem cell, or a root cell.

In one aspect, provided herein are genetically modified cells comprising a genetic modification, wherein said genetic modification results in an increased amount of

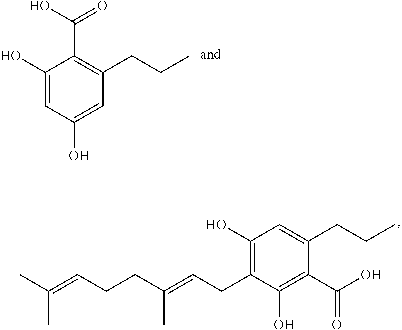 [SMILES: CCCc1cc(O)cc(O)c1C(=O)O ; CCCc1cc(O)c(C/C=C(\C)CCC=C(C)C)c(O)c1C(=O)O]

derivatives or analogs thereof, wherein said genetic modification does not result in a change of amount of

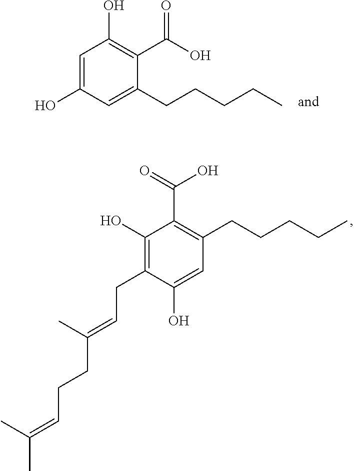

derivatives or analogs thereof, compared to an amount of the same compound in a comparable control cell without said genetic modification.

In some embodiments, said genetic modification further results in an increased amount of cannabigerol (CBG), derivative or analog thereof, compared to an amount of the same compound in a comparable control cell without said genetic modification.

In some embodiments, said genetic modification results in an increased amount of cannabinol (CBN), derivative or analog thereof, compared to an amount of the same compound in a comparable control cell without said genetic modification.

In some embodiments, said genetic modification results in an increased amount of tetrahydrocannabivarin (THCV), derivative or analog thereof, compared to an amount of the same compound in a comparable control cell without said genetic modification.

In some embodiments, said genetic modification comprises a disruption of gene encoding geranyl pyrophosphate synthase (GPPS), resulting in increased amount of geranyl pyrophosphate (GPP).

In some embodiments, genetic modification comprises a disruption of gene encoding polyketide synthase (PKS), resulting in increased amount of either Formula I or Formula II or both.

In some embodiments, the genetically modified cell is a plant cell, an algae cell, a agrobacterium cell, a E.coli cell, a yeast cell, an animal cell, or an insect cell.

In some embodiments, said genetically modified cell is a plant cell.

In some embodiments, said genetically modified cell is a cannabis plant cell.

In some embodiments, said genetically modified cell is a callus cell, a protoplast, an embryonic cell, a leaf cell, a seed cell, a stem cell, or a root cell.

In some embodiments, said modification is integrated in the genome of said cell.

In one aspect, provided herein are compositions comprising an endonuclease or polynucleotide encoding said endonuclease capable of introducing a genetic modification, wherein said genetic modification results in an increased amount of a compound of:

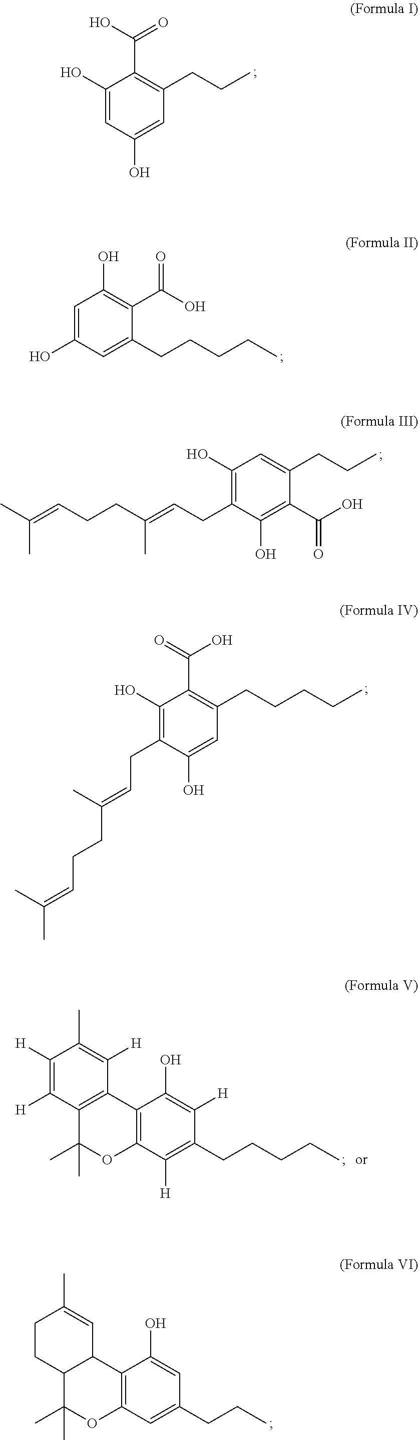

or derivatives or analogs thereof.

In one aspect, provided herein are compositions comprising an endonuclease or polynucleotide encoding said endonuclease capable of introducing a genetic modification, wherein said genetic modification results in an increased amount of

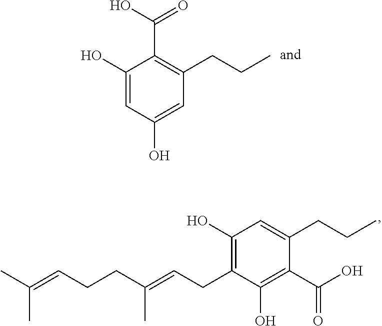 [SMILES: CCCc1cc(O)cc(O)c1C(=O)O ; CCCc1cc(O)c(C/C=C(\C)CCC=C(C)C)c(O)c1C(=O)O]

derivatives or analogs thereof, wherein said genetic modification does not result in a change of amount of

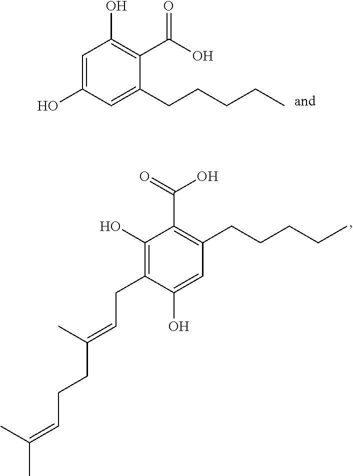

derivatives or analogs thereof, compared to a comparable control cell without said genetic modification.

In some embodiments, said genetic modification further results in an increased amount of cannabigerol (CBG), derivative or analog thereof, compared to an amount of the same compound in a comparable control cell without said genetic modification.

In some embodiments, said genetic modification results in an increased amount of cannabinol (CBN), derivative or analog thereof, compared to an amount of the same compound in a comparable control cell without said genetic modification.

In some embodiments, said genetic modification results in an increased amount of tetrahydrocannabivarin (THCV), derivative or analog thereof, compared to an amount of the same compound in a comparable control cell without said genetic modification.

In some embodiments, said modification is in a coding region of the THCAS gene.

In one aspect, provided herein are compositions comprising an endonuclease or polynucleotide encoding said endonuclease capable of introducing a genetic modification, wherein said genetic modification results in an increased amount of

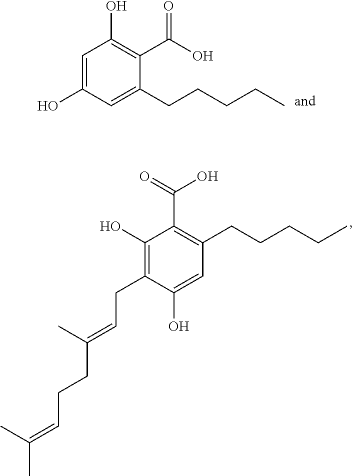

derivatives or analogs thereof, wherein said genetic modification does not result in a change of amount of

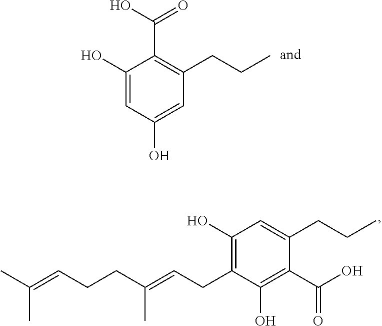 [SMILES: CCCc1cc(O)cc(O)c1C(=O)O ; CCCc1cc(O)c(C/C=C(\C)CCC=C(C)C)c(O)c1C(=O)O]

derivatives or analogs thereof, compared to a comparable control cell without said genetic modification.

In some embodiments, said genetic modification further results in an increased amount of cannabigerol (CBG), derivative or analog thereof, compared to an amount of the same compound in a comparable control cell without said genetic modification.

In some embodiments, said genetic modification results in an increased amount of cannabinol (CBN), derivative or analog thereof, compared to an amount of the same compound in a comparable control cell without said genetic modification.

In some embodiments, said genetic modification results in an increased amount of tetrahydrocannabivarin (THCV), derivative or analog thereof, compared to an amount of the same compound in a comparable control cell without said genetic modification.

In some embodiments, said modification is in a coding region of the THCAS gene.

In one aspect, provided herein are compositions comprising an endonuclease or polynucleotide encoding said endonuclease capable of introducing a genetic modification, wherein said genetic modification results in an increased amount of

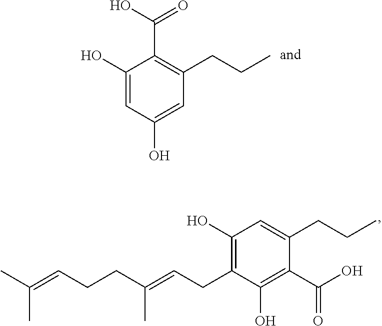 [SMILES: CCCc1cc(O)cc(O)c1C(=O)O ; CCCc1cc(O)c(C/C=C(\C)CCC=C(C)C)c(O)c1C(=O)O]

derivatives or analogs thereof, wherein said genetic modification does not result in a change of amount of

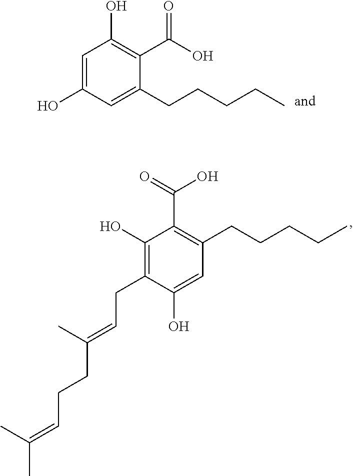

derivatives or analogs thereof, compared to a comparable control cell without said genetic modification.

In some embodiments, said genetic modification further results in an increased amount of cannabigerol (CBG), derivative or analog thereof, compared to an amount of the same compound in a comparable control cell without said genetic modification.

In some embodiments, said genetic modification results in an increased amount of cannabinol (CBN), derivative or analog thereof, compared to an amount of the same compound in a comparable control cell without said genetic modification.

In some embodiments, said genetic modification results in an increased amount of tetrahydrocannabivarin (THCV), derivative or analog thereof, compared to an amount of the same compound in a comparable control cell without said genetic modification.

In some embodiments, said modification is in a coding region of the THCAS gene.

In one aspect, provided herein are methods of making transgenic plants described herein.

In one aspect, provided herein are kits for genome editing comprising the composition described herein

In one aspect, provided herein are cells comprising the composition described herein.

In some embodiments, the genetically modified cell is a plant cell, an algae cell, a agrobacterium cell, a E. coli cell, a yeast cell, an insect cell, or an animal cell.

In some embodiments, said genetically modified cell is a plant cell.

In some embodiments, said genetically modified cell is a cannabis plant cell.

In some embodiments, said genetically modified cell is a callus cell, a protoplast, an embryonic cell, a leaf cell, a seed cell, a stem cell, or a root cell.

In one aspect, provided herein are plants comprising a cell described herein.

In one aspect, provided herein are pharmaceutical compositions comprising an extract of a transgenic plant described herein, a genetically modified cell described herein, a composition described herein, or a cell described herein.

In some embodiments, said method further comprises a pharmaceutically acceptable excipient, diluent, or carrier.

In some embodiments, said pharmaceutically acceptable excipient is a lipid.

In one aspect, provided herein are nutraceutical compositions comprising an extract of a transgenic plant described herein, a genetically modified described herein, a composition described herein, or a cell described herein.

In one aspect, provided herein are food supplement compositions comprising an extract of a transgenic plant described herein, a genetically modified described herein, a composition described herein, or a cell described herein.

In one aspect, provided herein are pharmaceutical compositions described herein, the nutraceutical compositions described herein, or the food supplements described herein in an oral form, a transdermal form, an oil formulation, an edible food, a food substrate, an aqueous dispersion, an emulsion, a solution, a suspension, an elixir, a gel, a syrup, an aerosol, a mist, a powder, a tablet, a lozenge, a gel, a lotion, a paste, a formulated stick, a balm, a cream, or an ointment.

In one aspect, provided herein are methods of treating a disease or condition comprising administering pharmaceutical composition, a nutraceutical composition, or a food supplement described herein.

In some embodiments, said disease or condition is selected from the group consisting of anorexia, emesis, pain, inflammation, multiple sclerosis, Parkinson's disease, Huntington's disease, Tourette's syndrome, Alzheimer's disease, epilepsy, glaucoma, osteoporosis, schizophrenia, cardiovascular disorders, cancer, and obesity.

In one aspect, provided herein are transgenic plants comprising a genetic modification, wherein said genetic modification results in an increased amount of cannabinol (CBN), derivative or analog thereof, compared to an amount of the same compound in a comparable control plant without said genetic modification.

In some embodiments, said genetic modification comprises a disruption of gene encoding THCA synthase.

In some embodiments, said disruption results in an increased amount of THCA synthase compared to a comparable control plant without said disruption.

In some embodiments, said disruption is in a promoter region of said gene.

In some embodiments, said genetic modification comprises a disruption of genes encoding CBDA synthase and CBCA synthase respectively.

In some embodiments, said disruption results in decreased amount of CBDA synthase and CBCA synthase compared to a comparable control plant without said disruption.

In some embodiments, said disruption is in a coding region of said genes.

In some embodiments, said genetic modification comprises a disruption of a third group of genes, wherein said disruption results in increased UV absorption of said transgenic plant compared to a comparable control without said disruption.

In some embodiments, said transgenic plant comprises 10% more THC measured by dry weight as compared to a comparable control plant without said genetic modification.

In some embodiments, said transgenic plant comprises 25% more THC measured by dry weight as compared to a comparable control plant without said genetic modification.

In some embodiments, said transgenic plant comprises 35% more THC measured by dry weight as compared to a comparable control plant without said genetic modification.

In some embodiments, said transgenic plant comprises 50% more THC measured by dry weight as compared to a comparable control plant without said genetic modification.

In some embodiments, said transgenic plant comprises 10% less CBCA measured by dry weight as compared to a comparable control plant without said genetic modification.

In some embodiments, said transgenic plant comprises 10% less CBDA measured by dry weight as compared to a comparable control plant without said genetic modification.

In one aspect, provided herein are transgenic plants comprising a genetic modification, wherein said genetic modification results in an increased amount of tetrahydrocannabivarin (THCV), derivative or analog thereof, compared to an amount of the same compound in a comparable control plant without said genetic modification.

In some embodiments, said genetic modification comprises a disruption of a first of group of genes, wherein said disruption results in an increased amount of Formula I, derivative or analog thereof.

In some embodiments, said genetic modification comprises a disruption of a second group of genes, wherein said disruption results in a decreased amount of

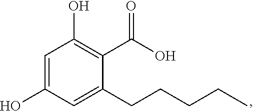 [SMILES: CCCCCc1cc(O)cc(O)c1C(=O)O]

derivative or analog thereof.

In some embodiments, said second group of genes comprises OAC and OLS.

In some embodiments, said disruption is in a coding region of said genes.

In some embodiments, said genetic modification comprises a disruption of a THCA synthase.

In some embodiments, said disruption results in an increased level of THCA synthase, derivative or analog thereof, compared to an amount of the same compound in a comparable control plant without said disruption.

In some embodiments, said disruption is in a promoter region of said genes.

In some embodiments, said genetic modification comprises a disruption of a third group of genes encoding CBCA synthase and CBDA synthase respectively.

In some embodiments, said disruption results in a decreased amount of CBCA synthase and CBDA synthase, derivatives or analogs thereof

In some embodiments, said disruption is in a coding region of said genes.

In some embodiments, said transgenic plant comprises 10% more tetrahydrocannabivarin (THCV) measure by dry weight as compared to a comparable control plant without said modification.

In some embodiments, said transgenic plant comprises 25% more tetrahydrocannabivarin (THCV) measure by dry weight as compared to a comparable control plant without said modification.

In some embodiments, said transgenic plant comprises 35% more tetrahydrocannabivarin (THCV) measure by dry weight as compared to a comparable control plant without said modification.

In some embodiments, said transgenic plant comprises 50% more tetrahydrocannabivarin (THCV) measure by dry weight as compared to a comparable control plant without said modification.

In some embodiments, said transgenic plant comprises 10% less cannabichromevarin (CBCV) measure by dry weight as compared to a comparable control plant without said modification.

In some embodiments, said transgenic plant comprises 10% less cannabidivarin (CBDV) measure by dry weight as compared to a comparable control plant without said modification.

In some embodiments, said genetic modification is conducted by an endonuclease.

In some embodiments, said genetic modification comprises an insertion, a deletion, a substitution, or a frameshift.

In some embodiments, said endonuclease comprises a CRISPR enzyme, TALE-Nuclease, transposon-based nuclease, Zinc finger nuclease, meganuclease, Mega-TAL or DNA guided nuclease.

In some embodiments, said DNA-guided nuclease comprises argonaute.

In some embodiments, said endonuclease is a CRISPR enzyme complexed with a guide polynucleotide that is complementary to a target sequence of at least one of genes encoding OAC, OLS, GOT, CBCA synthase, CBDA synthase, and THCA synthase.

In some embodiments, said target sequence is at least 18 nucleotides, at least 19 nucleotides, at least 20 nucleotides, at least 21 nucleotides, or at least 22 nucleotides in length.

In some embodiments, said target sequence is at most 17 nucleotides in length.

In some embodiments, said target sequence comprises a sequence selected from Table 2 or Table 3 or complementary thereof.

In some embodiments, said guide polynucleotide is a chemically modified.

In some embodiments, said guide polynucleotide is a single guide RNA (sgRNA).

In some embodiments, said guide polynucleotide is a chimeric single guide comprising RNA and DNA.

In some embodiments, said guide polynucleotide comprises a sequence selected from Table 2 or Table 3 or complementary thereof

In some embodiments, said CRISPR enzyme is a Cas protein.

In some embodiments, the Cas protein comprises Cas1, Cas1B, Cas2, Cas3, Cas4, Cas5, Cas5d, Cas5t, Cas5h, Cas5a, Cas6, Cas7, Cas8, Cas9, Cas10, Csy1 , Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, C2c1, C2c2, C2c3, Cpf1, CARF, DinG, homologues thereof, or modified versions thereof.

In some embodiments, said Cas protein is Cas9.

In some embodiments, said Cas9 recognizes a canonical PAM.

In some embodiments, said Cas9 recognizes a non-canonical PAM.

In some embodiments, said guide polynucleotide binds said target sequence 3-10 nucleotides from of PAM.

In some embodiments, said CRISPR enzyme complexed with said guide polynucleotide is introduced into said transgenic plant by an RNP.

In some embodiments, said CRISPR enzyme complexed with said guide polynucleotide is introduced into said transgenic plant by a vector comprising a nucleic acid encoding said CRISPR enzyme and said guide polynucleotide.

In some embodiments, said vector is a binary vector or a Ti plasmid.

In some embodiments, said vector further comprises a selection marker or a reporter gene.

In some embodiments, said RNP or vector is introduced into said transgenic plant via electroporation, agrobacterium mediated transformation, biolistic particle bombardment, or protoplast transformation.

In some embodiments, said RNP or vector further comprising a donor polynucleotide.

In some embodiments, said donor polynucleotide comprises homology to sequences flanking said target sequence.

In some embodiments, said donor polynucleotide introduces a stop codon into at least one of genes encoding OAC, OLS, GOT, CBCA synthase, CBDA synthase, and THCA synthase.

In some embodiments, said donor polynucleotide further comprises a barcode, a reporter gene, or a selection marker.

In one aspect, provided herein are methods for generating a transgenic plant, said method comprising: (a) contacting a plant cell with an endonuclease or a polypeptide encoding said endonuclease, wherein said endonuclease introduces a genetic modification resulting in an increased amount of a compound selected from:

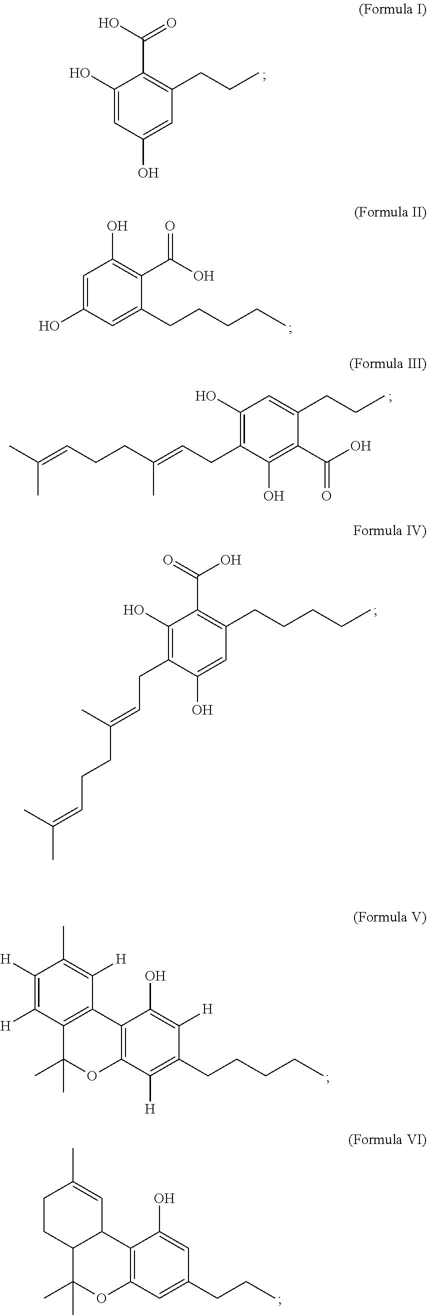

derivatives or analogs thereof, compared to an amount of the same compound in a comparable control plant without said genetic modification;

(b) culturing said plant cell in (a) to generate a transgenic plant.

In some embodiments, said genetic modification comprises a disruption of gene encoding geranyl pyrophosphate synthase (GPPS), resulting in increased amount of geranyl pyrophosphate (GPP).

In some embodiments, said genetic modification comprises a disruption of gene encoding polyketide synthase (PKS), resulting in increased amount of either Formula I or Formula II or both.

In some embodiments, said contacting is via electroporation, agrobacterium mediated transformation, biolistic particle bombardment, or protoplast transformation.

In some embodiments, the method further comprises further comprising culturing said plant cell in (a) to generate a callus, a cotyledon, a root, a leaf, or a fraction thereof

In some embodiments, said genetic modification results in an increased amount of cannabigerol (CBG), derivative or analog thereof, compared to an amount of the same compound in a comparable control plant without said genetic modification.

In some embodiments, said genetic modification comprises a disruption of a first group of genes, wherein said disruption results in an increased amount of

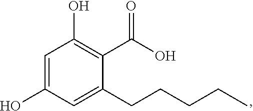 [SMILES: CCCCCc1cc(O)cc(O)c1C(=O)O]

derivative or analog thereof.

In some embodiments, said first group of genes comprises olivetolic acid cyclase (OAC) and olivetolic acid synthase (OLS).

In some embodiments, said genetic modification comprises a disruption of gene encoding prenyl-transferase, wherein said disruption results in an increased amount of prenyl-transferase compared to an amount of the same compound in a comparable control plant without said disruption.

In some embodiments, said prenyl-transferase is olivetolic acid geranyltransferase (GOT).

In some embodiments, said disruption is in a promoter region of said genes.

In some embodiments, wherein said genetic modification comprises a disruption of a second of group of genes encoding CBCA synthase, CBDA synthase, and THCA synthase.

In some embodiments, said disruption results in a decreased amount of CBCA synthase, CBDA synthase, THCA synthase, derivatives or analogs thereof, compared to an amount of the same amount in a comparable control plant without said disruption.

In some embodiments, said disruption is in a coding region of said genes.

In some embodiments, said modification results in 10% more Formula IV measured by dry weight in said transgenic plant as compared to a comparable control plant without said modification.

In some embodiments, said modification results in 25% more Formula IV measured by dry weight in said transgenic plant as compared to a comparable control plant without said modification.

In some embodiments, said modification results in 35% more Formula IV measured by dry weight in said transgenic plant as compared to a comparable control plant without said modification.

In some embodiments, said modification results in 50% more Formula IV measured by dry weight in said transgenic plant as compared to a comparable control plant without said modification.

In some embodiments, said modification results in 10% less cannabichromenic acid (CBCA) measured by dry weight in said transgenic plant as compared to a comparable control plant without said modification.

In some embodiments, said modification results in 10% less cannabidiolic acid (CBDA) measured by dry weight in said transgenic plant as compared to a comparable control plant without said modification.

In some embodiments, said modification results in 10% less tetrahydrocannabinolic acid (THCA) measured by dry weight in said transgenic plant as compared to a comparable control plant without said modification.

In some embodiments, said genetic modification results in an increased amount of cannabinol (CBN), derivative or analog thereof, compared to an amount of the same compound in a comparable control plant without said genomic modification.

In some embodiments, said genetic modification comprises a disruption of gene encoding THCA synthase.

In some embodiments, said disruption results in an increased amount of THCA synthase compared to an amount of the same amount in a comparable control plant without said disruption.

In some embodiments, said disruption is in a promoter region of said gene.

In some embodiments, said genetic modification comprises a disruption of genes encoding CBDA synthase and CBCA synthase respectively.

In some embodiments, said disruption results in decreased amount of CBDA synthase and CBCA synthase compared to an amount of the same compound in a comparable control plant without said disruption.

In some embodiments, said disruption is in a coding region of said genes.

In some embodiments, said genetic modification comprises a disruption of a third group of genes, wherein said disruption results in increased UV absorption of said transgenic plant compared to a comparable control without said disruption.

In some embodiments, said modification results in 10% more THC measured by dry weight in said transgenic plant as compared to a comparable control plant without said genetic modification.

In some embodiments, said modification results in 25% more THC measured by dry weight in said transgenic plant as compared to a comparable control plant without said genetic modification.

In some embodiments, said modification results in 35% more THC measured by dry weight in said transgenic plant as compared to a comparable control plant without said genetic modification.

In some embodiments, said modification results in 50% more THC measured by dry weight in said transgenic plant as compared to a comparable control plant without said genetic modification.

In some embodiments, said modification results in 10% less CBCA measured by dry weight in said transgenic plant as compared to a comparable control plant without said genetic modification.

In some embodiments, said modification results in 10% less CBDA measured by dry weight in said transgenic plant as compared to a comparable control plant without said genetic modification.

In some embodiments, said genetic modification results in an increased amount of tetrahydrocannabivarin (THCV), derivative or analog thereof, compared to an amount of the same compound in a comparable control plant without said genomic modification.

In some embodiments, said genetic modification comprises a disruption of a first of group of genes, wherein said disruption results in an increased amount of Formula I, derivative or analog thereof.

In some embodiments, said genetic modification comprises a disruption of a second group of genes, wherein said disruption results in a decreased amount of

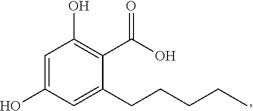 [SMILES: CCCCCc1cc(O)cc(O)c1C(=O)O]

derivative or analog thereof.

In some embodiments, said second group of genes comprises OAC and OLS.

In some embodiments, said disruption is in a coding region of said genes.

In some embodiments, said genetic modification comprises a disruption of a THCA synthase.

In some embodiments, said disruption results in an increased amount of THCA synthase, derivative or analog thereof, compared to an amount of the same compound in a comparable control plant without said disruption.

In some embodiments, said disruption is in a promoter region of said genes.

In some embodiments, said genetic modification comprises a disruption of a third group of genes encoding CBCA synthase and CBDA synthase respectively.

In some embodiments, said disruption results in a decreased amount of CBCA synthase and CBDA synthase, derivatives or analogs thereof

In some embodiments, said disruption is in a coding region of said genes.

In some embodiments, said modification results in 10% more tetrahydrocannabivarin (THCV) measure by dry weight in said transgenic plant as compared to a comparable control plant without said modification.

In some embodiments, said modification results in 25% more tetrahydrocannabivarin (THCV) measure by dry weight in said transgenic plant as compared to a comparable control plant without said modification.

In some embodiments, said modification results in 35% more tetrahydrocannabivarin (THCV) measure by dry weight in said transgenic plant as compared to a comparable control plant without said modification.

In some embodiments, said modification results in 50% more tetrahydrocannabivarin (THCV) measure by dry weight in said transgenic plant as compared to a comparable control plant without said modification.

In some embodiments, said modification results in 10% less cannabichromevarin (CBCV) measure by dry weight in said transgenic plant as compared to a comparable control plant without said modification.

In some embodiments, said modification results in 10% less cannabidivarin (CBDV) measure by dry weight in said transgenic plant as compared to a comparable control plant without said modification.

In one aspect, provided herein are methods for generating a transgenic plant, said method comprising: (a) contacting a plant cell with an endonuclease or a polypeptide encoding said endonuclease, wherein said endonuclease introduces a genetic modification resulting in an increased amount of cannabigerol (CBG), derivative or analog thereof, compared to an amount of the same compound in a comparable control plant without said genetic modification; (b) culturing said plant cell in (a) to generate a transgenic plant.

In some embodiments, said contacting is via electroporation, agrobacterium mediated transformation, biolistic particle bombardment, or protoplast transformation.

In some embodiments, the method further comprises culturing said plant cell in (a) to generate a callus, a cotyledon, a root, a leaf, or a fraction thereof

In some embodiments, said genetic modification results in an increased amount of cannabigerol (CBG), derivative or analog thereof, compared to an amount of the same compound in a comparable control plant without said genetic modification.

In some embodiments, said genetic modification comprises a disruption of a first group of genes, wherein said disruption results in an increased amount of

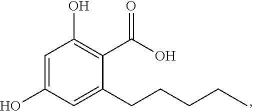 [SMILES: CCCCCc1cc(O)cc(O)c1C(=O)O]

derivative or analog thereof.

In some embodiments, said first group of genes comprises olivetolic acid cyclase (OAC) and olivetolic acid synthase (OLS).

In some embodiments, said genetic modification comprises a disruption of gene encoding prenyl-transferase, wherein said disruption results in an increased amount of prenyl-transferase compared to an amount of the same compound in a comparable control plant without said disruption.

In some embodiments, said prenyl-transferase is olivetolic acid geranyltransferase (GOT).

In some embodiments, said disruption is in a promoter region of said genes.

In some embodiments, said genetic modification comprises a disruption of a second of group of genes encoding CBCA synthase, CBDA synthase, and THCA synthase.

In some embodiments, said disruption results in a decreased amount of CBCA synthase, CBDA synthase, THCA synthase, derivatives or analogs thereof, compared to an amount of the same compound in a comparable control plant without said disruption.

In some embodiments, said disruption is in a coding region of said genes.

In some embodiments, said modification results in 10% more

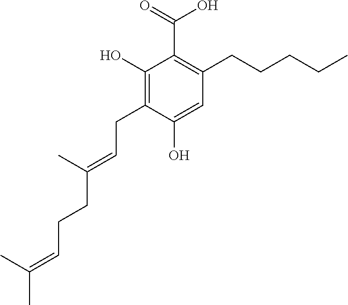 [SMILES: CCCCCc1cc(O)c(C/C=C(\C)CCC=C(C)C)c(O)c1C(=O)O]

measured by dry weight in said transgenic plant as compared to a comparable control plant without said modification.

In some embodiments, said modification results in 25% more

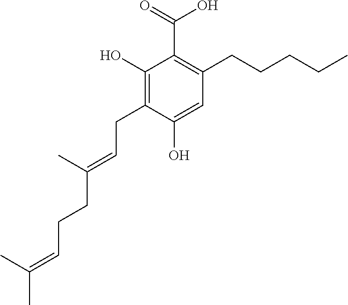 [SMILES: CCCCCc1cc(O)c(C/C=C(\C)CCC=C(C)C)c(O)c1C(=O)O]

measured by dry weight in said transgenic plant as compared to a comparable control plant without said modification.

In some embodiments, said modification results in 35% more

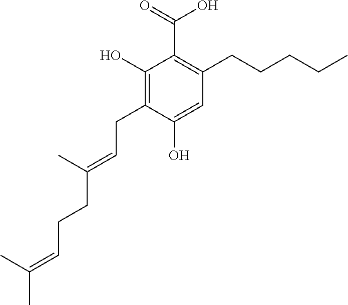 [SMILES: CCCCCc1cc(O)c(C/C=C(\C)CCC=C(C)C)c(O)c1C(=O)O]

measured by dry weight in said transgenic plant as compared to a comparable control plant without said modification.

In some embodiments, said modification results in 50% more

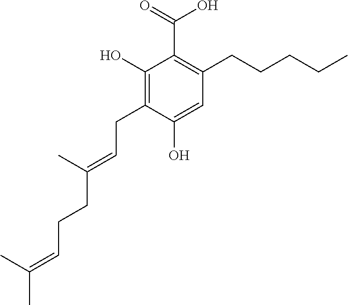 [SMILES: CCCCCc1cc(O)c(C/C=C(\C)CCC=C(C)C)c(O)c1C(=O)O]

measured by dry weight in said transgenic plant as compared to a comparable control plant without said modification.

In some embodiments, said modification results in 10% less cannabichromenic acid (CBCA) measured by dry weight in said transgenic plant as compared to a comparable control plant without said modification.

In some embodiments, said modification results in 10% less cannabidiolic acid (CBDA) measured by dry weight in said transgenic plant as compared to a comparable control plant without said modification.

In some embodiments, said modification results in 10% less tetrahydrocannabinolic acid (THCA) measured by dry weight in said transgenic plant as compared to a comparable control plant without said modification.

In one aspect, provided herein are methods for generating a transgenic plant, said method comprising: (a) contacting a plant cell with an endonuclease or a polypeptide encoding said endonuclease, wherein said endonuclease introduces a genetic modification resulting in an increased amount of cannabinol (CBN), derivative or analog thereof, compared to an amount of the same compound in a comparable control plant without said genetic modification; (b) culturing said plant cell in (a) to generate a transgenic plant.

In some embodiments, said contacting is via electroporation, agrobacterium mediated transformation, biolistic particle bombardment, or protoplast transformation.

In some embodiments, the method further comprises culturing said plant cell in (a) to generate a callus, a cotyledon, a root, a leaf, or a fraction thereof.

In some embodiments, said genetic modification results in an increased amount of cannabinol (CBN), derivative or analog thereof, compared to an amount of the same compound in a comparable control plant without said genomic modification.

In some embodiments, said genetic modification comprises a disruption of gene encoding THCA synthase.

In some embodiments, said disruption results in an increased amount of THCA synthase compared to an amount of the same compound in a comparable control plant without said disruption.

In some embodiments, said disruption is in a promoter region of said gene.

In some embodiments, said genetic modification comprises a disruption of genes encoding CBDA synthase and CBCA synthase respectively.

In some embodiments, said disruption results in decreased amount of CBDA synthase and CBCA synthase compared to an amount of the same compound in a comparable control plant without said disruption.

In some embodiments, said disruption is in a coding region of said genes.

In some embodiments, said genetic modification comprises a disruption of a third group of genes, wherein said disruption results in increased UV absorption of said transgenic plant compared to a comparable control without said disruption.

In some embodiments, said modification results in 10% more THC measured by dry weight in said transgenic plant as compared to a comparable control plant without said genetic modification.

In some embodiments, said modification results in 25% more THC measured by dry weight in said transgenic plant as compared to a comparable control plant without said genetic modification.

In some embodiments, said modification results in 35% more THC measured by dry weight in said transgenic plant as compared to a comparable control plant without said genetic modification.

In some embodiments, said modification results in 50% more THC measured by dry weight in said transgenic plant as compared to a comparable control plant without said genetic modification.

In some embodiments, said modification results in 10% less CBCA measured by dry weight in said transgenic plant as compared to a comparable control plant without said genetic modification.

In some embodiments, said modification results in 10% less CBDA measured by dry weight in said transgenic plant as compared to a comparable control plant without said genetic modification.

In one aspect, provided herein are methods for generating a transgenic plant, said method comprising: (a) contacting a plant cell with an endonuclease or a polypeptide encoding said endonuclease, wherein said endonuclease introduces a genetic modification resulting in an increased amount of tetrahydrocannabivarin (THCV), derivative or analog thereof, compared to an amount of the same compound in a comparable control plant without said genetic modification; (b) culturing said plant cell in (a) to generate a transgenic plant.

In some embodiments, said contacting is via electroporation, agrobacterium mediated transformation, biolistic particle bombardment, or protoplast transformation.

In some embodiments, the method further comprises culturing said plant cell in (a) to generate a callus, a cotyledon, a root, a leaf, or a fraction thereof

In some embodiments, said genetic modification results in an increased amount of tetrahydrocannabivarin (THCV), derivative or analog thereof, compared to an amount of the same compound in a comparable control plant without said genomic modification.

In some embodiments, said genetic modification comprises a disruption of a first of group of genes, wherein said disruption results in an increased amount of Formula I, derivative or analog thereof.

In some embodiments, said genetic modification comprises a disruption of a second group of genes, wherein said disruption results in a decreased amount of

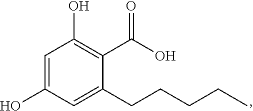 [SMILES: CCCCCc1cc(O)cc(O)c1C(=O)O]

derivative or analog thereof.

In some embodiments, said second group of genes comprises OAC and OLS.

In some embodiments, said disruption is in a coding region of said genes.

In some embodiments, said genetic modification comprises a disruption of a THCA synthase.

In some embodiments, said disruption results in an increased amount of THCA synthase, derivative or analog thereof, compared to an amount of the same compound in a comparable control plant without said disruption.

In some embodiments, said disruption is in a promoter region of said genes.

In some embodiments, said genetic modification comprises a disruption of a third group of genes encoding CBCA synthase and CBDA synthase respectively.

In some embodiments, said disruption results in a decreased amount of CBCA synthase and CBDA synthase, derivatives or analogs thereof

In some embodiments, said disruption is in a coding region of said genes.

In some embodiments, said modification results in 10% more tetrahydrocannabivarin (THCV) measure by dry weight in said transgenic plant as compared to a comparable control plant without said modification.

In some embodiments, said modification results in 25% more tetrahydrocannabivarin (THCV) measure by dry weight in said transgenic plant as compared to a comparable control plant without said modification.

In some embodiments, said modification results in 35% more tetrahydrocannabivarin (THCV) measure by dry weight in said transgenic plant as compared to a comparable control plant without said modification.

In some embodiments, said modification results in 50% more tetrahydrocannabivarin (THCV) measure by dry weight in said transgenic plant as compared to a comparable control plant without said modification.

In some embodiments, said modification results in 10% less cannabichromevarin (CBCV) measure by dry weight in said transgenic plant as compared to a comparable control plant without said modification.

In some embodiments, said modification results in 10% less cannabidivarin (CBDV) measure by dry weight in said transgenic plant as compared to a comparable control plant without said modification.

In some embodiments, said genetic modification is conducted by an endonuclease.

In some embodiments, said genetic modification comprises an insertion, a deletion, a substitution, or a frameshift.

In some embodiments, said endonuclease comprises a CRISPR enzyme, TALE-Nuclease, transposon-based nuclease, Zinc finger nuclease, meganuclease, Mega-TAL or DNA guided nuclease.

In some embodiments, said DNA-guided nuclease comprises argonaute.

In some embodiments, said endonuclease is a CRISPR enzyme complexed with a guide polynucleotide that is complementary to a target sequence of at least one of genes encoding OAC, OLS, GOT, CBCA synthase, CBDA synthase, and THCA synthase.

In some embodiments, said target sequence is at least 18 nucleotides, at least 19 nucleotides, at least 20 nucleotides, at least 21 nucleotides, or at least 22 nucleotides in length.

In some embodiments, said target sequence is at most 17 nucleotides in length.

In some embodiments, said target sequence comprises a sequence selected from Table 2 or Table 3 or complimentary thereof.

In some embodiments, said guide polynucleotide is a chemically modified.

In some embodiments, said guide polynucleotide is a single guide RNA (sgRNA).

In some embodiments, said guide polynucleotide is a chimeric single guide comprising RNA and DNA.

In some embodiments, said guide polynucleotide comprises a sequence selected from Table 2 or Table 3 or complimentary thereof

In some embodiments, said CRISPR enzyme is a Cas protein.

In some embodiments, said Cas protein comprises Cas1, Cas1B, Cas2, Cas3, Cas4, Cas5, Cas5d, Cas5t, Cas5h, Cas5a, Cas6, Cas7, Cas8, Cas9, Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, C2c1, C2c2, C2c3, Cpf1, CARF, DinG, homologues thereof, or modified versions thereof.

In some embodiments, said Cas protein is Cas9.

In some embodiments, said Cas9 recognizes a canonical PAM.

In some embodiments, said Cas9 recognizes a non-canonical PAM.

In some embodiments, said guide polynucleotide binds said target sequence 3-10 nucleotides from of PAM.

In some embodiments, said CRISPR enzyme complexed with said guide polynucleotide is introduced into said transgenic plant by an RNP.

In some embodiments, said CRISPR enzyme complexed with said guide polynucleotide is introduced into said transgenic plant by a vector comprising a nucleic acid encoding said CRISPR enzyme and said guide polynucleotide.

In some embodiments, said vector is a binary vector or a Ti plasmid.

In some embodiments, said vector further comprises a selection marker or a reporter gene.

In some embodiments, said RNP or vector is introduced into said transgenic plant via electroporation, agrobacterium mediated transformation, biolistic particle bombardment, or protoplast transformation.

In some embodiments, said RNP or vector further comprising a donor polynucleotide.

In some embodiments, said donor polynucleotide comprises homology to sequences flanking said target sequence.

In some embodiments, said donor polynucleotide introduces a stop codon into at least one of genes encoding OAC, OLS, GOT, CBCA synthase, CBDA synthase, and THCA synthase.

In some embodiments, said donor polynucleotide further comprises a barcode, a reporter gene, or a selection marker.

In one aspect, provided herein are genetically modified cell comprising a genetic modification, wherein said genetic modification results in an increased amount of

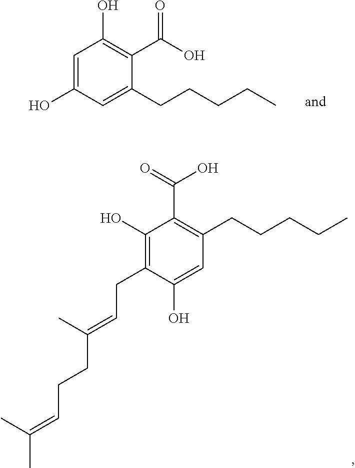

derivatives or analogs thereof, wherein said genetic modification does not result in a change of amount of

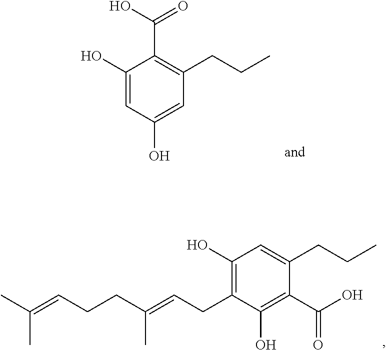 [SMILES: CCCc1cc(O)cc(O)c1C(=O)O ; CCCc1cc(O)c(C/C=C(\C)CCC=C(C)C)c(O)c1C(=O)O]

derivatives or analogs thereof, compared to an amount of the same compound in a comparable control cell without said genetic modification.

In some embodiments, said genetic modification further results in an increased amount of cannabigerol (CBG), derivative or analog thereof, compared to an amount of the same compound in a comparable control cell without said genetic modification.

In some embodiments, said genetic modification results in an increased amount of cannabinol (CBN), derivative or analog thereof, compared to an amount of the same compound in a comparable control cell without said genetic modification.

In some embodiments, said genetic modification results in an increased amount of tetrahydrocannabivarin (THCV), derivative or analog thereof, compared to an amount of the same compound in a comparable control cell without said genetic modification.

In some embodiments, said genetic modification comprises a disruption of gene encoding geranyl pyrophosphate synthase (GPPS), resulting in increased amount of geranyl pyrophosphate (GPP).

In some embodiments, genetic modification comprises a disruption of gene encoding polyketide synthase (PKS), resulting in increased amount of either Formula I or Formula II or both.

In some embodiments, the genetically modified cell is a plant cell, an algae cell, a agrobacterium cell, a E. coli cell, a yeast cell, an animal cell, or an insect cell.

In some embodiments, said genetically modified cell is a plant cell.

In some embodiments, said genetically modified cell is a cannabis plant cell.

In some embodiments, said genetically modified cell is a callus cell, a protoplast, an embryonic cell, a leaf cell, a seed cell, a stem cell, or a root cell.

In some embodiments, said modification is integrated in the genome of said cell.

In one aspect, provided herein are genetically modified cell comprising a genetic modification, wherein said genetic modification results in an increased amount of

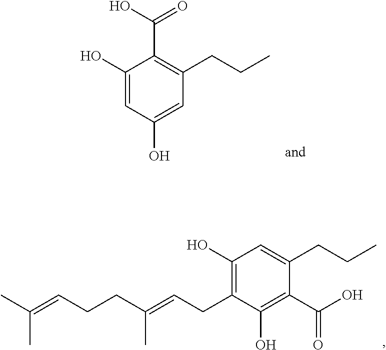 [SMILES: CCCc1cc(O)cc(O)c1C(=O)O ; CCCc1cc(O)c(C/C=C(\C)CCC=C(C)C)c(O)c1C(=O)O]

derivatives or analogs thereof, wherein said genetic modification does not result in a change of amount of

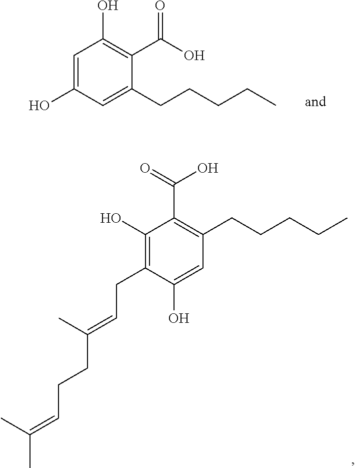

derivatives or analogs thereof, compared to an amount of the same compound in a comparable control cell without said genetic modification.

In some embodiments, said genetic modification further results in an increased amount of cannabigerol (CBG), derivative or analog thereof, compared to an amount of the same compound in a comparable control cell without said genetic modification.

In some embodiments, said genetic modification results in an increased amount of cannabinol (CBN), derivative or analog thereof, compared to an amount of the same compound in a comparable control cell without said genetic modification.

In some embodiments, said genetic modification results in an increased amount of tetrahydrocannabivarin (THCV), derivative or analog thereof, compared to an amount of the same compound in a comparable control cell without said genetic modification.

In some embodiments, said genetic modification comprises a disruption of gene encoding geranyl pyrophosphate synthase (GPPS), resulting in increased amount of geranyl pyrophosphate (GPP).

In some embodiments, genetic modification comprises a disruption of gene encoding polyketide synthase (PKS), resulting in increased amount of either Formula I or Formula II or both.

In some embodiments, the genetically modified cell is a plant cell, an algae cell, a agrobacterium cell, a E. coli cell, a yeast cell, an animal cell, or an insect cell.

In some embodiments, said genetically modified cell is a plant cell.

In some embodiments, said genetically modified cell is a cannabis plant cell.

In some embodiments, said genetically modified cell is a callus cell, a protoplast, an embryonic cell, a leaf cell, a seed cell, a stem cell, or a root cell.

In some embodiments, said modification is integrated in the genome of said cell.

### Incorporation by Reference

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A depicts a chemical structure of olivetolic acid (OA), a cannabinoid precursor of THC. FIG. 1B depicts the chemical structure of geranyl diphosphate (GFP), a cannabinoid precursor of THC. FIG. 1C depicts Δ⁹-tetrahydrocannabinoil. The bibenzopyran-numbering system is used.

FIG. 2 shows the biosynthesis of cannabigerolic acid (CBGA). The biosynthesis of the central intermediate CBGA is colored in dark green. The minor products CBNRA and CBGVA are shaded in light green. The precursor pathways are highlighted in light blue (GPP) and blue (OA). Me P, 2C-methyl-d-erythritol-4-phosphate; Do XP, 1-deoxy-d-xylulose-5-phosphate; MVA, mevalonate.

FIG. 3 shows the biosynthesis of cannabinoids. The enzymatically catalyzed reactions are highlighted in dark green. All nonenzyme-dependent modifications reactions are colored in light green. Biosynthesis of C3-cannabinoids starting from cannabigerovarinic acid (CBGVA) is carried out by the same enzymes.

FIG. 4 depicts an exemplary schematic of a method to enable cannabis plants to produce higher concentrations of individual cannabinoids, including rare cannabinoids. Genetic engineering can include genomic modification to augment rare cannabinoid DNA followed by introduction of enzymes in yeast to artificially create rare cannabinoids.

FIG. 5 shows a CRISPR cannabinoid engineering approach.

FIG. 6 depicts the biosynthesis of cannabigerolic acid (CBGA).

FIG. 7A shows conversion of CBGA to CBG. FIG. 7B shows a map of cannabinoid synthesis. C. sativa extracts are non-psychoactive until sufficient heat is supplied (at least about >105° C.) to cause a chemical reaction known as decarboxylation. Decarboxylation occurs slowly under ambient conditions, but the rate increases with temperature. High levels of decarboxylated cannabinoids in flowers can indicate that a sample has been stored improperly or is aging.

FIG. 8 shows a strategy for enhancing CBGA biosynthesis.

FIG. 9A shows a schematic of cannabinoid precursors. THCA synthase generates THCA from CBGA, but can also generate its homologue, THCVA, by using CBGVA as a substrate. The CBGVA precursor is generated by the GOT enzyme, utilising GPP as a substrate combined with Divarinic Acid. FIG. 9B depicts the biosynthesis of THCV.

FIG. 10 depicts a schematic of the biosynthesis of cannabinolic acid (CBNA). Decarboxylation of THCs produces CBN and occurs slowly under ambient conditions (the rate increases with temperature). Heat and light can cause THC to degrade to CBN.

FIGS. 11A and 11B show agrobacterium mediated transformation in callus cell from Finola plants resulting in expression of a representative transgene, namely GUS (blue with arrow pointed to),In some embodiments, the callus cells may be transformed with agrobacterium resulting in expression of THCAS transgene.

FIGS. 12A-12C show cotyledon inoculated with agrobacterium carrying an exemplary transgene GUS expression vector pCambia1301. FIGS. 12A and 12B show that GUS expression (blue; indicated by an arrow) is observed in cotyledon proximal site where callus regeneration occurs. In some embodiments, THCAS expression may be observed in cotyledon proximal sites where callus regeneration occurs when cotyledon is inoculated with agrobacterium carrying THCAS transgene. FIG. 12C shows that explant regenerated from primordia cells showing random GUS expression in regenerated explant. In some embodiments, an explant regenerated from primordia cells may display random THCAS gene.

FIGS. 13A-13D show that hypocotyls inoculated with pCambia:1301:GUS showed blue stain in regenerative tissues (b and d), and in regenerated explant (a and c) after 5 days on selection media.

FIG. 14 shows that Hemp isolated protoplasts were transfected with GUS expressing plasmid pCambia1301. GUS assay was conducted 72 hrs after transfection. Blue nuclei indicate GUS expression (indicated by black arrow).

FIG. 15 shows that Hemp Floral dipping was conducted by submerging female floral organs into Agrobacterium immersion solution for 10 min. Process was repeated 48 hrs later and inoculated plants were ready to be crossed with male pollen donors 24 hrs after the last inoculation.

FIGS. 16A-16C show that Cotyledon regeneration was achieved from a diversity of tissues. Primordia cells regenerate a long strong shoot (black arrow shown in FIG. 16A). In addition, callus regeneration from cotyledon proximal side also regenerate random numbers of shoots (white arrows shown in FIGS. 16B and 16C).

FIG. 17 shows that hypocotyl Regeneration showed high efficiency. Hypocotyl produced shoots and roots on plates and then were transferred to bigger pots where they could develop further. Once plants have developed strong roots, and the shoot is elongated, plantlets are transferred to compost for further growth.

FIG. 18 shows that agroinfiltration of hemp Finola leaves. Agrobacterium carrying the representative transgene GUS expression vector pCambia1302 was injected into the adaxial side of leaves using a 1 ml syringe. After 72 hrs, GUS assay was performed, and blues was observed in infiltrated leaves (indicated by black arrows).

FIGS. 19A-19C show maps of vectors disclosed herein.

### DETAILED DESCRIPTION

As used in the specification and claims, the singular forms “a,” “an,” and “the” include plural references unless the context clearly dictates otherwise. For example, the term “a chimeric transmembrane receptor polypeptide” includes a plurality of chimeric transmembrane receptor polypeptides.

The term “about” or “approximately” means within an acceptable error range for the particular value as determined by one of ordinary skill in the art, which can depend in part on how the value can be measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

As used herein, a “cell” can generally refer to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant, an algal cell, seaweeds, a fungal cell, an animal cell, a cell from an invertebrate animal, a cell from a vertebrate animal, a cell from a mammal, and the like. Sometimes a cell is not originating from a natural organism (e.g. a cell can be a synthetically made, sometimes termed an artificial cell).

As used herein, a “cannabinoid” can generally refer to a group of terpenophenolic compounds. Cannabinoids show affinity to cannabinoid receptors (CB1 and/or CB2) or are structurally related to tetrahydrocannabinol (THC). Cannabinoids can be differentiated into phytocannabinoids, synthetic cannabinoids, and endocannabinoids.

The term “gene,” as used herein, refers to a nucleic acid (e.g., DNA such as genomic DNA and cDNA) and its corresponding nucleotide sequence that can be involved in encoding an RNA transcript. The term as used herein with reference to genomic DNA includes intervening, non-coding regions as well as regulatory regions and can include 5′ and 3′ ends. In some uses, the term encompasses the transcribed sequences, including 5′ and 3′ untranslated regions (5′-UTR and 3′-UTR), exons and introns. In some genes, the transcribed region can contain “open reading frames” that encode polypeptides. In some uses of the term, a “gene” comprises only the coding sequences (e.g., an “open reading frame” or “coding region”) necessary for encoding a polypeptide. In some cases, genes do not encode a polypeptide, for example, ribosomal RNA genes (rRNA) and transfer RNA (tRNA) genes. In some cases, the term “gene” includes not only the transcribed sequences, but in addition, also includes non-transcribed regions including upstream and downstream regulatory regions, enhancers and promoters. A gene can refer to an “endogenous gene” or a native gene in its natural location in the genome of an organism. A gene can refer to an “exogenous gene” or a non-native gene. A non-native gene can refer to a gene not normally found in the host organism but which can be introduced into the host organism by gene transfer. A non-native gene can also refer to a gene not in its natural location in the genome of an organism. A non-native gene can also refer to a naturally occurring nucleic acid or polypeptide sequence that comprises mutations, insertions and/or deletions (e.g., non-native sequence).

The term “nucleotide,” as used herein, generally refers to a base-sugar-phosphate combination. A nucleotide can comprise a synthetic nucleotide. A nucleotide can comprise a synthetic nucleotide analog. Nucleotides can be monomeric units of a nucleic acid sequence (e.g. deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example, [aS]dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP. A nucleotide can be unlabeled or detectably labeled by well-known techniques. Labeling can also be carried out with quantum dots. Detectable labels can include, for example, radioactive isotopes, fluorescent labels, chemiluminescent labels, bioluminescent labels and enzyme labels. Fluorescent labels of nucleotides can include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif. FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g. biotin-11-dUTP, biotin-16-dUTP, biotin-20-dUTP).

The term “percent (%) identity,” as used herein, can refer to the percentage of amino acid (or nucleic acid) residues of a candidate sequence that are identical to the amino acid (or nucleic acid) residues of a reference sequence after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment, for purposes of determining percent identity, can be achieved in various ways that are within the skill in the art, for instance, using publicly available computer software such as BLAST, ALIGN, or Megalign (DNASTAR) software. Percent identity of two sequences can be calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence.

As used herein, the term “plant” includes a whole plant and any descendant, cell, tissue, or part of a plant. A class of plant that can be used in the present disclosure can be generally as broad as the class of higher and lower plants amenable to mutagenesis including angiosperms (monocotyledonous and dicotyledonous plants), gymnosperms, ferns and multicellular algae. Thus, “plant” includes dicot and monocot plants. The term “plant parts” include any part(s) of a plant, including, for example and without limitation: seed (including mature seed and immature seed); a plant cutting; a plant cell; a plant cell culture; a plant organ (e.g., pollen, embryos, flowers, fruits, shoots, leaves, roots, stems, and explants). A plant tissue or plant organ may be a seed, protoplast, callus, or any other group of plant cells that can be organized into a structural or functional unit. A plant cell or tissue culture may be capable of regenerating a plant having the physiological and morphological characteristics of the plant from which the cell or tissue was obtained, and of regenerating a plant having substantially the same genotype as the plant. In contrast, some plant cells are not capable of being regenerated to produce plants. Regenerable cells in a plant cell or tissue culture may be embryos, protoplasts, meristematic cells, callus, pollen, leaves, anthers, roots, root tips, silk, flowers, kernels, ears, cobs, husks, or stalks.

As used herein, the term “tetrahydrocannabinolic acid (THCA) synthase inhibitory compound” refers to a compound that suppresses or reduces an activity of THCA synthase enzyme activity, or expression of THCA synthase enzyme, such as for example synthesis of mRNA encoding a THCA synthase enzyme (transcription) and/or synthesis of a THCA synthase polypeptide from THCA synthase mRNA (translation). In some embodiments the selective THCA synthase inhibitory compound specifically inhibits a THCA synthase that decreases formation of delta-9-tetrahydrocannabinol (THC) and/or increases cannabidiol (CBD).

As used herein, the term “transgene” refers to a segment of DNA which has been incorporated into a host genome or is capable of autonomous replication in a host cell and is capable of causing the expression of one or more coding sequences. Exemplary transgenes will provide the host cell, or plants regenerated therefrom, with a novel phenotype relative to the corresponding non-transformed cell or plant. Transgenes may be directly introduced into a plant by genetic transformation, or may be inherited from a plant of any previous generation which was transformed with the DNA segment. In some cases, a transgene can be a barcode. In some cases, a transgene can be a marker.

As used herein, the term “transgenic plant” refers to a plant or progeny plant of any subsequent generation derived therefrom, wherein the DNA of the plant or progeny thereof contains an introduced exogenous DNA segment not naturally present in a non-transgenic plant of the same strain. The transgenic plant may additionally contain sequences which are native to the plant being transformed, but wherein the “exogenous” gene has been altered in order to alter the level or pattern of expression of the gene, for example, by use of one or more heterologous regulatory or other elements.

A vector can be a polynucleotide (e.g., DNA or RNA) used as a vehicle to artificially carry genetic material into a cell, where it can be replicated and/or expressed. In some aspects, a vector is a binary vector or a Ti plasmid. Such a polynucleotide can be in the form of a plasmid, YAC, cosmid, phagemid, BAC, virus, or linear DNA (e.g., linear PCR product), for example, or any other type of construct useful for transferring a polynucleotide sequence into another cell. A vector (or portion thereof) can exist transiently (i.e., not integrated into the genome) or stably (i.e., integrated into the genome) in the target cell. In some aspects, a vector can further comprise a selection marker or a reporter.

The practice of some methods disclosed herein employ, unless otherwise indicated, conventional techniques of immunology, biochemistry, chemistry, molecular biology, microbiology, cell biology, genomics and recombinant DNA, which are within the skill of the art. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)).

Described are genetically modified cannabis and/or hemp plants, portions of plants thereof, and cannabis and/or hemp plant derived products as well as expression cassettes, vectors, compositions, and materials and methods for producing the same. Cannabis contains many chemically distinct components, many of which have therapeutic properties that can be altered. Therapeutic components of medical cannabis are delta-9-tetrahydrocannabinol (THC) and cannabidiol (CBD). Provided herein are genetically modified cannabis having increased amount of cannabigerol (CBG), cannabinol (CBN), tetrahydrocannabivarin (THCV), other rare CBDs, or any combinations thereof. Provided herein are also methods of making genetically modified cannabis utilizing Clustered Regularly Interspaced Short Palindromic Repeats (CRISPR) technology and reagents for generating the genetically modified cannabis. Compositions and methods provided herein can be utilized for the generation of a substantially CBD-only plant strain. Compositions provided herein can be utilized for various uses including but not limited to therapeutic uses, preventative uses, palliative uses, and recreational uses.

### Genetically Modified Plants

Genomic modulation of the cannabinoid biosynthesis pathway can enable the redesigning of the cannabis plant metabolic pathway to produce altered levels of cannabinoids, including rare cannabinoids, and generate new cannabinoids and variant cannabinoids. Using gene editing, the production of early, intermediate, and late precursor compounds may be influenced and/or skewed to generate desired end products. Additionally, switching off specific pathways of the cannabinoid biosynthesis pathway using gene editing can produce novel profiles of cannabinoid compounds.

Plant secondary metabolite production results from tightly regulated biosynthetic pathways leading to the production of one or more bioactive metabolites that accumulate in the plant tissues at different concentrations. Metabolic engineering of these pathways can be used to generate plant lines with increased production of specific metabolite(s) of interest. Plant genetic engineering technologies can be applied to selectively modify cannabis secondary metabolism through the down regulation of key enzymes involved in THC biosynthesis. The down regulation or knock out of key steps in metabolic pathway can re-direct intermediates and energy to alternative metabolic pathways and result in increased production and accumulation of other end products. Since rare cannabinoids and other valuable pharmaceutical compounds produced by cannabis share specific steps and intermediates in secondary metabolism biosynthetic pathways, the reduction of THC or other components of the metabolic pathway can increase the production of compounds of interest, such as, rare cannabinoids.

Down regulation of key steps in metabolic pathway re-directs intermediates and energy to alternative metabolic pathways and results in increased production and accumulation of other end products. THC and other cannabis metabolites share a biosynthetic pathway; that cannabigerolic acid is a precursor of THC, CBD and Cannabichromene. In particular, THCA synthase catalyzes the production of delta-9-tetrahydrocannabinolic acid from cannabigerolic acid; delta-9-tetrahydrocannabinolic undergoes thermal conversion to form THC. CBDA synthase catalyzes the production of cannabidiolic acid from cannabigerolic acid; cannabidiolic acid undergoes thermal conversion to CBD. CBCA synthase catalyzes the production of cannabichromenic acid from cannabigerolic acid; cannabichromenic acid undergoes thermal conversion to cannabichromene.

In some cases, a reduction in the production of THC, CBD, or Cannabichromene may enhance production of the remaining metabolites in this shared pathway. For example, production of CBD and/or Cannabichromene can be enhanced by inhibiting production of THC. THC production may be inhibited by inhibiting expression and/or activity of tetrahydrocannabinolic acid (THCA) synthase enzyme. Described are certain embodiments of enhancing production of one or more secondary metabolites by disruption of the production of one or more metabolites having a shared biosynthetic pathway. Certain embodiments provide methods of enhancing production of one or more secondary metabolites that share steps and intermediates in the THC biosynthetic pathway by downregulation of THC production. In specific embodiments, there are provided methods of enhancing production of CBD and/or Cannabichromene by inhibiting or disrupting production of THC. Certain embodiments provide methods of enhancing production of one or more secondary metabolites which share steps and intermediates in the THC biosynthetic pathway by downregulation or knock out of expression and/or activity of THCA synthase. In specific embodiments, there are provided methods of enhancing production of CBD and/or Cannabichromene by downregulation of expression and/or activity of THCA synthase.

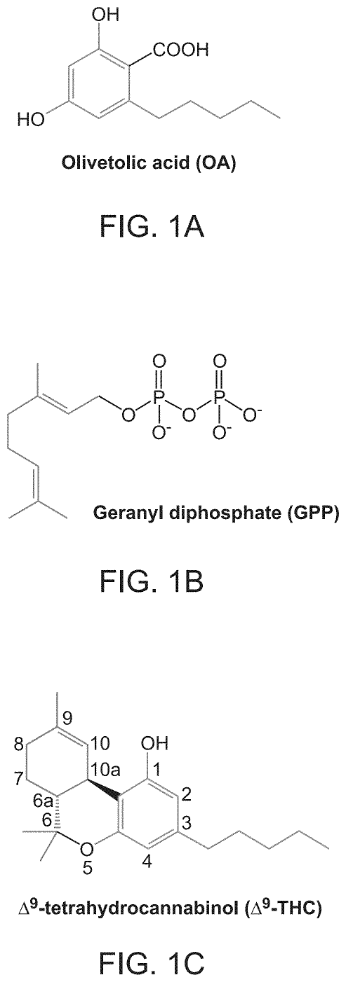

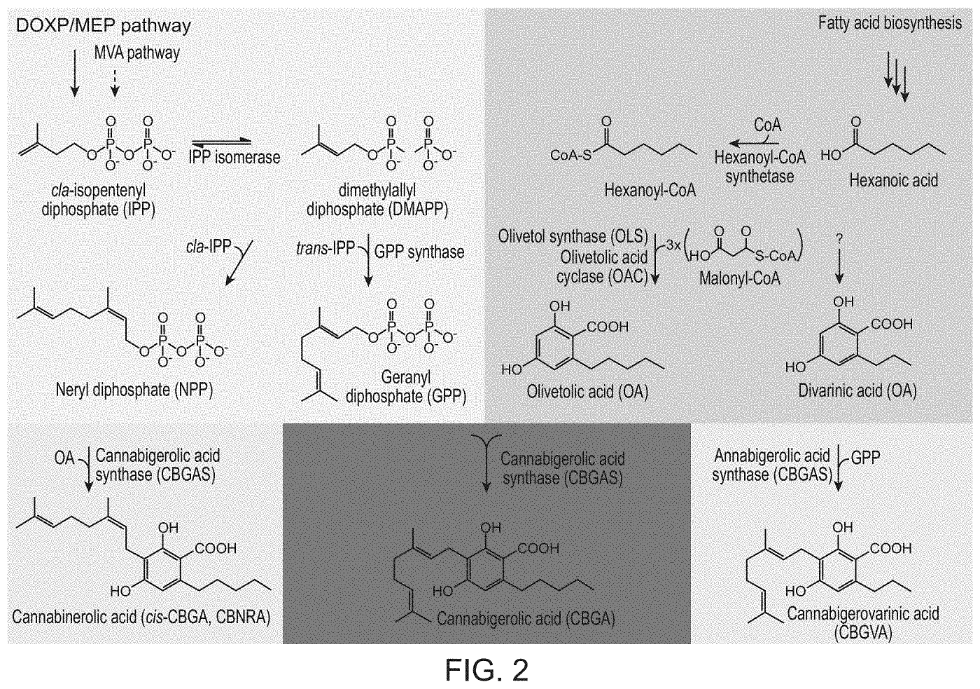

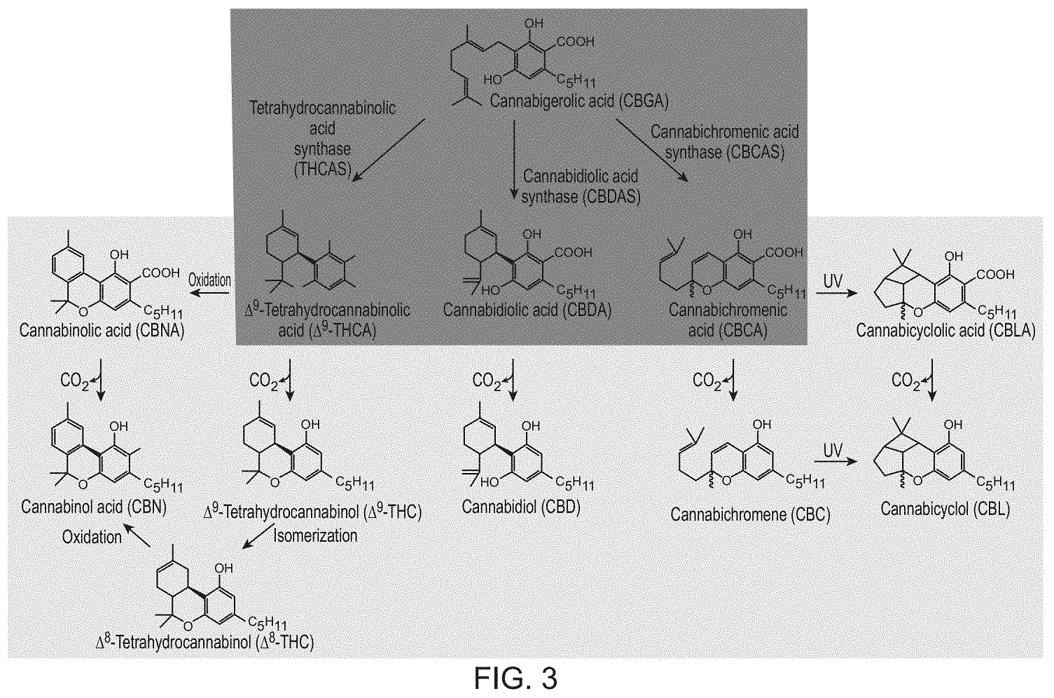

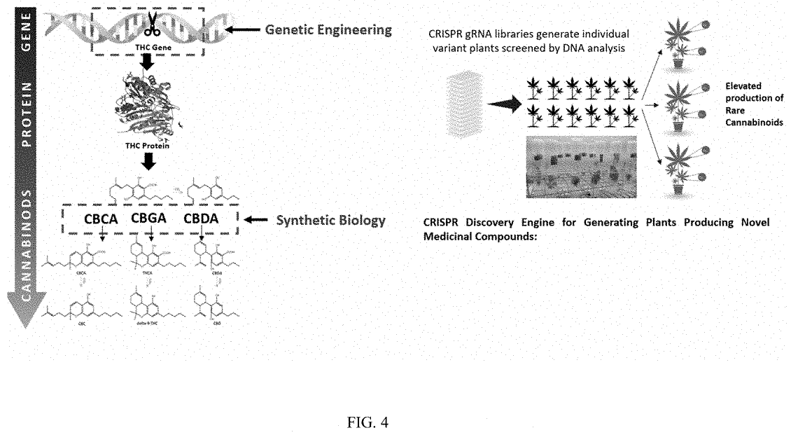

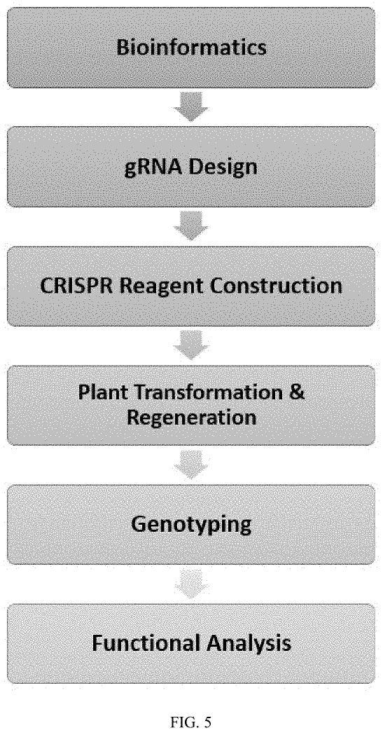

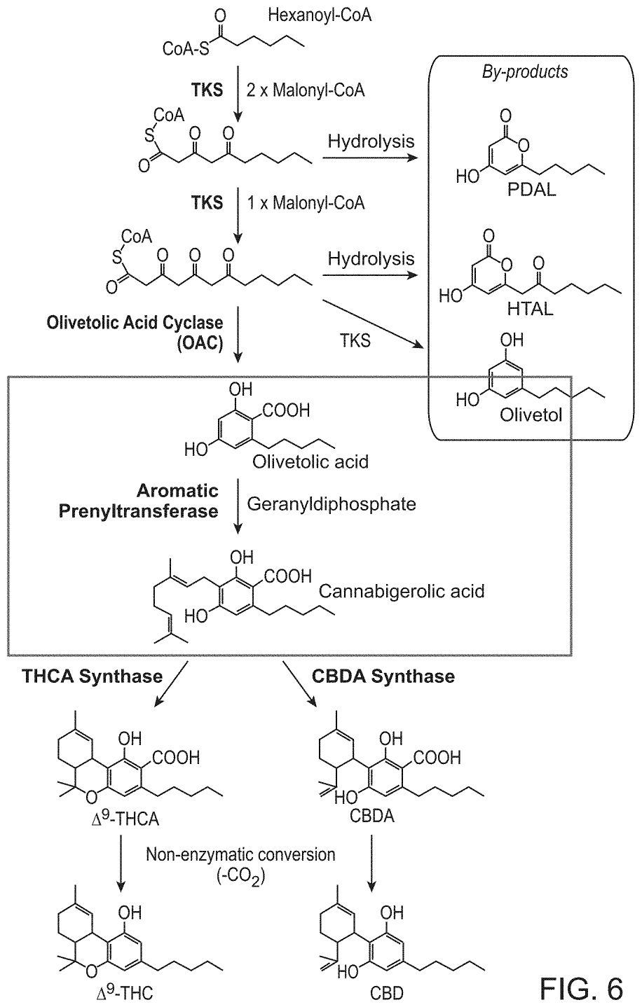

C. sativa has been intensively bred, resulting in extensive variation in morphology and chemical composition. It is perhaps best known for producing cannabinoids, a unique class of compounds that may function in chemical defense, but also have pharmaceutical and psychoactive properties. The general structure of cannabinoids and their precursors, olivetolic acid, and geranyl diphosphate are shown in FIG. 1A, FIG. 1B, and FIG. 1C. Cannabinoids are composed of two parts: a cyclic monoterpene part, and a diphenol (resorcin) part, carrying an alkyl chain. Although many cannabinoids are known, cannabigerolic acid synthase (CBGAS), tetrahydrocannabinolic acid synthase (THCAS), cannabidiolic acid synthase (CBDAS), Table 1, and cannabichromenic acid synthase (CBCAS) are implicated in cannabinoid biosynthesis. Cannabinoids have their biosynthetic origins in both polyketide (phenolic) and terpenoid metabolism and are termed terpenophenolics or prenylated polyketides. Cannabinoid biosynthesis occurs primarily in glandular trichomes that cover female flowers at a high density. Cannabinoids are formed by a three-step biosynthetic process: polyketide formation, aromatic prenylation and cyclization.

TABLE 1
CBCAS nucleic acid gene sequence

| SEQ ID | |
|---|---|
| NO | Sequence |
| 1 | ATGAATTGCTCAACATTCTCCTTTTGGTTTGTTTGCAAAATA |
| | ATATTTTTCTTTCTCTCATTCAATATCCAAATTTCAATAGCT |
| | AATCCTCAAGAAAACTTCCTTAAATGCTTCTCGGAATATAT |
| | TCCTAACAATCCAGCAAATCCAAAATTCATATACACTCAAC |
| | ACGACCAATTGTATATGTCTGTCCTGAATTCGACAATACAA |
| | AATCTTAGATTCACCTCTGATACAACCCCAAAACCACTCGT |
| | TATTGTCACTCCTTCAAATGTCTCCCATATCCAGGCCAGTAT |
| | TCTCTGCTCCAAGAAAGTTGGTTTGCAGATTCGAACTCGAA |
| | GCGGTGGCCATGATGCTGAGGGTTTGTCCTACATATCTCAAG |
| | TCCCATTTGCTATAGTAGACTTGAGAAACATGCATACGGTCA |
| | AAGTAGATATTCATAGCCAAACTGCGTGGGTTGAAGCCGGA |
| | GCTACCCTTGGAGAAGTTTATTATTGGATCAATGAGATGAAT |
| | GAGAATTTTAGTTTTCCTGGTGGGTATTGCCCTACTGTTGGCG |
| | TAGGTGGACACTTTAGTGGAGGAGGCTATGGAGCATTGATGC |
| | GAAATTATGGCCTTGCGGCTGATAATATCATTGATGCACACTT |
| | AGTCAATGTTGATGGAAAAGTTCTAGATCGAAAATCCATGGG |
| | AGAAGATCTATTTTGGGCTATACGTGGTGGAGGAGGAGAAAA |
| | CTTTGGAATCATTGCAGCATGTAAAATCAAACTTGTTGTTGTC |
| | CCATCAAAGGCTACTATATTCAGTGTTAAAAAGAACATGGAG |
| | ATACATGGGCTTGTCAAGTTATTTAACAAATGGCAAAATATT |
| | GCTTACAAGTATGACAAAGATTTAATGCTCACGACTCACTTC |
| | AGAACTAGGAATATTACAGATAATCATGGGAAGAATAAGAC |
| | TACAGTACATGGTTACTTCTCTTCCATTTTTCTTGGTGGAGTG |
| | GATAGTCTAGTTGACTTGATGAACAAGAGCTTTCCTGAGTTG |
| | GGTATTAAAAAAACTGATTGCAAAGAATTGAGCTGGATTGA |
| | TACAACCATCTTCTACAGTGGTGTTGTAAATTACAACACTGC |
| | TAATTTTAAAAAGGAAATTTTGCTTGATAGATCAGCTGGGAA |
| | GAAGACGGCTTTCTCAATTAAGTTAGACTATGTTAAGAAACT |
| | AATACCTGAAACTGCAATGGTCAAAATTTTGGAAAAATTATA |
| | TGAAGAAGAGGTAGGAGTTGGGATGTATGTGTTGTACCCTTA |
| | CGGTGGTATAATGGATGAGATTTCAGAATCAGCAATTCCATT |
| | CCCTCATCGAGCTGGAATAATGTATGAACTTTGGTACACTGC |
| | TACCTGGGAGAAGCAAGAAGATAACGAAAAGCATATAAACT |
| | GGGTTCGAAGTGTTTATAATTTCACAACTCCTTATGTGTCCCA |
| | AAATCCAAGATTGGCGTATCTCAATTATAGGGACCTTGATTTA |
| | GGAAAAACTAATCCTGAGAGTCCTAATAATTACACACAAGCAC |
| | GTATTTGGGGTGAAAAGTATTTTGGTAAAAATTTTAACAGGTT |
| | AGTTAAGGTGAAAACCAAAGCTGATCCCAATAATTTTTTTAGA |
| | AACGAACAAAGTATCCCACCTCTTCCACCGCGTCATCAT |

Genes in the cannabinoid biosynthesis pathway of C. sativa may be disrupted using the methods provided herein. There are over 113 known cannabinoids (Elsohly and Slade 2005), but the two most abundant natural derivatives are THC and cannabidiol (CBD). THCA and CBDA are both synthesized from cannabigerolic acid by the related enzymes THCA synthase (THCAS) and CBDA synthase (CBDAS), respectively. Expression of THCAS and CBDAS appear to be the major factor determining cannabinoid content. In addition to plant cannabis sativa, there are two classes of cannabinoids—the synthetic cannabinoids (e.g., WIN55212-2) and the endogenous cannabinoids (eCB), anandamide (ANA) and 2-arachidonoylglycerol (2-AG).

THC is responsible for the well-known psychoactive effects of cannabis and/or hemp consumption, but CBD, while non-intoxicating, also has therapeutic properties, and is specifically being investigated as a treatment for both schizophrenia (Osborne et al. 2017) and Alzheimer's disease (Watt and Karl 2017). Cannabis has traditionally been classified as having a drug (“marijuana”) or hemp chemotype based on the relative proportion of THC to CBD, but types grown for psychoactive use produce relatively large amounts of both. Cannabis containing high levels of CBD is increasingly grown for medical use. Examples of cannabinoids comprise compounds belonging to any of the following classes of molecules, their derivatives, salts, or analogs: Tetrahydrocannabinol (THC), Tetrahydrocannabivarin (THCV), Cannabichromene (CBC), Cannabichromanon (CBCN), Cannabidiol (CBD), Cannabielsoin (CBE), Cannabidivarin (CBDV), Cannbifuran (CBF), Cannabigerol (CBG), Cannabicyclol (CBL), Cannabinol (CBN), Cannabinodiol (CBND), Cannabitriol (CBT), Cannabivarin (CBV), and Isocanabinoids. In one embodiment, a cannabinoid that can be disrupted is chosen from Cannabigerolic Acid (CBGA), Cannabigerolic Acid monomethylether (CBGA), Cannabigerol (CBG), Cannabigerol monomethylether (CBGM), Cannabigerovarinic Acid (CBGVA),Cannabigerovarin (CBGV), Cannabichromenic Acid (CBCA), Cannabichromene (CBC), Cannabichromevarinic Acid (CBCVA), Cannabichromevarin (CBCV), Cannabidiolic Acid (CBDA), Cannabidiol (CBD), Cannabidiol monomethylether (CBDM), Cannabidiol-C4 (CBD-C4), Cannabidivarinic Acid (CBDVA), Cannabidivarin (CBDV), Cannabidiorcol (CBD-Ci), Tetrahydrocannabinolic acid A (THCA-A), Tetrahydrocannabinolic acid B (THCA-B), Tetrahydrocannabinolic Acid (THCA), Tetrahydrocannabinol (THC), Tetrahydrocannabinolic acid C (THCA-C4), Tetrahydrocannbinol C (THC-C4), Tetrahydrocannabivarinic acid (THCVA), Tetrahydrocannabivarin (THCV),Tetrahydrocannabiorcolic acid (THCA-C1), Tetrahydrocannabiorcol (THC-C1), A⁷-cis-iso-tetrahydrocannabivarin, A⁸-tetrahydrocannabinolic acid (A8-THCA), Cannabivarinodiolic (CBNDVA), Cannabivarinodiol (CBNDV), A etrahydrocannabinol (A⁸-THC), Δ⁹-ieirahydrocannabinol (A⁹-THC), Cannabicyclolic acid (CBLA), Cannabicyclol (CBL), Cannabicyclovarin (CBLV), Cannabielsoic acid A (CBEA-A), Cannabielsoic acid B (CBEA-B), Cannabielsoin (CBE), Cannabivarinselsoin (CBEV), Cannabivarinselsoinic Acid (CBEVA),Cannabielsoic Acid (CBEA), Cannabielvarinsoin (CBLV), Cannabielvarinsoinic Acid (CBLVA), Cannabinolic acid (CBNA), Cannabinol (CBN), Cannabivarinic Acid (CBNVA), Cannabinol methylether (CBNM), Cannabinol-C₄ (CBN-C₄), Cannabivarin (CBV), Cannabino-C₂ (CBN-C₂), Cannabiorcol (CBN-C1),Cannabinodiol (CBND), Cannabinodiolic Acid (CBNDA), Cannabinodivarin (CBDV), Cannabitriol (CBT), 10-Ethoxy-9-hydroxy-A^{8a}-tetrahydrocannabinol, 8,9-Dibydroxy-A^{6a(10a)}-tetrahydrocannabinol (8,9-Di-OH-CBT-C₅), Cannabitriolvarin (CBTV), Ethoxy-cannabitriolvarin (CBTVE), Dehydrocannabifuran (DCBF), Cannbifuran (CBF), Cannabichromanon (CBCN), Cannabicitran (CBT), 10-Oχo-Δ^{δ3/4ĺ|∧}-tetrahydrocannabinol (OTHC), A⁹-c s-tetrahydrocannabinoi (cis-THC), Cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), Trihydroxy-delta-9-tetrahydrocannabinol (triOH-THC), Yangonin, Epigallocatechin gallate, Dodeca-2E, 4E, 8Z, 10Z-tetraenoic acid isobutylamide, and Dodeca-2E,4E-dienoic acid isobutylamide.

In some aspects, a component of a cannabinoid pathway can be disrupted. For example, terpenes, including terpenoids, are a class of compounds that are produced by cannabis. As used herein, the term “terpene” means an organic compound built on an isoprenoid structural scaffold or produced by combining isoprene units. Often, terpene molecules found in plants may produce a distinct scent. In some cases, a compound in a cannabinoid pathway that can be disrupted is chosen from cannabinoids or terpenes. The structure of terpenes can be built with isoprene units. Flavonoids are larger carbon structures with two phenyl rings and a heterocyclic ring. In some cases, there can be an overlap in which a flavonoid could be considered a terpene. However, not all terpenes could be considered flavonoids. Within the context of this disclosure, the term terpene includes Hemiterpenes, Monoterpenols, Terpene esters, Diterpenes, Monoterpenes, Polyterpenes, Tetraterpenes, Terpenoid oxides, Sesterterpenes, Sesquiterpenes, Norisoprenoids, or their derivatives. Derivatives of terpenes include terpenoids in their forms of hemiterpenoids, monoterpenoids, sesquiterpenoids, sesterterpenoid, sesquarterpenoids, tetraterpenoids, Triterpenoids, tetraterpenoids, Polyterpenoids, isoprenoids, and steroids. They may be forms: α-, β-, γ-, oχo-, isomers, or combinations thereof. Examples of terpenes within the context of this disclosure include: 7,8-dihydroionone, Acetanisole, Acetic Acid, Acetyl Cedrene, Anethole, Anisole, Benzaldehyde, Bergamotene (α-cis-Bergamotene) (a-trans-Bergamotene), Bisabolol β-Bisabolol), Borneol, Bornyl Acetate, Butanoic/Butyric Acid, Cadinene (a-Cadinene) (γ-Cadinene), Cafestol, Caffeic acid, Camphene, Camphor, Capsaicin, Carene (Δ-3-Carene), Carotene, Carvacrol, Carvone, Dextro-Carvone, Laevo-Carvone, Caryophyllene (β-Caryophyllene), Caryophyllene oxide, Castoreum Absolute, Cedrene (a-Cedrene) (β-Cedrene), Cedrene Epoxide (a-Cedrene Epoxide), Cedrol, Cembrene, Chlorogenic Acid, Cinnamaldehyde (a-amyl-Cinnamaldehyde) (α-hexyl-Cinnamaldehyde), Cinnamic Acid, Cinnamyl Alcohol, Citronellal, Citronellol, Cryptone, Curcumene (α-Curcumene) (γ-Curcumene), Decanal, Dehydrovomifoliol, Diallyl Disulfide, Dihydroactinidiolide, Dimethyl Disulfide, Eicosane/lcosane, Elemene (β-Elemene), Estragole, Ethyl acetate, Ethyl Cinnamate, Ethyl maltol, Eucalyptol/1,8-Cineole, Eudesmol (a-Eudesmol) (βEudesmol) (γ-Eudesmol), Eugenol, Euphol, Farnesene, Farnesol, Fenchol (β-Fenchol), Fenchone, Geraniol, Geranyl acetate, Germacrenes, Germacrene B, Guaia-1 (10),11 -diene, Guaiacol, Guaiene (α-Guaiene), Gurjunene (α-Gurjunene), Herniarin, Hexanaldehyde, Hexanoic Acid, Humulene (a-Humulene) (β-Humulene), lonol (3-oxo-a-ionol) (β-lonol), lonone (a-lonone) (β-lonone), Ipsdienol, Isoamyl acetate, Isoamyl Alcohol, Isoamyl Formate, Isoborneol, Isomyrcenol, Isopulegol, Isovaleric Acid, Isoprene, Kahweol, Lavandulol, Limonene, γ-Linolenic Acid, Linalool, Longifolene, a-Longipinene, Lycopene, Menthol, Methyl butyrate, 3-Mercapto-2-Methylpentanal, Mercaptan/Thiols, β-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (β-Myrcene), γ-Muurolene, Nepetalactone, Nerol, Nerolidol, Neryl acetate, Nonanaldehyde, Nonanoic Acid, Ocimene, Octanal, Octanoic Acid, P-cymene, Pentyl butyrate, Phellandrene, Phenylacetaldehyde, Phenylethanethiol, Phenylacetic Acid, Phytol, Pinene, β-Pinene, Propanethiol, Pristimerin, Pulegone, Quercetin, Retinol, Rutin, Sabinene, Sabinene Hydrate, cis-Sabinene Hydrate, trans-Sabinene Hydrate, Safranal, α-Selinene, a-Sinensal, βSinensal, β-Sitosterol, Squalene, Taxadiene, Terpin hydrate, Terpineol, Terpine-4-ol, a-Terpinene, γ-Terpinene, Terpinolene, Thiophenol, Thujone, Thymol, a-Tocopherol, Tonka Undecanone, Undecanal, Valeraldehyde/Pentanal, Verdoxan, α-Ylangene, Umbelliferone, or Vanillin.

Terpenes known to be produced by cannabis include, without limitation, aromadendrene, bergamottin, bergamotol, bisabolene, borneol, 4-3-carene, caryophyllene, cineole/eucalyptol, p-cymene, dihydroj asmone, elemene, farnesene, fenchol, geranylacetate, guaiol, humulene, isopulegol, limonene, linalool, menthone, menthol, menthofuran, myrcene, nerylacetate, neomenthylacetate, ocimene, perillylalcohol, phellandrene, pinene, pulegone, sabinene, terpinene, terpineol, terpineol-4-ol, terpinolene, and derivatives, isomers, enantiomers, etc. of each thereof. In some cases, types and ratios of terpenes produced by a cannabis strain can be dependent on genetics and growth conditions (e.g., lighting, fertilization, soil, watering frequency/amount, humidity, carbon dioxide concentration, and the like), as well as age, maturation, and time of day. Terpenes have been shown to have medicinal properties and may be responsible for at least a portion of the medicinal value of cannabis. Some of the medical benefits attributable to one or more of the terpenes isolated from cannabis include treatment of sleep disorders, psychosis, anxiety, epilepsy and seizures, pain, microbial infections (fungal, bacterial, etc.), cancer, inflammation, spasms, gastric reflux, depression, and asthma. Some terpenes have been shown to: lower the resistance across the blood-brain barrier, act on cannabinoid receptors and other neuronal receptors, stimulate the immune system, and/or suppress appetite.

In some cases, cannabis plants and products may also comprise other pharmaceutically relevant compounds, including flavonoids and phytosterols (e.g., apigenin, quercetin, cannflavin A,. beta.-sitosterol and the like).

In some cases, provided herein can be a plant comprising a genome modification that can result in an increased amount of any one of:

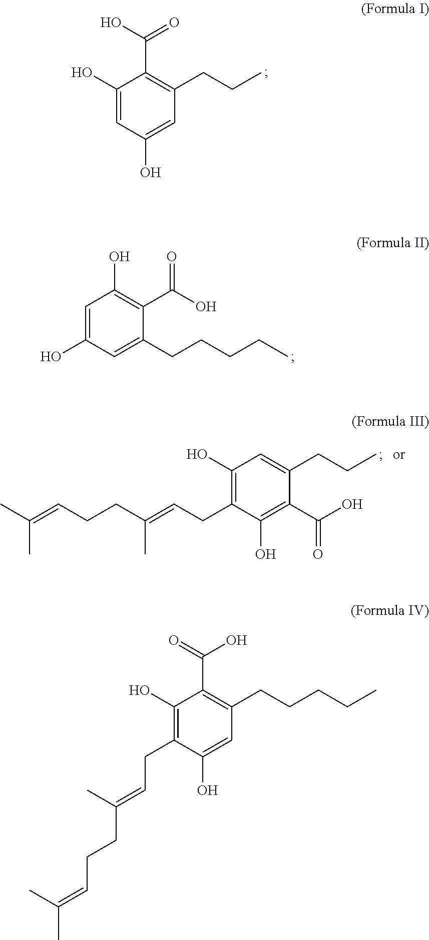

derivatives and analogs thereof, as compared to an amount of the same compound in a comparable control plant absent a genomic modification. In some cases, a transgenic plant can also comprise an increased amount of cannabigerol (CBG), a derivative or analog thereof, as compared to an amount of the same compound in a comparable control plant absent a genomic modification. An increased amount of CBG can be about 1 fold, 2 fold, 3 fold, 4 fold, 5 fold, 10 fold, 20 fold, 30 fold, 50 fold, 80 fold, 100 fold, 150 fold, 200 fold, 250 fold, 500 fold, 800 fold, or up to about 1000 fold as compared to a comparable plant absent genomic modification. For example, a modification can comprise a genetic disruption that results in an increased expression of Formula II, or a derivative or analog thereof. In some cases, Formula II can comprise genes such as OAC and OLS. In some cases, genes such as prenyl-transferase are genomically modified such that a disruption results in an increased amount of prenyl-transferase as compared to an amount of the same compound in a comparable control plant absent a genomic disruption. In some cases, prenyl-transferase can be olivetolic acid geranyltransferase (GOT). In some aspects, a transgenic plant provided herein has a disruption in a first group of genes that result in an increased amount of

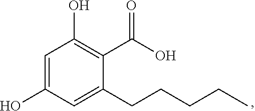 [SMILES: CCCCCc1cc(O)cc(O)c1C(=O)O]

derivative or analog thereof. A first group of genes can comprise olivetolic acid cyclase (OAC) and/or olivetolic acid synthase (OLS).

In some aspects, a gene or portion thereof associated with THC production may be disrupted. In other aspects, a gene or portion thereof associated with THC production of cannabis may be down regulated. In some aspects, a promoter of a gene or portion of a gene provided herein can be disrupted with systems provided herein. The DNA sequences encoding the THCA synthase gene in Cannabis and Hemp plants is mapped and annotated using the published genome sequence of Cannabis Sativa and Hemp (Finola).

Certain embodiments provide for cannabis and/or hemp plants and/or plant cells having enhanced production of one or more secondary metabolites that share steps and intermediates in the THC biosynthetic pathway and downregulated expression and/or activity of THCA synthase. In specific embodiments, there are provided cannabis and/or hemp plants and/or cells having enhanced production of CBD and/or Cannabichromene and downregulated expression and/or activity of a gene involved in the cannabinoid metabolic pathway. Provided herein can be enhancing production of one or more secondary metabolites by downregulation or disruption of the production of one or more metabolites having a shared biosynthetic pathway. Certain embodiments provide methods of enhancing production of one or more secondary metabolites that can share steps and intermediates in the THC biosynthetic pathway by downregulation and/or disruption of THC production. THC and other cannabis metabolites share a biosynthetic pathway; that cannabigerolic acid is a precursor of THC, CBD and cannabichromene. THCA synthase catalyzes the production of delta-9-tetrahydrocannabinolic acid from cannabigerolic acid; delta-9-tetrahydrocannabinolic undergoes thermal conversion to form THC. CBDA synthase catalyzes the production of cannabidiolic acid from cannabigerolic acid; cannabidiolic acid undergoes thermal conversion to CBD. CBCA synthase catalyzes the production of cannabichromenic acid from cannabigerolic acid; cannabichromenic acid undergoes thermal conversion to cannabichromene. A reduction in the production of THC, CBD, or cannabichromene will enhance production of the remaining metabolites in this shared pathway. For example, production of CBD and/or cannabichromene can be enhanced by inhibiting production of THC. THC production may be inhibited by inhibiting expression and/or activity of tetrahydrocannabinolic acid (THCA) synthase enzyme. In specific embodiments, there are provided methods of enhancing production of CBD and/or cannabichromene by inhibiting production of THC.

Also provided are plants and plant cells having modified production and/or disruption of one or more metabolites from a cannabinoid biosynthetic pathway. In certain embodiments, provided herein are cannabis and/or hemp plants and cells comprising an enhanced production and/or disruption of one or more secondary in a cannabinoid biosynthetic pathway. In certain embodiments, there are provided cannabis and/or hemp plants and cells having enhanced production of one or more secondary metabolites and downregulation of one or more other metabolites in the THC biosynthetic pathway. In certain embodiments, there are provided cannabis and/or hemp plants and cells having enhanced production of one or more secondary metabolites in the THC biosynthetic pathway and downregulated THC production. In specific embodiments, there are provided cannabis and/or hemp plants or portions thereof, and cells having enhanced production of CBD and/or cannabichromene and downregulated THC production as compared to unmodified plants.

Provided herein can also be genes that are overexpressed as compared to wildtype genes. Gene overexpression can be used to increase the production of intermediary compounds to generate a greater amount of a compound of interest. Any intermediary compound may be modulated for greater expression such as but not limited to: cannabigerolic acid (CBGA), highly functional tetrahydrocannabinolic acid (THCA), and cannabidiolic acid (CBDA) enzymes.

Gene overexpression can also be applied to increase the amount of cannflavins A and B by modulating their precursors luteolin and/or chrysoeriol. Alternatively provided herein can also be increasing the activity of CsPT3. Provided herein can also be increasing the conversion of chrysoeriol into cannflavins A or B.

Provided herein can be a method comprising enhancing CBGA biosynthesis. In some cases, upregulation of geranyl-pyrophosphate—olivetolic acid geranyltransferase (GOT) enzyme activity to increase synthesis of CBGA for example by CRISPR editing of the GOT promoter. Additionally, the conversion of CBGA to THC, CBD, and CBC can be blocked by CRISPR knock-out of any one of the synthase genes: THCAS, CBDAS, CBCAS, a synthase gene coding region, and/or their promoters. Sequence information regarding GOT is shown in Table 2. In some cases, GOT can be targeted utilizing genome editing methods provided herein. In some aspects, a disruption results in a decreased amount of CBCA synthase, CBDA synthase, THCA synthase, derivatives or analogues thereof as compared to an amount of the same compound of a comparable control plant absent a genomic disruption. In an aspect, disruption results in a decreased amount of CBCA synthase, CBDA synthase, THCA synthase, derivatives or analogues thereof compared to an amount of the same compound of a comparable control plant without said disruption wherein the decreased amount can be from about 1 fold, 2 fold, 3 fold, 4 fold, 5 fold, 8 fold, 10 fold, 20 fold, 30 fold, 40 fold, 50 fold, 60 fold, 70 fold, 80 fold, 90 fold, 100 fold, 120 fold, 140 fold, 160 fold, 180 fold, 200 fold, 250 fold, 300 fold, 350 fold, 400 fold, 500 fold, 700 fold, 800 fold, or about 1000 fold. In some cases, there can be from about 1%, 3%, 5%, 10%, 25%, 35%, 50%, 60%, 80%, 90%, 100%, 150%, 200%, 250%, 300%, 350%, or up to about 400% more formula IV measured by dry weight as compared to a comparable control plant without a genomic modification. In other cases, there can be from about 1%, 3%, 5%, 10%, 15%, 20%, 25%, 30%, 40%, 50%, 80%, 100%, 150%, or up to about 175% less cannabichromenic acid (CBCA) as measured by dry weight as compared to a comparable control plant without a genomic modification.

TABLE 2
Geranyl-pyrophosphate-olivetolic acid geranyltransferase (GOT)
gene sequence information. Gene information extracted from:
Vegara et al. Gene copy number is associated with phytochemistry
in Cannabis sativa. A single hit to the olivetolate geranyltransferase
gene found with the mRNA sequence.

| Gene | Assembly | Paralog | Number | Region | Start | End |
|---|---|---|---|---|---|---|
| Olivetolate | Pineapple | 003891 | 1 | Exon | 1 | 226 |
| Geranyl- | Banana | | 1 | Intron | 227 | 406 |
| transferase | Bubba Kush | | 2 | Exon | 407 | 539 |
| (GOT) | (PBBK) | | 2 | Intron | 540 | 3808 |
| | | | 3 | Exon | 3809 | 3955 |
| | | | 3 | Intron | 3956 | 4361 |
| | | | 4 | Exon | 4362 | 4591 |
| | | | 4 | Intron | 4592 | 5083 |
| | | | 5 | Exon | 5084 | 5154 |
| | | | 5 | Intron | 5155 | 5612 |
| | | | 6 | Exon | 5613 | 5704 |
| | | | 6 | Intron | 5705 | 5796 |
| | | | 7 | Exon | 5797 | 5916 |
| | | | 7 | Intron | 5917 | 6679 |
| | | | 8 | Exon | 6680 | 6741 |
| | | | 8 | Intron | 6742 | 6843 |
| | | | 9 | Exon | 6844 | 6876 |
| | | | 9 | Intron | 6877 | 7003 |
| | | | 10 | Exon | 7004 | 7077 |

Finally, production of Olivetolic Acid can be increased by upregulating (i) The Polyketide Cyclase enzyme Olivetolic Acid Cyclase (OAC) and/or (ii) The Polyketide Synthase enzyme Olivetolic Acid Synthase (OLS) by CRISPR editing of OAC and/or OLS promoters. Exemplary genomic regions of the OLS sequence that can be targeted for genome editing is shown in Table 3.

TABLE 3
Polyketide Synthase enzyme Olivetolic Acid Synthase (OLS)
gene sequence information. Gene information extracted
from: Vegara et al. Gene copy number is associated with
phytochemistry in Cannabis sativa. Hits found using C.
Sativa Olivetol synthase (NCBI accession AB164375.1).

| Gene | Assembly | Paralog | Number | Region | Start | End |
|---|---|---|---|---|---|---|
| Olivetolic | Purple | 15717 | 1 | Exon | 1 | 156 |
| Acid | Kush (PK) | | 1 | Intron | 157 | 317 |
| Synthase | | | 2 | Exon | 318 | 1319 |
| | | 16618 | 1 | Exon | 1 | 156 |
| | | | 1 | Intron | 157 | 308 |
| | | | 2 | Exon | 309 | 1310 |

In other cases, there can be from about 1%, 3%, 5%, 10%, 15%, 20%, 25%, 30%, 40%, 50%, 80%, 100%, 150%, or up to about 175% less cannabidiolic acid (CBDA) as measured by dry weight as compared to a comparable control plant without a genomic modification. In other cases, there can be from about 1%, 3%, 5%, 10%, 15%, 20%, 25%, 30%, 40%, 50%, 80%, 100%, 150%, or up to about 175% less tetrahydrocannabinolic acid (THCA) as measured by dry weight as compared to a comparable control plant without a genomic modification. Additionally, in some cases, an increased amount of cannabinol (CBN), a derivative, or analog thereof can be observed as compared to an amount of the same compound in a comparable control plant without a genetic modification. A genomic modification can comprise those provided herein such as but not limited to a disruption of a gene encoding a THCA synthase or portion thereof. In an aspect, a disruption results in an increased amount of THCA synthase as compared to an amount of the same compound in a comparable control plant without a genomic disruption. In some cases, a CBDA synthase and CBCA synthase are genomically disrupted resulting in a decreased amount of CBDA synthase and CBCA synthase as compared to a comparable control plant without a genomic disruption. In another aspect, a disruption provided herein can result in increased UV absorption of a transgenic plant provided herein as compared to a comparable control plant absent a disruption.

In some aspects, THCV biosynthesis can be enhanced. In an aspect, a transgenic plant provided herein can comprise an increased amount of tetrahydrocannabivarin (THCV), a derivative or analog thereof as compared to an amount of the same compound in a comparable control plant without a genetic modification. Engineering strategies for enhancing THCV biosynthesis comprise: A. Increasing production of THCVA substrate CBGVA by upregulation of: (i) GOT Enzyme activity to increase synthesis of CBGVA, and/or (ii) modulating enzymes producing the CBGVA precursors GPP and DA: Geranyl pyrophosphate synthase (GPPS) and polyketide synthase (PKS) enzyme plus Divarinic acid cyclase (DAC) respectively, by CRISPR editing of enzyme promoters. B. Increasing conversion of CBGVA to THCVA by upregulation of THC synthase enzyme. This modulation can increase THC and THCV yields. CRISPR editing can be performed to increase activity of the THC synthase promoter and/or CRIPSR knock-out of the competing synthesis pathways utilizing the precursor compounds of THC synthase, such as CBD synthase and CBC synthase knock-out. C. Blocking Olivetolic Acid production to prevent GOT enzyme from producing CBGA and depleting the pool of OAC substrate needed for CBGVA by CRISPR disruption of one or both of the genes needed for OAC production (Olivetolic Acid Cyclase (OAC) and Olivetolic Acid Synthase (OLS). Coding sequence information for OAC is provided in Table 4. Additionally, a genetic modification can comprise a disruption of a first of group of genes, for example pigment genes, wherein a disruption results in an increased amount of Formula I, a derivative, or analog thereof. In some cases, exemplary pigments can include any one of: chlorophyll, anthocyanins, such as the flavonoids, carotenoids, such as Beta-carotene, lycopene, alpha-carotene, beta-cryptoxanthin, lutein, and zeaxanthin.

TABLE 4
Cannabis sativa olivetolic acid cyclase mRNA,
complete cds. GenBank: JN679224.1

| SEQ ID | |
|---|---|
| NO | Sequence |
| 2 | AAAAAAGAAGAAGAAGAAGAAAGTTGAGAAAGA |
| | GAATGGCAGTGAAGCATTTGATTGTATTGAAGTT |
| | CAAAGATGAAATCACAGAAGCCCAAAAGGAAGAA |
| | TTTTTCAAGACGTATGTGAATCTTGTGAATATCATC |
| | CCAGCCATGAAAGATGTATACTGGGGTAAAGATGT |
| | GACTCAAAAGAATAAGGAAGAAGGGTACACTCACA |
| | TAGTTGAGGTAACATTTGAGAGTGTGGAGACTATTC |
| | AGGACTACATTATTCATCCTGCCCATGTTGGATTTGG |
| | AGATGTCTATCGTTCTTTCTGGGAAAAACTTCTCATT |
| | TTTGACTACACACCACGAAAGTAGACTATATATAGT |
| | AGCCGACCAAGCTGCCTTCATCTTCATCTTCTCAAATA |
| | ATATATCTAATATCTAATTATATAATAATAACTACTTA |
| | ATAAAAGACTGTGTTTATAACATTAAATA |
| | ATAATAATAATAAAGTCTTTTGTAGCT |

In some aspects, a genetic modification comprises a disruption of a second group of

genes, wherein a disruption results in a decreased amount of

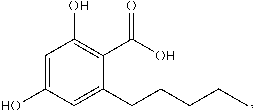 [SMILES: CCCCCc1cc(O)cc(O)c1C(=O)O]

a derivative, or analog thereof. A second group of genes can comprise: OAC, OLS, coding regions thereof, and combinations thereof. In an aspect, a disruption can comprise a disruption of a THCA synthase that results in an increased amount of THCA synthase, a derivative, or an analog thereof as compared to an amount of the same compound in a comparable control plant without a disruption. In an aspect, a genetic modification comprises a disruption of a third group of genes encoding CBCA synthase and CBDA synthase respectively. In some cases, a disruption results in a decreased amount of CBCA synthase and CBDA synthase, derivatives or analogs thereof. In some cases, a disruption can be in a coding region of a gene or portion of a gene. In some aspects, from about 1%, 35, 5%, 10%, 15%, 20%, 25%, 30%, 35%, 40%, 45%, 50%, 60%, 70%, 80%, 85%, 90%, 100%, 125%, 150%, or up to about 175% more tetrahydrocannabivarin (THCV) is observed as measured by dry weight as compared to a comparable control plant without a modification. In other cases, from about 1%, 35, 5%, 10%, 15%, 20%, 25%, 30%, 35%, 40%, 45%, 50%, 60%, 70%, 80%, 85%, 90%, 100%, 125%, or up to about 150% less cannabichromevarin (CBCV) and/or cannabidivarin (CBDV) is observed as measured by dry weight as compared to a comparable control plant without a modification.

In some cases, biosynthesis of cannabinolic acid (CBNA) can be modulated. Decarboxylation of THCs produces CBN and occurs slowly under ambient conditions (the rate increases with temperature). Heat and light can cause THC to degrade to CBN. Therefore, conditions can be genetically engineered to enhance this process or increase the precursors to in turn increase the degradation of THC. Strategies to enhance biosynthesis comprise: (i) Upregulation of the THC synthase enzyme. To increase the yield of THC and thus increase yield of CBN produced by its natural degradation. CRISPR editing to increase activity of the THC synthase promoter and/or CRIPSR knock-out of the competing synthesis pathways utilizing the precursor compounds of THC synthase, such as CBD synthase and CBC synthase knock-out. (ii) CRISPR genetic engineering of the Cannabis plant to increase its rate of THC to CBN Degradation. Such as modifying the genes that make the flowers and leaves absorb more UV light (such as pigment genes) to increase the light-mediated degradation of THC. (iii) Convert THC to CBN in plant tissue extracts. In some aspects, plant extracts of purified THC can be heated and oxidized to CBN, the precise conditions to optimize the process to obtain maximum conversion yields can be defined. In some cases, different species and strains of marijuana can produce different pigments in leaves and flowers of the marijuana plant due to varying levels of pigments in the cells and tissues. In some aspects, certain pigments or combinations of pigments result in elevated absorption of sunlight to cells and tissues, which in turn could enhance the conversion of THC to CBN in the presence of elevated levels of UV light entering (or reflecting less) from cells and tissues of plants provided herein. Exemplary pigments can include any one of: chlorophyll, anthocyanins, such as the flavonoids, carotenoids, such as Beta-carotene, lycopene, alpha-carotene, beta-cryptoxanthin, lutein, and zeaxanthin. In some cases, a transgenic plant provided herein can comprise from about 1%, 3%, 5%, 10%, 15%, 20%, 25%, 30%, 35%, 40%, 45%, 50%, 55%, 60%, 65%, 70%, 75%, or up to about 80% more THC as measured by dry weight as compared to a comparable control plant without a genetic modification. In another aspect, a transgenic plant provided herein can comprise from about 1%, 3%, 5%, 10%, 15%, 18%, 20%, 25%, 30%, 35%, 40%, 45%, 50%, 55%, 60%, 65%, 70%, 75%, 80%, 85%, 90%, or up to about 125% less CBCA as measured by dry weight as compared to a comparable control plant without a genetic modification. In another aspect, a transgenic plant provided herein can comprise from about 1%, 3%, 5%, 10%, 15%, 18%, 20%, 25%, 30%, 35%, 40%, 45%, 50%, 55%, 60%, 65%, 70%, 75%, 80%, 85%, 90%, or up to about 125% less CBDA as measured by dry weight as compared to a comparable control plant without a genetic modification.

In cases where a gene that encodes a protein of interest has not been identified, provided can be methods utilizing nucleotide sequence of genes have been discovered, partially or fully, and can be used to map the complete gene sequence to the Sativa genome build. In instances where a publicly available sequence is not available, a gene sequence based on sequencing of the gene in DNA isolated from Cannabis and hemp using guide sequences from paralogs and orthologs of the genes can be used.

In some aspects, the efficiency of genomic disruption of a cannabis and/or hemp plants or any part thereof, including but not limited to a cell, with any of the nucleic acid delivery platforms described herein, can result in disruption of a gene or portion thereof at about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or up to about 100% as measured by nucleic acid or protein analysis.

In some instances, by disrupting a compound involved in the cannabinoid biosynthesis pathway an increase in production of another compound involved in the same cannabinoid biosynthesis pathway may be observed. For example, disruption of a cannabinoid may lead to an increase of about 1 fold, 2 fold, 3 fold, 4 fold, 5 fold, 6 fold, 7 fold, 8 fold, 9 fold, 10 fold, 15 fold, 30 fold, 50 fold, 100 fold, 150 fold, 200 fold, 250 fold, 300 fold, 350 fold, 400 fold, 450 fold, or up to about 500 fold protein production of a different cannabinoid.

In one embodiment, the cannabis cultivar produces an assayable combined cannabidiolic acid and cannabidiol concentration of about 18% to about 60% by weight. In one embodiment, the cannabis cultivar produces an assayable combined cannabidiolic acid and cannabidiol concentration of about 20% to about 40% by weight. In one embodiment, the cannabis cultivar produces an assayable combined cannabidiolic acid and cannabidiol concentration of about 20% to about 30% by weight. In one embodiment, the cannabis cultivar produces an assayable combined cannabidiolic acid and cannabidiol concentration of about 25% to about 35% by weight. It should be understood that any sub value or subrange from within the values described above are contemplated for use with the embodiments described herein.

In some cases, included are methods for producing a medical cannabis composition, the method comprising obtaining a cannabis and/or hemp plant, growing the cannabis and/or hemp plant under plant growth conditions to produce plant tissue from the cannabis and/or hemp plant, and preparing a medical cannabis composition from the plant tissue or a portion thereof. In one aspect, described herein is a cannabis plant that can be a cannabis cultivar that produces substantially high levels of CBD (and/or CBDA) and substantially low levels of THC (and/or THCA) as compared to an unmodified comparable cannabis plant and/or cannabis cell.

Described are cannabis plants and/or plant cells having modified production of THC as compared to wild-type plants (for example, original cultivars). In certain embodiments, there is provided cannabis plants and/or cells having downregulated expression and/or activity of THCA synthase as compared to wild-type plants (for example, original cultivars). In certain embodiments the cannabis plants and/or cells produce reduced amounts or no THC. In certain embodiments of the cannabis plants and/or cells with reduced amounts or no THC, there is increased production of other metabolites on the THC biosynthesis pathway.

In certain embodiments, provided herein are cannabis plants and cells having enhanced production of one or more secondary metabolites in the THC biosynthetic pathway and downregulated or genomically disrupted THC production. In specific embodiments, there is provided cannabis plants and cells having enhanced production of CBD and/or Cannabichromene and downregulated or disrupted THC production.

In certain embodiments, there is provided cannabis plants and/or cells having enhanced production of one or more secondary metabolites which share steps and intermediates in the THC biosynthetic pathway and down-regulated expression and/or activity of THCA synthase. In specific embodiments, there is provided cannabis plants and/or cells having enhanced production of CBD and/or Cannabichromene and down-regulated expression and/or activity of THCA synthase.

Cannabis plants can be engineered to have modified expression and/or activity of other proteins in addition to THCA synthase. For example, the cannabis plants may also include modified expression and/or activity of other enzymes sharing intermediates with THCA synthase, such as CBDA synthase, CBCA synthase. Likewise, the cannabis plants of the invention may be crossed with plants having specific phenotypes. Cannabis plants with modified secondary metabolite production may be non-mutagenized, mutagenized, or transgenic, and the progeny thereof. In certain embodiments, the cannabis plants exhibiting modified secondary metabolite are the result of spontaneous mutations. In certain embodiments, the cannabis plants exhibiting modified secondary metabolite have been mutagenized by chemical or physical means. For example, ethylmethane sulfonate (EMS) may be used as a mutagen or radiation, such as x-ray, gamma-ray, and fast-neutron radiation may be used as a mutagen. In certain other embodiments, the cannabis plants exhibiting modified secondary metabolite are genetically engineered, for example with a Clustered Regularly Interspaced Short Palindromic Repeats (CRISPR) system.

In an aspect, provided herein can also be genetically engineered plants that produce mixtures of cannabinoids. In some cases, mixtures of cannabinoids can be at altered ratios as compared to their wildtype counterpart plants. For example, in some cases a ratio of THC to CBD may be 1:1. In some cases a ratio of THC to CBD may be 0:2, 0:3, 0:4, 0:5, 0:6, 0:7, 0:8, 0:00, 0:20, 0: 40, 0:50, 0:80, 0:100, 0:300, 0:500, 0:700, 0:900, 0:1000, 0.5:2, 0.5:3, 0.5:4, 0.5:5, 0.5:6, 0.5:7, 0.5:8, 0.5:10, 0.5:20, 0.5: 40, 0.5:50, 0.5:80, 0.5:100, 0.5:300, 0.5:500, 0.5:700, 0.5:900, 0.5:1000, 0:2, 0:3, 0:4, 0:5, 0:6, 0:7, 0:8, 0:00, 0:20, 0: 40, 0:50, 0:80, 0:100, 0:300, 0:500, 0:700, 0:900, 0:1000. Provided herein can also be methods of enhancing and/or synergism of administration of rare cannabinoids, terpenes, and botanical compounds. For example, a mixture can comprise a composition or compositions comprising a rare cannabinoid, a terpenes, a botanical compound, and any combination hereof

In some cases, compositions and methods provided herein can comprise evaluating a subject composition or method in a glutamate-GABA system. For example, a subject composition comprising a cannabinoid may modulate a glutamate-GABA system in a subject administered the cannabinoid composition. The expression of CB1 receptors varies between brain areas and neuronal cell types. In the hippocampus, GABAergic cells show high, whereas glutamatergic neurons a low CB1 receptor expression. The neuronal expression of CB2 receptors in the central nervous system is very low and restricted to some brainstem nuclei and to the cerebellum. CB2 receptor expression in astrocytes and microglia generally exceeds the expression of CB1 receptors. Thus, the primary receptors for cannabinoid signaling in the brain are CB1 on neurons and CB2 on glia cells. Accordingly, biological effects of cannabinoids are mainly mediated by two members of the G-protein-coupled receptor family, cannabinoid receptors 1 (CB₁R) and 2 (CB₂R).

In some instances, CB₁R can be prominently expressed in the central nervous system (CNS) and has drawn great attention as it participates in a variety of brain function modulations, including executive, emotional, reward, and memory processing via direct interactions with the endocannabinoid system and indirect effects on the glutamatergic, GABAergic and dopaminergic systems. Unlike CB₁R, CB₂R can be considered as a “peripheral” cannabinoid receptor. However, this concept has been challenged recently by the identification of functional CB2Rs throughout the central nervous system (CNS). When compared with CB₁R, brain CB₂R exhibits several unique features: (1) CB₂Rs have lower expression levels than CB₁Rs in the CNS, suggesting that CB₂Rs may not mediate the effects of cannabis under normal physiological conditions; (2) CB₂Rs are dynamic and inducible; thus, under some pathological conditions (e.g., addiction, inflammation, anxiety, epilepsy etc.), CB₂R expression can be upregulated in the brain, suggesting CB₂R involvement in various psychiatric and neurological diseases; (3) brain CB₂Rs are mainly expressed in neuronal somatodendritic areas (postsynaptic), while CB₁Rs are predominantly expressed in neuronal presynaptic terminals, suggesting an opposite role of CB₁Rs and CB₂Rs in regulation of neuronal firing and neurotransmitter release. Based on these characteristics, CB₂Rs have been considered to be an important substrate for neuroprotection, and targeting CB₂Rs can offer a novel therapeutic strategy for treating neuropsychiatric and neurological diseases without CB₁R-mediated side effects.

Various methods may be utilized to identify potential targets for gene editing in a cannabinoid biosynthesis pathway. In some cases, any one of: bioinformatics, gRNA design, CRISPR reagent construction, plant transformation, plant regeneration, and/or genotyping can be utilized. Bioinformatics can comprise gene mapping, gene alignment and copy number analysis, and gene annotation. gRNA design can comprise gRNA grouping to design clusters of guides for intended function, rank and selection of guides based on target gene specificity and off-targets within the cannabis genome. CRISPR reagent construction can comprise generation of infection-ready AGRO reagents to co-deliver Cas9 that has been cannabis codon optimized and gRNA. Plant transformation and regeneration can comprise infecting plant tissue with CRISPR AGRO (for example callus), techniques to isolate cannabis protoplasts and transform RNP reagents, and/or development of techniques to obtain growing plantlets from transformed tissue. Genotyping can comprise isolating plant DNA and analyzing a target sequence. Functional analysis can comprise analyzing cannabinoid content in plant tissue and quantifying relevant cannabinoids.

### Genetic Engineering

Provided herein can be systems of genomic engineering. Systems of genomic engineering can include any one of clustered regularly interspaced short palindromic repeats (CRISPR) enzyme, transcription activator-like effector (TALE)-nuclease, transposon-based nuclease, Zinc finger nuclease, meganuclease, argonaute, or Mega-TAL. In some aspects, a genome editing system can utilize a guiding polynucleic acid comprising DNA, RNA, or combinations thereof. In some cases, a guide can be a guide DNA or a guide RNA.

### I. Clustered Regularly Interspaced Short Palindromic Repeats (CRISPR)

In some cases, genetic engineering can be performed using a CRISPR system or portion thereof. A CRISPR system can be a multicomponent system comprising a guide polynucleotide or a nucleic acid encoding the guide polynucleotide and a CRISPR enzyme or a nucleic acid encoding the CRISPR enzyme. A CRISPR system can also comprise any modification of the CRISPR components or any portions of any of the CRISPR components.

Methods described herein can take advantage of a CRISPR system. There are at least five types of CRISPR systems which all incorporate guide RNAs and Cas proteins and encoding polynucleic acids. The general mechanism and recent advances of CRISPR system is discussed in Cong, L. et al., “Multiplex genome engineering using CRISPR systems,” Science, 339(6121): 819-823 (2013); Fu, Y. et al., “High-frequency off-target mutagenesis induced by CRISPR-Cas nucleases in human cells,” Nature Biotechnology, 31, 822-826 (2013); Chu, V T et al. “Increasing the efficiency of homology-directed repair for CRISPR-Cas9-induced precise gene editing in mammalian cells,” Nature Biotechnology 33, 543-548 (2015); Shmakov, S. et al., “Discovery and functional characterization of diverse Class 2 CRISPR-Cas systems,” Molecular Cell, 60, 1-13 (2015); Makarova, K S et al., “An updated evolutionary classification of CRISPR-Cas systems,”, Nature Reviews Microbiology, 13, 1-15 (2015). Site-specific cleavage of a target DNA occurs at locations determined by both 1) base-pairing complementarity between the guide RNA and the target DNA (also called a protospacer) and 2) a short motif in the target DNA referred to as the protospacer adjacent motif (PAM). A PAM can be a canonical PAM or a non-canonical PAM. For example, an engineered cell, such as a plant cell, can be generated using a CRISPR system, e.g., a type II CRISPR system. A Cas enzyme used in the methods disclosed herein can be Cas9, which catalyzes DNA cleavage. Enzymatic action by Cas9 derived from Streptococcus pyogenes or any closely related Cas9 can generate double stranded breaks at target site sequences which hybridize to about 20 nucleotides of a guide sequence and that have a protospacer-adjacent motif (PAM) following the about 20 nucleotides of the target sequence. In some aspects, less than 20 nucleotides can be hybridized. In some aspects, more than 20 nucleotides can be hybridized. Provided herein can be genomically disrupting activity of a THCA synthase comprising introducing into a cannabis and/or hemp plant or a cell thereof at least one RNA-guided endonuclease comprising at least one nuclear localization signal or nucleic acid encoding at least one RNA-guided endonuclease comprising at least one nuclear localization signal, at least one guiding nucleic acid encoding at least one guide RNA. In some aspects, a modified plant or portion thereof can be cultured.

### Clustered Regularly Interspaced Short Palindromic Repeats (CRISPR) Enzyme

A CRISPR enzyme can comprise or can be a Cas enzyme. In some aspects, a nucleic acid that encodes a Cas protein or portion thereof can be utilized in embodiments provided herein. Non-limiting examples of Cas enzymes can include Cas1, Cas1B, Cas2, Cas3, Cas4, Cas5, Cas5d, Cas5t, Cas5h, Cas5a, Cas6, Cas7, Cas8, Cas9, Cas10, Csy1 , Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, C2c1, C2c2, C2c3, Cpf1, CARF, DinG, homologues thereof, or modified versions thereof. In some cases, a catalytically dead Cas protein can be used, for example a dCas9. An unmodified CRISPR enzyme can have DNA cleavage activity, such as Cas9. A CRISPR enzyme can direct cleavage of one or both strands at a target sequence, such as within a target sequence and/or within a complement of a target sequence. In some aspects, a target sequence is at least about 18 nucleotides, at least 19 nucleotides, at least 20 nucleotides, at least 21 nucleotides, or at least 22 nucleotides in length. In some cases, a target sequence is at most 17 nucleotides in length. In some aspects, a target can be selected from a sequence comprising homology from about 50%, 60%, 70%, 80%, 90%, 95%, 96%, 97%, 98%, 99%, or up to about 100% to any one of: SEQ ID NO: 1 to SEQ ID NO: 7.

In some aspects, a target sequence can be found within an intron or exon of a gene. In some cases, a CRISPR system can target an exon of a gene involved in a cannabinoid biosynthesis pathway. For example, a CRISPR enzyme can direct cleavage of one or both strands within or within about 1, 2, 3, 4, 5, 6, 7, 8, 9, 10, 15, 20, 25, 50, 100, 200, 500, or more base pairs from the first or last nucleotide of a target sequence. For example, a CRISPR enzyme can direct cleavage of one or both strands within or within about 1, 2, 3, 4, 5, 6, 7, 8, 9, 10, 15, 20, 25, 50, 100, 200, 500, or more base pairs from a PAM sequence. In some cases, a guide polynucleotide binds a target sequence from 3 to 10 nucleotides from a PAM. A vector that encodes a CRISPR enzyme that is mutated with respect to a corresponding wild-type enzyme such that the mutated CRISPR enzyme lacks the ability to cleave one or both strands of a target polynucleotide containing a target sequence can be used. A Cas protein can be a high-fidelity Cas protein such as Cas9HiFi. In some cases, a Cas protein can be modified. For example, a Cas protein modification can comprise N7-Methyl-Gppp (2′-O-Methyl-A).

Cas9 can refer to a polypeptide with at least or at least about 50%, 60%, 70%, 80%, 90%, 100% sequence identity and/or sequence similarity to a wild type exemplary Cas9 polypeptide (e.g., Cas9 from S. pyogenes). Cas9 can refer to a polypeptide with at most or at most about 50%, 60%, 70%, 80%, 90%, 100% sequence identity and/or sequence similarity to a wild type exemplary Cas9 polypeptide (e.g., from S. pyogenes). Cas9 can refer to the wild type or a modified form of the Cas9 protein that can comprise an amino acid change such as a deletion, insertion, substitution, variant, mutation, fusion, chimera, or any combination thereof. In some cases, a CRISPR enzyme, such as Cas, can be codon optimized for expression in a plant.

A polynucleotide encoding an endonuclease (e.g., a Cas protein such as Cas9) can be codon optimized for expression in particular cells, such as plant cells. This type of optimization can entail the mutation of foreign-derived (e.g., recombinant) DNA to mimic the codon preferences of the intended host organism or cell while encoding the same protein.

An endonuclease can comprise an amino acid sequence having at least or at least about 50%, 60%, 70%, 75%, 80%, 85%, 90%, 95%, 99%, or 100%, amino acid sequence identity to the nuclease domain of a wild type exemplary site-directed polypeptide (e.g., Cas9 from S. pyogenes).

S. pyogenes Cas9 (SpCas9), can be used as a CRISPR endonuclease for genome engineering. In some cases, a different endonuclease may be used to target certain genomic targets. In some cases, synthetic SpCas9-derived variants with non-NGG PAM sequences may be used. Additionally, other Cas9 orthologues from various species have been identified and these “non-SpCas9s” bind a variety of PAM sequences that could also be useful for the present invention. For example, the relatively large size of SpCas9 (approximately 4kb coding sequence) means that plasmids carrying the SpCas9 cDNA may not be efficiently expressed in a cell. Conversely, the coding sequence for Staphylococcus aureus Cas9 (SaCas9) is approximately 1 kilobase shorter than SpCas9, possibly allowing it to be efficiently expressed in a cell.

Alternatives to S. pyogenes Cas9 may include RNA-guided endonucleases from the Cpf1 family. Unlike Cas9 nucleases, the result of Cpf1-mediated DNA cleavage is a double-strand break with a short 3′ overhang. Cpfl's staggered cleavage pattern may open up the possibility of directional gene transfer, analogous to traditional restriction enzyme cloning, which may increase the efficiency of gene editing. Like the Cas9 variants and orthologues described above, Cpf1 may also expand the number of sites that can be targeted by CRISPR to AT-rich regions or AT-rich genomes that lack the NGG PAM sites favored by SpCas9.

In some aspects Cas sequence can contain a nuclear localization sequence (NLS). A nuclear localization sequence can be from SV40. An NLS can be from at least one of: SV40, nucleoplasmin, importin alpha, C-myc, EGL-13, TUS, hnRNPA1, Mata2, or PY-NLS. An NLS can be on a C-terminus or an N-terminus of a Cas protein. In some cases, a Cas protein may contain from 1 to 5 NLS sequences. A Cas protein can contain 1, 2, 3, 4, 5, 6, 7, 8, 9, or up to 10 NLS sequences. A Cas protein, such as Cas9, may contain two NLS sequences. A Cas protein may contain a SV40 and nuceloplasmin NLS sequence. A Cas protein may also contain at least one untranslated region.

In some aspects, a vector that encodes a CRISPR enzyme can contain a nuclear localization sequences (NLS) sequence. In some cases, a vector can comprise one or more NLSs. In some cases, a vector can contain about 1, 2, 3, 4, 5, 6, 7, 8, 9, or 10 NLSs. For example, a CRISPR enzyme can comprise more than or more than about 1, 2, 3, 4, 5, 6, 7, 8, 9, 10 NLSs at or near the ammo-terminus, more than or more than about 1, 2, 3, 4, 5, 6, 7, 8, 9, 10, NLSs at or near the carboxyl-terminus, or any combination of these (e.g., one or more NLS at the ammo-terminus and one or more NLS at the carboxyl terminus). When more than one NLS is present, each can be selected independently of others, such that a single NLS can be present in more than one copy and/or in combination with one or more other NLSs present in one or more copies.

An NLS can be monopartite or bipartite. In some cases, a bipartite NLS can have a spacer sequence as opposed to a monopartite NLS. An NLS can be from at least one of: SV40, nucleoplasmin, importin alpha, C-myc, EGL-13, TUS, hnRNPA1, Mata2, or PY-NLS. An NLS can be located anywhere within the polypeptide chain, e.g., near the N- or C-terminus. For example, the NLS can be within or within about 1, 2, 3, 4, 5, 10, 15, 20, 25, 30, 40, 50 amino acids along a polypeptide chain from the N- or C-terminus. Sometimes the NLS can be within or within about 50 amino acids or more, e.g., 100, 200, 300, 400, 500, 600, 700, 800, 900, or 1000 amino acids from the N- or C-terminus.

Any functional concentration of Cas protein can be introduced to a cell. For example, 15 micrograms of Cas mRNA can be introduced to a cell. In other cases, a Cas mRNA can be introduced from 0.5 micrograms to 100 micrograms. A Cas mRNA can be introduced from 0.5, 5, 10, 15, 20, 25, 30, 35, 40, 45, 50, 55, 60, 65, 70, 75, 80, 85, 90, 95, or 100 micrograms.

In some cases, a dual nickase approach may be used to introduce a double stranded break or a genomic break. Cas proteins can be mutated at known amino acids within either nuclease domains, thereby deleting activity of one nuclease domain and generating a nickase Cas protein capable of generating a single strand break. A nickase along with two distinct guide RNAs targeting opposite strands may be utilized to generate a double stranded break (DSB) within a target site (often referred to as a “double nick” or “dual nickase” CRISPR system). This approach may dramatically increase target specificity, since it is unlikely that two off-target nicks will be generated within close enough proximity to cause a DSB.

A nuclease, such as Cas9, can be tested for identity and potency prior to use. For example, identity and potency can be determined using at least one of spectrophotometric analysis, RNA agarose gel analysis, LC-MS, endotoxin analysis, and sterility testing. In some cases, a nuclease sequence, such as a Cas9 sequence can be sequenced to confirm its identity. In some cases, a Cas protein, such as a Cas9 protein, can be sequenced prior to clinical or therapeutic use. For example, a purified in vitro transcription product can be assessed by polyacrylamide gel electrophoresis to verify no other mRNA species exist or substantially no other mRNA species exist within a clinical product other than Cas9. Additionally, purified mRNA encoding a Cas protein, such as Cas9, can undergo validation by reverse-transcription followed by a sequencing step to verify identity at a nucleotide level. A purified in vitro transcription product can be assessed by polyacrylamide gel electrophoresis (PAGE) to verify that an mRNA is the size expected for Cas9 and substantially no other mRNA species exist within a clinical or therapeutic product.

In some cases, an endotoxin level of a nuclease, such as Cas9, can be determined. A clinically/therapeutically acceptable level of an endotoxin can be less than 3 EU/mL. A clinically/therapeutically acceptable level of an endotoxin can be less than 2 EU/mL. A clinically/therapeutically acceptable level of an endotoxin can be less than 1 EU/mL. A clinically/therapeutically acceptable level of an endotoxin can be less than 0.5 EU/mL.

In some cases, a nuclease, such as Cas9, can undergo sterility testing. A clinically/therapeutically acceptable level of a sterility testing can be 0 or denoted by no growth on a culture. A clinically/therapeutically acceptable level of a sterility testing can be less than 0.5%, 0.3%, 0.1%, or 0.05% growth.

### Guiding Polynucleic Acid

A guiding polynucleic acid can be DNA or RNA. A guiding polynucleic acid can be single stranded or double stranded. In some cases, a guiding polynucleic acid can contains regions of single stranded areas and double stranded areas. A guiding polynucleic acid can also form secondary structures. As used herein, the term “guide RNA (gRNA),” and its grammatical equivalents can refer to an RNA which can be specific for a target DNA and can form a complex with a Cas protein. A guide RNA can comprise a guide sequence, or spacer sequence, that specifies a target site and guides an RNA/Cas complex to a specified target DNA for cleavage. For example, a guide RNA can target a CRISPR complex to a target gene or portion thereof and perform a targeted double strand break. Site-specific cleavage of a target DNA occurs at locations determined by both 1) base-pairing complementarity between a guide RNA and a target DNA (also called a protospacer) and 2) a short motif in a target DNA referred to as a protospacer adjacent motif (PAM). In some cases, gRNAs can be designed using an algorithm which can identify gRNAs located in early exons within commonly expressed transcripts.

In some cases, a guide polynucleotide can be complementary to a target sequence of a gene encoding: OAC, OLS, GOT, CBCA synthase, CBDA synthase, and/or THCA synthase. In some aspects, a gRNA or gDNA can bind a target sequence selected from a sequence comprising homology from about 50%, 60%, 70%, 80%, 90%, 95%, 96%, 97%, 98%, 99%, or up to about 100% to any one of: SEQ ID NO: 1 to SEQ ID NO: 7. In another aspect, a gRNA or gDNA can bind a target sequence described in a genome from Table 2 and/or Table 3.

Functional gene copies, gene variants and pseudogenes are mapped and aligned to produce a sequence template for CRISPR design. In some cases, multiple guide RNAs targeting sequences conserved across aligned copies of THCA synthase are designed to disrupt the early coding sequence and introduce mutations in the coding sequence, such as frameshift mutation indels. In some cases, a guide RNAs can be selected that has a low occurrence of off-target sites elsewhere in the Cannabis and hemp genome.

In an aspect, a CRISPR gRNA library may be generated and utilized to screen variant plants by DNA analysis. Multiplex CRISPR engineering can generate diverse genotypes of novel cannabinoid-producing cannabis plants. In some cases, these plants produce elevated levels of minor, rare, and/or poorly researched cannabinoids.

In some cases, a gRNA can be designed to target at exon of a gene involved in a cannabinoid biosynthesis pathway. In some cases, gRNAs can be designed to disrupt an early coding sequence. In an aspect, subject guide RNAs can be clustered into two categories: those intended to disrupt the production of functional proteins by targeting coding sequences having early positions within these genes to introduce frameshift mutation indels (KO Guides); and those which target sequences spread within gene regulatory regions (Expression modulating guides). Additionally, guide RNAs can be selected that have the lowest occurrence of off-target sites elsewhere in the cannabis and hemp genome.

In some cases, a gRNA can be selected based on the pattern of indels it inserts into a target gene. Candidate gRNAs can be ranked by off-target potential using a scoring system that can take into account: (a) the total number of mismatches between the gRNA sequence and any closely matching genomic sequences; (b) the mismatch position(s) relative to the PAM site which correlate with a negative effect on activity for mismatches falling close to the PAM site; (c) the distance between mismatches to account for the cumulative effect of neighboring mismatches in disrupting guide-DNA interactions; and any combination thereof. In some cases, a greater number of mismatches between a gRNA and a genomic target site can yield a lower potential for CRISPR-mediated cleavage of that site. In some cases, a mismatch position is directly adjacent to a PAM site. In other cases, a mismatch position can be from 1 nucleotide up to 100 kilobases away from a PAM site. Candidate gRNAs comprising mismatches may not be adjacent to a PAM in some cases. In other cases, at least two candidate gRNAs comprising mismatches may bind a genome from 1 nucleotide up to 100 kilobases away from each other. A mismatch can be a substitution of a nucleotide. For example, in some cases a G will be substituted for a T. Mismatches between a gRNA and a genome may allow for reduced fidelity of CRISPR gene editing. In some cases, a positive scoring gRNA can be about 110 nucleotides in length and may contain no mismatches to a complementary genome sequence. In other cases, a positive scoring gRNA can be about 110 nucleotides in length and may contain up to 3 mismatches to a complementary genome sequence. In other cases, a positive scoring gRNA can be about 110 nucleotides in length and may contain up to 20 mismatches to a complementary genome sequence. In some cases, a guiding polynucleic acid can contain internucleotide linkages that can be phosphorothioates. Any number of phosphorothioates can exist. For example from 1 to about 100 phosphorothioates can exist in a guiding polynucleic acid sequence. In some cases, from 1 to 10 phosphorothioates are present. In some cases, 8 phosphorothioates exist in a guiding polynucleic acid sequence.

In some cases, top scoring gRNAs can be designed and selected and an on-target editing efficiency of each can be assessed experimentally in plant cells. In some cases, an editing efficiency as determined by TiDE analysis can exceed at least about 20%. In other cases, editing efficiency can be from about 20% to from about 50%, from about 50% to from about 80%, from about 80% to from about 100%. In some cases, a percent indel can be determined in a trial GMP run. For example, a final cellular product can be analyzed for on-target indel formation by Sanger sequencing and TIDE analysis. Genomic DNA can be extracted from about 1×10⁶ cells from both a control and experimental sample and subjected to PCR using primers flanking a gene that has been disrupted, such as a gene involved in a cannabinoid biosynthesis pathway. Sanger sequencing chromatograms can be analyzed using a TIDE software program that can quantify indel frequency and size distribution of indels by comparison of control and knockout samples.

A method disclosed herein also can comprise introducing into a cell or plant embryo at least one guide RNA or nucleic acid, e.g., DNA encoding at least one guide RNA. A guide RNA can interact with a RNA-guided endonuclease to direct the endonuclease to a specific target site, at which site the 5′ end of the guide RNA base pairs with a specific protospacer sequence in a chromosomal sequence.

A guide RNA can comprise two RNAs, e.g., CRISPR RNA (crRNA) and transactivating crRNA (tracrRNA). A guide RNA can sometimes comprise a single-guide RNA (sgRNA) formed by fusion of a portion (e.g., a functional portion) of crRNA and tracrRNA. A guide RNA can also be a dual RNA comprising a crRNA and a tracrRNA. A guide RNA can comprise a crRNA and lack a tracrRNA. Furthermore, a crRNA can hybridize with a target DNA or protospacer sequence.

As discussed above, a guide RNA can be an expression product. For example, a DNA that encodes a guide RNA can be a vector comprising a sequence coding for the guide RNA. A guide RNA can be transferred into a cell or organism by transfecting the cell or plant embryo with an isolated guide RNA or plasmid DNA comprising a sequence coding for the guide RNA and a promoter. In some aspects, a promoter can be selected from the group consisting of a leaf-specific promoter, a flower-specific promoter, a THCA synthase promoter, a CaMV35S promoter, a FMV35S promoter, and a tCUP promoter. A guide RNA can also be transferred into a cell or plant embryo in other way, such as using particle bombardment.

A guide RNA can be isolated. For example, a guide RNA can be transfected in the form of an isolated RNA into a cell or plant embryo. A guide RNA can be prepared by in vitro transcription using any in vitro transcription system. A guide RNA can be transferred to a cell in the form of isolated RNA rather than in the form of plasmid comprising encoding sequence for a guide RNA.

A guide RNA can comprise a DNA-targeting segment and a protein binding segment. A DNA-targeting segment (or DNA-targeting sequence, or spacer sequence) comprises a nucleotide sequence that can be complementary to a specific sequence within a target DNA (e.g., a protospacer). A protein-binding segment (or protein-binding sequence) can interact with a site-directed modifying polypeptide, e.g. an RNA-guided endonuclease such as a Cas protein. By “segment” it is meant a segment/section/region of a molecule, e.g., a contiguous stretch of nucleotides in an RNA. A segment can also mean a region/section of a complex such that a segment may comprise regions of more than one molecule. For example, in some cases a protein-binding segment of a DNA-targeting RNA is one RNA molecule and the protein-binding segment therefore comprises a region of that RNA molecule. In other cases, the protein-binding segment of a DNA-targeting RNA comprises two separate molecules that are hybridized along a region of complementarity.

A guide RNA can comprise two separate RNA molecules or a single RNA molecule. An exemplary single molecule guide RNA comprises both a DNA-targeting segment and a protein-binding segment.

An exemplary two-molecule DNA-targeting RNA can comprise a crRNA-like (“CRISPR RNA” or “targeter-RNA” or “crRNA” or “crRNA repeat”) molecule and a corresponding tracrRNA-like (“trans-acting CRISPR RNA” or “activator-RNA” or “tracrRNA”) molecule. A first RNA molecule can be a crRNA-like molecule (targeter-RNA), that can comprise a DNA-targeting segment (e.g., spacer) and a stretch of nucleotides that can form one half of a double-stranded RNA (dsRNA) duplex comprising the protein-binding segment of a guide RNA. A second RNA molecule can be a corresponding tracrRNA-like molecule (activator-RNA) that can comprise a stretch of nucleotides that can form the other half of a dsRNA duplex of a protein-binding segment of a guide RNA. In other words, a stretch of nucleotides of a crRNA-like molecule can be complementary to and can hybridize with a stretch of nucleotides of a tracrRNA-like molecule to form a dsRNA duplex of a protein-binding domain of a guide RNA. As such, each crRNA-like molecule can be said to have a corresponding tracrRNA-like molecule. A crRNA-like molecule additionally can provide a single stranded DNA-targeting segment, or spacer sequence. Thus, a crRNA-like and a tracrRNA-like molecule (as a corresponding pair) can hybridize to form a guide RNA. A subject two-molecule guide RNA can comprise any corresponding crRNA and tracrRNA pair.

A DNA-targeting segment or spacer sequence of a guide RNA can be complementary to sequence at a target site in a chromosomal sequence, e.g., protospacer sequence such that the DNA-targeting segment of the guide RNA can base pair with the target site or protospacer. In some cases, a DNA-targeting segment of a guide RNA can comprise from or from about 10 nucleotides to from or from about 25 nucleotides or more. For example, a region of base pairing between a first region of a guide RNA and a target site in a chromosomal sequence can be or can be about 10, 11, 12, 13, 14, 15, 16, 17, 18, 19, 20, 22, 23, 24, 25, or more than 25 nucleotides in length. Sometimes, a first region of a guide RNA can be or can be about 19, 20, or 21 nucleotides in length.

A guide RNA can target a nucleic acid sequence of or of about 20 nucleotides. A target nucleic acid can be less than or less than about 20 nucleotides. A target nucleic acid can be at least or at least about 5, 10, 15, 16, 17, 18, 19, 20, 21, 22, 23, 24, 25, 30 or more nucleotides. A target nucleic acid can be at most or at most about 5, 10, 15, 16, 17, 18, 19, 20, 21, 22, 23, 24, 25, 30 or more nucleotides. A target nucleic acid sequence can be or can be about 20 bases immediately 5′ of the first nucleotide of the PAM. A guide RNA can target a nucleic acid sequence of a gene that encodes a protein involved in the cannabinoid biosynthesis pathway. Exemplary proteins involved in the cannabinoid biosynthesis pathway are shown in Table 5 along with their genomic sequences. A guiding polynucleic acid, such as a gRNA, can bind to at least a portion of a genomic sequence provided in Table 5. In some cases, a gRNA can bind to a genomic sequence comprising at least or at least about 50%, 60%, 65%, 70%, 75%, 80%, 85%, 90%, 95%, 98%, or up to about 100% identity to a sequence provided in Table 3. In some cases, a guiding polynucleic acid, such as a guide RNA, can bind a genomic region from about 1 base pair to about 20 base pairs away from a PAM. A guide can bind a genomic region from about 1, 2, 3, 4,5 6, 7, 8, 9, 10, 11, 12, 13, 14, 15, 16, 17, 18, 19, or up to about 20 base pairs away from a PAM.

In some aspects, any one of the proteins provided in Table 5, involved in cannabinoid biosynthesis of C. sativia L may be disrupted using methods provided herein. Additionally, any precursor or target of the provided proteins involved in cannabinoid biosynthesis may be disrupted using methods provided herein. Further included are nucleic acid molecules, such as guide RNA (gRNA), that hybridize to the provided sequences in Table 5, sequences that encode for precursors thereof, or sequences that encode for targets thereof.

TABLE 5
Genomic sequences of proteins involved in cannabinoid biosynthesis
in C. sativa L that can be targeted with subject gRNA

| | | SEQ | |
|---|---|---|---|
| | | ID | |
| Enzyme | Abbreviation | NO: | Sequence |
| livetol | LS | 3 | ATGAATCATCTTCGTGCTGAGGGTCCGGCCTCCGTTC |
| synthase | | | TCGCCATTGGCACCGCCAATCCGGAGAACATTTTATT |
| | | | ACAAGATGAGTTTCCTGACTACTATTTTCGCGTCACCA |
| | | | AAAGTGAACACATGACTCAACTCAAAGAAAAGTTTCG |
| | | | AAAAATATGTGACAAAAGTATGATAAGGAAACGTAAC |
| | | | TGTTTCTTAAATGAAGAACACCTAAAGCAAAACCCAAG |
| | | | ATTGGTGGAGCACGAGATGCAAACTCTGGATGCACGTC |
| | | | AAGACATGTTGGTAGTTGAGGTTCCAAAACTTGGGAAG |
| | | | GATGCTTGTGCAAAGGCCATCAAAGAATGGGGTCAACC |
| | | | CAAGTCTAAAATCACTCATTTAATCTTCACTAGCGCATC |
| | | | AACCACTGACATGCCCGGTGCAGACTACCATTGCGCTA |
| | | | AGCTTCTCGGACTGAGTCCCTCAGTGAAGCGTGTGATG |
| | | | ATGTATCAACTAGGCTGTTATGGTGGTGGAACCGTTCTA |
| | | | CGCATTGCCAAGGACATAGCAGAGAATAACAAAGGCGC |
| | | | ACGAGTTCTCGCCGTGTGTTGTGACATAATGGCTTGCTTG |
| | | | TTTCGTGGGCCTTCAGAGTCTGACCTCGAATTACTAGTGG |
| | | | GACAAGCTATCTTTGGTGATGGGGCTGCTGCGGTGATTGT |
| | | | TGGAGCTGAACCCGATGAGTCAGTTGGGGAAAGGCCGAT |
| | | | ATTTGAGTTGGTGTCAACTGGGCAAACAATCTTACCAAAC |
| | | | TCGGAAGGAACTATTGGGGGACATATAAGGGAAGCAGGA |
| | | | CTGATATTTGATTTACATAAGGATGTGCCTATGTTGATCTC |
| | | | TAATAATATTGAGAAATGTTTGATTGAGGCATTTACTCCTA |
| | | | TTGGGATTAGTGATTGGAACTCCATATTTTGGATTACACAC |
| | | | CCAGGTGGGAAAGCTATTTTGGACAAAGTGGAGGAGAAGT |
| | | | TGCATCTAAAGAGTGATAAGTTTGTGGATTCACGTCATGTG |
| | | | CTGAGTGAGCATGGGAATATGTCTAGCTCAACTGTCTTGTT |
| | | | TGTTATGGATGAGTTGAGGAAGAGGTCGTTGGAGGAAGGG |
| | | | AAGTCTACCACTGGAGATGGATTTGAGTGGGGTGTTCTTTT |
| | | | TGGGTTTGGACCAGGTTTGACTGTCGAAAGAGTGGTCGTGCGT |
| | | | AGTGTTCCCATCAAATATTAA |
| livctolic | AC | 4 | MAVKHLIVLKFKDEITEAQKEEFFKTYVNLVNIIPAMKDVYW |
| acid cyclase | | | GKDVTQKNKEEGYTHIVEVTFESVETIQDYIIHPAHVGFGDVYRSF |
| | | | WEKLLIFDYTPRK |
| Cannabigerolic | CBGAS | 5 | ATGGGACTCTCATCAGTTTGTACCTTTTCATTTCAAACTAATTA |
| acid | | | CCATACTTTATTAAATCCTCACAATAATAATCCCAAAACCTCAT |
| synthase | | | TATTATGTTATCGACACCCCAAAACACCAATTAAATACTCTTA |
| | | | C |
| | | | AATAATTTTCCCTCTAAACATTGCTCCACCAAGAGTTTFCATCT |
| | | | ACAAAACAAATGCTCAGAATCATTATCAATCGCAAAAAATTCC |
| | | | ATTAGGGCAGCTACTACAAATCAAACTGAGCCTCCAGAATCT |
| | | | GATAATCATTCAGTAGCAACTAAAATTTTAAACTTTGGGAAGG |
| | | | CATGTTGGAAACTTCAAAGACCATATACAATCATAGCATTTAC |
| | | | T |
| | | | TCATGCGCTTGTGGATTGTTTGGGAAAGAGTTGTTGCATAACA |
| | | | C |
| | | | AAATTTAATAAGTTGGTCTCTGATGTTCAAGGCATTCTTTTTTT |
| | | | T |
| | | | GGTGGCTATATTATGCATTGCTTCTTTTACAACTACCATCAATC |
| | | | A |
| | | | GATTTACGATCTTCACATTGACAGAATAAACAAGCCTGATCTA |
| | | | C |
| | | | CACTAGCTTCAGGGGAAATATCAGTAAACACAGCTTGGATTAT |
| | | | GAGCATAATTGTGGCACTGTTTGGATTGATAATAACTATAAAA |
| | | | ATGAAGGGTGGACCACTCTATATATTTGGCTACTGTTTTGGTAT |
| | | | TTTTGGTGGGATTGTCTATTCTGTTCCACCATTTAGATGGAAGC |
| | | | AAAATCCTTCCACTGCATTTCTTCTCAATTTCCTGGCCCATATT |
| | | | ATTACAAATTTCACATTTTATTATGCCAGCAGAGCAGCTCTTG |
| | | | GCCTACCATTTGAGTTGAGGCCTTCTTTTACTTTCCTGCTAGCA |
| | | | TTTATGAAATCAATGGGTTCAGCTTTGGCTTTAATCAAAGATG |
| | | | C |
| | | | TTCAGACGTTGAAGGCGACACTAAATTTGGCATATCAACCTTG |
| | | | G |
| | | | CAAGTAAATATGGTTCCAGAAACTTGACATTATTTTGTTCGG |
| | | | A |
| | | | ATTGTTCTCCTATCCTATGTGGCTGCTATACTTGCTGGGATTAT |
| | | | C |
| | | | TGGCCCCAGGCTTTCAACAGTAACGTAATGTTACTTTCTCATGC |
| | | | AATCTTAGCATTTTGGTTAATCCTCCAGACTCGAGATTTTG |
| | | | CGTTAACAAATTACGACCCGGAAGCAGGCAGAAGATTTT |
| | | | ACGAGTTCATGTGGAAGCTTTATTATGCTGAATATTTAGTATAT |
| | | | GTTTTCATATAA |
| Cannabichromenic | CBCAS | 6 | ATGAATTGCTCAACATTCTCCTTTTGGTTTGTTTG |
| acid synthase | | | CAAAATAATATTTTTCTTTCTCTCATTCAATATCC |
| | | | AAATTTCAATAGCTAATCCTCAAGAAAACTTCCTT |
| | | | AAATGCTTCTCGGAATATATTCCTAACAATCCAGC |
| | | | AAATCCAAAATTCATATACACTCAACACGACCAAT |
| | | | TGTATATGTCTGTCCTGAATTCGACAATACAAAAT |
| | | | CTTAGATTCACCTCTGATACAACCCCAAAACCACT |
| | | | CGTTATTGTCACTCCTTCAAATGTCTCCCATATCC |
| | | | AGGCCAGTATTCTCTGCTCCAAGAAAGTTGGTTTG |
| | | | CAGATTCGAACTCGAAGCGGTGGCCATGATGCTGA |
| | | | GGGTTTGTCCTACATATCTCAAGTCCCATTTGCTAT |
| | | | AGTAGACTTGAGAAACATGCATACGGTCAAAGTAG |
| | | | ATATTCATAGCCAAACTGCGTGGGTTGAAGCCGGA |
| | | | GCTACCCTTGGAGAAGTTTATTATTGGATCAATGA |
| | | | GATGAATGAGAATTTTAGTTTTCCTGGTGGGTATTG |
| | | | CCCTACTGTTGGCGTAGGTGGACACTTTAGTGGAG |
| | | | GAGGCTATGGACATTGATGCGAAATTATGGCCTTG |
| | | | CGGCTGATAATATCATTGATGCACACTTACTTCAATG |
| | | | TTGATGGAAAAGTTCTAGATCGAAAATCCATGGGA |
| | | | GAAGATCTATTTTGGGCTATACGTGGTGGAGGAGGA |
| | | | GAAAACTTTGGAATCATTGCAGCATGTAAAATCAA |
| | | | ACTTGTTGTTGTCCCATCAAAGGCTACTATATTCAGT |
| | | | GTTAAAAAGAACATGGAGATACATGGGCTTGTCAAG |
| | | | TTATTTAACAAATGGCAAAATATTGCTTACAAG |
| | | | TATGACAAAGATTTAATGCTCACGACTACTTCA |
| | | | GAACTAGGAATATTACAGATAATCATGGGAAGA |
| | | | ATAAGACTACAGTACATGGTTACTTCTCTTCCAT |
| | | | TTTTCTTGGTGGAGTGGATAGTCTAGTTGACTTG |
| | | | ATGAACAAGAGCTTTCCTGAGTTGGGTATTAAAA |
| | | | AAACTGATTGCAAAGAATTGAGCTGGATTGATAC |
| | | | AACCATCTTCTACAGTGGTGTTGTAAATTACAACA |
| | | | CTGCTAATTTTAAAAAGGAAATTTTGCTTGATAGA |
| | | | TCAGCTGGGAAGAAGACGGCTTTCTCAATTAAGT |
| | | | TAGACTATGTTAAGAAACTAATACCTGAAACTGC |
| | | | AATGGTCAAAATTTTGGAAAAATTATATGAAGA |
| | | | AGAGGTAGGAGTTGGGATGTATGTGTTGTACCCT |
| | | | TACGGTGGTATAATGGATGAGATTTCAGAATCAG |
| | | | CAATTCCATTCCCTCATCGAGCTGGAATAATGTAT |
| | | | GAACTTTGGTACACTGCTACCTGGGAGAAGCAAG |
| | | | AAGATAACCTAAAAGCATATAAACTGGGTTCGAAG |
| | | | TGTTTATAATTTCACAACTCCTTATGTGTCCCAAAA |
| | | | TCCAAGATTGGCGTATCTCAATTATAGGGACCTTG |
| | | | ATTTAGGAAAAACTAATCCTGAGAGTCCTAATAAT |
| | | | TACACACAAGCACGTATTTGGGGTGAAAAGTATTT |
| | | | TGGTAAAAATTTTAACAGGTTAGTTAAGGTGAAAA |
| | | | CCAAAGCTGATCCCAATAATTTTTTTAGAAACGAAC |
| | | | AAAGTATCCCACCTCTTCCACCGCGTCATCAT |
| Cannabidiolic | CBDAS | 7 | ATGAAGTGCTCAACATTCTCCTTTTGGTTTGTTTGCAAGAT |
| acid | | | AATATTTTTCTTTTTCTCATTCAATATCCAAACTTCCATTGC |
| synthase | | | TAATCCTCGAGAAAACTTCCTTAAATGCTTCTCGCAATATA |
| | | | TTCCCAATAATGCAACAAATCTAAAACTCGTATACACTCAA |
| | | | AACAACCCATTGTATATGTCTGTCCTAAATTCGACAATACA |
| | | | CAATCTTAGATTCACCTCTGACACAACCCCAAAACCACTTG |
| | | | TTATCGTCACTCCTTCACATGTCTCTCATATCCAAGGCACTA |
| | | | TTCTATGCTCCAAGAAAGTTGGCTTGCAGATTCGAACTCGA |
| | | | AGTGGTGGTCATGATTCTGAGGGCATGTCCTACATATCTCA |
| | | | AGTCCCATTTGTTATAGTAGACTTGAGAAACATGCGTTCAA |
| | | | TCAAAATAGATGTTCATAGCCAAACTGCATGGGTTGAAGCC |
| | | | GGAGCTACCCTTGGAGAAGTTTATTATTGGGTTAATGAGAA |
| | | | AAATGAGAATCTTAGTTTGGCGGCTGGGTATTGCCCTACTG |
| | | | TTTGCGCAGGTGGACACTTTGGTGGAGGAGGCTATGGACCA |
| | | | TTGATGAGAAACTATGGCCTCGCGGCTGATAATATCATTGA |
| | | | TGCACACTTAGTCAACGTTCATGGAAAAGTGCTAGATCGA |
| | | | AAATCTATGGGGGAAGATCTCTTTTGGGCTTTACGTGGTG |
| | | | GTGGAGCAGAAAGCTTCGGAATCATTGTAGCATGGAAAA |
| | | | TTAGACTGGTTGCTGTCCCAAAGTCTACTATGTTTAGTGTT |
| | | | AAAAAGATCATGGAGATACATGAGCTTGTCAAGTTAGTTA |
| | | | ACAAATGGCAAAATATTGCTTACAAGTATGACAAAGATTT |
| | | | ATTACTCATGACTCACTTCATAACTAGGAACATTACAGATA |
| | | | ATCAAGGGAAGAATAAGACAGCAATACACACTTACTTCTC |
| | | | TTCAGTTTTCCTTGGTGGAGTGGATAGTCTAGTCGACTTGA |
| | | | TGAACAAGAGTTTTCCTGAGTTGGGTATTAAAAAAACGGA |
| | | | TTGCAGACAATTGAGCTGGATTGATACTATCATCTTCTATA |
| | | | GTGGTGTTGTAAATTACGACACTGATAATTTTAACAAGGA |
| | | | AATTTTGCTTGATAGATCCGCTGGGCAGAACGGTGCTTTCA |
| | | | AGATTAAGTTAGACTACGTTAAGAAACCAATTCCAGAATC |
| | | | TGTATTTGTCCAAATTTTGGAAAAATTATATGAAGAAGATA |
| | | | TAGGAGCTGGGATGTATGCGTTGTACCCTTACGGTGGTATA |
| | | | ATGGATGAGATTTCAGAATCAGCAATTCCATTCCCTCATCG |
| | | | AGCTGGAATCTTGTATGAGTTATGGTACATATGTAGTTGGG |
| | | | AGAAGCAAGAAGATAACGAAAAGCATCTAAACTGGATTAG |
| | | | AAATATTTATAACTTCATGACTCCTTATGTGTCCAAAAATCC |
| | | | AAGATTGGCATATCTCAATTATAGAGACCTTGATATAGGAA |
| | | | TAAATGATCCCAAGAATCCAAATAATTACACACAAGCACGT |
| | | | ATTTGGGGTGAGAAGTATTTTGGTAAAAATTTTGACAGGCT |
| | | | AGTAAAAGTGAAAACCCTGGTTGATCCCAATAACTTTTTTA |
| | | | GAAACGAACAAAGCATCCCACCTCTTCCACGGCATCGTCA |
| | | | TTAA |
| Tetrahydro- | THCAS | 8 | AAAAAAATCATTAGGACTGAAGAAAAATGAATTGCTCAG |
| cannabinolic | | | CATTTTCCTTTTGGTTTGTTTGCAAAATAATATTTTTCTTT |
| acid | | | CTCTCATTCCATATCCAAATTTCAATAGCTAATCCTCGAG |
| synthase | | | AAAACTTCCTTAAATGCTTCTCAAAACATATTCCCAACA |
| | | | ATGTAGCAAATCCAAAACTCGTATACACTCAACACGACC |
| | | | AATTGTATATGTCTATCCTGAATTCGACAATACAAAATC |
| | | | TTAGATTCATCTCTGATACAACCCCAAAACCACTCGTTAT |
| | | | TGTCACTCCTTCAAATAACTCCCATATCCAAGCAACTATT |
| | | | TTATGCTCTAAGAAAGTTGGCTTGCAGATTCGAACTCGAA |
| | | | GCGGTGGCCATGATGCTGAGGGTATGTCCTACATATCTCA |
| | | | AGTCCCATTTGTTGTAGTAGACTTGAGAAACATGCATTCG |
| | | | ATCAAAATAGATGTTCATAGCCAAACTGCGTGGGTTGAAG |
| | | | CCGGAGCTACCCTTGGAGAAGTTTATTATTGGATCAATGA |
| | | | GAAGAATGAGAATCTTAGTTTTCCTGGTGGGTATTGCCCT |
| | | | ACTGTTGGCGTAGGTGGACACTTTAGTGGAGGAGGCTATG |
| | | | GAGCATTGATGCGAAATTATGGCCTTGCGGCTGATAATATT |
| | | | ATTGATGCACACTTAGTCAATGTTGATGGAAAAGTTCTAGA |
| | | | TCGAAAATCCATGGGAGAAGATCTGTTTTGGGCTATACGTG |
| | | | GTGGTGGAGGAGAAAACTTTGGAATCATTGCAGCATGGAAA |
| | | | ATCAAACTGGTTGCTGTCCCATCAAAGTCTACTATATTCAGT |
| | | | GTTAAAAAGAACATGGAGATACATGGGCTTGTCAAGTTATTT |
| | | | AACAAATGGCAAAATATTGCTTACAAGTATGACAAAGATTTA |
| | | | GTACTCATGACTCACTTCATAACAAAGAATATTACAGATAAT |
| | | | CATGGGAAGAATAAGACTACAGTACATGGTTACTTCTCTTCA |
| | | | ATTTTTCATGGTGGAGTGGATAGTCTAGTCGACTTGATGAAC |
| | | | AAGAGCTTTCCTGAGTTGGGTATTAAAAAAACTGATTGCA |
| | | | AAGAATTTAGCTGGATTGATACAACCATCTTCTACAGTGG |
| | | | TGTTGTAAATTTTAACACTGCTAATTTTAAAAAGGAAATTT |
| | | | TGCTTGATAGATCAGCTGGGAAGAAGACGGCTTTCTCAATT |
| | | | AAGTTAGACTATGTTAAGAAACCAATTCCAGAAACTGCAA |
| | | | TGGTCAAAATTTTGGAAAAATTATATGAAGAAGATGTAGG |
| | | | AGCTGGGATGTATGTGTTGTACCCTTACGGTGGTATAATGG |
| | | | AGGAGATTTCAGAATCAGCAATTCCATTCCCTCATCGAGCT |
| | | | GGAATAATGTATGAACTTTGGTACACTGCTTCCTGGGAGAA |
| | | | GCAAGAAGATAATGAAAAGCATATAAACTGGGTTCGAAGT |
| | | | GTTTATAATTTTACGACTCCTTATGTGTCCCAAAATCCAAGA |
| | | | TTGGCGTATCTCAATTATAGGGACCTTGATTTAGGAAAAAC |
| | | | TAATCATGCGAGTCCTAATAATTACACACAAGCACGTATTT |
| | | | GGGGTGAAAAGTATTTTGGTAAAAATTTTAACAGGTTAGTT |
| | | | AAGGTGAAAACTAAAGTTGATCCCAATAATTTTTTTAGAAA |
| | | | CGAACAAAGTATCCCACCTCTTCCACCGCATCATCATTAATT |
| | | | ATCTTTAAATAGATATATTTCCCTTATCAATTAGTTAATCATT |
| | | | ATACCATACATACATTTATTGTATATAGTTTATCTACTCATAT |
| | | | TATGTATGCTCCCAAGTATGAAAATCTACATTAGAACTGTGT |
| | | | AGACAATCATAAGATATATTTAATAAAATAAATTGTCTTTC |
| | | | TTATTTCAATAGCAAATAAAATAATATTATTTTAAAAAAAAAA |
| | | | AAAAAAA |

A guide nucleic acid, for example, a guide RNA, can refer to a nucleic acid that can hybridize to another nucleic acid, for example, the target nucleic acid or protospacer in a genome of a cell. A guide nucleic acid can be RNA. A guide nucleic acid can be DNA. The guide nucleic acid can be programmed or designed to bind to a sequence of nucleic acid site-specifically. A guide nucleic acid can comprise a polynucleotide chain and can be called a single guide nucleic acid. A guide nucleic acid can comprise two polynucleotide chains and can be called a double guide nucleic acid.

A guide nucleic acid can comprise one or more modifications to provide a nucleic acid with a new or enhanced feature. A guide nucleic acid can comprise a nucleic acid affinity tag. A guide nucleic acid can comprise synthetic nucleotide, synthetic nucleotide analog, nucleotide derivatives, and/or modified nucleotides. A guide nucleic acid can comprise a nucleotide sequence (e.g., a spacer), for example, at or near the 5′ end or 3′ end, that can hybridize to a sequence in a target nucleic acid (e.g., a protospacer). A spacer of a guide nucleic acid can interact with a target nucleic acid in a sequence-specific manner via hybridization (i.e., base pairing). A spacer sequence can hybridize to a target nucleic acid that is located 5′ or 3′ of a protospacer adjacent motif (PAM). The length of a spacer sequence can be at least or at least about 5, 10, 15, 16, 17, 18, 19, 20, 21, 22, 23, 24, 25, 30 or more nucleotides. The length of a spacer sequence can be at most or at most about 5, 10, 15, 16, 17, 18, 19, 20, 21, 22, 23, 24, 25, 30 or more nucleotides.

A guide RNA can also comprise a dsRNA duplex region that forms a secondary structure. For example, a secondary structure formed by a guide RNA can comprise a stem (or hairpin) and a loop. A length of a loop and a stem can vary. For example, a loop can range from about 3 to about 10 nucleotides in length, and a stem can range from about 6 to about 20 base pairs in length. A stem can comprise one or more bulges of 1 to about 10 nucleotides. The overall length of a second region can range from about 16 to about 60 nucleotides in length. For example, a loop can be or can be about 4 nucleotides in length and a stem can be or can be about 12 base pairs. A dsRNA duplex region can comprise a protein-binding segment that can form a complex with an RNA-binding protein, such as an RNA-guided endonuclease, e.g. Cas protein.

A guide RNA can also comprise a tail region at the 5′ or 3′ end that can be essentially single-stranded. For example, a tail region is sometimes not complementarity to any chromosomal sequence in a cell of interest and is sometimes not complementarity to the rest of a guide RNA. Further, the length of a tail region can vary. A tail region can be more than or more than about 4 nucleotides in length. For example, the length of a tail region can range from or from about 5 to from or from about 60 nucleotides in length.

A guide RNA can be introduced into a cell or embryo as an RNA molecule. For example, an RNA molecule can be transcribed in vitro and/or can be chemically synthesized. A guide RNA can then be introduced into a cell or embryo as an RNA molecule. A guide RNA can also be introduced into a cell or embryo in the form of a non-RNA nucleic acid molecule, e.g., DNA molecule. For example, a DNA encoding a guide RNA can be operably linked to promoter control sequence for expression of the guide RNA in a cell or embryo of interest. A RNA coding sequence can be operably linked to a promoter sequence that is recognized by RNA polymerase III (Pol III).

A DNA molecule encoding a guide RNA can also be linear. A DNA molecule encoding a guide RNA can also be circular. A DNA sequence encoding a guide RNA can also be part of a vector. Some examples of vectors can include plasmid vectors, phagemids, cosmids, artificial/mini-chromosomes, transposons, and viral vectors. For example, a DNA encoding a RNA-guided endonuclease is present in a plasmid vector. Other non-limiting examples of suitable plasmid vectors include pUC, pBR322, pET, pBluescript, and variants thereof. Further, a vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like.

When both a RNA-guided endonuclease and a guide RNA are introduced into a cell as DNA molecules, each can be part of a separate molecule (e.g., one vector containing fusion protein coding sequence and a second vector containing guide RNA coding sequence) or both can be part of a same molecule (e.g., one vector containing coding (and regulatory) sequence for both a fusion protein and a guide RNA).

A Cas protein, such as a Cas9 protein or any derivative thereof, can be pre-complexed with a guide RNA to form a ribonucleoprotein (RNP) complex. The RNP complex can be introduced into plant cells. Introduction of the RNP complex can be timed. The cell can be synchronized with other cells at G1, S, and/or M phases of the cell cycle. The RNP complex can be delivered at a cell phase such that HDR is enhanced. The RNP complex can facilitate homology directed repair.

A guide RNA can also be modified. The modifications can comprise chemical alterations, synthetic modifications, nucleotide additions, and/or nucleotide subtractions. The modifications can also enhance CRISPR genome engineering. A modification can alter chirality of a gRNA. In some cases, chirality may be uniform or stereopure after a modification. A guide RNA can be synthesized. The synthesized guide RNA can enhance CRISPR genome engineering. A guide RNA can also be truncated. Truncation can be used to reduce undesired off-target mutagenesis. The truncation can comprise any number of nucleotide deletions. For example, the truncation can comprise 1, 2, 3, 4, 5, 10, 15, 20, 25, 30, 40, 50 or more nucleotides. A guide RNA can comprise a region of target complementarity of any length. For example, a region of target complementarity can be less than 20 nucleotides in length. A region of target complementarity can be more than 20 nucleotides in length. A region of target complementarity can target from about 5 bp to about 20 bp directly adjacent to a PAM sequence. A region of target complementarity can target about 13 bp directly adjacent to a PAM sequence. The polynucleic acids as described herein can be modified. A modification can be made at any location of a polynucleic acid. More than one modification can be made to a single polynucleic acid. A polynucleic acid can undergo quality control after a modification. In some cases, quality control may include PAGE, HPLC, MS, or any combination thereof. A modification can be a substitution, insertion, deletion, chemical modification, physical modification, stabilization, purification, or any combination thereof. A polynucleic acid can also be modified by 5′adenylate, 5′ guanosine-triphosphate cap, 5′N⁷-Methylguanosine-triphosphate cap, 5′triphosphate cap, 3′phosphate, 3′thiophosphate, 5′phosphate, 5′thiophosphate, Cis-Syn thymidine dimer, trimers, C12 spacer, C3 spacer, C6 spacer, dSpacer, PC spacer, rSpacer, Spacer 18, Spacer 9,3′-3′ modifications, 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′deoxyribonucleoside analog purine, 2′deoxyribonucleoside analog pyrimidine, ribonucleoside analog, 2′-0-methyl ribonucleoside analog, sugar modified analogs, wobble/universal bases, fluorescent dye label, 2′fluoro RNA, 2′O-methyl RNA, methylphosphonate, phosphodiester DNA, phosphodiester RNA, phosphothioate DNA, phosphorothioate RNA, UNA, pseudouridine-5′-triphosphate, 5-methylcytidine-5′-triphosphate, or any combination thereof. In some cases, a modification can be permanent. In other cases, a modification can be transient. In some cases, multiple modifications are made to a polynucleic acid. A polynucleic acid modification may alter physio-chemical properties of a nucleotide, such as their conformation, polarity, hydrophobicity, chemical reactivity, base-pairing interactions, or any combination thereof. In some aspects a gRNA can be modified. In some cases, a modification is on a 5′ end, a 3′ end, from a 5′ end to a 3′ end, a single base modification, a 2′-ribose modification, or any combination thereof. A modification can be selected from a group consisting of base substitutions, insertions, deletions, chemical modifications, physical modifications, stabilization, purification, and any combination thereof. In some cases, a modification is a chemical modification.

In some cases, a modification is a 2-O-methyl 3 phosphorothioate addition denoted as “m”. A phosphothioate backbone can be denoted as “(ps).” A 2-O-methyl 3 phosphorothioate addition can be performed from 1 base to 150 bases. A 2-O-methyl 3 phosphorothioate addition can be performed from 1 base to 4 bases. A 2-O-methyl 3 phosphorothioate addition can be performed on 2 bases. A 2-O-methyl 3 phosphorothioate addition can be performed on 4 bases. A modification can also be a truncation. A truncation can be a 5-base truncation. In some cases, a modification may be at C terminus and N terminus nucleotides.

A modification can also be a phosphorothioate substitute. In some cases, a natural phosphodiester bond may be susceptible to rapid degradation by cellular nucleases and; a modification of internucleotide linkage using phosphorothioate (PS) bond substitutes can be more stable towards hydrolysis by cellular degradation. A modification can increase stability in a polynucleic acid. A modification can also enhance biological activity. In some cases, a phosphorothioate enhanced RNA polynucleic acid can inhibit RNase A, RNase Tl, calf serum nucleases, or any combinations thereof. These properties can allow the use of PS-RNA polynucleic acids to be used in applications where exposure to nucleases is of high probability in vivo or in vitro. For example, phosphorothioate (PS) bonds can be introduced between the last 3-5 nucleotides at the 5′- or 3′-end of a polynucleic acid which can inhibit exonuclease degradation. In some cases, phosphorothioate bonds can be added throughout an entire polynucleic acid to reduce attack by endonucleases.

In another embodiment, down-regulating the activity of a THCA synthase or portion thereof comprises introducing into a cannabis and/or hemp plant or a cell thereof (i) at least one RNA-guided endonuclease comprising at least one nuclear localization signal or nucleic acid encoding at least one RNA-guided endonuclease comprising at least one nuclear localization signal, (ii) at least one guide RNA or DNA encoding at least one guide RNA, and, optionally, (iii) at least one donor polynucleotide such as a barcode; and culturing the cannabis and/or hemp plant or cell thereof such that each guide RNA directs an RNA-guided endonuclease to a targeted site in the chromosomal sequence where the RNA-guided endonuclease introduces a double-stranded break in the targeted site, and the double-stranded break is repaired by a DNA repair process such that the chromosomal sequence is modified, wherein the targeted site is located in the THCA synthase gene and the chromosomal modification interrupts or interferes with transcription and/or translation of the THCA synthase gene.

In some cases, a GUIDE-Seq analysis can be performed to determine the specificity of engineered guide RNAs. The general mechanism and protocol of GUIDE-Seq profiling of off-target cleavage by CRISPR system nucleases is discussed in Tsai, S. et al., “GUIDE-Seq enables genome-wide profiling of off-target cleavage by CRISPR system nucleases,” Nature, 33: 187-197 (2015). To assess off-target frequencies by next generation sequencing cells can be transfected with Cas9 mRNA and a guiding RNA. Genomic DNA can be isolated from transfected cells from about 72 hours post transfection and PCR amplified at potential off-target sites. A potential off-target site can be predicted using the Wellcome Trust Sanger Institute Genome Editing database (WGE) algorithm. Candidate off-target sites can be chosen based on sequence homology to an on-target site. In some cases, sites with about 4 or less mismatches between a gRNA and a genomic target site can be utilized. For each candidate off-target site, two primer pairs can be designed. PCR amplicons can be obtained from both untreated (control) and Cas9/gRNA-treated cells. PCR amplicons can be pooled. NGS libraries can be prepared using TruSeq Nano DNA library preparation kit (Illumina). Samples can be analyzed on an Illumina HiSeq machine using a 250 bp paired-end workflow. In some cases, from about 40 million mappable NGS reads per gRNA library can be acquired. This can equate to an average number of about 450,000 reads for each candidate off-target site of a gRNA. In some cases, detection of CRISPR-mediated disruption can be at a frequency as low as 0.1% at any genomic locus.

Computational predictions can be used to select candidate gRNAs likely to be the safest choice for a targeted gene. Candidate gRNAs can then tested empirically using a focused approach steered by computational predictions of potential off-target sites. In some cases, an assessment of gRNA off-target safety can employ a next-generation deep sequencing approach to analyze the potential off-target sites predicted by the CRISPR design tool for each gRNA. In some cases, gRNAs can be selected with fewer than 3 mismatches to any sequence in the genome (other than the perfect matching intended target). In some cases, a gRNA can be selected with fewer than 50, 40, 30, 20, 10, 5, 4, 3, 2, or 1 mismatch(es) to any sequence in a genome. In some cases, a computer system or software can be utilized to provide recommendations of candidate gRNAs with predictions of low off-target potential.

In some cases, potential off-target sites can be identified with at least one of: GUIDE-Seq and targeted PCR amplification, and next generation sequencing. In addition, modified cells, such as Cas9/gRNA-treated cells can be subjected to karyotyping to identify any chromosomal re-arrangements or translocations.

A gRNA can be introduced at any functional concentration. For example, a gRNA can be introduced to a cell at 10 micrograms. In other cases, a gRNA can be introduced from 0.5 micrograms to 100 micrograms. A gRNA can be introduced from 0.5, 5, 10, 15, 20, 25, 30, 35, 40, 45, 50, 55, 60, 65, 70, 75, 80, 85, 90, 95, or 100 micrograms.

A guiding polynucleic acid can have any frequency of bases. For example, a guiding polynucleic acid can have 29 As, 17 Cs, 23 Gs, 23 Us, 3 mGs, 1 mCs, and 4 mUs. A guiding polynucleic acid can have from about 1 to about 100 nucleotides. A guiding polynucleic acid can have from about 1 to 30 of a single polynucleotide. A guiding polynucleic acid can have from about 1 to 10, 10 to 20, or from 20 to 30 of a single nucleotide.

A guiding polynucleic acid can be tested for identity and potency prior to use. For example, identity and potency can be determined using at least one of spectrophotometric analysis, RNA agarose gel analysis, LC-MS, endotoxin analysis, and sterility testing. In some cases, identity testing can determine an acceptable level for clinical/therapeutic use. For example, an acceptable spectrophotometric analysis result can be 14±2 μL/vial at 5.0±0.5 mg/mL. an acceptable spectrophotometric analysis result can also be from about 10-20±2 μL/vial at 5.0±0.5 mg/mL or from about 10-20±2 μL/vial at about 3.0 to 7.0±0.5 mg/mL. An acceptable clinical/therapeutic size of a guiding polynucleic acid can be about 100 bases. A clinical/therapeutic size of a guiding polynucleic acid can be from about 5 bases to about 150 bases. A clinical/therapeutic size of a guiding polynucleic acid can be from about 20 bases to about 150 bases. A clinical/therapeutic size of a guiding polynucleic acid can be from about 40 bases to about 150 bases. A clinical/therapeutic size of a guiding polynucleic acid can be from about 60 bases to about 150 bases. A clinical/therapeutic size of a guiding polynucleic acid can be from about 80 bases to about 150 bases. A clinical/therapeutic size of a guiding polynucleic acid can be from about 100 bases to about 150 bases. A clinical/therapeutic size of a guiding polynucleic acid can be from about 110 bases to about 150 bases. A clinical/therapeutic size of a guiding polynucleic acid can be from about 120 bases to about 150 bases.

In some cases, a mass of a guiding polynucleic acid can be determined. A mass can be determined by LC-MS assay. A mass can be about 32,461.0 amu. A guiding polynucleic acid can have a mass from about 30,000 amu to about 50,000 amu. A guiding polynucleic acid can have a mass from about 30,000 amu to 40,000 amu, from about 40,000 amu to about 50,000 amu. A mass can be of a sodium salt of a guiding polynucleic acid.

In some cases, an endotoxin level of a guiding polynucleic acid can be determined. A clinically/therapeutically acceptable level of an endotoxin can be less than 3 EU/mL. A clinically/therapeutically acceptable level of an endotoxin can be less than 2 EU/mL. A clinically/therapeutically acceptable level of an endotoxin can be less than 1 EU/mL. A clinically/therapeutically acceptable level of an endotoxin can be less than 0.5 EU/mL.

In some cases, a guiding polynucleic acid can go sterility testing. A clinically/therapeutically acceptable level of a sterility testing can be 0 or denoted by no growth on a culture. A clinically/therapeutically acceptable level of a sterility testing can be less than 0.5% growth.

Guiding polynucleic acids can be assembled by a variety of methods, e.g., by automated solid-phase synthesis. A polynucleic acid can be constructed using standard solid-phase DNA/RNA synthesis. A polynucleic acid can also be constructed using a synthetic procedure. A polynucleic acid can also be synthesized either manually or in a fully automated fashion. In some cases, a synthetic procedure may comprise 5′-hydroxyl oligonucleotides can be initially transformed into corresponding 5′-H-phosphonate mono esters, subsequently oxidized in the presence of imidazole to activated 5′-phosphorimidazolidates, and finally reacted with pyrophosphate on a solid support. This procedure may include a purification step after the synthesis such as PAGE, HPLC, MS, or any combination thereof.

### Donor Sequences

In some cases, a donor sequence may be introduced to a genome of a cannabis and/or a hemp plant or portion thereof. In some cases, a donor is inserted into a genomic break. In some aspects, a donor comprises homology to sequencing flanking a target sequence. Methods of introducing a donor sequence are known to the skilled artisan but may include the use of homology arms. For example, a donor sequence can comprise homology arms to at least a portion of a genome that comprises a genomic break. In some cases, a donor sequence is randomly inserted into a genome of a cannabis or hemp plant cell genome.

In some cases, a donor sequence can be introduced in a site directed fashion using homologous recombination. Homologous recombination permits site specific modifications in endogenous genes and thus inherited or acquired mutations may be corrected, and/or novel alterations may be engineered into the genome. Homologous recombination and site-directed integration in plants are discussed in, for example, U.S. Pat. Nos. 5,451,513, 5,501,967 and 5,527,695.

In some aspects, a donor sequence comprises a promoter sequence. Increasing expression of designed gene products may be achieved by synthetically increasing expression by modulating promoter regions or inserting stronger promoters upstream of desired gene sequences. In some aspects, a promoter such as 35s and Ubil0 that are highly functional in Arabidopsis and other plants may be introduced. In some cases, a promoter that is highly functional in cannabis and/or hemp is introduced.

In some cases, a donor can be a barcode. A barcode can comprise a non-natural sequence. In some aspects, a barcode contains natural sequences. In some aspects, a barcode can be utilized to allow for identification of transgenic plants via genotyping. In some aspects, a donor sequence can be a marker. Selectable marker genes can include, for example, photosynthesis (atpB, tscA, psaA/B, petB, petA, ycf3, rpoA, rbcL), antibiotic resistance (rrnS, rrnL, aadA, nptII, aphA-6), herbicide resistance (psbA, bar, AHAS (ALS), EPSPS, HPPD, sul) and metabolism (BADH, codA, ARG8, ASA2) genes. The sul gene from bacteria has herbicidal sulfonamide-insensitive dihydropteroate synthase activity and can be used as a selectable marker when the protein product is targeted to plant mitochondria (U.S. Pat. No. 6,121,513). In some embodiments, the sequence encoding the marker may be incorporated into the genome of the cannabis and/or hemp. In some embodiments, the incorporated sequence encoding the marker may by subsequently removed from the transformed cannabis and/or hemp genome. Removal of a sequence encoding a marker may be facilitated by the presence of direct repeats before and after the region encoding the marker. Removal of the sequence encoding the marker can occur via the endogenous homologous recombination system of the organelle or by use of a site-specific recombinase system such as cre-lox or FLP/FRT.

In some cases, a marker can refer to a label capable of detection, such as, for example, a radioisotope, fluorescent compound, bioluminescent compound, a chemiluminescent compound, metal chelator, or enzyme. Examples of detectable markers include, but are not limited to, the following: fluorescent labels (e.g., FITC, rhodamine, lanthanide phosphors), enzymatic labels (e.g., horseradish peroxidase, β-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags).

Selectable or detectable markers normally comprise DNA segments that allow a cell, or a molecule marked with a “tag” inside a cell of interest, to be identified, often under specific conditions. Such markers can encode an activity, selected from, but not limited to, the production of RNA, peptides, or proteins, or the marker can provide a bonding site for RNA, peptides, proteins, inorganic and organic compounds or composites, etc. By way of example, selectable markers comprise, without being limited thereto, DNA segments that comprise restriction enzyme cleavage points, DNA segments comprising a fluorescent probe, DNA segments that encode products that provide resistance to otherwise toxic compounds, comprising antibiotics, e.g. spectinomycin, ampicillin, kanamycin, tetracycline, BASTA, neomycin-phosphotransferase II (NEO) and hygromycin-phosphotransferase (HPT), DNA segments that encode products that a plant target cell of interest would not have under natural conditions, e.g. tRNA genes, auxotrophic markers and the like, DNA segments that encode products that can be readily identified, in particular optically observable markers, e.g. phenotype markers such as—galactosidases, GUS, fluorescent proteins, e.g. green fluorescent protein (GFP) and other fluorescent proteins, e.g. blue (CFP), yellow (YFP) or red (RFP) fluorescent proteins, and surface proteins, wherein those fluorescent proteins that exhibit a high fluorescence intensity are of particular interest, because these proteins can also be identified in deeper tissue layers if, instead of a single cell, a complex plant target structure or a plant material or a plant comprising numerous types of tissues or cells can be to be analyzed, new primer sites for PCR, the recording of DNA sequences that cannot be modified in accordance with the present disclosure by restriction endonucleases or other DNA modified enzymes or effector domains, DNA sequences that are used for specific modifications, e.g. epigenetic modifications, e.g. methylations, and DNA sequences that carry a PAM motif, which can be identified by a suitable CRISPR system in accordance with the present disclosure, and also DNA sequences that do not have a PAM motif, such as can be naturally present in an endogenous plant genome sequence.

In one embodiment, a donor comprises a selectable, screenable, or scoreable marker gene or portion thereof. In some cases, a marker serves as a selection or screening device may function in a regenerable plant tissue to produce a compound that would confer upon the plant tissue resistance to an otherwise toxic compound. Genes of interest for use as a selectable, screenable, or scoreable marker would include but are not limited to gus, green fluorescent protein (gfp), luciferase (lux), genes conferring tolerance to antibiotics like kanamycin (Dekeyser et al., 1989) or spectinomycin (e.g. spectinomycin aminoglycoside adenyltransferase (aadA), genes that encode enzymes that give tolerance to herbicides like glyphosate (e.g. 5-enolpyruvylshikimate-3-phosphate synthase (EPSPS); glyphosate oxidoreductase (GOX); glyphosate decarboxylase; or glyphosate N-acetyltransferase (GAT), dalapon (e.g. dehI encoding 2,2-dichloropropionic acid dehalogenase conferring tolerance to 2,2-dichloropropionic acid, bromoxynil (haloarylnitrilase (Bxn) for conferring tolerance to bromoxynil, sulfonyl herbicides (e.g. acetohydroxyacid synthase or acetolactate synthase conferring tolerance to acetolactate synthase inhibitors such as sulfonylurea, imidazolinone, triazolopyrimidine, pyrimidyloxybenzoates and phthalide; encoding ALS, GST-II), bialaphos or phosphinothricin or derivatives (e.g. phosphinothricin acetyltransferase (bar) conferring tolerance to phosphinothricin or glufosinate, atrazine (encoding GST-III), dicamba (dicamba monooxygenase), or sethoxydim (modified acetyl-coenzyme A carboxylase for conferring tolerance to cyclohexanedione (sethoxydim) and aryloxyphenoxypropionate (haloxyfop), among others. Other selection procedures can also be implemented including positive selection mechanisms (e.g. use of the manA gene of E. coli, allowing growth in the presence of mannose), and dual selection (e.g. simultaneously using 75-1 00 ppm spectinomycin and 3-10 ppm glufosinate, or 75 ppm spectinomycin and 0.2-0.25 ppm dicamba). Use of spectinomycin at a concentration of about 25-1000 ppm, such as at about 150 ppm, can be also contemplated. In an embodiment, a detectable marker can be attached by spacer arms of various lengths to reduce potential steric hindrance.

In some cases, a donor polynucleotide comprises homology to sequences flanking a target sequence. In some cases, a donor polynucleotide introduces a stop codon into a gene provided herein for example a gene encoding at least one of: OAC, OLS, GOT, CBCA synthase, CBDA synthase, and THCA synthase. In some cases, a donor polynucleotide comprises a barcode, a reporter, or a selection marker.

### Transformation

Appropriate transformation techniques can include but are not limited to: electroporation of plant protoplasts; liposome-mediated transformation; polyethylene glycol (PEG) mediated transformation; transformation using viruses; micro-injection of plant cells; micro-projectile bombardment of plant cells; vacuum infiltration; and Agrobacterium tumeficiens mediated transformation. Transformation means introducing a nucleotide sequence into a plant in a manner to cause stable or transient expression of the sequence.

Following transformation, plants may be selected using a dominant selectable marker incorporated into the transformation vector. In certain embodiments, such marker confers antibiotic or herbicide resistance on the transformed plants, and selection of transformants can be accomplished by exposing the plants to appropriate concentrations of the antibiotic or herbicide. After transformed plants are selected and grown to maturity, those plants showing a modified trait are identified. The modified trait can be any of those traits described above. Additionally, expression levels or activity of the polypeptide or polynucleotide of the invention can be determined by analyzing mRNA expression using Northern blots, RT-PCR, RNA seq or microarrays, or protein expression using immunoblots or Western blots or gel shift assays.

Suitable methods for transformation of plant or other cells for use with the current invention are believed to include virtually any method by which DNA can be introduced into a cell, such as by direct delivery of DNA such as by PEG-mediated transformation of protoplasts, by desiccation/inhibition-mediated DNA uptake, by electroporation, by agitation with silicon carbide fibers, by Agrobacterium-mediated transformation and by acceleration of DNA coated particles. Through the application of techniques such as these, the cells of virtually any plant species may be stably transformed, and these cells developed into transgenic plants.

### Agrobacterium-Mediated Transformation

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA, for example comprising CRISPR systems or donors sequences, into plant cells is well known in the art. Agrobacterium-mediated transformation can be efficient in dicotyledonous plants and can be used for the transformation of dicots, including Arabidopsis, tobacco, tomato, alfalfa and potato. Indeed, while Agrobacterium-mediated transformation has been routinely used with dicotyledonous plants for a number of years. In some cases, agrobacterium-mediated transformation can be used in monocotyledonous plants. For example, Agrobacterium-mediated transformation techniques have now been applied to rice, wheat, barley, alfalfa and maize. In some aspects, Agrobacterium-Mediated Transformation can be used to transform a cannabis and/or hemp plant or cell thereof.

Modern Agrobacterium transformation vectors are capable of replication in E. coli as well as Agrobacterium, allowing for convenient manipulations as described. Moreover, recent technological advances in vectors for Agrobacterium-mediated gene transfer have improved the arrangement of genes and restriction sites in the vectors to facilitate the construction of vectors capable of expressing various polypeptide coding genes. In some aspects, a vector can have convenient multi-linker regions flanked by a promoter and a polyadenylation site for direct expression of inserted polypeptide coding genes and are suitable for purposes described herein. In addition, Agrobacterium containing both armed and disarmed Ti genes can be used for the transformations.

### Electroporation

In some aspects, a cannabis and/or hemp plant or cell thereof may be modified using electroporation. To effect transformation by electroporation, one may employ either friable tissues, such as a suspension culture of cells or embryogenic callus or alternatively one may transform immature embryos or other organized tissue directly. In this technique, one would partially degrade the cell walls of the chosen cells, such as cannabis and/or hemp cells, by exposing them to pectin-degrading enzymes (pectolyases) or mechanically wounding in a controlled manner.

Any transfection system can be utilized. In some cases, a Neon transfection system may be utilized. A Neon system can be a three-component electroporation apparatus comprising a central control module, an electroporation chamber that can be connected to a central control module by a 3-foot-long electrical cord, and a specialized pipette. In some cases, a specialized pipette can be fitted with exchangeable and/or disposable sterile tips. In some cases, an electroporation chamber can be fitted with exchangeable/disposable sterile electroporation cuvettes. In some cases, standard electroporation buffers supplied by a manufacturer of a system, such as a Neon system, can be replaced with GMP qualified solutions and buffers. In some cases, a standard electroporation buffer can be replaced with GMP grade phosphate buffered saline (PBS). A self-diagnostic system check can be performed on a control module prior to initiation of sample electroporation to ensure the Neon system is properly functioning. In some cases, a transfection can be performed in a class 1,000 biosafety cabinet within a class 10,000 clean room in a cGMP facility. In some cases, electroporation pulse voltage may be varied to optimize transfection efficiency and/or cell viability. In some cases, electroporation pulse width may be varied to optimize transfection efficiency and/or cell viability. In some cases, the number of electroporation pulses may be varied to optimize transfection efficiency and/or cell viability. In some cases, electroporation may comprise a single pulse. In some cases, electroporation may comprise more than one pulse. In some cases, electroporation may comprise 2 pulses, 3 pulses, 4 pulses, 5 pulses 6 pulses, 7 pulses, 8 pulses, 9 pulses, or 10 or more pulses.

In some aspects, protoplasts of plants may be used for electroporation transformation.

### Microprojectile Bombardment

Another method for delivering transforming DNA segments to plant cells in accordance with the invention is microprojectile bombardment. In this method, particles may be coated with nucleic acids and delivered into cells by a propelling force. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. In some aspects, DNA-coated particles may increase the level of DNA delivery via particle bombardment. For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into plant cells by acceleration is the Biolistics Particle Delivery System, which can be used to propel particles coated with DNA or cells through a screen, such as a stainless steel or Nytex screen, onto a filter surface covered with monocot plant cells cultured in suspension. The screen disperses the particles so that they are not delivered to the recipient cells in large aggregates.

### Other Transformation Methods

Additional transformation methods include but are not limited to calcium phosphate precipitation, polyethylene glycol treatment, electroporation, and combinations of these treatments.

To transform plant strains that cannot be successfully regenerated from protoplasts, other ways to introduce DNA into intact cells or tissues can be utilized. For example, regeneration of plants from immature embryos or explants can be affected as described. Also, silicon carbide fiber-mediated transformation may be used with or without protoplasting. Transformation with this technique can be accomplished by agitating silicon carbide fibers together with cells in a DNA solution. DNA passively enters as the cells are punctured.

In some cases, a starting cell density for genomic editing may be varied to optimize editing efficiency and/or cell viability. In some cases, the starting cell density for genomic editing may be less than about 1×10⁵ cells. In some cases, the starting cell density for electroporation may be at least about 1×10⁵ cells, at least about 2×10⁵ cells, at least about 3×10⁵ cells, at least about 4×10⁵ cells, at least about 5×10⁵ cells, at least about 6×10⁵ cells, at least about 7×10⁵ cells, at least about 8×10⁵ cells, at least about 9×10⁵ cells, at least about 1×10⁶ cells, at least about 1.5×10⁶ cells, at least about 2×10⁶ cells, at least about 2.5×10⁶ cells, at least about 3×10⁶ cells, at least about 3.5×10⁶ cells, at least about 4×10⁶ cells, at least about 4.5×10⁶ cells, at least about 5×10⁶ cells, at least about 5.5×10⁶ cells, at least about 6×10⁶ cells, at least about 6.5×10⁶ cells, at least about 7×10⁶ cells, at least about 7.5×10⁶ cells, at least about 8×10⁶ cells, at least about 8.5×10⁶ cells, at least about 9×10⁶ cells, at least about 9.5×10⁶ cells, at least about 1×10⁷ cells, at least about 1.2×10⁷ cells, at least about 1.4×10⁷ cells, at least about 1.6×10⁷ cells, at least about 1.8×10⁷ cells, at least about 2×10⁷ cells, at least about 2.2×10⁷ cells, at least about 2.4×10⁷ cells, at least about 2.6×10⁷ cells, at least about 2.8×10⁷ cells, at least about 3×10⁷ cells, at least about 3.2×10⁷ cells, at least about 3.4×10⁷ cells, at least about 3.6×10⁷ cells, at least about 3.8×10⁷ cells, at least about 4×10⁷ cells, at least about 4.2×10⁷ cells, at least about 4.4×10⁷ cells, at least about 4.6×10⁷ cells, at least about 4.8×10⁷ cells, or at least about 5×10⁷ cells.

The efficiency of genomic disruption of plants or any part thereof, including but not limited to a cell, with any of the nucleic acid delivery platforms described herein, can result in disruption of a gene or portion thereof at about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or up to about 100% as measured by nucleic acid or protein analysis.

### Plant Breeding

In some embodiments, the plants of the present disclosure can be used to produce new plant varieties. In some embodiments, the plants are used to develop new, unique and superior varieties or hybrids with desired phenotypes. In some embodiments, selection methods, e.g., molecular marker assisted selection, can be combined with breeding methods to accelerate the process. In some embodiments, a method comprises (i) crossing any one of the plants provided herein comprising the expression cassette as a donor to a recipient plant line to create a FI population; (ii) selecting offspring that have expression cassette. Optionally, the offspring can be further selected by testing the expression of the gene of interest. In some embodiments, complete chromosomes of a donor plant are transferred. For example, the transgenic plant with an expression cassette can serve as a male or female parent in a cross pollination to produce offspring plants by receiving a transgene from a donor plant thereby generating offspring plants having an expression cassette. In a method for producing plants having the expression cassette, protoplast fusion can also be used for the transfer of the transgene from a donor plant to a recipient plant. Protoplast fusion is an induced or spontaneous union, such as a somatic hybridization, between two or more protoplasts (cells in which the cell walls are removed by enzymatic treatment) to produce a single bi- or multi-nucleate cell. The fused cell that may even be obtained with plant species that cannot be interbred in nature is tissue cultured into a hybrid plant exhibiting the desirable combination of traits. More specifically, a first protoplast can be obtained from a plant having the expression cassette. A second protoplast can be obtained from a second plant line, optionally from another plant species or variety', preferably from the same plant species or variety, that comprises commercially desirable characteristics, such as, but not limited to disease resistance, insect resistance, valuable grain characteristics (e.g., increased seed weight and/or seed size) etc. The protoplasts are then fused using traditional protoplast fusion procedures, which are known in the art to produce the cross. Alternatively, embryo rescue may be employed in the transfer of the expression cassette from a donor plant to a recipient plant. Embryo rescue can be used as a procedure to isolate embryos from crosses wherein plants fail to produce viable seed. In this process, the fertilized ovary' or immature seed of a plant is tissue cultured to create new' plants (see Pierik, 1999, In vitro culture of higher plants, Springer, ISBN 079235267x, 9780792352679, which is incorporated herein by reference in its entirety). In some embodiments, the recipient plant is an elite line having one or more certain desired traits. Examples of desired traits include but are not limited to those that result in increased biomass production, production of specific chemicals, increased seed production, improved plant material quality, increased seed oil content, etc. Additional examples of desired traits include pest resistance, vigor, development time (time to harvest), enhanced nutrient content, novel growth patterns, aromas or colors, salt, heat, drought and cold tolerance, and the like. Desired traits also include selectable marker genes (e.g., genes encoding herbicide or antibiotic resistance used only to facilitate detection or selection of transformed cells), hormone biosynthesis genes leading to the production of a plant hormone (e.g., auxins, gibberellins, cytokinins, abscisic acid and ethylene that are used only for selection), or reporter genes (e.g. luciferase, b-giucuromdase, chloramphenicol acetyl transferase (CAT, etc.). The recipient plant can also be a plant with preferred chemical compositions, e.g., compositions preferred for medical use or industrial applications. Classical breeding methods can be used to produce new varieties of cannabis. Newly developed Fl hybrids can be reproduced via asexual reproduction.

In some cases, population improvement methods may be utilized. Population improvement methods fall naturally into two groups, those based on purely phenotypic selection, normally called mass selection, and those based on selection with progeny testing. Interpopulation improvement utilizes the concept of open breeding populations; allowing genes to flow from one population to another. Plants in one population (cultivar, strain, ecotype, or any germplasm source) are crossed either naturally (e.g., by wind) or by hand or by bees (commonly Apis meflifera L. or Megachile rotundata F.) with plants from other populations. Selection can be applied to improve one (or sometimes both) population(s) by isolating plants comprising desirable traits from both sources.

In another aspect, mass selection can be utilized. In mass selection, desirable individual plants are chosen, harvested, and the seed composited without progeny testing to produce the following generation. Since selection is based on the maternal parent only, and there is no control over pollination, mass selection amounts to a form of random mating with selection. As stated herein, the purpose of mass selection is to increase the proportion of superior genotypes m the population. While mass selection is sometimes used, progeny testing is generally preferred for poly crosses, because of their operational simplicity and obvious relevance to the objective, namely exploitation of general combining ability in a synthetic.

In some embodiments, breeding may utilize molecular markers. Molecular markers are designed and made, based on the genome of the plants of the present application. In some embodiments, the molecular markers are selected from Isozyme Electrophoresis, Restriction Fragment Length Polymorphisms (RFLPs), Randomly-Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs). Amplified Fragment Length Polymorphisms (AFLPs), and Simple Sequence Repeats (SSRs) which are also referred to as Microsatellites, etc. Methods of developing molecular markers and their applications are described by Avise (Molecular markers, natural history, and evolution, Publisher: Sinauer Associates, 2004, ISBN 0878930418, 9780878930418), Snvastava et al. (Plant biotechnology and molecular markers, Publisher: Springer, 2004, ISBN1402019114, 9781402019111), and Vienne (Molecular markers in plant genetics and biotechnology, Publisher: Science Publishers, 2003), each of winch is incorporated by reference in its entirety for all purposes. The molecular markers can be used in molecular marker assisted breeding. For example, the molecular markers can be utilized to monitor the transfer of the genetic material in some embodiments, the transferred genetic material is a gene of interest, such as genes that contribute to one or more favorable agronomic phenotypes when expressed in a plant cell, a plant part, or a plant.

Provided herein can also be methods for generating transgenic plants. In some aspects, methods provided herein can comprise (a) contacting a plant cell with an endonuclease or a polypeptide encoding an endonuclease. In some cases, an endonuclease introduces a genetic modification in a genome of a plant cell resulting in an increased amount of a compound selected from:

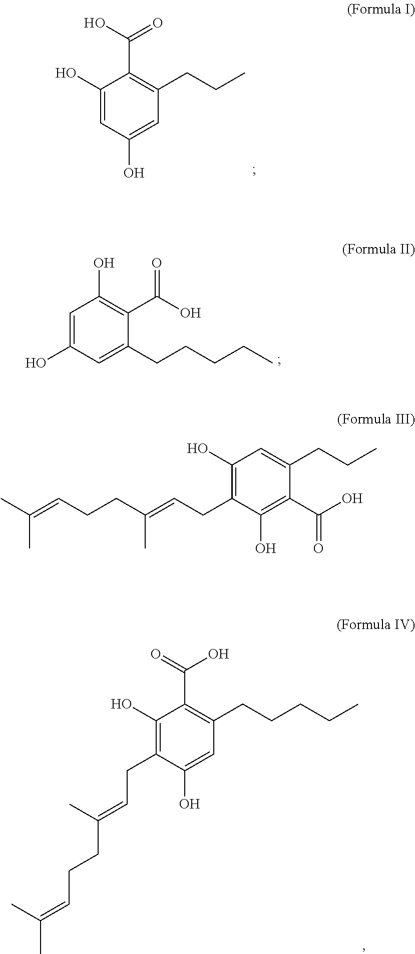

derivatives or analogs thereof, as compared to an amount of the same compound in a comparable control plant without a genetic modification. In some aspects, a method can further comprise culturing a plant cell that has been genetically modified as previously described to generate a transgenic plant. In some aspects, culturing a transgenic plant cell can result in generation of a callus, a cotyledon, a root, a leaf, or a fraction thereof. Methods of making transgenic plants can include electroporation, agrobacterium mediated transformation, biolistic particle bombardment, or protoplast transformation.

In some aspects, provided herein can also be a method for generating transgenic plants comprising contacting a plant cell with an endonuclease or a polypeptide encoding an endonuclease. An endonuclease can introduce a genetic modification resulting in an increased amount of a cannabigerol (CBG), a derivative, or analogue thereof as compared to an amount of the same compound in a comparable control plant absent a genetic modification. In some aspects, a method can further comprise culturing a plant cell to generate a transgenic plant.

Provided herein can also be methods for generating a transgenic plant comprising contacting a plant cell with an endonuclease or a polypeptide encoding an endonuclease. An endonuclease can introduce a genetic modification resulting in an increased amount of cannabinol (CBN), a derivative, or analogue thereof as compared to an amount of the same compound in a comparable control plant without a genetic modification and further comprising culturing a plant cell in to generate a transgenic plant. Similarly, a method provide herein can comprise contacting a plant cell with an endonuclease or a polypeptide encoding an endonuclease under conditions such that an endonuclease introduces a genetic modification resulting in an increased amount of tetrahydrocannabivarin (THCV), a derivative, or an analogue thereof as compared to an amount of the same compound in a comparable control plant without a genetic modification.

In some cases, a method for generating a transgenic plant can comprise introducing a genetic modification that results in an increased amount of cannabigerol (CBG), derivative or analog thereof in a transgenic plant as compared to an amount of the same compound in a comparable control plant absent a genetic modification. In some aspects, a genetic modification comprises a disruption of a first group of genes such that a disruption results in an increased amount of

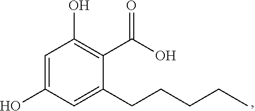 [SMILES: CCCCCc1cc(O)cc(O)c1C(=O)O]

derivative or analog thereof. A first group of genes can comprise olivetolic acid cyclase (OAC) and/or olivetolic acid synthase (OLS). Genomic modifications can include any one of genes provided herein such as but not limited to genes encoding CBCA synthase, CBDA synthase, and THCA synthase. Genomic modifications can result in decreased amounts of CBCA synthase, CBDA synthase, THCA synthase, derivatives or analogs thereof as compared to an amount of the same compound in a comparable control plant absent a disruption. Methods comprising modifications of plant cell genomes can result in: 5%, 10%, 15%, 20%, 25%, 30%, 35%, 40%, 45%, 50%, 55%, 60%, 65%, 70%, 75%, or up to about 80% more

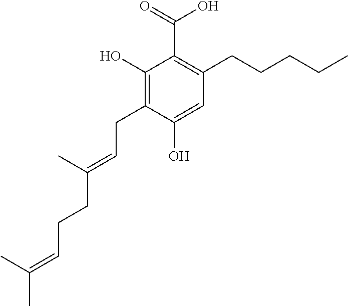 [SMILES: CCCCCc1cc(O)c(C/C=C(\C)CCC=C(C)C)c(O)c1C(=O)O]

as measured by dry weight in a transgenic plant as compared to a comparable control plant without a genomic modification. Methods comprising modifications can also result in from about 1%, 5%, 10%, 15%, 20%, 25%, 30%, 35%, 40%, 45%, 50%, 55%, 60%, 65%, 70%, 80%, 90%, 100%, or up to about 200% less CBCA, CBDA, THCA as measured by dry weight in a transgenic plant as compared to a comparable control plant without a modification.

Provided herein can also be cells obtained from transgenic plants provided herein. Cells from transgenic plants can be genetically modified. Cells from transgenic plants can be obtained from any portion of a transgenic plant such as but not limited to: a callus cell, a protoplast, an embryonic cell, a leaf cell, a seed cell, a stem cell, or a root cell. In some aspects, a genetically modified cell can be a plant cell, an algae cell, an agrobacterium cell, a E. coli cell, a yeast cell, an animal cell, or an insect cell. In some cases, a genetically modified cell is a plant cell, for example a cannabis plant cell. A genetically modified cell can comprise a modification that can be integrated into a genome of a cell.

Additionally, provided herein can also be compositions comprising an endonuclease or polynucleotide encoding provided endonucleases capable of introducing a genetic modification. In some aspects, genetic modifications can result in increased amounts of

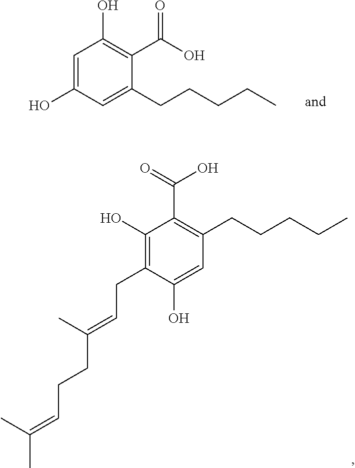

derivatives or analogs thereof. In some cases, a genetic modification may not result in a change of an amount of

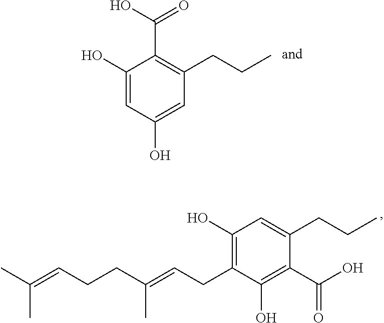 [SMILES: CCCc1cc(O)cc(O)c1C(=O)O ; CCCc1cc(O)c(C/C=C(\C)CCC=C(C)C)c(O)c1C(=O)O]

derivatives or analogs thereof as compared to a comparable control cell without a genetic modification.

Provided herein can also be a composition comprising an endonuclease or polynucleotide encoding an endonuclease capable of introducing a genetic modification. In some aspects, a genetic modification results in an increased amount of

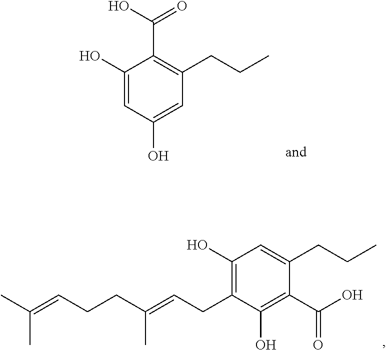 [SMILES: CCCc1cc(O)cc(O)c1C(=O)O ; CCCc1cc(O)c(C/C=C(\C)CCC=C(C)C)c(O)c1C(=O)O]

derivatives or analogs thereof such that a genetic modification may not result in a change of an amount of

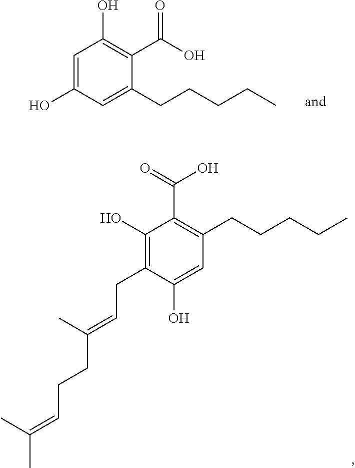

derivatives or analogs thereof, as compared to a comparable control cell without a genetic modification.

### Pharmacological Compositions and Methods

Provided herein can be pharmacological compositions comprising cannabis and/or hemp and modified versions thereof. Provided herein can also be pharmacological reagents, methods of using, and method of making pharmacological compositions comprising cannabis and/or hemp and portions of cannabis plants and/or hemp plants. Provided herein are also pharmacologically-suitable modified plants and portions thereof

In some cases, cannabis and/or hemp can be used as a pharmaceutical. Some of the medical benefits attributable to one or more of the cannabinoids isolated from cannabis and/or hemp include treatment of pain, nausea, AIDS-related weight loss and wasting, multiple sclerosis, allergies, infection, depression, migraine, bipolar disorders, hypertension, post-stroke neuroprotection, epilepsy, and fibromyalgia, as well as inhibition of tumor growth, angiogenesis and metastasis. Cannabis and/or hemp may also be useful for treating conditions such as glaucoma, Parkinson's disease, Huntington's disease, migraines, inflammation, Crohn's disease, dystonia, rheumatoid arthritis, emesis due to chemotherapy, inflammatory bowel disease, atherosclerosis, posttraumatic stress disorder, cardiac reperfusion injury, prostate carcinoma, and Alzheimer's disease. Cannabis and/or hemp can be used as antioxidants and neuroprotectants. Cannabis and/or hemp can also be used for the treatment of diseases associated with immune dysfunction, particularly HIV disease and neoplastic disorders. Cannabinoids can be useful as vasoconstrictors. THC-CBD composition for use in treating or preventing Cognitive Impairment and Dementia. In some aspects, cannabinoids can be used for the manufacture of a medicament for use in the treatment of cancer. Additional uses can also include use of cannabinoid composition for the treatment of neuropathic pain. In some aspects, a method of treating tissue injury in a patient with colitis can comprise administering a cannabinoid.

While a wide range of medical uses has been identified, the benefits achieved by cannabinoids for a disease or condition are believed to be attributable to a subgroup of cannabinoids or to individual cannabinoids. That is to say that different subgroups or single cannabinoids have beneficial effects on certain conditions, while other subgroups or individual cannabinoids have beneficial effects on other conditions. For example, THC is the main psychoactive cannabinoid produced by cannabis and is well-characterized for its biological activity and potential therapeutic application in a broad spectrum of diseases. CBD, another major cannabinoid constituent of cannabis, acts as an inverse agonist of the CB1 and CB2 cannabinoid receptors. Unlike THC, CBD does nor or can have substantially lower levels of psychoactive effects in humans. In some aspects, CBD can exert analgesic, antioxidant, anti-inflammatory, and immunomodulatory effects.

Provided herein can also be methods of treating disease or conditions comprising administering pharmaceutical compositions, nutraceutical compositions, and/or the food supplements to a subject in need thereof. In some cases, a disease or condition can be selected from the group consisting of anorexia, emesis, pain, inflammation, multiple sclerosis, Parkinson's disease, Huntington's disease, Tourette's syndrome, Alzheimer's disease, epilepsy, glaucoma, osteoporosis, schizophrenia, cardiovascular disorders, cancer, and/or obesity.

Provided herein are also extracts from specialty cannabis plants. Cannabis extracts or products or the present disclosure include: Kief—refers to tnchomes collected from cannabis. The trichomes of cannabis are the areas of cannabinoid and terpene accumulation. Kief can be gathered from containers where cannabis flowers have been handled. It can he obtained from mechanical separation of the trichomes from inflorescence tissue through methods such as grinding flowers, or collecting and sifting through dust after manicuring or handling cannabis. Kief can be pressed into hashish for convenience or storage. Hash—sometimes known as hashish, is often composed of preparations of cannabis trichomes. Hash pressed from kief is often solid. Bubble Hash—sometimes called bubble melt hash can take on paste-like properties with varying hardness and pliability. Bubble hash is usually made via water separation in which cannabis material is placed in a cold-water bath and stirred for a long time (around 1 hour). Once the mixture settles it can be sifted to collect the hash. Solvent reduced oils—also sometimes known as hash oil, honey oil, or full melt hash among other names. This type of cannabis oil is made by soaking plant material in a chemical solvent. After separating plant material, the solvent can be boiled or evaporated off, leaving the oil behind. Butane Hash Oil is produced by passing butane over cannabis and then letting the butane evaporate. Budder or Wax is produced through isopropyl extraction of cannabis. The resulting substance is a wax like golden brown paste. Another common extraction solvent for creating cannabis oil is C02. Persons having skill in the art will be familiar with C02 extraction techniques and devices, including those disclosed in US 20160279183, US 2015/01505455, U.S. Pat. NO. 9,730,911, and US 2018/0000857. Tinctures—are alcoholic extracts of cannabis. These are usually made by mixing cannabis material with high proof ethanol and separating out plant material. E-juice—are cannabis extracts dissolved in either propylene glycol, vegetable glycerin, or a combination of both. Some E-juice formulations will also include polyethylene glycol and flavorings. E-juice tends to be less viscous than solvent reduced oils and is commonly consumed on e-cigarettes or pen vaporizers. Rick Simpson Oil (ethanol extractions)—are extracts produced by contacting cannabis with ethanol and later evaporating the vast majority of ethanol away to create a cannabinoid paste. In some embodiments, the extract produced from contacting the cannabis with ethanol is heated so as to decarboxylate the extract. While these types of extracts have become a popular form of consuming cannabis, the extraction methods often lead to material with little or no Terpene Profile. That is, the harvest, storage, handling, and extraction methods produce an extract that is rich in cannabinoids, but often devoid of terpenes.

In some embodiments, genetically modified compositions provided herein, such as plants and plant cells can be extracted via methods that preserve the cannabinoid and terpenes. In other embodiments, said methods can be used with any cannabis plants. The extracts of the present invention are designed to produce products for human or animal consumption via inhalation (via combustion, vaporization and nebulization), buccal absorption within the mouth, oral administration, and topical application delivery methods. The present invention teaches an optimized method at which we extract compounds of interest, by extracting at the point when the drying harvested plant has reached 15% water weight, which minimizes the loss of terpenes and plant volatiles of interest. Stems are typically still ‘cool’ and ‘rubbery’ from evaporation taking place. This timeframe (or if frozen at this point in process) allow extractor to minimize terpene loss to evaporation. There is a direct correlation between cool/slow, -'dry and preservation of essential oils. Thus, there is a direct correlation to EO loss in flowers that dry too fast, or too hot conditions or simply dry out too much (<10% H20). The chemical extraction of Specialty Cannabis can be accomplished employing polar and non-polar solvents m various phases at varying pressures and temperatures to selectively or comprehensively extract terpenes, cannabinoids and other compounds of flavor, fragrance or pharmacological value for use individually or combination in the formulation of our products. The extractions can be shaped and formed into single or multiple dose packages, e.g., dabs, pellets and loads. The solvents employed for selective extraction of our cultivars may include water, carbon dioxide, 1,1,1,2-tetrafluoroethane, butane, propane, ethanol, isopropyl alcohol, hexane, and limonene, in combination or series. We can also extract compounds of interest mechanically by sieving the plant parts that produce those compounds. Measuring the plant part i.e. trichome gland head, to be sieved via optical or electron microscopy can aid the selection of the optimal sieve pore size, ranging from 30 to 130 microns, to capture the plant part of interest. The chemical and mechanical extraction methods of the present invention can be used to produce products that combine chemical extractions with plant parts containing compounds of interest. The extracts of the present invention may also be combined with pure compounds of interest to the extractions, e.g. cannabinoids or terpenes to further enhance or modify the resulting formulation's fragrance, flavor or pharmacology. In some embodiments, the extractions are supplemented with terpenes or cannabinoids to adjust for any loss of those compounds during extraction processes. In some embodiments, the cannabis extracts of the present invention mimic the chemistry of the cannabis flower material. In some embodiments, the cannabis extracts of the present invention will about the same cannabinoid and Terpene Profile of the dried flowers of the Specialty Cannabis of the present invention.

In some aspects, extracts of the present invention can be used for vaporization, production of e-juice or tincture for e-cigarettes, or for the production of other consumable products such as edibles or topical spreads. Cannabis edibles such as candy, brownies, and other foods can be a method of consuming cannabis for medicinal and recreational purposes. In some embodiments, modified plants provided herein and cannabinoid compositions of the present disclosure can be used to make cannabis edibles. Most cannabis edible recipes begin with the extraction of cannabinoids and terpenes, which are then used as an ingredient in various edible recipes. In one embodiment, the cannabis extract used to make edibles out of transgenic plants can be cannabis butter. Cannabis butter can be made by melting butter in a container with cannabis and letting it simmer for about half an hour, or until the butter turns green. The butter can be chilled and used in normal recipes. Other extraction methods for edibles include extraction into cooking oil, milk, cream, flour (grinding cannabis and blending with flour for baking). Lipid rich extraction mediums/edibles are believed to facilitate absorption of cannabinoids into the blood stream. THC absorbed by the body is converted by the liver into 11-hydroxy-THC. This modification increases the ability of the THC molecule to bind to the CB1 receptor and also facilitates crossing of the brain blood barrier thereby increasing the potency and duration of its effects.

In some aspects, provided herein can also be nutraceutical compositions. Nutraceutical compositions can comprise extracts of transgenic plants, plants generated from genetically modified cells, compositions comprising genetic modifications, and/or cells provided herein. In some aspects, food supplements comprising compositions provided herein and/or generated from genetically modified plants provided herein.

Pharmaceutical compositions provided herein can also comprise extracts of transgenic plants, genetically modified cells, and pharmaceutically acceptable excipients, diluents, and/or carriers. In some aspects, excipients can be lipids.

Pharmaceutical compositions provided herein can be introduced as oral forms, transdermal forms, oil formulations, edible foods, food substrates, aqueous dispersions, emulsions, solutions, suspensions, elixirs, gels, syrups, aerosols, mists, powders, tablets, lozenges, lotions, pastes, formulated sticks, balms, creams, and/or ointments.

Provided herein can also be kits for genome editing comprising compositions provided herein. Kits can include containers, instructions, and the like. Kits can also include plants, seeds, and instructions for utilizing the same.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### Example 1

### Method for Modulating Compound Yield for Generating Transgenic Plants

### Over-Expression Approach

Gene overexpression can be used to increase the production of intermediary compounds to generate a greater amount of a compound of interest. Any intermediary compound may be modulated for greater expression such as but not limited to: cannabigerolic acid (CBGA), highly functional tetrahydrocannabinolic acid (THCA), and cannabidiolic acid (CBDA) enzymes.

The same strategy can be applied to increase the amount of cannflavins A and B by modulating their precursors luteolin and/or chrysoeriol. In some embodiments, provided herein are methods of increasing the activity of CsPT3. In some embodiments, provided herein are methods of increasing the conversion of chrysoeriol into cannflavins A or B.

### Example 2

### Transcriptional Activation of Compounds of the Cannabinoid Biosynthesis Pathway

To activate compounds of the cannabinoid biosynthesis pathway a dCas9-VP64 system comprising the deactivated CRISPR-associated protein 9 (dCas9) fused with four tandem repeats of the transcriptional activator VP16 (VP64) is employed. Any intermediary compound may be activated for greater expression such as but not limited to: cannabigerolic acid (CBGA), highly functional tetrahydrocannabinolic acid (THCA), CsPT3, and cannabidiolic acid (CBDA) enzymes.

The amount of cannflavins A and B is also modulated via their precursors luteolin and/or chrysoeriol.

Assembly of a CRISPR-Act2.0 T-DNA Vector with Triplex GRNAs

Step1. Cloning guide RNA (gRNA) into gRNA2.0 expression vectors. Linearize guide RNA expression plasmids (pYPQ131A/B/C/D2.0, 132A/B/C/D2.0 and 133A/B/C/D2.0; pYPQ141A/B/C/D2.0 for single gRNA)

TABLE 6
Reaction mixture for first digestion with BglII
and SalI. Reaction is run at 37° C., 3 hrs.

| Reagent (Concentration) | Amount | |
|---|---|---|
| H2O | 14 | μl |
| gRNA plasmid (~100 ng/μl) | 20 | μl |
| 10X NEB buffer 3.1 | 4 | μl |
| BglII (10 u/μl; NEB) | 1 | μl |
| SalI-HF (10 u/μl; NEB) | 1 | μl |
| Total | 40 | μl |

Purify 1^{st} digestion products using Qiagen PCR purification kit, elute DNA with 35 μl ddH2O, set up digestion reaction as follow:

TABLE 7
Reaction mixture for second digestion with Esp3I
(BsmBI). Reaction is run at 37° C., O/N

| Reagent (Concentration) | Amount | |
|---|---|---|
| Digested gRNA plasmid (from step 1) | 32 | μl |
| 10X OPTIZYME buffer 4 | 4 | μl |
| DTT (20 mM) | 2 | μl |
| EPS3I (10 u/μl; Thermo Scientific) | 2 | μl |
| Total | 40 | μl |

Inactivate enzymes at 80° C. denature for 20 min, purify the vector using Qiagen PCR purification kit, and quantify DNA concentration using Nanodrop.

Cloning Oligos into Linearized pYPQ13N-2.0 V4ector

Table 8 Oligo phosphorylation and annealing

| Reagent (Concentration) | Amount | |
|---|---|---|
| sgRNA oligo forward (100 μM) | 1 | μl |
| sgRNA oligo reverse (100 μM) | 1 | μl |
| 10X T4 Polynucleotide Kinase buffer | 1 | μl |
| T4 Polynucleotide Kinase (10 u/μl; NEB) | 0.5 | μl |
| ddH2O | 6.5 | μl |
| Total | 10 | μl |

Phosphorylate and anneal the oligos using 37° C. for 30 min; 95° C. for 5 min; ramp down to 25° C. at 5° C. min⁻¹ (i.e., 0.08° C./second) using a thermocycler (alternatively: cool down in boiled water).

TABLE 9
Ligate oligos into linearized gRNA expression vector and transformation
of E. coli DH5α cells. Reaction is run at RT for 1 hr.

| Reagent (Concentration) | Amount | |
|---|---|---|
| ddH2O | 6.5 | ul |
| 10X NEB T4 ligase buffer | 1 | μl |
| Linearized gRNA2.0 plasmid | 1 | μl |
| Diluted annealed Oligos (1:200 dilution) | 1 | μl |
| T4 ligase | 0.5 | μl |
| Total | 10 | μl |

Transform E. coli DH5a cells and plate transformed cells on a Tet⁺ (5 ng/ul) LB plate; 37° C., 0/N. Mini-prep two independent clones and verify gRNAs by Sanger sequencing with primer pTC14-F2 (for pYPQ131, 132 and 133 based vectors) or M13-F (for pYPQ141 based vectors).

Step2. Golden Gate Assembly of 3 gRNA2.0 cassettes. Set up Golden Gate reaction:

TABLE 10
Assembly of 3 guide RNAs

| Reagent (Concentration) | Amount | |
|---|---|---|
| H2O | 4 | μl |
| 10X T4 DNA ligase buffer (NEB) | 1 | μl |
| pYPQ143 (100 ng/μl) | 1 | μl |
| pYPQ131-gRNA1 (100 ng/μl) | 1 | μl |
| pYPQ132-gRNA2 (100 ng/μl) | 1 | μl |
| pYPQ133-gRNA3 (100 ng/μl) | 1 | μl |
| BsaI (NEB) | 0.5 | μl |
| T4 DNA ligase (NEB) | 0.5 | μl |
| Total | 10 | μl |

Run Golden Gate program in a thermocycler as follows:

| | | |
|---|---|---|
| 37° C., 5 min | | |
| | {close oversize brace} | 10 cycles |
| 16° C., 10 min | | |
| 50° C., 5 min | | |
| 80° C., 5 min | | |
| Hold at 10° C. | | |

Transform E. coli DH5a cells and plate transformed cells onto a Spe⁺ (100 μg/ml) LB plate. Mini-prep two independent clones and verify by restriction digestion

Step 3. Gateway Assembly of Multiplex CRISPR-Cas9 system into a binary vector. Set up Gateway LR reaction as following:

TABLE 11
Reaction is run at RT for 1 hr (O/N recommended)

| Reagent (Concentration) | Amount |
|---|---|
| Cas9 entry vector pYPQ173 (25 ng/μl) | 2 μl |
| Guide RNA entry vector (25 ng/μl) | 2 μl |
| Destination vector (100 ng/μl) | 2 μl |
| LR Clonase II | 1 μl |
| Total | 7 μl |

Transform E. coli DH5α cells and plate transformed cells on a Kan⁺ (50 82 g/ml) LB plate. Mini-prep two independent clones and verify by restriction digestion

### Example 3

### Production of THC and/or CBD Enhancement Via Gene Editing of Competitors for THC's and CBD's Common Source Material

For the production of THC and CBD, a common precursor may be in existence for other compounds. By disabling those genes that participate on the production of less/un attractive compounds, production of the compounds of interest may be enhanced.

### Example 4

### Transformation of Cannabis and/or Hemp

Seeds were disinfected using ethanol 70% for 30 sec and 5% bleach for 5-10 min. Seeds were then washed using sterile water 4 times. Subsequently seeds were germinated on half-strength ½ MS medium supplemented with 10 g·L-1 sucrose, 5.5 g·L-1 agar (pH 6.8) at 25 +/− 2 C under 16/8 photoperiod and 36-52 uM×m-1×s1 intensity. Young leaves were selected at about 0.5-10 mm for initiation of shoot culture. Explants were disinfected using 0.5% NaOCL (15% v/v bleach) and 0.1% tween 20 for 20 min (Optional as plantlets were growing in an aseptic environment). Additionally, a different tissue was tested, for example young cotyledons 2-3 days old.

### Callus Induction

Leaves were cultivated on MS media supplemented with 3% sucrose and 0.8% Bacteriological agar (PH 5. 8). Leaves were autoclaved after measuring pH). Added filtered sterilized 0.5 uM NAA*+1 uM TDZ* and plates kept at 25 +/− 2 C in the dark. NAA/TDZ was replaced with 2-4D and Kinetin at different concentrations. Copper sulphate and additional myo-inositol and proline were tested for callus quality. In addition, Glutamine was added to MS media prior pH measurement to increase callus generation and quality. The callus was broken in smaller pieces and allowed to grow as in for 2-3 days before inoculation.

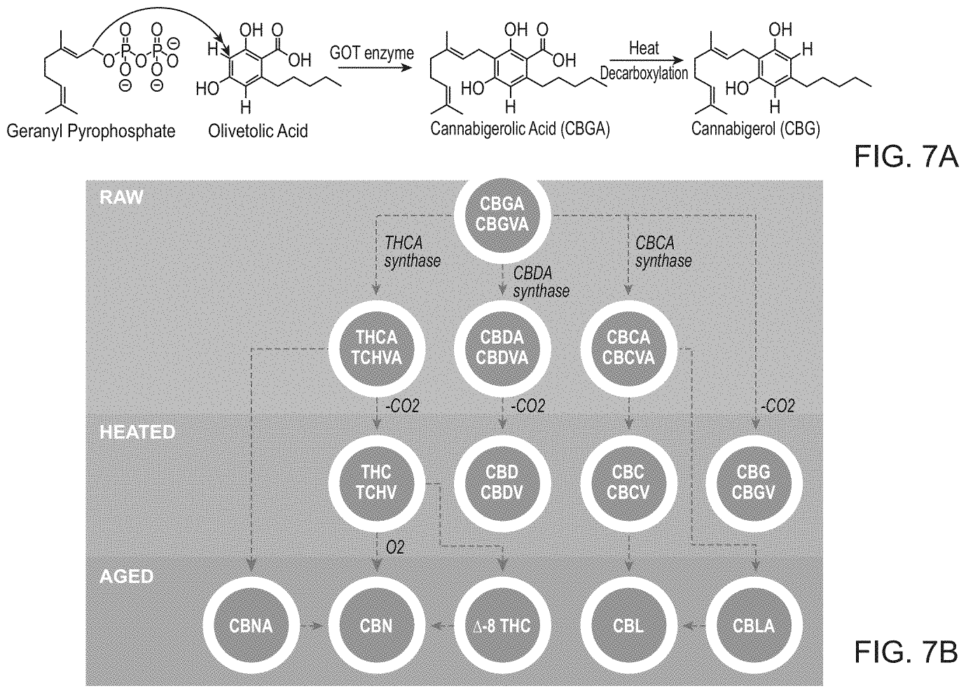

Callus were generated using leaf tissue from 1 month old in-vitro Finola plants. The protocol disclosed below are focused on the transformation of callus in conditions that promote healthy tissue formation without hyperhydricity (excessive hydration, low lignification, impaired stomatal function and reduced mechanical strength of tissue culture-generated plants). Prior to CRISPR delivery and genome modification in the callus tissue, protocols disclosed below were being modified using the GUS (beta-glucuronidase) reporter gene system to identify conditions for maximal expression of transgenes and successful regeneration of plants. FIGS. 7A and 7B show that Hemp callus inoculated with agrobacterial carrying the GUS expressing vector pCambia1301 following staining with X-Gluc to visualize the cells that were successfully transformed with the DNA. In some embodiments, a skilled artisan may be able to use the protocols disclosed herein to regenerate plants with CRISPR mediated THCAS gene over-expressing in suitable vector.

### Callus Generation Protocol was Performed as Outlined Below

Disinfect seeds using ethanol 70% for 30 sec and 5% bleach for 5-10 min. Wash seeds using abundant sterile water 4 times.

Germinate seeds on half-strength ½ MS medium supplemented with 15 g·L-1 sucrose, 5.5 g·L-1 agar (pH 6.8) at 25 +/− 2 C under 16/8 photoperiod.

Select young leaves 0.5-10 mm for initiation of shoot culture. Disinfect explants using 0.5% NaOCL (15% v/v bleach) and 0.1% tween 20 for 20 min (Optional as plantlets are growing in an aseptic environment).

Callus induction: Cultivate leaves on MS media +3% sucrose and 0.8% TYPE E agar (Sigma)+0.15mg/l IAA+0.1mg/l TDZ+0.001 mg/l Pyridoxine+10 mg/l myo-inositol+0.001 mg/l nicotinic acid+0.01 mg/l Thiamine+0.5 mg/l AgNO3 (CI.1.98.3) and place them at 25 C +/− 2 and 16 H photoperiod and 52 uM/m/s light intensity for 4 weeks.

Break the callus in smaller pieces and let them grow as in 4 for one week before inoculation.

| | | Sucrose | IAA | TDZ | Pyridoxine | Myo- | Nicotinic | Thiamine | AgNO3 |
|---|---|---|---|---|---|---|---|---|---|
| | MSg/l | g/l | mg/l | mg/l | mg/l | inositolmg/l | acid mg/l | mg/l | mg/l |
| CI.1.98.3 | 4.92 | 30 | 0.15 | 0.1 | 0.001 | 10 | 0.001 | 0.01 | 0.5 |

### Callus Inoculation and Regeneration Protocol was Performed as Outlined Below

Grow LBA4404/AGL1:desired vector to 10 in LB+Rif and Spec media at 28 C 24 Hrs.

Transfer 200 ul for previous culture into 100 ml MGL without antibiotic and incubate at 28 C 24 Hr.

Spin culture at 3000 rpm and 4 C and resuspend it in cells in MS+10 g/l glucose+15 g/l sucrose and pH 5.8) to obtain OD600≈0.6-0.8. Agrobacterium cells were activated by treating with 200 μM acetosyringone (AS) for 45-60 min in dark before infection.

Calli were added into the agrobacterium for 15-20 min with continuous shaking at 28 C.

Infected calli were transferred to sterile filter paper and dry, then transferred to co-culture media at 25 C for 48 Hrs.

After 2-3 days of co-cultivation, the infected calli were washed 3 times in sterile water and then washed once in sterile water containing 400 mg/l Timentine and again in sterile water containing 200 mg/l Timentine to remove Agrobacterium.

The washed calli were dried on sterile filter papers and cultured on callus selection medium containing 160 mg/l Timentine and 50 mg/l Hyg). Kept in dark for selecting transgenic calli for 15 days.

After first round of selection for 20 days, brownish or black coloured calli were discarded and white calli were transferred to fresh selection medium for second selection cycle for 15 days.

This step allowed the proliferation of micro calli and when small micro calli started growing on the mother calli, each micro callus was gently separated from the mother calli and transferred to fresh selection medium for the third selection 15 days. Healthy calli were selected for regeneration and PCR analysis.

Shoot regeneration: After three selection cycles, healthy callus were transferred to MS+3% sucrose and 0.8% TYPE E agar (Sigma)+0.5 uMTDZ plus selective antibiotic (depending on vector used) and 160 mg/l of Timentin for shoot regeneration. Placed them at 25 C +/− 2 and 16 H photoperiod and 52 uM/m/s light intensity (Acclimation process could be used by placing tissue paper on top to avoid excessive light for at least 1-2 weeks).

Once shoots were observed to be well stablished, 2-3 weeks, plantlets were transferred to Rooting media containing: half MS media+3% sucrose, 0.8% TYPE E agar (Sigma), auxins 2.5 uM IBA and selective antibiotic (depending on vector used) and 160 mg/l of Timentin., shoots were placed at 25 +/− 2 C, 16 h photoperiod and 52 uM×m-1×s-1 intensity.

Stablished plants were transferred to soil. Explants had the roots cleaned from any rest of agar. Plantlets were preincubated in coco natural growth medium (Canna Continental) in thermocups (Walmart store, Inc) for 10 days. The cups were covered with polythene bags to maintain humidity, kept in a growth room and later acclimatized in sterile potting mix (fertilome; Canna Continental) in large pots. All the plants were kept under strict controlled environmental conditions (25±3° C. temperature and 55±5% RH). Initially, plants were kept under cool fluorescent light for 10 days and later exposed to full spectrum grow lights (18-hour photoperiod, ˜700±24 μmol.m-2 s-1 at plant canopy level.

### Callus Transformation

Agrobacterium culture was prepared from glycerol stock/single colony on agar plate transfer Agrobacterium colonies carrying the vector of interest into liquid LB media+15 uM acetoseryngone (plus selection antibiotic: this will depend on vector and Agrobacterium strain used). Shake culture overnight at 28 C. Additionally, different Agrobacterium inoculation media may be tested. Once Agrobacterium liquid culture containing antibiotic reaches an OD600=0.5 approx., Agrobacterium liquid culture was centrifuged at 4000 rpm maximum for 15 min at 4° C. The Agrobacterium pellet was collected and resuspended it in inoculation media comprising LB media adjusting OD600 to approximately 0.3 without antibiotics. After pellet resuspension, the culture was left for 1-2 hours before inoculation. The calli were mixed into the culture and incubated in a shaker, 150rpm, for 15-30 min. The reaction mixture was monitored, as excessive OD can generate contamination. Inoculation media was tested to increase efficiency of Agrobacterium infection. Calli were collected in sterilized filter paper and allowed to dry and placed on a single sterile filter paper which is placed on a petri dish containing callus induction media (MS media containing 3% sucrose and 0.8% Bacteriological agar (pH 5.8, autoclave). Afterwards, it was filtered and sterilized (0.5 uM NAA and 1 uM TDZ) and placed at 25 C+/− 2 in the dark for 2-3 days. Excessive Agrobacterium Contamination was monitored during the incubation. Additionally, NAA/TDZ was replaced with 2-4D and Kinetin at different concentrations. In some cases, copper sulphate, myo-inositol, and proline were tested for callus quality. In addition, Glutamine was added to MS media prior to pH measurement to increase callus generation and quality.

The callus MS media+3% sucrose and 0.8% bacteriological agar (pH 5.8) was transferred and autoclaved. Filtered, sterilized 0.5 uM NAA+1 uM TDZ (Replace NAA/TDZ with 2-4D and Kinetin at different concentrations. In this step, Copper sulphate and additional myo-inositol and proline were tested for callus quality. In addition, Glutamine may be added to MS media prior to pH measurement to increase callus generation and quality. If Agrobacterium overgrows and threatens to overwhelm calli, calli disinfection may be conducted before continuing callus induction, was added along with a selective antibiotic (depending on vector used) and 160-200 mg/l of Timentin to inhibit Agrobacterium growth. The reaction mixture was placed at 25 C +/− 2 in the dark. The selection media was renewed every week. Growth of callus was monitored as well as health. Two weeks after selection started, callus was transferred to shooting media.

### Cotyledon Inoculation

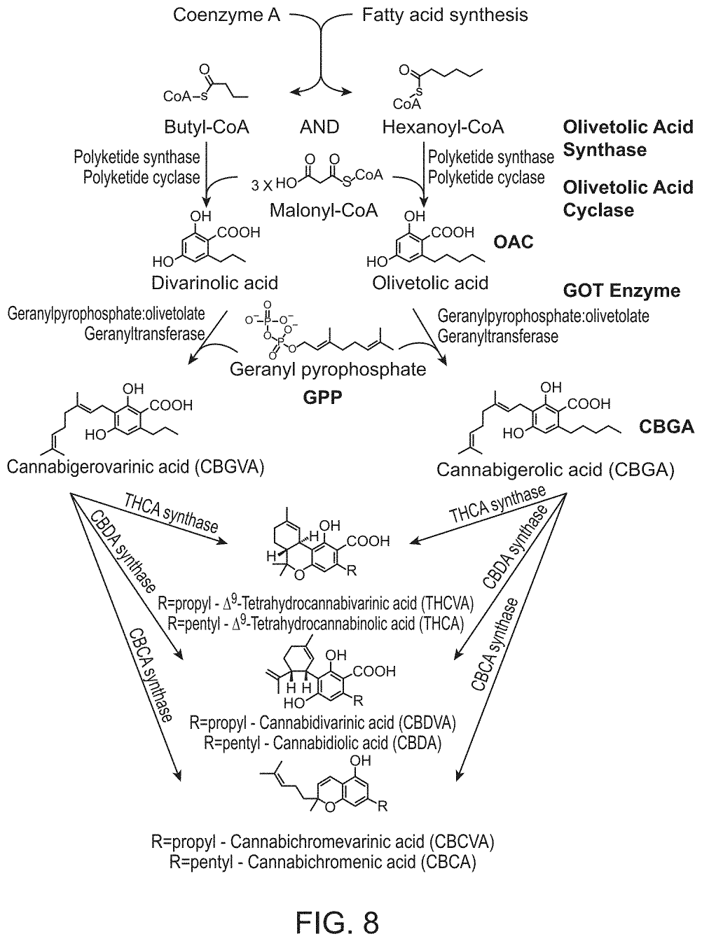

Cotyledon was the embryonic leaf in seed-bearing plants and represent the first leaves to appear from a germinating seed. Protocols disclosed below have been developed for the excision of cotyledon from 5 to 7-day old plantlets prior to submerging into a suspension of agrobacterium carrying the GUS reporter vector pCambia1301. After 7 days on Hygromycin selection agar plates, the tissue was stained with X-Gluc and GUS expression visualized. The blue staining indicated by black arrows shown in FIGS. 8A-8C was observed in callus forming areas, areas where plant regeneration is expected to occur (ongoing evaluation).

### Cotyledon and Hypocotyls Inoculation Protocol was Performed as Outlined Below

Grow AGL1:desired vector (from glycerol stock/colony) in LB+Rifampicin (Rif) and Kanamycin (Kan) media at 28 C 48 Hrs.

Transfer 200 ul for previous culture into 100 ml LB+Rif and Kan media at 28 C for 24 Hrs.

Spin down culture at 4 C and resuspend cells in MS+10 g/l glucose+15 g/l sucrose and pH 5.8) to obtain OD₆₀₀˜0.6-0.8. Agrobacterium cells were activated by treating with 200 μM acetosyringone (AS) for 45-60 min in dark before infection.

Add cotyledon/hypocotyl into the agrobacterium for 15-20 min with continuous shaking at 28 C.

Transfer infected explants to sterile filter paper and dry. Transfer to co-culture media* at 25 C for 48 Hrs.

After 2-3 days of co-cultivation, the infected explants were washed 3 times in sterile water and then washed once in sterile water containing 400 mg/l Timentine (Tim) and again in sterile water containing 200 mg/l Timentine to remove Agrobacterium.

The washed explants were dried on sterile filter papers and cultured on Regeneration-selection containing 160mg/l Timentine and 5 mg/l Hygromycin (Hyg). Kept under 16 hr photoperiod for 15 days and 25 C.

After first round of selection for 15 days, brownish or black coloured explants were discarded.

For hypocotyls, shooting/rooting may occur during the first 15 days on selection media.

For Cotyledon, callus may be formed in the proximal side and shoots may be already visible.

Healthy explants were transferred to fresh regeneration-selection media* for second selection cycle for 15 days (A third cycle may be needed depending explant appearance and development).

After selection:

Hypocotyl: Those explants generating shoots and roots can be transferred to compost for acclimatization.

Cotyledon: Shoots formed from callus may be transferred to rooting media*. *Cotyledon Co-culture/Regeneration-Selection media (Tim 160 mg/l +Hyg 5 mg/L).

| | | | | TDZ | NAA | AgNO3 |
|---|---|---|---|---|---|---|
| Cultivars | MS | Agar | Sucrose | mg/l | mg/l | mg/l |
| Co-cultivation/ | 4.93 g/l | 8 g/l | 30 g/l | 0.6 | 0.3 | 5 |
| Regeneration | | | | | | |
| | | | | IBA | | AgNO3 |
| | MS | Agar | Sucrose | mg/l | | mg/l |
| Rooting | 2.46 | 8 g/l | 30 g/l | 1 | | 5 |

*Hypocotyl Co-culture/Regeneration-Selection media (Tim 160 mg/l + Hyg 5 mg/L).

| | | | | | | Nicotinic | Myo- |
|---|---|---|---|---|---|---|---|
| Cultivars | ½MS | Gelrite | Sucrose | Thiamine | Pyridoxine | acid | inositol |
| Co-cultivation**/ | 2.46 g/l | 3.5 g/l | 1.5% | 0.01 mg/l | 0.001 mg/l | 0.001 mg/l | 10 mg/l |
| Regeneration**/ | | | | | | | |
| rooting | | | | | | | |

**Add 3 mM MES and 5 mg/l AgNO3 to avoid browning and enhance shoot proliferation.

### Hypocotyl Inoculation

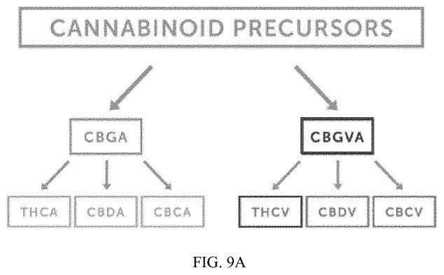

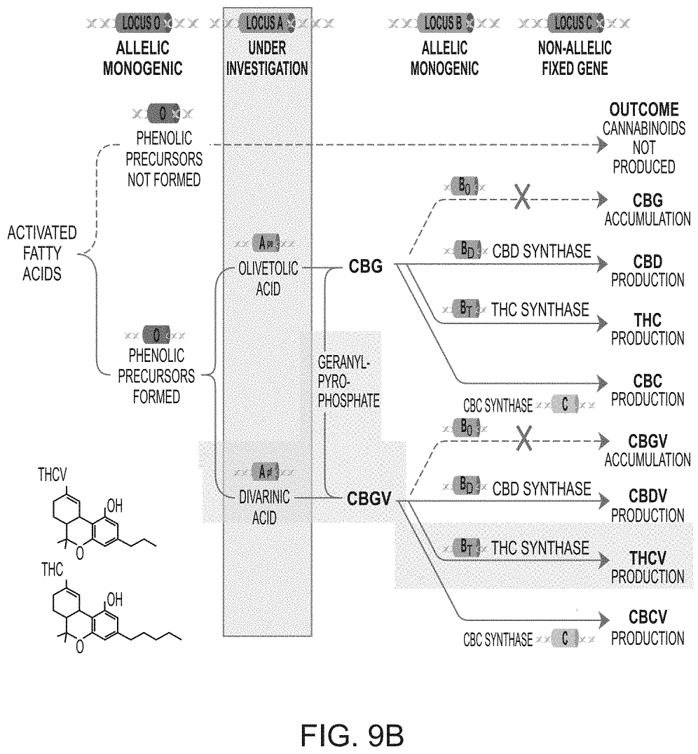

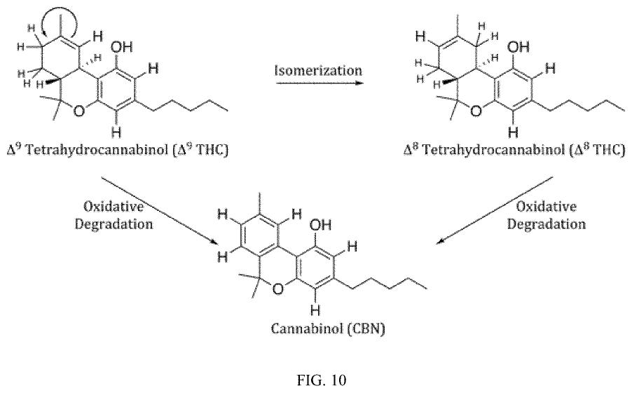

The hypocotyl is part of the stem of an embryonic plant, beneath the stalks of the seed leaves or cotyledons, and directly above the root. Hypocotyls were excised from 5-7 days old plantlets and submerged into a suspension of agrobacterium carrying the GUS reporter vector pCambia1301. After 3 days on Timentine growth-media, inoculated hypocotyls were transferred to Hygromycin selection plates for 5 days. Then the tissue was stained with X-Gluc and GUS expression visualized. The blue staining was observed in regenerated explants (indicated by white arrows shown in FIGS. 9A and 9C) and regenerative tissue (indicated by white arrows shown in FIGS. 9B and 9D).

### Protoplast Isolation and Transformation

Protocols have been developed for the successful isolation of healthy viable protoplasts from Hemp and Cannabis leaves. The Isolated protoplast transfection conditions were developed using PEG-transfection of plasmid DNA. Initial evaluation of transformation efficiencies were performed with the GUS reporter gene vector and conditions identified for successful introduction and expression of the plasmids.

### Floral Dipping

Floral dipping has been used successfully in model plant systems such as Arabidopsis Thaliana, as a method for direct introduction of Agrobacterium into the flowers of growing plantlets. The immature female flowers, containing the sexual organs were immersed into an Agrobacterium suspension carrying the desired vector (either GUS reporter or CRISPR gRNA). After two rounds of dipping, female flowers were crossed with male pollen to obtain seeds in an attempt to produce seeds carrying the transformed DNA in the germline. Seeds may be grown on selective media to confirm transformation and integration of the drug selection marker and transmission of the CRISPR modified genome.

### Callus Regeneration

Multiple experiments were conducted to identify growth conditions to obtain Cannabis and Hemp callus tissue with the quality and viability to enable regeneration of mature plants.

TABLE 12
showing the different growth factors and nutrients test in various combinations

| MS source | Sugar Source | Agar Type | Cytokinins | Auxins | Nitrogen | Vitamins | Additives |
|---|---|---|---|---|---|---|---|
| MS basal | Sucrose | Agar | BAP | NAA | Glutamine | Thiamine | CuSO4 |
| MSB5 | Maltrose | Type E | Kin | IAA | Caseine | Pyridoxine | AgNO3 |
| | | Agar | | | | | |
| | | BactoAgar | Zea | IBA | | Nicotonic acid | |
| | | Gelrite | TDZ | 2-4D | | Myo-Inositol | |
| | | | | Dicamba | | | |

Two callus generation protocols and media compositions showed promising looking callus with the ideal characteristics for regeneration: Granular, breakable and dry.

From first protocol 1.31 listed below performed the best and was expanded to protocols 1.97 to 1.104, and from this method, 1.97 and 1.98 enabled the generation of callus with the ideal characteristics.

| | MS | Sucrose | Agar type | IAA | IBA | NAA | TDZ | Caseine | Myo-inos | Prolien | Thiamine | CuSO4 |
|---|---|---|---|---|---|---|---|---|---|---|---|---|
| | g/L | g/L | E g/L | mg/L | mg/L | mg/L | mg/L | g/L | mg/L | mg/L | mg/L | mg/L |
| Cl.1.31 | 4.92 | 30 | 8 | 0.09 | | | 0.22 | | | | | |
| Cl.1.32 | 4.92 | 30 | 8 | 0.18 | | | 0.22 | | | | | |
| Cl.1.33 | 4.92 | 30 | 8 | 0.26 | | | 0.22 | | | | | |
| Cl.1.34 | 4.92 | 30 | 8 | 0.36 | | | 0.22 | | | | | |
| Cl.1.35 | 4.92 | 30 | 8 | | 0.1 mg/l | | 0.22 | | | | | |
| Cl.1.36 | 4.92 | 30 | 8 | | 0.2 mg/l | | 0.22 | | | | | |
| Cl.1.37 | 4.92 | 30 | 8 | | 0.3 mg/l | | 0.22 | | | | | |
| Cl.1.38 | 4.92 | 30 | 8 | | 0.4 mg/l | | 0.22 | | | | | |
| Cl.1.97 | 4.92 | 30 | 8 | 0.09 | | | 0.05 | | | | | |
| Cl.1.98 | 4.92 | 30 | 8 | 0.09 | | | 0.1 | | | | | |
| Cl.1.99 | 4.92 | 30 | 8 | 0.09 | | | 0.22 | | | | | |
| Cl.1.100 | 4.92 | 30 | 8 | 0.09 | | | 0.44 | | | | | |
| Cl.1.101 | 4.92 | 30 | 8 | 0.09 | | | 0.05 | 1 | 350 | 690 | 1 | 1.25 |
| Cl.1.102 | 4.92 | 30 | 8 | 0.09 | | | 0.1 | 1 | 350 | 690 | 1 | 1.25 |
| Cl.1.103 | 4.92 | 30 | 8 | 0.09 | | | 0.22 | 1 | 350 | 690 | 1 | 1.25 |
| Cl.1.104 | 4.92 | 30 | 8 | 0.09 | | | 0.44 | 1 | 350 | 690 | 1 | 1.25 |

Two callus generation protocols and media compositions showed promising looking callus with the ideal characteristics for regeneration: Granular, breakable and dry. From first protocol 1.31 performed the best and was expanded to protocols 1.97 to 1.104, and from this method, 1.97 and 1.98 enabled the generation of callus with the ideal characteristics.

| | MS | Sucrose | Gelrite | IAA | TDZ | Pyridoxine | Myo-inos | Nicotinic | Thiamine | AgNO3 |
|---|---|---|---|---|---|---|---|---|---|---|
| | g/L | g/L | g/L | mg/L | mg/L | g/L | mg/L | acid mg/L | mg/L | mg/L |
| Cl.1.98.1 | 4.92 | 30 | 3.5 | 0.05 mg/l | 0.1 mg/l | 0.001 (25 ul) | 10 (1428 ul) | 0.001(25 ul) | 0.01(50 ul) | 0.5 mg/l |
| Cl.1.98.2 | 4.92 | 30 | 3.5 | 0.05 mg/l | 0.1 mg/l | | | | | 0.5 mg/l |
| Cl.1.98.3 | 4.92 | 30 | 3.5 | 0.15 mg/l | 0.1 mg/l | 0.001 (25 ul) | 10 (14.28 ul) | 0.001(25 ul) | 0.01(50 ul) | 0.5 mg/l |
| Cl.1.98.4 | 4.92 | 30 | 3.5 | 0.15 mg/l | 0.1 mg/l | | | | | 0.5 mg/l |

### Cotyledon Regeneration

Regeneration of mature plants from cotyledon tissue is a proven method for fast regeneration when compared to callus formation in other plants. Regeneration was observed from two distinct sources: direct from meristem and indirect from small callus formation.

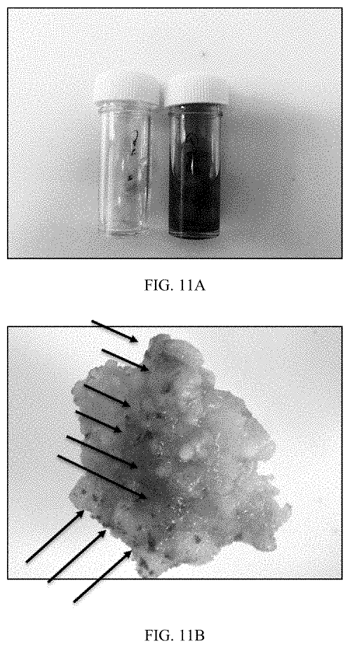

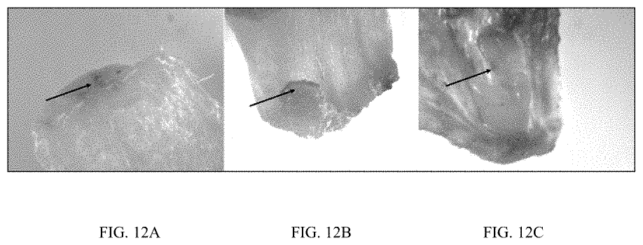

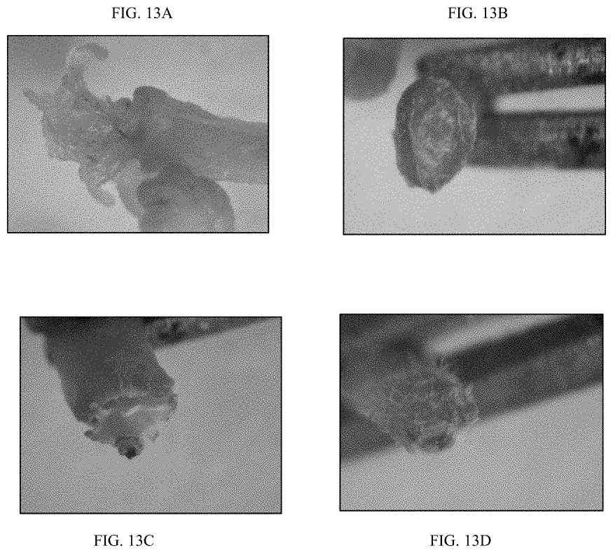

Protocols were developed that have demonstrated early regeneration capacities as shown in FIGS. 12A-12C.

### Hypocotyl Regeneration

Regeneration protocols were developed to now show Hypocotyl to be highly regenerative, forming adult plants without vitrification problems. Hypocotyl excised from 5-7 days old plantlets regenerated roots and small shoots in the first 5-7 days. Once shoots and roots were regenerated, plantlets were transferred to bigger pots where they remain for 3-4 weeks before transferring them to compost.

| Cultivar | MS | Sucrose | Gelrite | Myo-inositol | Pyridoxine | Nicotinic Acid | Thiamine |
|---|---|---|---|---|---|---|---|
| Finola (Hemp) | ½ | 1.5% | 3.5 g/L | 10 mg/L | 0.001 gl/L | 0.001 mg/L | 0.01 mg/L |

### Example 5

### Shoot Regeneration and Other Regeneration Methods

### Shoot Regeneration

Agrobacterium treated callus are transferred to MS+3% sucrose and 0.8% Bacteriological agar (pH 5.8. Autoclaved at this point. Filtered sterilized 0.5 uM TDZ is added along with a selective antibiotic (depending on vector used) and 160-200 mg/l of Timentin for shoot regeneration. The reaction mixture is placed at 25 C +/−2 and 16/8H photoperiod and 36-52 uM/m/s light intensity (Acclimation process could be used by placing tissue paper on top to avoid excessive light for at least 1-2 weeks).

Once shoots are observed and established, approximately 2-3 weeks, plantlets are transferred to Rooting media containing: half MS media+3% sucrose, 0.8% Bacteriological agar (ph 5.8. and autoclave). Filtered sterilized 2.5 uM IBA and selective antibiotic are added (depending on vector used) along with 160-200 mg/l of Timentin. The reaction mixture is placed at 25 +/− 2 C, 16/8 h photoperiod and 36-52 uM×m-1×s-1 intensity. Established plants are planted in soil. Explant's roots are cleaned from agar. Plantlets are covered once in the pot using a plastic sleeve to maintain humidity. Plants are kept under controlled environmental conditions (25±3° C. temperature and 36-55±5% RH).

### Method 1: Protoplast Extraction Transfection and Regeneration in Cannabis Reagents

Enzyme solution: 20 mM MES (pH 5.7) containing 1.5% (wt/vol) cellulase R10, 0.4% (wt/vol) macerozyme R10, 0.4 M mannitol and 20 mM KCl is prepared. The solution is warmed at 55° C. for 10 min to inactivate DNAse and proteases and enhance enzyme solubility. Cool it to room temperature (25° C.) and add 10 mM CaCl₂, 1-5 mM β-mercaptoethanol (optional) and 0.1% BSA. Addition of 1-5 mM β-mercaptoethanol is optional, and its use should be determined according to the experimental purpose. Additionally, before the enzyme powder is added, the MES solution is preheated at 70° C. for 3-5 min. The final enzyme solution should be clear light brown. Filter the final enzyme solution through a 0.45-μm syringe filter device into a Petri dish (100×25 mm² for 10 ml enzyme solution).

WI solution: 4 mM MES (pH 5.7) containing 0.5 M mannitol and 20 mM KCl is prepared. The prepared WI solution can be stored at room temperature (22-25° C.).

W5 solution: 2 mM MES (pH 5.7) containing 154 mM NaCl, 125 mM CaCl ₂ and 5 mM KCl is prepared. The prepared W5 solution can be stored at room temperature.

MMG solution: 4 mM MES (pH 5.7) containing 0.4 M mannitol and 15 mM MgCl₂. The prepared MMG solution can be stored at room temperature.

PEG—calcium transfection solution 20-40% (wt/vol) PEG4000 in ddH2O containing 0.2 M mannitol and 100 mM CaCl₂. PEG solution is prepared at least 1 h before transfection to completely dissolve PEG. The PEG solution can be stored at room temperature and used within 5 d. However, freshly prepared PEG solution gives relatively better protoplast transfection efficiency. Do not autoclave PEG solution.

Protoplast lysis buffer: 25 mM Tris-phosphate (pH 7.8) containing 1 mM DTT, 2 mM DACTAA, 10% (vol/vol) glycerol and 1% (vol/vol) Triton X-100. The lysis buffer is prepared fresh.

MUG substrate mix for GUS assay 10 mM Tris-HCl (pH 8) containing 1 mM MUG and 2 mM MgCl₂. The prepared GUS assay substrate can be stored at −20° C.

### Plant Growth

Plant growth can take from about 3-4 weeks. In brief, seeds are disinfected using ethanol 70% for 30 sec and 5% bleach for 5-10 min. Seeds are washed using sterile water 4 times. Seeds are germinated on half-strength ½ MS medium supplemented with 10 g·L-1 sucrose, 5.5 g·L-1 agar (pH 6.8) at 25 +/− 2 C under 16/8 photoperiod. Young leaves are selected, 0.5-10 mm (Additionally, other tissues may be considered such as cotyledons, petioles) for initiation of shoot culture. Explants are disinfected using 0.5% NaOCL (15% v/v bleach) and 0.1% tween 20 for 20 min (Optional as plantlets are growing in an aseptic environment). Plant growth was monitored for contamination. Additionally, different tissues such as young leaves or coleoptiles can be tested.

### Protoplast Isolation

Protoplast isolation can take about 4-5 hrs. In brief, well-expanded leaves are chosen from 3-4-week-old plants (usually about five to seven. Plant age is tested at this time.) before flowering. The selection of healthy leaves at the proper developmental stage is considered a factor in protoplast experiments. Protoplasts prepared from leaves recovered from stress conditions (e.g., drought, flooding, extreme temperature and constant mechanical perturbation) may look similar to those from healthy leaves. However, low transfection efficiency may occur with the protoplasts from stressed leaves. High stress—induced cellular status can also be a problem in the study of stress, defense and hormonal signaling pathways.

0.5-1-mm leaf strips are cut from the middle part of a leaf using a fresh sharp razor blade without tissue crushing at the cutting site. A good preparation yields approximately 10⁷ protoplasts per gram fresh weight (approximately 100-150 leaves digested in 40-60 ml of enzyme solution). For routine experiments, 10-20 leaves digested in 5-10 ml enzyme solution will give 0.5-1 ×10⁶ protoplasts, enough for more than 25-100 samples (1-2×10⁴protoplasts per sample). The blade is changed after cutting four to five leaves. Leaves are cut on a piece of clean white paper (8″×11″) on top of the solid and clean laboratory bench, which provides for good support and easy inspection of wounded/crushed tissue (juicy and dark green stain).

Leaf strips are transferred quickly and gently into the prepared enzyme solution (10-20 leaves in 5-10 ml.) by dipping both sides of the strips (completely submerged) using a pair of flat-tip forceps. In some cases, immediate dipping and submerging of leaf strips is a factor considered for protoplast yield. When leaf strips are dried out on the paper during cutting, the enzyme solution cannot penetrate and protoplast yield can be decreased. Afterwards, infiltrate leaf strips are vacuumed for 30 min in the dark using a desiccator. The digestion is continued, without shaking, in the dark for at least 3 h at room temperature. The enzyme solution should turn green after a gentle swirling motion, which indicates the release of protoplasts. Digestion time depends on the experimental goals, desirable responses and materials used, and can be optimized empirically. After 3 h digestion, most protoplasts are released from leaf strips in case of Col-0. However, the protoplast isolation efficiency differs significantly for different ecotypes and genotypes. The digesting time has to be optimized for each ecotype and genotype. Prolonged incubation of leaves (16-18 h) in the dark is stressful and might eliminate physiological responses of leaf cells. However, the stress might be important for the dedifferentiation and regeneration processes recommended in other protoplast protocols. The release of protoplasts in the solution is monitored under the microscope; the size of Arabidopsis mesophyll protoplasts is approximately 30-50 μm.

The enzyme/protoplast solution is diluted with an equal volume of W5 solution before filtration to remove undigested leaf tissues. A clean 75-μm nylon mesh with water is used to remove ethanol (the mesh is normally kept in 95% ethanol) then excess water is removed before protoplast filtration. Filter the enzyme solution containing protoplasts after wetting the 75-μm nylon mesh with W5 solution. The solution is centrifuged, the flow-through at 100 g-200 g, to pellet the protoplasts in a 30-ml round-bottomed tube for 1-2 min. Supernatant is removed. The protoplast pellet is resuspended by gentle swirling. A higher speed (200 g) of centrifugation may help to increase protoplast recovery. Protoplasts are resuspended at 2×10⁵ ml⁻¹ in (2×10⁵ per ml of W5) W5 solution after counting cells under the microscope (×100) using a hemocytometer. The protoplasts are kept on ice for 30 min. Additionally, protoplasts can be kept at room temperature. Although the protoplasts can be kept on ice for at least 24 h, freshly prepared protoplasts should be used for the study of gene expression regulation, signal transduction and protein trafficking, processing and localization. Protoplasts settle at the bottom of the tube by gravity after 15 min. the W5 solution is removed as much as possible without touching the protoplast pellet. Re-suspend protoplasts at 2×10⁵ per ml of MMG solution and kept at room temperature.

### DNA-PEG—Calcium Transfection

A transfection is performed by adding 10 μl DNA (10-20 μg of plasmid DNA of 5-10 kb in size) to a 2-ml microfuge tube. 100 μl MMG/protoplasts is added (2×10⁴ protoplasts) and mixed gently. 110 μl of PEG solution is added, and then mixed completely by gently tapping the tube. The transfection mixture is maintained at room temperature for up to 15 min (5 min is sufficient). The transfection mixture is maintained in 400-440 μl W5 solution at room temperature and well mixed by gently rocking or inverting to stop the transfection process. The reaction mixture is centrifuged at 100 g for 2 min at room temperature using a bench-top centrifuge and supernatant removed. Protoplasts are resuspended gently with 1 ml WI in each well of a 6-well tissue culture plate.

Additionally, high transfection efficiency can be achieved using 10-20% PEG final concentration. The optimal PEG concentration is determined empirically for each experimental purpose. Visual reporters such as GFP are used to determine optimal DNA transfection conditions. If protoplasts are derived from healthy leaf materials, most protoplasts should remain intact throughout the isolation, transfection, culture and harvesting procedures.

### Protoplast Culture and Harvest

Protoplasts are incubated at room temperature (20-25° C.) for the desired period of time.

### Method 2: Protoplast Regeneration After Transfection

### Reagents

0.2 M 4-morpholineethanesulfonic acid (MES, pH 5.7; Sigma, cat. no. M8250), sterilize using a 0.45-μm filter

0.8 M mannitol (Sigma, cat. no. M4125), sterilize using a 0.45-μm filter

1 M CaCl₂ (Sigma, cat. no. C7902), sterilize using a 0.45-μm filter

2 M KCl (Sigma, cat. no. P3911), sterilize using a 0.45-μm filter

2 M MgCl₂ (Sigma, cat. no. M9272), sterilize using a 0.45-μm filter

β-Mercaptoethanol (Sigma, cat. no. M6250)

10% (wt/vol) BSA (Sigma, cat. no. A-6793), sterilize using a 0.45-μm filter

Cellulase R10 (Yakult Pharmaceutical Ind. Co., Ltd., Japan)

Macerozyme R10 (Yakult Pharmaceutical Ind. Co., Ltd., Japan)

1 M Tris—phosphate (pH 7.8), sterilize using a 0.45-μm filter

100 mM trans-1,2-diaminocyclo-hexane-N,N,N′,N′-tetraacetic acid (DACTAA; Sigma, cat. no. D-1383)

50% (vol/vol) glycerol (Fisher, cat. no. 15892), sterilize using a 0.45-μm filter

20% (vol/vol) Triton X-100 (Sigma, cat. no. T-8787)

1 M DTT (Sigma, cat. no. D-9779)

LUC assay system (Promega, cat. no. E1501)

1 M Tris—HCl (pH 8.0) (US Biological, cat. no. T8650), sterilize using a 0.45-μm filter

0.1 M 4-methylumbelliferyl glucuronide (MUG; Gold BioTechnology, Inc., cat. no. MUG-1G)

0.2 M Na₂CO₃ (Sigma, cat. no. 57795)

1 M methylumbelliferone (MU; Fluka, cat. no. 69580)

Metro-Mix 360 (Sun Gro Horticulture, Inc.)

Jiffy? (Jiffy Products Ltd., Canada)

Arabidopsis accessions: Col-0 and Ler (ABRC)

After transfection, protoplast is transferred into a 5 cm diameter petri dish containing liquid callus medium (1/2MS medium supplemented with 0.4 M mannitol, 30 g/L sucrose, 1 mg/L NAA and 3 mg/L kinetin (pH5.8) and incubate 2-3 weeks in the dark at room temperature. After this time the proliferating calli form dust-like calli). Calli are embedded in solid callus medium (1/2MS medium supplemented with 0.4 M mannitol, 30 g/L sucrose, 1 mg/L NAA and 3 mg/L kinetin+0.4% agar, pH 5.8) in a 9 cm diameter petri dish for 3-4 weeks at 25 C. In the callus stage, the explants are incubated in the dark (gray background). Calli larger than 3 mm are embedded in solid shooting medium (MS medium supplemented with 2 mg/L kinetin, 0.3 mg/L IAA, 0.4 M mannitol, and 30 g/L sucrose +0.4% Agar, pH 5.8) for shoot induction at 25 C and 16/8 photoperiod (3000 lux) for a month. After one month, the multiple shoots which contain leaves or are of a size larger than 5 mm are transferred to fresh shooting medium (pH 5.8) for 2-3 weeks for shoot proliferation at 25 C and 16/8 photoperiod (3000 lux). After this time multiple shoots with leaves are transferred to solidified rooting medium (MS medium supplemented with 0.1 mg/L IAA, and 30 g/L sucrose+0.4% agar, pH 5.8) 25 C and 16/8 photoperiod (3000 lux).

### Agroinfiltration

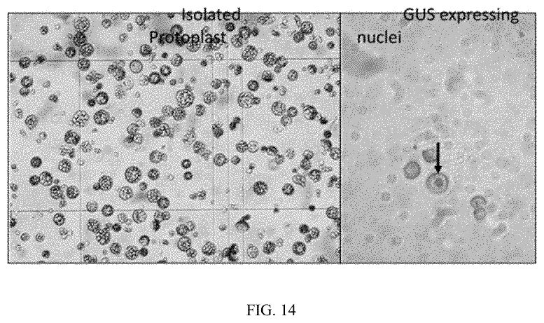

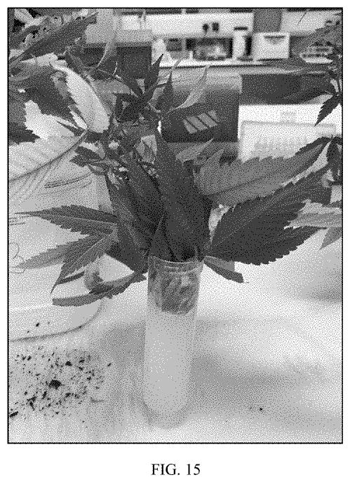

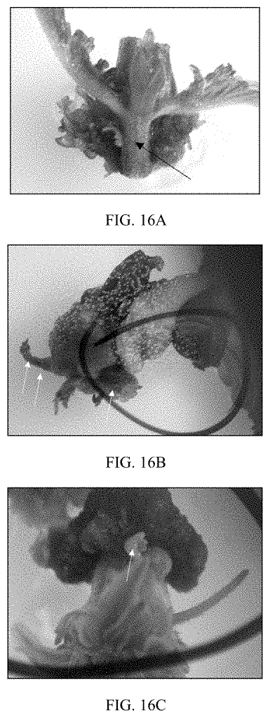

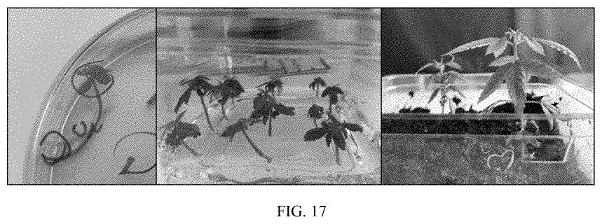

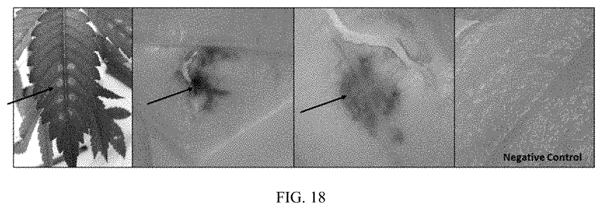

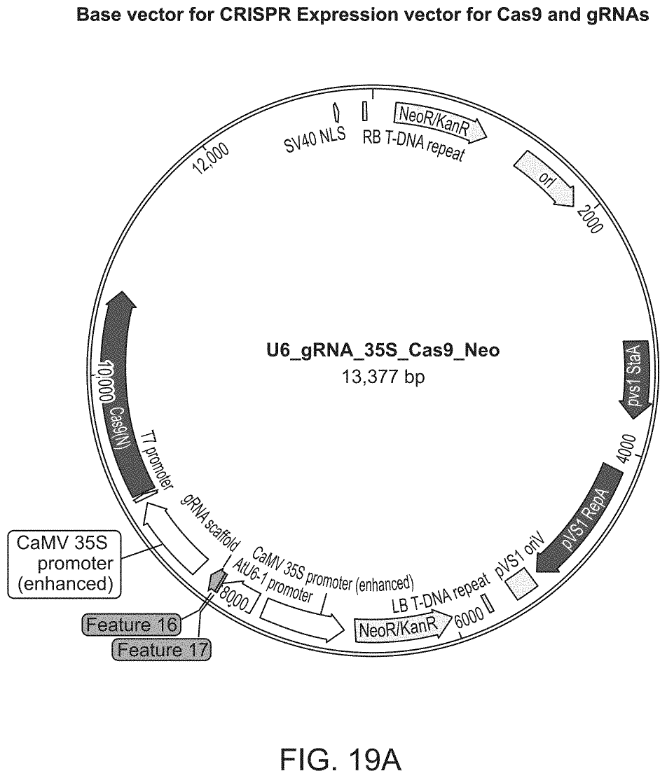

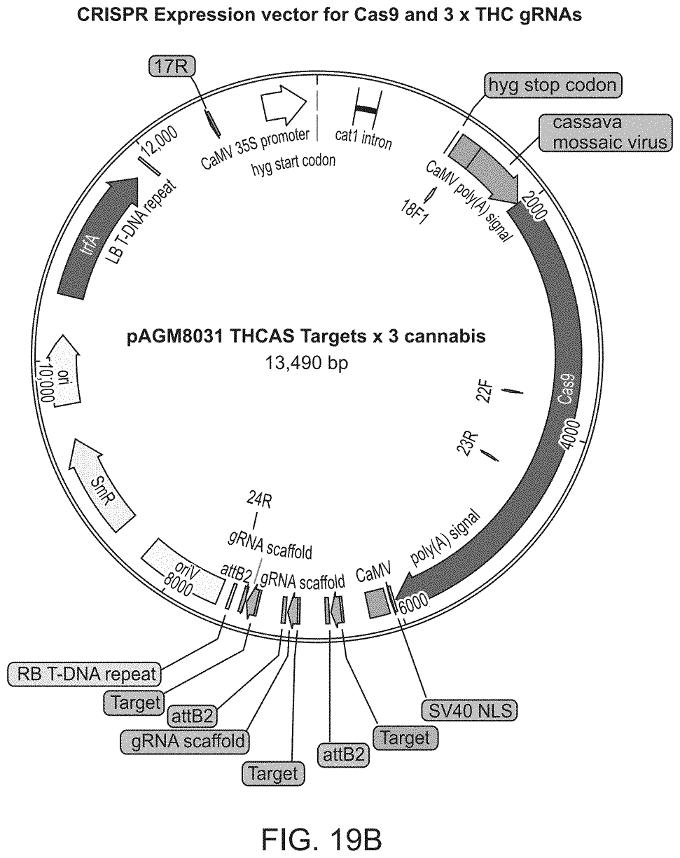

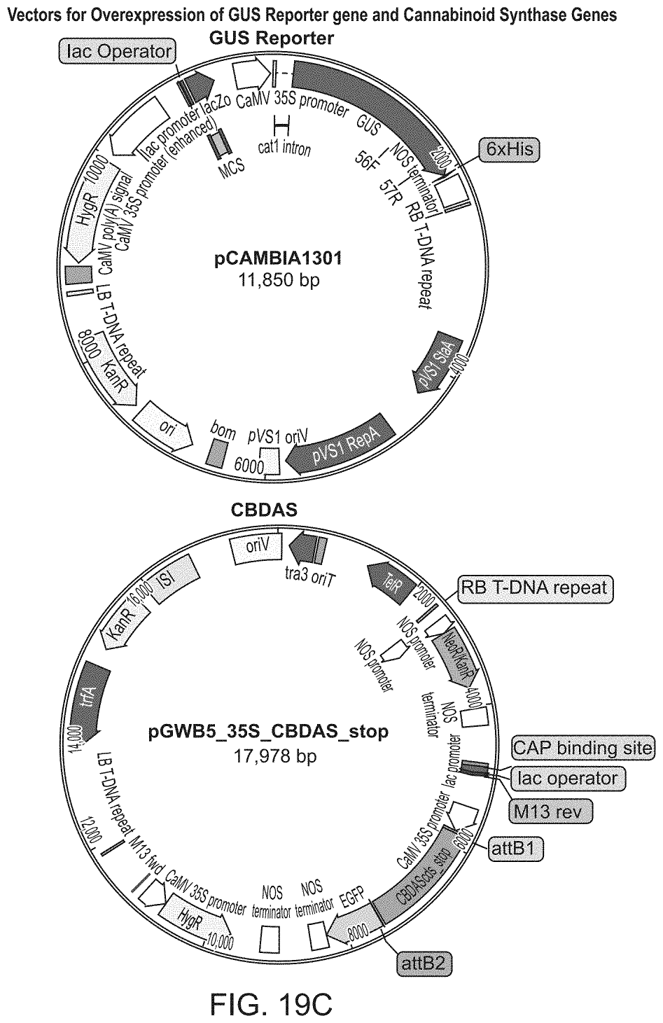

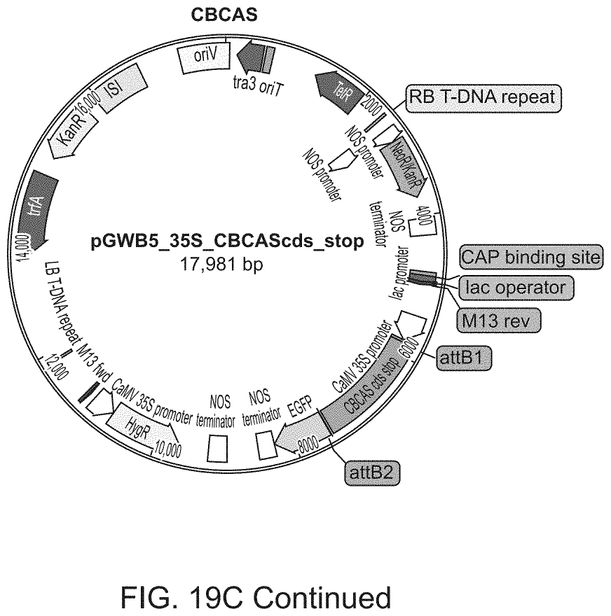

Agroinfiltration is a fast method to test Agrobacterium reagents in plant tissue. Protocols are developed to test the GUS reporter and CRISPR vectors in Agrobacterium in Cannabis and Hemp leaf tissue to demonstrate the agrobacterium can deliver the desired vector and that the vector expressed, enabling reporter gene expression and/or gene editing. The protocol comprises of infiltrating the Agrobacterium with a syringe into the adaxial part of the leave as shown in FIG. 14.

Disclosed below are protocols for agroinfiltration:

For plant growth conditions, first, sow cannabis seeds in water-soaked soil mix in a plant pot or in agar plate. Cover the pot with cling film and place it in a growth chamber with 16 h photoperiod cycle at 25/22° C. day and night respectively. Grow until the seedlings have two true leaves (around 7-10 days). Carefully transplant seedlings to the final destination in seed trays. Grow plants for approximately 3-4 more weeks inside the growth chamber. After this, plants are ready for infiltration.

With respect to agrobacterium cultures, this protocol can be used with, at least, three different commonly used strains of Agrobacterium: LBA4404, GV3101 and AGL1. For example, AGL1 has proven to be the most efficient. First, using a glycerol stock and a sterile toothpick, streak the Agrobacterium clone(s) to be used in LB solid plates supplemented with the appropriate antibiotics. Place the plates inside a 28° C. incubator for 48 h to obtain fresh and single colonies. The day before starting the infiltration, start liquid Agrobacterium cultures in LB liquid medium using the fresh colonies on the plates. Pick Agrobacterium biomass from a single colony, using a sterile toothpick, place it inside a sterile Erlenmeyer flask with 100 ml LB liquid media supplemented with the appropriate antibiotics, and culture them at 28° C. and 180 rpm overnight.

For the step of infiltration, pour saturated cultures into 50 ml Falcon tubes to prepare agrobacterium. Spin down cells at 4,000×g for 10 min. Discard LB medium supernatant by decanting. Eliminate as much supernatant as possible and resuspend with vortex the cell pellets using 1 volume of freshly prepared infiltration buffer. After resuspension, leave cultures for 2-4 h in darkness at room temperature. Subsequently, prepare a 1/20 dilution of the saturated culture, measure OD600 and calculate necessary volume to have a final OD600 of 0.05. Dilute using infiltration buffer.

Once the agrobacterium is prepared, fill a 1 or 2 ml needleless syringe with the resuspended culture at a final OD600 of 0.05. Perform the infiltration by pressing the syringe (without needle) on the abaxial side of the leaf while exerting counter-pressure with a fingertip on the adaxial side. Observe how the liquid spreads within the leaf if the infiltration is successful. Infiltrate whole leaves (ca. 100 μl of bacterial suspension/leave). Dry the excess of culture from the leaf surface using tissue paper. Two to four days after infiltration, observe fluorescence of infiltrated proteins or harvest infiltrated leaves to do a protein extraction.

Infiltration solution (100 ml)

| | | |
|---|---|---|
| | | Final |
| Reagent | Volume | concentration |

| | | | | |
|---|---|---|---|---|
| 1M MES | 1 | ml | 10 | mM |
| 1M MgCl₂ | 1 | ml | 10 | mM |
| 0.1M acetosyringone | 100 | μl | 0.1 | mM |

The MES solution can be prepared with sterile deionized water by adding 17.5 g MES to sterile deionized water. Then adjust the pH of the solution to 5.6 and sterilize the solution by filtration. The infiltration solution can be stored at room temperature. The MgCl₂ solution can be prepared by adding 20.3 g MgCl₂to sterile deionized water. The MgCl₂ solution may be sterilized by autoclaving and stored at room temperature. The acetosyringone solution can be prepared by adding 0.196 g acetosyringone to 10 ml DMSO. The acetosyringone solution can be prepared as 1 ml aliquots and stored at −20° C.

For Cannabis protoplasting, BSA (10 mg/ml ): 0.1 g in 10 ml H20 (need to be frozen), MgCl₂ 500 mM, CaCl₂ 1M, KCL 1M, KOH 1M, NaCl 5M are solutions needed for needed for protoplast extraction in Cannabis. MES-KOH 100 mM (50 ml-pH 5.6) is prepared by adding 0.976g MES to about 1 ml 1M KOH. Mannitol 1M (50 ml) may be prepared in multiple stocks by adding 9.11 g Mannitol to water (heat to 55 C to dissolve), which may be stored frozen. Plasmolysis buffer (0.6 M Mannitol — 10 ml) may be made fresh by adding 6 ml Mannitol 1M (0.6 M final conc.) to 4 ml water. Enzyme solution (20 ml) comprising 0.3g Cellulase RS (sigma C0615) (1.5% final), 0.15 g Macerozyme R10 (Calbiochem) (0.75% final), 1 ml KCL 1M (10 mM final concentration), 0.8 ml water, 12 ml 1M Mannitol (0.6 M final conc.), 4 ml MES-KOH 100 (20 mM final conc.) may be made up fresh before each protoplasting and can be sterilized by filtration. The enzyme solution may be incubated for 10 mins at 55 C (water bath) to inactivate proteases and enhance enzyme solubility. After the enzyme solution is cooled then add 200 μl 1M CaCl₂ (10 mM final conc.) and 2 ml 10 mg/ml BSA (0.1% BSA final). For W5 solution (50 ml): make 2×50ml 40.5 ml water, 6.25 ml CaCl_{2 1}M (125mM final), 1.54 ml NaCl 5M (154 mM final), 1 ml MES-KOH 100 (2 mM final), and 0.25 ml KCL 1M (5 mM final). For W1 Solution (50 ml ): prepare 4 mM MES (pH 5.7) containing 0.5 M mannitol and 20 mM KCl. The prepared W1 solution can be stored at room temperature (22-25° C.). Prepare MMG solution (50 ml) by mixing 26.5 ml water, 20 ml Mannitol 1M (0.4 M Final), 1.5 ml MgCl₂ 500 mM (15 mM final), 2 ml MES-KOH (4 mM final), and PEG-CTS (5 ml). The PEG-CTS (5 ml) solution can be made 30 mins before by adding in order of 1 ml Mannitol 1M (0.2 M final conc.), 0.5 ml CaCl_{2 1}M (100 mM final conc), 2 g PEG 4000 (40% wt/vol final conc.), and water (up to 5 ml). Vortex can be used to mix the solution without heat.

For protoplast isolation protocols, switch on 55° C. incubator, then thaw 1 M Mannitol (55° C.), and make up fresh enzyme solution. Cut 10-20 shoots from 9-12 day old plants into big beaker with distilled water and swirl. Bunch up leaves in petri dish and cut 0.5 -1 mm leaf strips with fresh razor blade. Pour in 10 ml of Plasmolysis buffer (0.6 M Mannitol) and incubate for 10 mins (dark). Remove Plasmolysis buffer with 5 ml pipette without sucking up leaf strips and discard. Transfer tissue to 125 ml glass beaker using the razor blade and add all 20 ml of enzyme solution. Gently swirl to mix then wrap in foil. Place beaker in dessicator (dark). Turn on pump and incubate for 30 minutes. Incubate in dark for 4 hours at 23° C. with gentle shaking (60 RPM). Add 20 ml of room temp W5 to enzyme solution and swirl for 10 s to release protoplasts. Place a 40 μm nylon mesh in a non-skirted 50 ml tube. Swirl enzyme solution round and gently pour slowly through mesh (keep tube on a slight angle to limit fall of liquid). With the remaining 30 ml of W5, wash the leaf strips in the mesh 3-5 times with W5 solution and catch in a fresh non-skirted 50 ml tube. Balance and centrifuge both tubes 3 mins at 80×G—discard supernatant carefully. Resuspend both pellets in 10 ml W5 solution (Combine into one tube then swirl and remove a drop for the haemocytometer). Count protoplasts with haemocytometer (10× mag). (Place cover slip on slide and add protoplast drop to top and bottom to be drawn in by capillary action). Spin down again 3 mins at 80×G. Make the PEG-CTS solution. This should be dissolved and vortexed 30 mins before use. It may require 10 mins or vortexing but it needs to be as fresh as possible. Remove supernatant from protoplasts—Intact protoplasts will have settled by gravity in 30 mins. Try and remove as much liquid as possible without sucking up all the protoplasts. Resuspend protoplasts from second spin (11) to ˜1×10⁶ cell per ml in MMG Transformation. Pipette 10-20 μl plasmid (10-20 μg) into 2 ml Eppendorf. Add 100 μl protoplast (˜100,000 cells) to DNA, mix gently but well by moving tube nearly horizontal and tapping tube. Add 110 μl PEG-CTS. Mix gently as before by tapping tube. Incubate at 23 C for 10 mins in dark. Add 880 μl W5 solution to stop the transformation and mix by inverting tube. Spin at 80×G (1100 RPM in a minispin) for 3 mins and remove supernatant. Resuspend gently in 2 ml of W1 solution. Incubate in the dark at 23 C for 48 hours and remove most of supernatant to leave 200 μl of settled protoplasts.

Further, Table 13 lists several vectors that may be used to delivery CRISPR and gRNA.

TABLE 13
Vector sequences

| SEQ ID | | |
|---|---|---|
| NO | Name | Sequence |
| 9 | pAGM8031:AtU3promoter: | AGCCTGAACTCACCGCGACGTCTGTCGAGAAGTTTCT |
| | gRNA::Cassava | GATCGAAAAGTTCGACAGCGTCTCCGACCTGATGCA |
| | promoter:CAS9:3xTHCAS/ | GCTCTCGGAGGGCGAAGAATCTCGTGCTTTCAGCTTC |
| | CBCAS targets | GATGTAGGAGGGCGTGGATATGTCCTGCGGGTAAAT |
| | | AGCTGCGCCGATGGTTTCTACAAAGATCGTTATGTTT |
| | | ATCGGCACTTTGCATCGGCCGCGCTCCCGATTCCGGA |
| | | AGTGCTTGACATTGGGGAATTCAGCGAGAGCCTGAC |
| | | CTATTGCATCTCCCGCCGTGCACAGGGTGTCACGTTG |
| | | CAAGACCTGCCTGAAACCGAACTGCCCGCTGTTCTGC |
| | | AGGTAAATTTCTAGTTTTTCTCCTTCATTTTCTTGGTT |
| | | AGGACCCTTTTCTCTTTTTATTTTTTTGAGCTTTGATC |
| | | TTTCTTTAAACTGATCTATTTTTTAATTGATTGGTTAT |
| | | GGTGTAAATATTACATAGCTTTAACTGATAATCTGAT |
| | | TACTTTATTTCGTGTGTCTATGATGATGATGATAACT |
| | | GCAGCCGGTCGCGGAGGCCATGGATGCGATCGCTGC |
| | | GGCCGATCTTAGCCAGACGAGCGGGTTCGGCCCATT |
| | | CGGACCGCAAGGAATCGGTCAATACACTACATGGCG |
| | | TGATTTCATATGCGCGATTGCTGATCCCCATGTGTAT |
| | | CACTGGCAAACTGTGATGGACGACACCGTCAGTGCG |
| | | TCCGTCGCGCAGGCTCTCGATGAGCTGATGCTTTGGG |
| | | CCGAGGACTGCCCCGAAGTCCGGCACCTCGTGCACG |
| | | CGGATTTCGGCTCCAACAATGTCCTGACGGACAATG |
| | | GCCGCATAACAGCGGTCATTGACTGGAGCGAGGCGA |
| | | TGTTCGGGGATTCCCAATACGAGGTCGCCAACATCTT |
| | | CTTCTGGAGGCCGTGGTTGGCTTGTATGGAGCAGCA |
| | | GACGCGCTACTTCGAGCGGAGGCATCCGGAGCTTGC |
| | | AGGATCGCCGCGGCTCCGGGCGTATATGCTCCGCATT |
| | | GGTCTTGACCAACTCTATCAGAGCTTGGTTGACGGCA |
| | | ATTTCGATGATGCAGCTTGGGCGCAGGGTCGATGCG |
| | | ACGCAATCGTCCGATCCGGAGCCGGGACTGTCGGGC |
| | | GTACACAAATCGCCCGCAGAAGCGCGGCCGTCTGGA |
| | | CCGATGGCTGTGTAGAAGTACTCGCCGATAGTGGAA |
| | | ACCGACGCCCCAGCACTCGTCCGAGGGCAAAGGAAT |
| | | AGGCTTCTCTAGCTAGAGTCGATCGACAAGCTCGAG |
| | | TTTCTCCATAATAATGTGTGAGTAGTTCCCAGATAAG |
| | | GGAATTAGGGTTCCTATAGGGTTTCGCTCATGTGTTG |
| | | AGCATATAAGAAACCCTTAGTATGTATTTGTATTTGT |
| | | AAAATACTTCTATCAATAAAATTTCTAATTCCTAAAA |
| | | CCAAAATCCAGTACTAAAATCCAGATCGCTGCAAgcaa |
| | | gaattcaagcttggagccagaaggtaattatccaagatgtagcatcaagaatccaatgta |
| | | acgggaaaaactatggaagtattatgtaagctcagcaagaagcagatcaatatgcggc |
| | | acatatgcaacctatgttcaaaaatgaagaatgtacagatacaagatcctatactgccag |
| | | aatacgaagaagaatacgtagaaattgaaaaagaagaaccaggcgaagaaaagaatc |
| | | ttgatgacgtaagcactgacgacaacaatgaaaagaagaagataaggtcggtgattgtg |
| | | aaagagacatagaggacacatgtaaggtggaaaatgtaagggcggaaagtaaccttat |
| | | cacaaaggaatcttatcccccactacttatcatttatatttaccgtgtcatattgcccttga |
| | | gttttcctatataaggaaccaagttcggcatttgtgaaaacaagaaaaaatttggtgtaag |
| | | ctattttctttgaagtactgaggatacaacttcagagaaatttgtaagtttgtaatggacaag |
| | | aagtactccattgggctcgatatcggcacaaacagcgtcggctgggccgtcattacgga |
| | | cgagtacaaggtgccgagcaaaaaattcaaagttctgggcaataccgatcgccacagc |
| | | ataaagaagaacctcattggcgccctcctgttcgactccggggagacggccgaagcca |
| | | cgcggctcaaaagaacagcacggcgcagatatacccgcagaaagaatcggatctgct |
| | | acctgcaggagatctttagtaatgagatggctaaggtggatgactctacttccataggct |
| | | ggaggagtcctattggtggaggaggataaaaagcacgagcgccacccaatctaggc |
| | | aatatcgtggacgaggtggcgtaccatgaaaagtacccaaccatatatcatctgaggaa |
| | | gaagcttgtagacagtactgataaggctgacttgcggttgatctatctcgcgctggcgca |
| | | tatgatcaaatttcggggacacttcctcatcgagggggacctgaacccagacaacagc |
| | | gatgtcgacaaactctttatccaactggacagacttacaatcagcttacgaagagaacc |
| | | cgatcaacgcatccggagttgacgccaaagcaatcctgagcgctaggctgtccaaatc |
| | | ccggcggctcgaaaacctcatcgcacagctccctggggagaagaagaacggcctgtt |
| | | tggtaatcttatcgccctgtcactcgggctgacccccaactttaaatctaacttcgacctgg |
| | | ccgaagatgccaagcttcaactgagcaaagacacctacgatgatgatctcgacaatctg |
| | | ctggcccagatcggcgaccagtacgcagacctattaggcggcaaagaacctgtcaga |
| | | cgccattctgctgagtgatattctgcgagtgaacacggagatcaccaaagctccgctga |
| | | gcgctagtatgatcaagcgctatgatgagcaccaccaagacttgactttgctgaaggcc |
| | | cttgtcagacagcaactgcctgagaagtacaaggaaattttcttcgatcagtctaaaaatg |
| | | gctacgccggatacattgacggcggagcaagccaggaggaattttacaaatttattaag |
| | | cccatcttggaaaaaatggacggcaccgaggagctgctggtaaagcttaacagagaa |
| | | gatctgttgcgcaaacagcgcactttcgacaatggaagcatcccccaccagattcacct |
| | | gggcgaactgcacgctatcctcaggcggcaagaggatactacccctattgaaagataa |
| | | cagggaaaagattgagaaaatcctcacatttcggataccctactatgtaggccccctcgc |
| | | ccggggaaattccagattcgcgtggatgactcgcaaatcagaagagactatcactccct |
| | | ggaacttcgaggaagtcgtggataagggggcctctgcccagtccttcatcgaaaggat |
| | | gactaactttgataaaaatctgcctaacgaaaaggtgcttcctaaacactctctgctgtac |
| | | gagtacttcacagtttataacgagctcaccaaggtcaaatacgtcacagaagggatgag |
| | | aaagccagcattcctgtctggagagcagaagaaagctatcgtggacctcctcttcaaga |
| | | cgaaccggaaagttaccgtgaaacagctcaaagaagattatttcaaaaagattgaatgtt |
| | | tcgactctgttgaaatcagcggagtggaggatcgcttcaacgcatccctgggaacgtat |
| | | cacgatctcctgaaaatcattaaagacaaggacttcctggacaatgaggagaacgagg |
| | | acattcttgaggacattgtcctcacccttacgagtagaagatagggagatgattgaaga |
| | | acgcttgaaaacttacgctcatctcttcgacgacaaagtcatgaaacagctcaagaggc |
| | | gccgatatacaggatgggggcggctgtcaagaaaactgatcaatgggatccgagaca |
| | | agcagagtggaaagacaatcctggattttcttaagtccgatggatttgccaaccggaact |
| | | tcatgcagttgatccatgatgactctctcacctttaaggaggacatccagaaagcacaag |
| | | tttctggccagggggacagtctccacgagcacatcgctaatcttgcaggtagcccagct |
| | | atcaaaaagggaatactgcagaccgttaaggtcgtggatgaactcgtcaaagtaatggg |
| | | aaggcataagcccgagaatatcgttatcgagatggcccgagagaaccaaactaccca |
| | | gaagggacagaagaacagtagggaaaggatgaagaggattgaagagggtataaaag |
| | | aactggggtcccaaatccttaaggaacacccagttgaaaacacccagcttcagaatga |
| | | gaagctctacctgtactacctgcagaacggcagggacatgtacgtggatcaggaactg |
| | | gacatcaatcggctctccgactacgacgtggatcatatcgtgccccagtcttttctcaaag |
| | | atgattctattgataataaagtgttgacaagatccgataaaaatagagggaagagtgata |
| | | acgtcccctcagaagaagttgtcaagaaaatgaaaaattattggcggcagctgctgaac |
| | | gccaaactgatcacacaacggaagttcgataatctgactaaggctgaacgaggtggcc |
| | | tgtctgagttggataaagccggcttcatcaaaaggcagcttgagagacacgccagatc |
| | | accaagcacgtggcccaaattctcgattcacgcatgaacaccaagtacgatgaaaatga |
| | | caaactgattcgagaggtgaaagttattactctgaagtctaagctggtttcagatttcagaa |
| | | aggactacagattataaggtgagagagatcaacaattaccaccatgcgcatgatgccta |
| | | cctgaatgcagtggtaggcactgcacttatcaaaaaatatcccaagcttgaatctgaattt |
| | | gtttacggagactataaagtgtacgatgttaggaaaatgatcgcaaagtctgagcagga |
| | | aataggcaaggccaccgctaagtacttatttacagcaatattatgaattattcaagaccg |
| | | agattacactggccaatggagagattcggaagcgaccacttatcgaaacaaacggaga |
| | | aacaggagaaatcgtgtgggacaagggtagggatttcgcgacagtccggaaggtcct |
| | | gtccatgccgcaggtgaacatcgttaaaaagaccgaagtacagaccggaggcttctcc |
| | | aaggaaagtatcctcccgaaaaggaacagcgacaagctgatcgcacgcaaaaaagat |
| | | tgggaccccaagaaatacggcggattcgattctcctacagtcgcttacagtgtactggtt |
| | | gtggccaaagtggagaaagggaagtctaaaaaactcaaaagcgtcaaggaactgctg |
| | | ggcatcacaatcatggagcgatcaagcttcgaaaaaaaccccatcgactttctcgaggc |
| | | gaaaggatataaagaggtcaaaaaagacctcatcattaagcttcccaagtactctctcttt |
| | | gagcttgaaaacggccggaaacgaatgctcgctagtgcgggcgagctgcagaaaggt |
| | | aacgagctggcactgccctctaaatacgttaatttcttgtatctggccagccactatgaaa |
| | | agctcaaaggatctcccgaagataatgagcagaagcagctgttcgtggaacaacacaa |
| | | acactaccttgatgagatcatcgagcaaataagcgaattctccaaaagagtgatcctcgc |
| | | cgacgctaacctcgataaggtgctttctgcttacaataagcacagggataagcccatca |
| | | gggagcaggcagaaaacattatccacttgtttactctgaccaacttgggcgcgcctgca |
| | | gccttcaagtacttcgacaccaccatagacagaaagcggtacacctctacaaaggagg |
| | | tcctggacgccacactgattcatcagtcaattacggggctctatgaaacaagaatcgacc |
| | | tctctcagctcggtggagacagcagggctgaccccaagaagaagaggaaggtgtgag |
| | | cttctctagctagagtcgatcgacaagctcgagtactccataataatgtgtgagtagacc |
| | | cagataagggaattagggttcctatagggtttcgctcatgtgttgagcatataagaaaccc |
| | | ttagtatgtatttgtatttgtaaaatacttctatcaataaaatttctaattcctaaaaccaaaatc |
| | | cagtactaaaatccagatcgctactaggagcatcttcattcttaagatatgaagataatctt |
| | | caaaaggcccctgggaatctgaaagaagagaagcaggcccatttatatgggaaagaa |
| | | caatagtatttcttatataggcccatttaagttgaaaacaatcttcaaaagtcccacatcgct |
| | | tagataagaaaacgaagctgagtttatatacagctagagtcgaagtagtgcttgCCTC |
| | | TGTTCCCCAGAGGGCAgttttagagctagaaatagcaagttaaaataagg |
| | | ctagtccgttatcaacttgaaaaagtggcaccgagtcggtgctattactagacccagctt |
| | | tcttgtacaaagttggcattacgctttacgaattcccatggggagcatcttcattcttaagat |
| | | atgaagataatcttcaaaaggcccctgggaatctgaaagaagagaagcaggcccattta |
| | | tatgggaaagaacaatagtatttcttatataggcccatttaagttgaaaacaatcttcaaaa |
| | | gtcccacatcgcttagataagaaaacgaagctgagtttatatacagctagagtcgaagta |
| | | gtgcttgCTGTTCCCCAGAGGGCAGGGgttttagagctagaaatagca |
| | | agttaaaataaggctagtccgttatcaacttgaaaaagtggcaccgagtcggtgctttatt |
| | | ctagacccagctacttgtacaaagaggcattacgctcagagaattcgcatgcggagca |
| | | tcttcattcttaagatatgaagataatcttcaaaaggcccctgggaatctgaaagaagaga |
| | | agcaggcccatttatatgggaaagaacaatagtatacttatataggcccatttaagttgaa |
| | | aacaatcttcaaaagtcccacatcgcttagataagaaaacgaagctgagtttatatacag |
| | | ctagagtcgaagtagtgcttgAACCTCAAGCACGAGAACTTgttttag |
| | | agctagaaatagcaagttaaaataaggctagtccgttatcaacttgaaaaagtggcacc |
| | | gagtcggtgctattactagacccagctacttgtacaaagaggcattacgcttgtgtgag |
| | | accgaggatgcacatgtgaccgagggacacgaagtgatccgtttaaactatcagtgttt |
| | | gacaggatatattggcgggtaaacctaagagaaaagagcgtttattagaataatcggat |
| | | atttaaaagggcgtgaaaaggtttatccgttcgtccatttgtatgtgccagccgcctttgcg |
| | | acgctcaccgggctggttgccctc gccgctgggctggcggcc gtctatggccctgcaa |
| | | acgcgccagaaacgccgtcgaagccgtgtgcgagacaccgcggccgccggcgttgt |
| | | ggatacctcgcggaaaacttggccctcactgacagatgaggggcggacgttgacactt |
| | | gaggggccgactcacccggcgcggcgttgacagatgaggggcaggctcgatttcgg |
| | | ccggcgacgtggagctggccagcctcgcaaatcggcgaaaacgcctgattttacgcg |
| | | agtttcccacagatgatgtggacaagcctggggataagtgccctgcggtattgacacttg |
| | | aggggcgcgactactgacagatgaggggcgcgatccttgacacttgaggggcagagt |
| | | gctgacagatgaggggcgcacctattgacatttgaggggctgtccacaggcagaaaat |
| | | ccagcatttgcaagggtaccgcccgtattcggccaccgctaacctgtatttaacctgctt |
| | | ttaaaccaatatttataaaccttgtattaaccagggctgcgccctgtgcgcgtgaccgcg |
| | | cacgccgaaggggggtgcccccccttctcgaaccctcccggcccgctaacgcgggc |
| | | ctcccatccccccaggggctgcgcccctcggccgcgaacggcctcaccccaaaaatg |
| | | gcagcgctggccaattcccgaggcacgaacccagtggacataagcctgttcggttcgt |
| | | aagctgtaatgcaagtagcgtatgcgctcacgcaactggtccagaaccttgaccgaac |
| | | gcagcggtggtaacggcgcagtggcggttacatggcttgttatgactgtattaggggta |
| | | cagtctatgcctcgggcatccaagcagcaagcgcgttacgccgtgggtcgatgtttgat |
| | | gttatggagcagcaacgatgttacgcagcagggcagtc gccctaaaacaaagttaaac |
| | | atcatgggggaagcggtgatcgccgaagtatcgactcaactatcagaggtagaggcgt |
| | | catcgagcgccatctcgaaccgacgttgctggccgtacatagtacggctccgcagtgg |
| | | atggcggcctgaagccacacagcgatattgatttgctggttacggtgacc gtaaggctt |
| | | gatgaaacaacgcggcgagctttgatcaacgaccttttggaaacttcggcttcccctgga |
| | | gagagcgagattctccgcgctgtagaagtcaccattgttgtgcacgacgacatcattcc |
| | | gtggcgttatccagctaagcgcgaactgcaatttggagaatggcagcgcaatgacattc |
| | | ttgcaggtatcttcgagccagccacgatcgacattgatctggctatcttgctgacaaaag |
| | | caagagaacatagcgttgccttggtaggtccagcggcggaggaactctttgatccggtt |
| | | cctgaacaggatctatttgaggcgctaaatgaaaccttaacgctatggaactcgccgcc |
| | | cgactgggctggcgatgagcgaaatgtagtgcttacgttgtcccgcatttggtacagcg |
| | | cagtaaccggcaaaatcgcgccgaaggatgtcgctgccgactgggcaatggagcgc |
| | | ctgccggcccagtatcagcccgtcatacttgaagctagacaggcttatcttggacaaga |
| | | agaagatcgcttggcctcgcgcgcagatcagttggaagaatttgtccattacgtgaaag |
| | | gcgagatcaccaaggtagtcggcaaataatgtctagctagaaattcgttcaagccgacg |
| | | ccgcttcgcggcgcggcttaactcaagcgttagatgcactaagcacataattgctcaca |
| | | gccaaactatcaggtcaagtctgcttttattattataagcgtgcataataagccctacacaa |
| | | attgggagatatatcatgctgtcagaccaagtttactcatatatactttagattgatttaaaac |
| | | ttcattataatttaaaaggatctaggtgaagatcctattgataatctcatgaccaaaatccct |
| | | taacgtgagttacgttccactgagcgtcagaccccgtagaaaagatcaaaggatcttat |
| | | gagatcctattactgcgcgtaatctgctgcttgcaaacaaaaaaaccaccgctaccagc |
| | | ggtggtagtagccggatcaagagctaccaactctattccgaaggtaactggcttcagc |
| | | agagcgcagataccaaatactgtccttctagtgtagccgtagttaggccaccacttcaag |
| | | aactctgtagcaccgcctacatacctcgctctgctaatcctgttaccagtggctgctgcca |
| | | gtggcgataagtcgtgtcttaccgggttggactcaagacgatagttaccggataaggcg |
| | | cagcggtcgggctgaacggggggttcgtgcacacagcccagcttggagcgaacgac |
| | | ctacaccgaactgagatacctacagcgtgagctatgagaaagcgccacgcttcccgaa |
| | | gggagaaaggcggacaggtatccggtaagcggcagggtcggaacaggagagcgca |
| | | cgagggagcttccagggggaaacgcctggtatctttatagtcctgtcgggtttcgccac |
| | | ctctgacttgagcgtcgatttttgtgatgctcgtcaggggggcggagcctatggaaaaac |
| | | gccagcaacgcggcctttttacggttcctggcagatcctagatgtggcgcaacgatgcc |
| | | ggcgacaagcaggagcgcaccgacttcttccgcatcaagtgttttggctctcaggccga |
| | | ggcccacggcaagtatttgggcaaggggtcgctggtattcgtgcagggcaagattcgg |
| | | aataccaagtacgagaaggacggccagacggtctacgggaccgacttcattgccgata |
| | | aggtggattatctggacaccaaggcaccaggcgggtcaaatcaggaataagggcaca |
| | | ttgccccggcgtgagtcggggcaatcccgcaaggagggtgaatgaatcggacgtttga |
| | | ccggaaggcatacaggcaagaactgatcgacgcggggttttccgccgaggatgccga |
| | | aaccatcgcaagccgcaccgtcatgcgtgcgccccgcgaaaccttccagtccgtcgg |
| | | ctcgatggtccagcaagctacggccaagatcgagcgcgacagcgtgcaactggctcc |
| | | ccctgccctgcccgcgccatcggccgccgtggagcgttcgcgtcgtcttgaacaggag |
| | | gcggcaggtttggcgaagtcgatgaccatcgacacgcgaggaactatgacgaccaag |
| | | aagcgaaaaaccgccggcgaggacctggcaaaacaggtcagcgaggccaagcagg |
| | | ccgcgttgctgaaacacacgaagcagcagatcaaggaaatgcagctttccttgttcgat |
| | | attgcgccgtggccggacacgatgcgagcgatgccaaacgacacggcccgctctgcc |
| | | ctgttcaccacgcgcaacaagaaaatcccgcgcgaggcgctgcaaaacaaggtcattt |
| | | tccacgtcaacaaggacgtgaagatcacctacaccggcgtcgagctgcgggccgacg |
| | | atgacgaactggtgtggcagcaggtgttggagtacgcgaagcgcacccctatcggcg |
| | | agccgatcaccttcacgttctacgagctttgccaggacctgggctggtcgatcaatggc |
| | | cggtattacacgaaggccgaggaatgcctgtcgcgcctacaggcgacggcgatgggc |
| | | ttcacgtccgaccgcgttgggcacctggaatcggtgtcgctgctgcaccgcttccgcgt |
| | | cctggaccgtggcaagaaaacgtcccgttgccaggtcctgatcgacgaggaaatcgtc |
| | | gtgctgtttgctggcgaccactacacgaaattcatatgggagaagtaccgcaagctgtc |
| | | gccgacggcccgacggatgacgactatttcagctcgcaccgggagccgtacccgctc |
| | | aagctggaaaccttccgcctcatgtgcggatcggattccacccgcgtgaagaagtggc |
| | | gcgagcaggtcggcgaagcctgcgaagagttgcgaggcagcggcctggtggaaca |
| | | cgcctgggtcaatgatgacctggtgcattgcaaacgctagggccttgtggggtcagttc |
| | | cggctgggggttcagcagcccctgctcggatctgttggaccggacagtagtcatggttg |
| | | atgggctgcctgtatcgagtggtgattttgtgccgagctgccggtcggggagctgttgg |
| | | ctggctggtggcaggatatattgtggtgtaaacaaattgacgcttagacaacttaataaca |
| | | cattgcggacgtttttaatgtactggggttgaacactctgtgggtctcaTGCCGAAT |
| | | TCGGATCCGGAGGAATTCCAATCCCACAAAAATCTG |
| | | AGCTTAACAGCACAGTTGCTCCTCTCAGAGCAGAAT |
| | | CGGGTATTCAACACCCTCATATCAACTACTACGTTGT |
| | | GTATAACGGTCCACATGCCGGTATATACGATGACTG |
| | | GGGTTGTACAAAGGCGGCAACAAACGGCGTTCCCGG |
| | | AGTTGCACACAAGAAATTTGCCACTATTACAGAGGC |
| | | AAGAGCAGCAGCTGACGCGTACACAACAAGTCAGCA |
| | | AACAGACAGGTTGAACTTCATCCCCAAAGGAGAAGC |
| | | TCAACTCAAGCCCAAGAGCTTTGCTAAGGCCCTAAC |
| | | AAGCCCACCAAAGCAAAAAGCCCACTGGCTCACGCT |
| | | AGGAACCAAAAGGCCCAGCAGTGATCCAGCCCCAAA |
| | | AGAGATCTCCTTTGCCCCGGAGATTACAATGGACGA |
| | | TTTCCTCTATCTTTACGATCTAGGAAGGAAGTTCGAA |
| | | GGTGAAGGTGACGACACTATGTTCACCACTGATAAT |
| | | GAGAAGGTTAGCCTCTTCAATTTCAGAAAGAATGCT |
| | | GACCCACAGATGGTTAGAGAGGCCTACGCAGCAAGT |
| | | CTCATCAAGACGATCTACCCGAGTAACAATCTCCAG |
| | | GAGATCAAATACCTTCCCAAGAAGGTTAAAGATGCA |
| | | GTCAAAAGATTCAGGACTAATTGCATCAAGAACACA |
| | | GAGAAAGACATATTTCTCAAGATCAGAAGTACTATT |
| | | CCAGTATGGACGATTCAAGGCTTGCTTCATAAACCA |
| | | AGGCAAGTAATAGAGATTGGAGTCTCTAAAAAGGTA |
| | | GTTCCTACTGAATCTAAGGCCATGCATGGAGTCTAAG |
| | | ATTCAAATCGAGGATCTAACAGAACTCGCCGTCAAG |
| | | ACTGGCGAACAGTTCATACAGAGTCTTTTACGACTCA |
| | | ATGACAAGAAGAAAATCTTCGTCAACATGGTGGAGC |
| | | ACGACACTCTGGTCTACTCCAAAAATGTCAAAGATA |
| | | CAGTCTCAGAAGATCAAAGGGCTATTGAGACTTTTC |
| | | AACAAAGGATAATTTCGGGAAACCTCCTCGGATTCC |
| | | ATTGCCCAGCTATCTGTCACTTCATCGAAAGGACAGT |
| | | AGAAAAGGAAGGTGGCTCCTACAAATGCCATCATTG |
| | | CGATAAAGGAAAGGCTATCATTCAAGATCTCTCTGC |
| | | CGACAGTGGTCCCAAAGATGGACCCCCACCCACGAG |
| | | GAGCATCGTGGAAAAAGAAGAGGTTCCAACCACGTC |
| | | TACAAAGCAAGTGGATTGATGTGACATCTCCACTGA |
| | | CGTAAGGGATGACGCACAATCCCACTATCCTTCGCA |
| | | AGACCCTTCCTCTATATAAGGAAGTTCATTTCATTTG |
| | | GAGAGGACACGCTCGAGTATAAGAGCTCATTTTTAC |
| | | AACAATTACCAACAACAACAAACAACAAACAACATT |
| | | ACAATTACATTTACAATTATCGATACAATGAAAA |
| 10 | U6:gRNA::35S:CAS9::Neomycin | ctcgagcttctactgggcggttttatggacagcaagcgaaccggaattgccagctgggg |
| | | cgccctctggtaaggagggaagccctgcaaagtaaactggatggctactcgccgcca |
| | | aggatctgatggcgcaggggatcaagctctgatcaagagacaggatgaggatcgtttc |
| | | gcatgattgaacaagatggattgcacgcaggttctccggccgcttgggtggagaggct |
| | | attcggctatgactgggcacaacagacaatcggctgctctgatgccgccgtgaccggc |
| | | tgtcagcgcaggggcgcccggactattgtcaagaccgacctgtccggtgccctgaat |
| | | gaactgcaagacgaggcagcgcggctatcgtggctggccacgacgggcgttccttgc |
| | | gcagctgtgctcgacgttgtcactgaagcgggaagggactggctgctattgggcgaag |
| | | tgccggggcaggatctcctgtcatctcaccttgctcctgccgagaaagtatccatcatgg |
| | | ctgatgcaatgcggcggctgcatacgcttgatccggctacctgcccattcgaccaccaa |
| | | gcgaaacatcgcatcgagcgagcacgtactcggatggaagccggtcttgtcgatcagg |
| | | atgatctggacgaagagcatcaggggctcgcgccagccgaactgttcgccaggctca |
| | | aggcgagcatgcccgacggcgaggatctcgtcgtgacccatggcgatgcctgcttgc |
| | | cgaatatcatggtggaaaatggccgcttactggattcatcgactgtggccggctgggtg |
| | | tggcggaccgctatcaggacatagcgttggctacccgtgatattgctgaagagcttggc |
| | | ggcgaatgggctgaccgcttcctcgtgctttacggtatcgccgctcccgattcgcagcg |
| | | catcgccttctatcgccttcttgacgagttcttctgaattattaacgcttacaatttcctgatg |
| | | cggtattttctccttacgcatctgtgcggtatttcacaccgcatacaggtggcacttttc gg |
| | | ggaaatgtgcgcggaacccctatagtttatttactaaatacattcaaatatgtatccgctca |
| | | tgagacaataaccctgataaatgcttcaataatagcacgtgctaaaacttcatttttaattta |
| | | aaaggatctaggtgaagatcctattgataatctcatgaccaaaatcccttaacgtgagtta |
| | | cgttccactgagcgtcagaccccgtagaaaagatcaaaggatcttcttgagatcctatat |
| | | ctgcgcgtaatctgctgcttgcaaacaaaaaaaccaccgctaccagcggtggtttgtttg |
| | | ccggatcaagagctaccaactctattccgaaggtaactggcttcagcagagcgcagat |
| | | accaaatactgtccttctagtgtagccgtagttaggccaccacttcaagaactctgtagca |
| | | ccgcctacatacctcgctctgctaatcctgttaccagtggctgctgccagtggc gataag |
| | | tcgtgtcttaccgggttggactcaagacgatagttaccggataaggcgcagcggtcgg |
| | | gctgaacggggggttcgtgcacacagcccagcttggagcgaacgacctacaccgaac |
| | | tgagatacctacagcgtgagctatgagaaagcgccacgcttcccgaagggagaaagg |
| | | cggacaggtatccggtaagcggcagggtcggaacaggagagcgcacgagggagct |
| | | tccagggggaaacgcctggtatctttatagtcctgtcgggtttcgccacctctgacttgag |
| | | cgtcgattatgtgatgctcgtcaggggggcggagcctatggaaaaacgccagcaacg |
| | | cggcctattacggacctgggcttagctggccttagctcacatgttcttgactcttcgcga |
| | | tgtacgggccagatatgtcgaccgacatgtcgcacaagtcctaagttacgcgacaggct |
| | | gccgccctgcccttacctggcgttacttgtcgcgtgattagtcgcataaagtagaatact |
| | | tgcgactagaaccggagacattacgccatgaacaagagcgccgccgctggcctgctg |
| | | ggctatgcccgcgtcagcaccgacgaccaggacttgaccaaccaacgggccgaact |
| | | gcacgcggccggctgcaccaagctgttttccgagaagatcaccggcaccaggcgcga |
| | | ccgcccggagctggccaggatgcttgaccacctacgccctggcgacgttgtgacagtg |
| | | accaggctagaccgcctggcccgcagcacccgcgacctactggacattgccgagcg |
| | | catccaggaggccggcgcgggcctgcgtagcctggcagagccgtgggccgacacc |
| | | accacgccggccggccgcatggtgttgaccgtgttcgccggcattgccgagttcgagc |
| | | gttccctaatcatcgaccgcacccggagcgggcgcgaggccgccaaggcgcgaggc |
| | | gtgaagtaggcccccgccctaccctcaccccggcacagatcgcgcacgcccgcgag |
| | | ctgatcgaccaggaaggccgcaccgtgaaagaggcggctgcactgcttggcgtgcat |
| | | cgctcgaccctgtaccgcgcacttgagcgcagcgaggaagtgacgcccaccgaggc |
| | | caggcggcgcggtgccttccgtgaggacgcattgaccgaggccgacgccctggcgg |
| | | ccgccgagaatgaacgccaagaggaacaagcatgaaaccgcaccaggacggccag |
| | | gacgaaccgtttttcattaccgaagagatcgaggcggagatgatcgcggccgggtacg |
| | | tgttcgagccgcccgcgcacgtctcaaccgtgcggctgcatgaaatcctggccggtttg |
| | | tctgatgccaagctcgcggcctggccggcgagcttggccgctgaagaaaccgagcgc |
| | | cgccgtctaaaaaggtgatgtgtatttgagtaaaacagcttgcgtcatgcggtcgctgcg |
| | | tatatgatgcgatgagtaaataaacaaatacgcaaggggaacgcatgaaggttatcgct |
| | | gtacttaaccagaaaggcgggtcaggcaagacgaccatcgcaacccatctagcccgc |
| | | gccctgcaactcgccggggccgatgttctgttagtcgattccgatccccagggcagtgc |
| | | ccgcgattgggcggccgtgcgggaagatcaaccgctaaccgttgtcggcatcgaccg |
| | | cccgacgattgaccgcgacgtgaaggccatcggccggcgcgacttcgtagtgatcga |
| | | cggagcgccccaggcggcggacttggctgtgtccgcgatcaaggcagccgacttcgt |
| | | gctgattccggtgcagccaagcccttacgacatatgggccaccgccgacctggtggag |
| | | ctggttaagcagcgcattgaggtcacggatggaaggctacaagcggcctttgtcgtgtc |
| | | gcgggcgatcaaaggcacgcgcatcggcggtgaggttgccgaggcgctggccgggt |
| | | acgagctgcccattcttgagtcccgtatcacgcagcgcgtgagctacccaggcactgc |
| | | cgccgccggcacaaccgttcttgaatcagaacccgagggcgacgctgcccgcgaggt |
| | | ccaggcgctggccgctgaaattaaatcaaaactcatttgagttaatgaggtaaagagaa |
| | | aatgagcaaaagcacaaacacgctaagtgccggccgtccgagcgcacgcagcagca |
| | | aggctgcaacgaggccagcctggcagacacgccagccatgaagcgggtcaactac |
| | | agttgccggcggaggatcacaccaagctgaagatgtacgcggtacgccaaggcaag |
| | | accattaccgagctgctatctgaatacatcgcgcagctaccagagtaaatgagcaaatg |
| | | aataaatgagtagatgaattttagcggctaaaggaggcggcatggaaaatcaagaaca |
| | | accaggcaccgacgccgtggaatgccccatgtgtggaggaacgggcggttggccag |
| | | gcgtaagcggctgggttgtctgccggccctgcaatggcactggaacccccaagcccg |
| | | aggaatcggcgtgagcggtcgcaaaccatccggcccggtacaaatcggcgcggcgc |
| | | tgggtgatgacctggtggagaagttgaaggcggcgcaggccgcccagcggcaacgc |
| | | atcgaggcagaagcacgccccggtgaatcgtggcaagcggccgctgatcgaatccg |
| | | caaagaatcccggcaaccgccggcagccggtgcgccgtcgattaggaagccgccca |
| | | agggcgacgagcaaccagattattcgaccgatgctctatgacgtgggcacccgcgat |
| | | agtcgcagcatcatggacgtggccgttttccgtctgtcgaagcgtgaccgacgagctgg |
| | | cgaggtgatccgctacgagcttccagacgggcacgtagaggtttccgcagggccggc |
| | | cggcatggcgagtgtgtgggattacgacctggtactgatggcggtacccatctaaccg |
| | | aatccatgaaccgataccgggaagggaagggagacaagcccggccgcgtgttccgt |
| | | ccacacgttgcggacgtactcaagttctgccggcgagccgatggcggaaagcagaaa |
| | | gacgacctggtagaaacctgcattcggttaaacaccacgcacgttgccatgcagcgtac |
| | | gaagaaggccaagaacggccgcctggtgacggtatccgagggtgaagccttgattag |
| | | ccgctacaagatcgtaaagagcgaaaccgggcggccggagtacatcgagatcgaga |
| | | agctgattggatgtaccgcgagatcacagaaggcaagaacccggacgtgctgacggtt |
| | | caccccgattactttttgatcgatcccggcatcggccgttactctaccgcctggcacgcc |
| | | gcgccgcaggcaaggcagaagccagatggttgttcaagacgatctacgaacgcagtg |
| | | gcagcgccggagagttcaagaagttctgtttcaccgtgcgcaagctgatcgggtcaaat |
| | | gacctgccggagtacgatttgaaggaggaggcggggcaggctggcccgatcctagtc |
| | | atgcgctaccgcaacctgatcgagggcgaagcatccgccggttcctaatgtacggagc |
| | | agatgctagggcaaattgccctagcaggggaaaaaggtcgaaaaggtctctttcctgtg |
| | | gatagcacgtacattgggaacccaaagccgtacattgggaaccggaacccgtacattg |
| | | ggaacccaaagccgtacattgggaaccggtcacacatgtaagtgactgatataaaaga |
| | | gaaaaaaggcgatttttccgcctaaaactctttaaaacttattaaaactcttaaaacccgcc |
| | | tggcctgtgcataactgtctggccagcgcacagccgaagagctgcaaaaagcgcctac |
| | | ccttcggtcgctgcgctccctacgccccgccgcttcgcgtcggcctatcgcggccgctg |
| | | gccgctcaaaaatggctggcctacggccaggcaatctaccagggcgcggacaagcc |
| | | gcgccgtcgccactcgaccgccggcgcccacatcaaggcacctctagatggcaggat |
| | | atattgtggtgtaaacagtttaaacagtgttttactcctcatattaacttcggtcattagaggc |
| | | cacgatttgacacatttttactcaaaacaaaatgtagcatatctcttataatttcaaattcaac |
| | | acacaacaaataagagaaaaaacaaataatattaatttgagaatgaacaaaaggaccat |
| | | atcattcattaactcttctccatccataccatttcacagttcgatagcgaaaaccgaataaa |
| | | aaacacagtaaattacaagcacaacaaatggtacaagaaaaacagttttcccaatgccat |
| | | aatactcgaacgtccggagttatcagaagaactcgtcaagaaggcgatagaaggcgat |
| | | gcgctgcgaatcgggagcggcgataccgtaaagcacgaggaagcggtcagcccatt |
| | | cgccgccaagctcttcagcaatatcacgggtagccaacgctatgtcctgatagcggtcc |
| | | gccacacccagccggccacagtcgatgaatccagaaaagcggccattttccaccatga |
| | | tattcggcaagcaggcatcgccatgggtcacgacgagatcctcgccgtcgggcatgcg |
| | | cgccttgagcctggcgaacagttcggctggcgcgagcccctgatgctcttcgtccagat |
| | | catcctgatcgacaagaccggcttccatccgagtacgtgctcgctcgatgcgatgtacg |
| | | cttggtggtcgaatgggcaggtagccggatcaagcgtatgcagccgccgcattgcatc |
| | | agccatgatggatactttctcggcaggagcaaggtgagatgacaggagatcctgcccc |
| | | ggcacttcgcccaatagcagccagtcccttcccgcttcagtgacaacgtcgagcacag |
| | | ctgcgcaaggaacgcccgtcgtggccagccacgatagccgcgctgcctcgtcctgca |
| | | gttcattcagggcaccggacaggtcggtcttgacaaaaagaaccgggcgcccctgcg |
| | | ctgacagccggaacacggcggcatcagagcagccgattgtctgttgtgcccagtcata |
| | | gccgaatagcctctccacccaagcggccggagaacctgcgtgcaatccatcttgttcaa |
| | | tccaagctcccattgttggtacccagcttgggtctagtcgtattaagagatagatttgtaga |
| | | gagagactggtgatttcagcgtgtcctctccaaatgaaatgaacttccttatatagaggaa |
| | | ggtcttgcgaaggatagtgggattgtgcgtcatcccttacgtcagtggagatatcacatc |
| | | aatccacttgctttgaagacgtggaggaacgtcttctttttccacgatgctcctcgtgggt |
| | | gggggtccatctagggaccactgtcggcagaggcatcttgaacgatagcctacctttat |
| | | cgcaatgatggcatttgtaggtgccaccttccttttctactgtccttttgatgaagtgacaga |
| | | tagctgggcaatggaatccgaggaggtttcccgatattaccctttgttgaaaagtctcaat |
| | | agccctttggtcttctgagactgtatctttgatattcttggagtagacgagagtgtcgtgctc |
| | | caccatgttatcacatcaatccacttgctttgaagacgtggttggaacgtcttctttttccac |
| | | gatgctcctcgtgggtgggggtccatctagggaccactgtcggcagaggcatcttgaa |
| | | cgatagcctttcctttatcgcaatgatggcatttgtaggtgccaccttccttttctactgtcctt |
| | | ttgatgaagtgacagatagctgggcaatggaatccgaggaggtttcccgatattaccctt |
| | | tgttgaaaagtctcaatagccctttggtcttctgagactgtatctttgatattcttggagtaga |
| | | cgagagtgtcgtgctccaccattacataggcccatcggagctaacgcagtgaattcaga |
| | | aatctcaaaattccggcagaacaattttgaatctcgatccgtagaaacgagacggtcatt |
| | | gttttagttccaccacgattatatttgaaatttacgtgagtgtgagtgagacttgcataagaa |
| | | aataaaatctttagttgggaaaaaattcaataatataaatgggcttgagaaggaagcgag |
| | | ggataggcctttttctaaaataggcccatttaagctattaacaatcttcaaaagtaccacag |
| | | cgcttaggtaaagaaagcagctgagtttatatatggttagagacgaagtagtgattggat |
| | | ggcaggtggaagaatggacacctgcgagagttttagagctagaaatagcaagttaaaa |
| | | taaggctagtccgttatcaacttgaaaaagtggcaccgagtcggtgctttttttacagtga |
| | | aagcttactgcgttagctccgatgggcctatgtaatggtggagcacgacactctcgtcta |
| | | ctccaagaatatcaaagatacagtctcagaagaccaaagggctattgagacttttcaaca |
| | | aagggtaatatcgggaaacctcctcggattccattgcccagctatctgtcacttcatcaaa |
| | | aggacagtagaaaaggaaggtggcacctacaaatgccatcattgcgataaaggaaag |
| | | gctatcgttcaagatgcctctgccgacagtggtcccaaagatggacccccacccacga |
| | | ggagcatcgtggaaaaagaagacgttccaaccacgtcttcaaagcaagtggattgatgt |
| | | gataacatggtggagcacgacactctcgtctactccaagaatatcaaagatacagtctca |
| | | gaagaccaaagggctattgagacttttcaacaaagggtaatatcgggaaacctcctcgg |
| | | attccattgcccagctatctgtcacttcatcaaaaggacagtagaaaaggaaggtggca |
| | | cctacaaatgccatcattgcgataaaggaaaggctatcgttcaagatgcctctgccgaca |
| | | gtggtcccaaagatggacccccacccacgaggagcatcgtggaaaaagaagacgttc |
| | | caaccacgtcttcaaagcaagtggattgatgtgatatctccactgacgtaagggatgacg |
| | | cacaatcccactatccttcgcaagaccttcctctatataaggaagttcatttcataggaga |
| | | ggacacgctgaaatcaccagtctctctctacaaatctatctcttaatacgactcactatagg |
| | | gagacccaagctggctagcaacaatggataagaagtactctatcggactcgatatcgg |
| | | aactaactctgttggatgggctgtgatcaccgatgagtacaaggtgccatctaagaagtt |
| | | caaggttctcggaaacaccgataggcactctatcaagaaaaaccttatcggtgctctcct |
| | | cttcgattctggtgaaactgctgaggctaccagactcaagagaaccgctagaagaaggt |
| | | acaccagaagaaagaacaggatctgctacctccaagagattactctaacgagatggct |
| | | aaagtggatgattcattcttccacaggctcgaagagtcattcctcgtggaagaagataag |
| | | aagcacgagaggcaccctatcttcggaaacatcgttgatgaggtggcataccacgaga |
| | | agtaccctactatctaccacctcagaaagaagctcgttgattctactgataaggctgatct |
| | | caggctcatctacctcgctctcgctcacatgatcaagttcagaggacacttcctcatcga |
| | | gggtgatctcaaccctgataactctgatgtggataagagttcatccagctcgtgcagacc |
| | | tacaaccagcttacgaagagaaccctatcaacgcttcaggtgtggatgctaaggctatc |
| | | ctctctgctaggctctctaagtcaagaaggcttgagaacctcattgctcagctccctggtg |
| | | agaagaagaacggacttacggaaacttgatcgctctctctctcggactcacccctaactt |
| | | caagtctaacttcgatctcgctgaggatgcaaagctccagctctcaaaggatacctacga |
| | | tgatgatctcgataacctcctcgctcagatcggagatcagtacgctgatttgacctcgctg |
| | | ctaagaacctctctgatgctatcctcctcagtgatatcctcagggtgaacaccgagatca |
| | | ccaaggctccactactgcttctatgatcaagagatacgatgagcaccaccaggatctca |
| | | cacttctcaaggctcttgttagacagcagctcccagagaagtacaaagaaatcttcttcg |
| | | atcagtctaagaacggatacgctggttacatcgatggtggtgcatctcaagaagagttct |
| | | acaagttcatcaagccaatcttggagaagatggatggaaccgaggaactcctcgtgaa |
| | | gctcaatagagaggatctccttaggaagcagaggaccttcgataacggatctatccctc |
| | | atcagatccacctcggagagttgcacgctatccttagaaggcaagaggatactacccat |
| | | tcctcaaggataacagagagaagattgagaagatcctcaccttcagaatcccttactacg |
| | | tgggacctctcgctagaggaaactcaagattcgcttggatgaccagaaagtctgaggaa |
| | | accatcaccccttggaacttcgaagaggtggtggataagggtgctagtgctcagtctac |
| | | atcgagaggatgaccaacttcgataagaaccttcctaacgagaaggtgctccctaagca |
| | | ctctagctctacgagtacttcaccgtgtacaacgagttgaccaaggttaagtacgtgacc |
| | | gagggaatgaggaagcctgcttttttgtcaggtgagcaaaagaaggctatcgttgatctc |
| | | ttgttcaagaccaacagaaaggtgaccgtgaagcagctcaaagaggattacttcaagaa |
| | | aatcgagtgcttcgattcagtggaaatctctggtgttgaggataggttcaacgcatctctc |
| | | ggaacctaccacgatctcctcaagatcattaaggataaggatacttggataacgaggaa |
| | | aacgaggatatcttggaggatatcgacttaccctcaccctcttcgaggatagagagatg |
| | | atagaagaaaggctcaagacctacgctcatctcttcgatgataaggtgatgaagcagttg |
| | | aagagaagaagatacactggaggggaaggctctcaagaaagctcattaacggaatca |
| | | gggataagcagtctggaaagacaatccttgatacctcaagtctgatggattcgctaaca |
| | | gaaacttcatgcagctcatccacgatgattctctcacctttaaagaggatatccagaagg |
| | | ctcaggtttcaggacagggtgatagtctccatgagcatatcgctaacctcgctggatccc |
| | | ctgcaatcaagaagggaatcctccagactgtgaagattgtggatgagttggtgaaggtg |
| | | atgggacacaagcctgagaacatcgtgatcgaaatggctagagagaaccagaccact |
| | | cagaagggacagaagaactctagggaaaggatgaagaggatcgaggaaggtatcaa |
| | | agagcttggatctcagatcctcaaagagcaccctgagagaacactcagctccagaacg |
| | | agaagctctacctctactacttgcagaacggaagggatatgtatgtggatcaagagcttg |
| | | atattaacaggctctctgattacgatgttgatcatatcgtgccacagtcttttatcaaagatg |
| | | attctatcgataacaaggtgctcactaggtctgataagaacaggggtaagagtgataac |
| | | gtgccaagtgaagaggagtgaagaaaatgaagaactattggaggcagctcctcaacg |
| | | ctaagctcatcactcagagaaagttcgataacttgaccaaggctgagaggggaggact |
| | | ctctgaattggataaggcaggattcatcaagagacagctcgtggaaaccaggcagatc |
| | | accaaacatgtggcacagatcctcgattctaggatgaacaccaagtacgatgagaacg |
| | | ataagttgatcagggaagtgaaggttatcaccctcaagtcaaagctcgtgtctgatttcag |
| | | aaaggataccaattctacaaggtgagggaaatcaacaactaccaccacgctcacgatg |
| | | cttaccttaacgctgagttggaaccgctctcatcaagaagtatccaaagttggagtctga |
| | | gttcgtgtacggtgattataaggtgtacgatgtgaggaagatgatcgctaagtctgagca |
| | | agagatcggaaaggctaccgctaagtatacttctactctaacatcatgaatacttcaaga |
| | | ccgagatcactctcgctaacggtgagatcagaaagaggccactcatcgagacaaacg |
| | | gtgaaacaggtgagatcgtgtgggataagggaagggatttcgctacc gttagaaaggt |
| | | gctctctatgcctcaggtgaacatcgttaagaaaaccgaggtgcagaccggtggattct |
| | | ctaaagagtctatcctccctaagaggaactctgataagctcattgctaggaagaaggatt |
| | | gggaccctaagaaatacggtggtacgattctcctaccgtggcttactctgactcgttgtg |
| | | gctaaggagagaagggaaagagtaagaagctcaagtctgttaaggaacttctcggaat |
| | | cactatcatggaaaggtcatctacgagaagaacccaatcgataccttgaggctaaggg |
| | | atacaaagaggttaagaaggatctcatcatcaagctcccaaagtactcacttacgagag |
| | | gagaacggtagaaagaggatgctcgcttctgctggtgagcttcaaaagggaaacgag |
| | | cttgctctcccatctaagtacgttaactactttacctcgcttctcactacgagaagttgaag |
| | | ggatctccagaagataacgagcagaagcaacttacgttgagcagcacaagcactactt |
| | | ggatgagatcatcgagcagatcagtgagttctctaaaagggtgatcctcgctgatgcaa |
| | | acctcgataaggtgagtctgcttacaacaagcacagagataagcctatcagggaacag |
| | | gcagagaacatcatccatctcttcacccttaccaacctcggtgctcctgctgctacaagt |
| | | acttcgatacaaccatcgataggaagagatacacctctaccaaagaagtgctcgatgct |
| | | accctcatccatcagtctatcactggactctacgagactaggatcgatctctcacagcttg |
| | | gaggtgatcctaagaagaaaagaaaggttagatcttgatgacccgggtctccataataat |
| | | gtgtgagtagacccagataagggaattagggacctatagggtacgctcatgtgttgag |
| | | catataagaaacccttagtatgtatagtatagtaaaatacttctatcaataaaatttctaattc |
| | | ctaaaaccaaaatccagtactaaaatccagatcccccgaattaaggccttgacaggatat |
| | | attggcgggtaaacctaagagaaaagagcgtttattagaataacggatatttaaaactcg |
| | | ag |
| 11 | pCambia1301:35S:GUS | GATCTGAGGGTAAATTTCTAGTTTTTCTCCTTCATTTT |
| | | CTTGGTTAGGACCCTTTTCTCTTTTTATTTTTTTGAGC |
| | | TTTGATCTTTCTTTAAACTGATCTATTTTTTAATTGAT |
| | | TGGTTATGGTGTAAATATTACATAGCTTTAACTGATA |
| | | ATCTGATTACTTTATTTCGTGTGTCTATGATGATGAT |
| | | GATAGTTACAGAACCGACGACTCGTCCGTCCTGTAG |
| | | AAACCCCAACCCGTGAAATCAAAAAACTCGACGGCC |
| | | TGTGGGCATTCAGTCTGGATCGCGAAAACTGTGGAA |
| | | TTGATCAGCGTTGGTGGGAAAGCGCGTTACAAGAAA |
| | | GCCGGGCAATTGCTGTGCCAGGCAGTTTTAACGATC |
| | | AGTTCGCCGATGCAGATATTCGTAATTATGCGGGCA |
| | | ACGTCTGGTATCAGCGCGAAGTCTTTATACCGAAAG |
| | | GTTGGGCAGGCCAGCGTATCGTGCTGCGTTTCGATGC |
| | | GGTCACTCATTACGGCAAAGTGTGGGTCAATAATCA |
| | | GGAAGTGATGGAGCATCAGGGCGGCTATACGCCATT |
| | | TGAAGCCGATGTCACGCCGTATGTTATTGCCGGGAA |
| | | AAGTGTACGTATCACCGTTTGTGTGAACAACGAACT |
| | | GAACTGGCAGACTATCCCGCCGGGAATGGTGATTAC |
| | | CGACGAAAACGGCAAGAAAAAGCAGTCTTACTTCCA |
| | | TGATTTCTTTAACTATGCCGGAATCCATCGCAGCGTA |
| | | ATGCTCTACACCACGCCGAACACCTGGGTGGACGAT |
| | | ATCACCGTGGTGACGCATGTCGCGCAAGACTGTAAC |
| | | CACGCGTCTGTTGACTGGCAGGTGGTGGCCAATGGT |
| | | GATGTCAGCGTTGAACTGCGTGATGCGGATCAACAG |
| | | GTGGTTGCAACTGGACAAGGCACTAGCGGGACTTTG |
| | | CAAGTGGTGAATCCGCACCTCTGGCAACCGGGTGAA |
| | | GGTTATCTCTATGAACTCGAAGTCACAGCCAAAAGC |
| | | CAGACAGAGTCTGATATCTACCCGCTTCGCGTCGGCA |
| | | TCCGGTCAGTGGCAGTGAAGGGCCAACAGTTCCTGA |
| | | TTAACCACAAACCGTTCTACTTTACTGGCTTTGGTCG |
| | | TCATGAAGATGCGGACTTACGTGGCAAAGGATTCGA |
| | | TAACGTGCTGATGGTGCACGACCACGCATTAATGGA |
| | | CTGGATTGGGGCCAACTCCTACCGTACCTCGCATTAC |
| | | CCTTACGCTGAAGAGATGCTCGACTGGGCAGATGAA |
| | | CATGGCATCGTGGTGATTGATGAAACTGCTGCTGTCG |
| | | GCTTTCAGCTGTCTTTAGGCATTGGTTTCGAAGCGGG |
| | | CAACAAGCCGAAAGAACTGTACAGCGAAGAGGCAG |
| | | TCAACGGGGAAACTCAGCAAGCGCACTTACAGGCGA |
| | | TTAAAGAGCTGATAGCGCGTGACAAAAACCACCCAA |
| | | GCGTGGTGATGTGGAGTATTGCCAACGAACCGGATA |
| | | CCCGTCCGCAAGGTGCACGGGAATATTTCGCGCCAC |
| | | TGGCGGAAGCAACGCGTAAACTCGACCCGACGCGTC |
| | | CGATCACCTGCGTCAATGTAATGTTCTGCGACGCTCA |
| | | CACCGATACCATCAGCGATCTCTTTGATGTGCTGTGC |
| | | CTGAACCGTTATTACGGATGGTATGTCCAAAGCGGC |
| | | GATTTGGAAACGGCAGAGAAGGTACTGGAAAAAGA |
| | | ACTTCTGGCCTGGCAGGAGAAACTGCATCAGCCGAT |
| | | TATCATCACCGAATACGGCGTGGATACGTTAGCCGG |
| | | GCTGCACTCAATGTACACCGACATGTGGAGTGAAGA |
| | | GTATCAGTGTGCATGGCTGGATATGTATCACCGCGTC |
| | | TTTGATCGCGTCAGCGCCGTCGTCGGTGAACAGGTAT |
| | | GGAATTTCGCCGATTTTGCGACCTCGCAAGGCATATT |
| | | GCGCGTTGGCGGTAACAAGAAAGGGATCTTCACTCG |
| | | CGACCGCAAACCGAAGTCGGCGGCTTTTCTGCTGCA |
| | | AAAACGCTGGACTGGCATGAACTTCGGTGAAAAACC |
| | | GCAGCAGGGAGGCAAACAAGCTAGCCACCACCACCA |
| | | CCACCACGTGTGAATTACAGGTGACCAGCTCGAATTT |
| | | CCCCGATCGTTCAAACATTTGGCAATAAAGTTTCTTA |
| | | AGATTGAATCCTGTTGCCGGTCTTGCGATGATTATCA |
| | | TATAATTTCTGTTGAATTACGTTAAGCATGTAATAAT |
| | | TAACATGTAATGCATGACGTTATTTATGAGATGGGTT |
| | | TTTATGATTAGAGTCCCGCAATTATACATTTAATACG |
| | | CGATAGAAAACAAAATATAGCGCGCAAACTAGGATA |
| | | AATTATCGCGCGCGGTGTCATCTATGTTACTAGATCG |
| | | GGAATTAAACTATCAGTGTTTGACAGGATATATTGGC |
| | | GGGTAAACCTAAGAGAAAAGAGCGTTTATTAGAATA |
| | | ACGGATATTTAAAAGGGCGTGAAAAGGTTTATCCGT |
| | | TCGTCCATTTGTATGTGCATGCCAACCACAGGGTTCC |
| | | CCTCGGGATCAAAGTACTTTGATCCAACCCCTCCGCT |
| | | GCTATAGTGCAGTCGGCTTCTGACGTTCAGTGCAGCC |
| | | GTCTTCTGAAAACGACATGTCGCACAAGTCCTAAGTT |
| | | ACGCGACAGGCTGCCGCCCTGCCCTTTTCCTGGCGTT |
| | | TTCTTGTCGCGTGTTTTAGTCGCATAAAGTAGAATAC |
| | | TTGCGACTAGAACCGGAGACATTACGCCATGAACAA |
| | | GAGCGCCGCCGCTGGCCTGCTGGGCTATGCCCGCGT |
| | | CAGCACCGACGACCAGGACTTGACCAACCAACGGGC |
| | | CGAACTGCACGCGGCCGGCTGCACCAAGCTGTTTTCC |
| | | GAGAAGATCACCGGCACCAGGCGCGACCGCCCGGAG |
| | | CTGGCCAGGATGCTTGACCACCTACGCCCTGGCGAC |
| | | GTTGTGACAGTGACCAGGCTAGACCGCCTGGCCCGC |
| | | AGCACCCGCGACCTACTGGACATTGCCGAGCGCATC |
| | | CAGGAGGCCGGCGCGGGCCTGCGTAGCCTGGCAGAG |
| | | CCGTGGGCCGACACCACCACGCCGGCCGGCCGCATG |
| | | GTGTTGACCGTGTTCGCCGGCATTGCCGAGTTCGAGC |
| | | GTTCCCTAATCATCGACCGCACCCGGAGCGGGCGCG |
| | | AGGCCGCCAAGGCCCGAGGCGTGAAGTTTGGCCCCC |
| | | GCCCTACCCTCACCCCGGCACAGATCGCGCACGCCC |
| | | GCGAGCTGATCGACCAGGAAGGCCGCACCGTGAAAG |
| | | AGGCGGCTGCACTGCTTGGCGTGCATCGCTCGACCCT |
| | | GTACCGCGCACTTGAGCGCAGCGAGGAAGTGACGCC |
| | | CACCGAGGCCAGGCGGCGCGGTGCCTTCCGTGAGGA |
| | | CGCATTGACCGAGGCCGACGCCCTGGCGGCCGCCGA |
| | | GAATGAACGCCAAGAGGAACAAGCATGAAACCGCA |
| | | CCAGGACGGCCAGGACGAACCGTTTTTCATTACCGA |
| | | AGAGATCGAGGCGGAGATGATCGCGGCCGGGTACGT |
| | | GTTCGAGCCGCCCGCGCACGTCTCAACCGTGCGGCT |
| | | GCATGAAATCCTGGCCGGTTTGTCTGATGCCAAGCTG |
| | | GCGGCCTGGCCGGCCAGCTTGGCCGCTGAAGAAACC |
| | | GAGCGCCGCCGTCTAAAAAGGTGATGTGTATTTGAG |
| | | TAAAACAGCTTGCGTCATGCGGTCGCTGCGTATATGA |
| | | TGCGATGAGTAAATAAACAAATACGCAAGGGGAACG |
| | | CATGAAGGTTATCGCTGTACTTAACCAGAAAGGCGG |
| | | GTCAGGCAAGACGACCATCGCAACCCATCTAGCCCG |
| | | CGCCCTGCAACTCGCCGGGGCCGATGTTCTGTTAGTC |
| | | GATTCCGATCCCCAGGGCAGTGCCCGCGATTGGGCG |
| | | GCCGTGCGGGAAGATCAACCGCTAACCGTTGTCGGC |
| | | ATCGACCGCCCGACGATTGACCGCGACGTGAAGGCC |
| | | ATCGGCCGGCGCGACTTCGTAGTGATCGACGGAGCG |
| | | CCCCAGGCGGCGGACTTGGCTGTGTCCGCGATCAAG |
| | | GCAGCCGACTTCGTGCTGATTCCGGTGCAGCCAAGC |
| | | CCTTACGACATATGGGCCACCGCCGACCTGGTGGAG |
| | | CTGGTTAAGCAGCGCATTGAGGTCACGGATGGAAGG |
| | | CTACAAGCGGCCTTTGTCGTGTCGCGGGCGATCAAA |
| | | GGCACGCGCATCGGCGGTGAGGTTGCCGAGGCGCTG |
| | | GCCGGGTACGAGCTGCCCATTCTTGAGTCCCGTATCA |
| | | CGCAGCGCGTGAGCTACCCAGGCACTGCCGCCGCCG |
| | | GCACAACCGTTCTTGAATCAGAACCCGAGGGCGACG |
| | | CTGCCCGCGAGGTCCAGGCGCTGGCCGCTGAAATTA |
| | | AATCAAAACTCATTTGAGTTAATGAGGTAAAGAGAA |
| | | AATGAGCAAAAGCACAAACACGCTAAGTGCCGGCCG |
| | | TCCGAGCGCACGCAGCAGCAAGGCTGCAACGTTGGC |
| | | CAGCCTGGCAGACACGCCAGCCATGAAGCGGGTCAA |
| | | CTTTCAGTTGCCGGCGGAGGATCACACCAAGCTGAA |
| | | GATGTACGCGGTACGCCAAGGCAAGACCATTACCGA |
| | | GCTGCTATCTGAATACATCGCGCAGCTACCAGAGTA |
| | | AATGAGCAAATGAATAAATGAGTAGATGAATTTTAG |
| | | CGGCTAAAGGAGGCGGCATGGAAAATCAAGAACAA |
| | | CCAGGCACCGACGCCGTGGAATGCCCCATGTGTGGA |
| | | GGAACGGGCGGTTGGCCAGGCGTAAGCGGCTGGGTT |
| | | GTCTGCCGGCCCTGCAATGGCACTGGAACCCCCAAG |
| | | CCCGAGGAATCGGCGTGAGCGGTCGCAAACCATCCG |
| | | GCCCGGTACAAATCGGCGCGGCGCTGGGTGATGACC |
| | | TGGTGGAGAAGTTGAAGGCCGCGCAGGCCGCCCAGC |
| | | GGCAACGCATCGAGGCAGAAGCACGCCCCGGTGAAT |
| | | CGTGGCAAGCGGCCGCTGATCGAATCCGCAAAGAAT |
| | | CCCGGCAACCGCCGGCAGCCGGTGCGCCGTCGATTA |
| | | GGAAGCCGCCCAAGGGCGACGAGCAACCAGATTTTT |
| | | TCGTTCCGATGCTCTATGACGTGGGCACCCGCGATAG |
| | | TCGCAGCATCATGGACGTGGCCGTTTTCCGTCTGTCG |
| | | AAGCGTGACCGACGAGCTGGCGAGGTGATCCGCTAC |
| | | GAGCTTCCAGACGGGCACGTAGAGGTTTCCGCAGGG |
| | | CCGGCCGGCATGGCCAGTGTGTGGGATTACGACCTG |
| | | GTACTGATGGCGGTTTCCCATCTAACCGAATCCATGA |
| | | ACCGATACCGGGAAGGGAAGGGAGACAAGCCCGGC |
| | | CGCGTGTTCCGTCCACACGTTGCGGACGTACTCAAGT |
| | | TCTGCCGGCGAGCCGATGGCGGAAAGCAGAAAGACG |
| | | ACCTGGTAGAAACCTGCATTCGGTTAAACACCACGC |
| | | ACGTTGCCATGCAGCGTACGAAGAAGGCCAAGAACG |
| | | GCCGCCTGGTGACGGTATCCGAGGGTGAAGCCTTGA |
| | | TTAGCCGCTACAAGATCGTAAAGAGCGAAACCGGGC |
| | | GGCCGGAGTACATCGAGATCGAGCTAGCTGATTGGA |
| | | TGTACCGCGAGATCACAGAAGGCAAGAACCCGGACG |
| | | TGCTGACGGTTCACCCCGATTACTTTTTGATCGATCC |
| | | CGGCATCGGCCGTTTTCTCTACCGCCTGGCACGCCGC |
| | | GCCGCAGGCAAGGCAGAAGCCAGATGGTTGTTCAAG |
| | | ACGATCTACGAACGCAGTGGCAGCGCCGGAGAGTTC |
| | | AAGAAGTTCTGTTTCACCGTGCGCAAGCTGATCGGGT |
| | | CAAATGACCTGCCGGAGTACGATTTGAAGGAGGAGG |
| | | CGGGGCAGGCTGGCCCGATCCTAGTCATGCGCTACC |
| | | GCAACCTGATCGAGGGCGAAGCATCCGCCGGTTCCT |
| | | AATGTACGGAGCAGATGCTAGGGCAAATTGCCCTAG |
| | | CAGGGGAAAAAGGTCGAAAAGGTCTCTTTCCTGTGG |
| | | ATAGCACGTACATTGGGAACCCAAAGCCGTACATTG |
| | | GGAACCGGAACCCGTACATTGGGAACCCAAAGCCGT |
| | | ACATTGGGAACCGGTCACACATGTAAGTGACTGATA |
| | | TAAAAGAGAAAAAAGGCGATTTTTCCGCCTAAAACT |
| | | CTTTAAAACTTATTAAAACTCTTAAAACCCGCCTGGC |
| | | CTGTGCATAACTGTCTGGCCAGCGCACAGCCGAAGA |
| | | GCTGCAAAAAGCGCCTACCCTTCGGTCGCTGCGCTCC |
| | | CTACGCCCCGCCGCTTCGCGTCGGCCTATCGCGGCCG |
| | | CTGGCCGCTCAAAAATGGCTGGCCTACGGCCAGGCA |
| | | ATCTACCAGGGCGCGGACAAGCCGCGCCGTCGCCAC |
| | | TCGACCGCCGGCGCCCACATCAAGGCACCCTGCCTC |
| | | GCGCGTTTCGGTGATGACGGTGAAAACCTCTGACAC |
| | | ATGCAGCTCCCGGAGACGGTCACAGCTTGTCTGTAA |
| | | GCGGATGCCGGGAGCAGACAAGCCCGTCAGGGCGCG |
| | | TCAGCGGGTGTTGGCGGGTGTCGGGGCGCAGCCATG |
| | | ACCCAGTCACGTAGCGATAGCGGAGTGTATACTGGC |
| | | TTAACTATGCGGCATCAGAGCAGATTGTACTGAGAG |
| | | TGCACCATATGCGGTGTGAAATACCGCACAGATGCG |
| | | TAAGGAGAAAATACCGCATCAGGCGCTCTTCCGCTT |
| | | CCTCGCTCACTGACTCGCTGCGCTCGGTCGTTCGGCT |
| | | GCGGCGAGCGGTATCAGCTCACTCAAAGGCGGTAAT |
| | | ACGGTTATCCACAGAATCAGGGGATAACGCAGGAAA |
| | | GAACATGTGAGCAAAAGGCCAGCAAAAGGCCAGGA |
| | | ACCGTAAAAAGGCCGCGTTGCTGGCGTTTTTCCATAG |
| | | GCTCCGCCCCCCTGACGAGCATCACAAAAATCGACG |
| | | CTCAAGTCAGAGGTGGCGAAACCCGACAGGACTATA |
| | | AAGATACCAGGCGTTTCCCCCTGGAAGCTCCCTCGTG |
| | | CGCTCTCCTGTTCCGACCCTGCCGCTTACCGGATACC |
| | | TGTCCGCCTTTCTCCCTTCGGGAAGCGTGGCGCTTTC |
| | | TCATAGCTCACGCTGTAGGTATCTCAGTTCGGTGTAG |
| | | GTCGTTCGCTCCAAGCTGGGCTGTGTGCACGAACCCC |
| | | CCGTTCAGCCCGACCGCTGCGCCTTATCCGGTAACTA |
| | | TCGTCTTGAGTCCAACCCGGTAAGACACGACTTATCG |
| | | CCACTGGCAGCAGCCACTGGTAACAGGATTAGCAGA |
| | | GCGAGGTATGTAGGCGGTGCTACAGAGTTCTTGAAG |
| | | TGGTGGCCTAACTACGGCTACACTAGAAGGACAGTA |
| | | TTTGGTATCTGCGCTCTGCTGAAGCCAGTTACCTTCG |
| | | GAAAAAGAGTTGGTAGCTCTTGATCCGGCAAACAAA |
| | | CCACCGCTGGTAGCGGTGGTTTTTTTGTTTGCAAGCA |
| | | GCAGATTACGCGCAGAAAAAAAGGATCTCAAGAAG |
| | | ATCCTTTGATCTTTTCTACGGGGTCTGACGCTCAGTG |
| | | GAACGAAAACTCACGTTAAGGGATTTTGGTCATGCA |
| | | TTCTAGGTACTAAAACAATTCATCCAGTAAAATATAA |
| | | TATTTTATTTTCTCCCAATCAGGCTTGATCCCCAGTA |
| | | AGTCAAAAAATAGCTCGACATACTGTTCTTCCCCGAT |
| | | ATCCTCCCTGATCGACCGGACGCAGAAGGCAATGTC |
| | | ATACCACTTGTCCGCCCTGCCGCTTCTCCCAAGATCA |
| | | ATAAAGCCACTTACTTTGCCATCTTTCACAAAGATGT |
| | | TGCTGTCTCCCAGGTCGCCGTGGGAAAAGACAAGTT |
| | | CCTCTTCGGGCTTTTCCGTCTTTAAAAAATCATACAG |
| | | CTCGCGCGGATCTTTAAATGGAGTGTCTTCTTCCCAG |
| | | TTTTCGCAATCCACATCGGCCAGATCGTTATTCAGTA |
| | | AGTAATCCAATTCGGCTAAGCGGCTGTCTAAGCTATT |
| | | CGTATAGGGACAATCCGATATGTCGATGGAGTGAAA |
| | | GAGCCTGATGCACTCCGCATACAGCTCGATAATCTTT |
| | | TCAGGGCTTTGTTCATCTTCATACTCTTCCGAGCAAA |
| | | GGACGCCATCGGCCTCACTCATGAGCAGATTGCTCC |
| | | AGCCATCATGCCGTTCAAAGTGCAGGACCTTTGGAA |
| | | CAGGCAGCTTTCCTTCCAGCCATAGCATCATGTCCTT |
| | | TTCCCGTTCCACATCATAGGTGGTCCCTTTATACCGG |
| | | CTGTCCGTCATTTTTAAATATAGGTTTTCATTTTCTCC |
| | | CACCAGCTTATATACCTTAGCAGGAGACATTCCTTCC |
| | | GTATCTTTTACGCAGCGGTATTTTTCGATCAGTTTTTT |
| | | CAATTCCGGTGATATTCTCATTTTAGCCATTTATTATT |
| | | TCCTTCCTCTTTTCTACAGTATTTAAAGATACCCCAA |
| | | GAAGCTAATTATAACAAGACGAACTCCAATTCACTG |
| | | TTCCTTGCATTCTAAAACCTTAAATACCAGAAAACAG |
| | | CTTTTTCAAAGTTGTTTTCAAAGTTGGCGTATAACAT |
| | | AGTATCGACGGAGCCGATTTTGAAACCGCGGTGATC |
| | | ACAGGCAGCAACGCTCTGTCATCGTTACAATCAACA |
| | | TGCTACCCTCCGCGAGATCATCCGTGTTTCAAACCCG |
| | | GCAGCTTAGTTGCCGTTCTTCCGAATAGCATCGGTAA |
| | | CATGAGCAAAGTCTGCCGCCTTACAACGGCTCTCCCG |
| | | CTGACGCCGTCCCGGACTGATGGGCTGCCTGTATCGA |
| | | GTGGTGATTTTGTGCCGAGCTGCCGGTCGGGGAGCT |
| | | GTTGGCTGGCTGGTGGCAGGATATATTGTGGTGTAA |
| | | ACAAATTGACGCTTAGACAACTTAATAACACATTGC |
| | | GGACGTTTTTAATGTACTGAATTAACGCCGAATTAAT |
| | | TCGGGGGATCTGGATTTTAGTACTGGATTTTGGTTTT |
| | | AGGAATTAGAAATTTTATTGATAGAAGTATTTTACAA |
| | | ATACAAATACATACTAAGGGTTTCTTATATGCTCAAC |
| | | ACATGAGCGAAACCCTATAGGAACCCTAATTCCCTT |
| | | ATCTGGGAACTACTCACACATTATTATGGAGAAACTC |
| | | GAGCTTGTCGATCGACAGATCCGGTCGGCATCTACTC |
| | | TATTTCTTTGCCCTCGGACGAGTGCTGGGGCGTCGGT |
| | | TTCCACTATCGGCGAGTACTTCTACACAGCCATCGGT |
| | | CCAGACGGCCGCGCTTCTGCGGGCGATTTGTGTACGC |
| | | CCGACAGTCCCGGCTCCGGATCGGACGATTGCGTCG |
| | | CATCGACCCTGCGCCCAAGCTGCATCATCGAAATTGC |
| | | CGTCAACCAAGCTCTGATAGAGTTGGTCAAGACCAA |
| | | TGCGGAGCATATACGCCCGGAGTCGTGGCGATCCTG |
| | | CAAGCTCCGGATGCCTCCGCTCGAAGTAGCGCGTCT |
| | | GCTGCTCCATACAAGCCAACCACGGCCTCCAGAAGA |
| | | AGATGTTGGCGACCTCGTATTGGGAATCCCCGAACA |
| | | TCGCCTCGCTCCAGTCAATGACCGCTGTTATGCGGCC |
| | | ATTGTCCGTCAGGACATTGTTGGAGCCGAAATCCGC |
| | | GTGCACGAGGTGCCGGACTTCGGGGCAGTCCTCGGC |
| | | CCAAAGCATCAGCTCATCGAGAGCCTGCGCGACGGA |
| | | CGCACTGACGGTGTCGTCCATCACAGTTTGCCAGTGA |
| | | TACACATGGGGATCAGCAATCGCGCATATGAAATCA |
| | | CGCCATGTAGTGTATTGACCGATTCCTTGCGGTCCGA |
| | | ATGGGCCGAACCCGCTCGTCTGGCTAAGATCGGCCG |
| | | CAGCGATCGCATCCATAGCCTCCGCGACCGGTTGTA |
| | | GAACAGCGGGCAGTTCGGTTTCAGGCAGGTCTTGCA |
| | | ACGTGACACCCTGTGCACGGCGGGAGATGCAATAGG |
| | | TCAGGCTCTCGCTAAACTCCCCAATGTCAAGCACTTC |
| | | CGGAATCGGGAGCGCGGCCGATGCAAAGTGCCGATA |
| | | AACATAACGATCTTTGTAGAAACCATCGGCGCAGCT |
| | | ATTTACCCGCAGGACATATCCACGCCCTCCTACATCG |
| | | AAGCTGAAAGCACGAGATTCTTCGCCCTCCGAGAGC |
| | | TGCATCAGGTCGGAGACGCTGTCGAACTTTTCGATCA |
| | | GAAACTTCTCGACAGACGTCGCGGTGAGTTCAGGCT |
| | | TTTTCATATCTCATTGCCCCCCGGGATCTGCGAAAGC |
| | | TCGAGAGAGATAGATTTGTAGAGAGAGACTGGTGAT |
| | | TTCAGCGTGTCCTCTCCAAATGAAATGAACTTCCTTA |
| | | TATAGAGGAAGGTCTTGCGAAGGATAGTGGGATTGT |
| | | GCGTCATCCCTTACGTCAGTGGAGATATCACATCAAT |
| | | CCACTTGCTTTGAAGACGTGGTTGGAACGTCTTCTTT |
| | | TTCCACGATGCTCCTCGTGGGTGGGGGTCCATCTTTG |
| | | GGACCACTGTCGGCAGAGGCATCTTGAACGATAGCC |
| | | TTTCCTTTATCGCAATGATGGCATTTGTAGGTGCCAC |
| | | CTTCCTTTTCTACTGTCCTTTTGATGAAGTGACAGAT |
| | | AGCTGGGCAATGGAATCCGAGGAGGTTTCCCGATAT |
| | | TACCCTTTGTTGAAAAGTCTCAATAGCCCTTTGGTCT |
| | | TCTGAGACTGTATCTTTGATATTCTTGGAGTAGACGA |
| | | GAGTGTCGTGCTCCACCATGTTATCACATCAATCCAC |
| | | TTGCTTTGAAGACGTGGTTGGAACGTCTTCTTTTTCC |
| | | ACGATGCTCCTCGTGGGTGGGGGTCCATCTTTGGGAC |
| | | CACTGTCGGCAGAGGCATCTTGAACGATAGCCTTTCC |
| | | TTTATCGCAATGATGGCATTTGTAGGTGCCACCTTCC |
| | | TTTTCTACTGTCCTTTTGATGAAGTGACAGATAGCTG |
| | | GGCAATGGAATCCGAGGAGGTTTCCCGATATTACCC |
| | | TTTGTTGAAAAGTCTCAATAGCCCTTTGGTCTTCTGA |
| | | GACTGTATCTTTGATATTCTTGGAGTAGACGAGAGTG |
| | | TCGTGCTCCACCATGTTGGCAAGCTGCTCTAGCCAAT |
| | | ACGCAAACCGCCTCTCCCCGCGCGTTGGCCGATTCAT |
| | | TAATGCAGCTGGCACGACAGGTTTCCCGACTGGAAA |
| | | GCGGGCAGTGAGCGCAACGCAATTAATGTGAGTTAG |
| | | CTCACTCATTAGGCACCCCAGGCTTTACACTTTATGC |
| | | TTCCGGCTCGTATGTTGTGTGGAATTGTGAGCGGATA |
| | | ACAATTTCACACAGGAAACAGCTATGACCATGATTA |
| | | CGAATTCGAGCTCGGTACCCGGGGATCCTCTAGAGT |
| | | CGACCTGCAGGCATGCAAGCTTGGCACTGGCCGTCG |
| | | TTTTACAACGTCGTGACTGGGAAAACCCTGGCGTTAC |
| | | CCAACTTAATCGCCTTGCAGCACATCCCCCTTTCGCC |
| | | AGCTGGCGTAATAGCGAAGAGGCCCGCACCGATCGC |
| | | CCTTCCCAACAGTTGCGCAGCCTGAATGGCGAATGCT |
| | | AGAGCAGCTTGAGCTTGGATCAGATTGTCGTTTCCCG |
| | | CCTTCAGTTTAGCTTCATGGAGTCAAAGATTCAAATA |
| | | GAGGACCTAACAGAACTCGCCGTAAAGACTGGCGAA |
| | | CAGTTCATACAGAGTCTCTTACGACTCAATGACAAG |
| | | AAGAAAATCTTCGTCAACATGGTGGAGCACGACACA |
| | | CTTGTCTACTCCAAAAATATCAAAGATACAGTCTCAG |
| | | AAGACCAAAGGGCAATTGAGACTTTTCAACAAAGGG |
| | | TAATATCCGGAAACCTCCTCGGATTCCATTGCCCAGC |
| | | TATCTGTCACTTTATTGTGAAGATAGTGGAAAAGGA |
| | | AGGTGGCTCCTACAAATGCCATCATTGCGATAAAGG |
| | | AAAGGCCATCGTTGAAGATGCCTCTGCCGACAGTGG |
| | | TCCCAAAGATGGACCCCCACCCACGAGGAGCATCGT |
| | | GGAAAAAGAAGACGTTCCAACCACGTCTTCAAAGCA |
| | | AGTGGATTGATGTGATATCTCCACTGACGTAAGGGA |
| | | TGACGCACAATCCCACTATCCTTCGCAAGACCCTTCC |
| | | TCTATATAAGGAAGTTCATTTCATTTGGAGAGAACAC |
| | | GGGGGACTCTTGACCATGGTA |
| 12 | pGWB5:35S:CBCAScds:stop | tgagcgtcgcaaaggcgctcggtcttgccttgctcgtcggtgatgtacttcaccagctcc |
| | | gcgaagtcgctcttcttgatggagcgcatggggacgtgcttggcaatcacgcgcaccc |
| | | ccc ggccgttttagcggctaaaaaagtcatggctctgccctcgggc ggaccacgccca |
| | | tcatgaccttgccaagctcgtcctgcttctcttcgatcttcgccagcagggcgaggatcgt |
| | | ggcatcaccgaaccgcgccgtgcgcgggtcgtcggtgagccagagtacagcaggcc |
| | | gcccaggcggcccaggtcgccattgatgcgggccagctcgcggacgtgctcatagtc |
| | | cacgacgcccgtgattttgtagccctggccgacggccagcaggtaggccgacaggct |
| | | catgccggccgccgccgccttttcctcaatcgctcttcgttcgtctggaaggcagtacac |
| | | cttgataggtgggctgcccttcctggttggcttggtttcatcagccatccgcttgccctcat |
| | | ctgttacgccggcggtagccggccagcctcgcagagcaggattcccgttgagcaccg |
| | | ccaggtgcgaataagggacagtgaagaaggaacacccgctcgcgggtgggcctactt |
| | | cacctatcctgcccggctgacgccgttggatacaccaaggaaagtctacacgaaccctt |
| | | tggcaaaatcctgtatatcgtgcgaaaaaggatggatataccgaaaaaatcgctataatg |
| | | accccgaagcagggttatgcagcggaaaagcgccacgcttcccgaagggagaaagg |
| | | cggacaggtatccggtaagcggcagggtcggaacaggagagcgcacgagggagct |
| | | tccagggggaaacgcctggtatctttatagtcctgtcgggtttcgccacctctgacttgag |
| | | cgtcgattatgtgatgctcgtcaggggggcggagcctatggaaaaacgccagcaacg |
| | | cggcctattacggacctggccttagctggccttagctcacatgttctacctgcgttatcc |
| | | cctgattctgtggataaccgtattaccgcctttgagtgagctgataccgctcgccgcagc |
| | | cgaacgaccgagcgcagcgagtcagtgagcgaggaagcggaagagcgccagaag |
| | | gccgccagagaggccgagcgcggccgtgaggcttggacgctagggcagggcatga |
| | | aaaagcccgtagcgggctgctacgggcgtctgacgcggtggaaagggggaggggat |
| | | gttgtctacatggctctgctgtagtgagtgggttgcgctccggcagcggtcctgatcaat |
| | | cgtcaccctttctcggtccttcaacgttcctgacaacgagcctccttttcgccaatccatcg |
| | | acaatcaccgcgagtccctgctc gaacgctgcgtcc ggaccggcttcgtcgaaggcgt |
| | | ctatcgcggcccgcaacagcggcgagagcggagcctgttcaacggtgccgccgcgc |
| | | tcgccggcatcgctgtcgccggcctgctcctcaagcacggccccaacagtgaagtagc |
| | | tgattgtcatcagcgcattgacggcgtccccggccgaaaaacccgcctcgcagaggaa |
| | | gcgaagctgcgcgtcggccgtttccatctgcggtgcgcccggtcgcgtgccggcatg |
| | | gatgcgcgcgccatcgcggtaggcgagcagcgcctgcctgaagctgcgggcattccc |
| | | gatcagaaatgagcgccagtcgtcgtcggctctcggcaccgaatgcgtatgattctccg |
| | | ccagcatggcttcggccagtgcgtcgagcagcgcccgcttgttcctgaagtgccagta |
| | | aagcgccggctgctgaacccccaaccgttccgccagtttgcgtgtcgtcagaccgtcta |
| | | cgccgacctcgttcaacaggtccagggcggcacggatcactgtattcggctgcaacttt |
| | | gtcatgcttgacactttatcactgataaacataatatgtccaccaacttatcagtgataaag |
| | | aatccgcgcgttcaatcggaccagcggaggctggtccggaggccagacgtgaaacc |
| | | caacatacccctgatcgtaattctgagcactgtcgcgctcgacgctgtcggcatcggcct |
| | | gattatgccggtgctgccgggcctcctgcgcgatctggttcactcgaacgacgtcaccg |
| | | cccactatggcattctgctggcgctgtatgcgaggtgcaatttgcctgcgcacctgtgct |
| | | gggcgcgctgtcggatcgtttcgggcggcggccaatcttgctcgtctcgctggccggc |
| | | gccagatctggggaaccctgtggttggcatgcacatacaaatggacgaacggataaac |
| | | cttttcacgcccttttaaatatccgattattctaataaacgctcttttctcttaggtttacccgcc |
| | | aatatatcctgtcaaacactgatagtttaaactgaaggcgggaaacgacaatctgatcat |
| | | gagcggagaattaagggagtcacgttatgacccccgccgatgacgcgggacaagcc |
| | | gttttacgtttggaactgacagaaccgcaacgttgaaggagccactcagccgcgggttt |
| | | ctggagtttaatgagctaagcacatacgtcagaaaccattattgcgcgttcaaaagtcgc |
| | | ctaaggtcactatcagctagcaaatatttcttgtcaaaaatgctccactgacgttccataaa |
| | | ttcccctcggtatccaattagagtctcatattcactctcaatccaaataatctgcaccggat |
| | | ctggatcgtttcgcatgattgaacaagatggattgcacgcaggttctccggccgcttggg |
| | | tggagaggctattcggctatgactgggcacaacagacaatcggctgctctgatgccgc |
| | | cgtgttccggctgtcagcgcaggggcgcccggttctttttgtcaagaccgacctgtccg |
| | | gtgccctgaatgaactgcaggacgaggcagcgcggctatcgtggctggccacgacg |
| | | ggcgttccttgcgcagctgtgctcgacgttgtcactgaagcgggaagggactggctgct |
| | | attgggcgaagtgccggggcaggatctcctgtcatctcaccttgctcctgccgagaaag |
| | | tatccatcatggctgatgcaatgcggcggctgcatacgcttgatccggctacctgcccat |
| | | tcgaccaccaagcgaaacatcgcatcgagcgagcacgtactcggatggaagccggtc |
| | | ttgtcgatcaggatgatctggacgaagagcatcaggggctcgcgccagccgaactgtt |
| | | cgccaggctcaaggcgcgcatgcccgacggcgatgatctcgtcgtgacccatggcga |
| | | tgcctgcttgccgaatatcatggtggaaaatggccgcttttctggattcatcgactgtggc |
| | | cggctgggtgtggcggaccgctatcaggacatagcgttggctacccgtgatattgctga |
| | | agagcttggcggcgaatgggctgaccgcttcctcgtgctttacggtatcgccgctcccg |
| | | attcgcagcgcatcgccttctatcgccttcttgacgagttcttctgagcgggactctgggg |
| | | ttcgaaatgaccgaccaagcgacgcccaacctgccatcacgagatttcgattccaccgc |
| | | cgccttctatgaaaggttgggcttcggaatcgttttccgggacgccggctggatgatcct |
| | | ccagcgcggggatctcatgctggagttcttcgcccacgggatctctgcggaacaggcg |
| | | gtcgaaggtgccgatatcattacgacagcaacggccgacaagcacaacgccacgatc |
| | | ctgagcgacaatatgatcgggcccggcgtccacatcaacggcgtcggcggcgactgc |
| | | ccaggcaagaccgagatgcaccgcgatatcttgctgcgttcggatattttcgtggagttc |
| | | ccgccacagacccggatgatccccgatcgttcaaacatttggcaataaagtttcttaaga |
| | | ttgaatcctgttgccggtcttgcgatgattatcatataatttctgttgaattacgttaagcatgt |
| | | aataattaacatgtaatgcatgacgttatttatgagatgggtttttatgattagagtcccgca |
| | | attatacatttaatacgcgatagaaaacaaaatatagcgcgcaaactaggataaattatcg |
| | | cgcgcggtgtcatctatgttactagatcgggcctcctgtcaatgctggcggcggctctgg |
| | | tggtggttctggtggcggctctgagggtggtggctctgagggtggcggttctgagggtg |
| | | gcggctctgagggaggcggttccggtggtggctctggttccggtgattttgattatgaaa |
| | | agatggcaaacgctaataagggggctatgaccgaaaatgccgatgaaaacgcgctac |
| | | agtctgacgctaaaggcaaacttgattctgtcgctactgattacggtgctgctatcgatgg |
| | | tttcattggtgacgtttccggccttgctaatggtaatggtgctactggtgattttgctggctct |
| | | aattcccaaatggctcaagtcggtgacggtgataattcacctttaatgaataatttccgtca |
| | | atatttaccttccctccctcaatcggttgaatgtcgcccttttgtctttggcccaatacgcaa |
| | | accgcctctccccgcgcgttggccgattcattaatgcagctggcacgacaggtttcccg |
| | | actggaaagcgggcagtgagcgcaacgcaattaatgtgagttagctcactcattaggca |
| | | ccccaggctttacactttatgcttccggctcgtatgagtgtggaattgtgagcggataaca |
| | | atttcacacaggaaacagctatgaccatgattacgccaagcttgcatgcctgcaggtccc |
| | | cagattagccttttcaatttcagaaagaatgctaacccacagatggttagagaggcttacg |
| | | cagcaggtctcatcaagacgatctacccgagcaataatctccaggaaatcaaataccttc |
| | | ccaagaaggttaaagatgcagtcaaaagattcaggactaactgcatcaagaacacaga |
| | | gaaagatatatttctcaagatcagaagtactattccagtatggacgattcaaggcttgcttc |
| | | acaaaccaaggcaagtaatagagattggagtctctaaaaaggtagttcccactgaatca |
| | | aaggccatggagtcaaagattcaaatagaggacctaacagaactcgccgtaaagactg |
| | | gc gaacagttcatacagagtctcttacgactcaatgacaagaagaaaatcttcgtcaaca |
| | | tggtggagcacgacacacttgtctactccaaaaatatcaaagatacagtctcagaagac |
| | | caaagggcaattgagacttttcaacaaagggtaatatccggaaacctcctcggattccat |
| | | tgcccagctatctgtcactttattgtgaagatagtggaaaaggaaggtggctcctacaaat |
| | | gccatcattgcgataaaggaaaggccatcgttgaagatgcctctgccgacagtggtccc |
| | | aaagatggacccccacccacgaggagcatcgtggaaaaagaagacgttccaaccac |
| | | gtcttcaaagcaagtggattgatgtgatatctccactgacgtaagggatgacgcacaatc |
| | | ccactatccttcgcaagacccttcctctatataaggaagttcatttcatttggagagaacac |
| | | gggggactctaatcaaacaagtagtacaaaaaagctgaacgagaaacgtaaaatgatg |
| | | aattgctcaacattctccttttggtttgtttgcaaaataatatttactactctcattcaatatcca |
| | | aatttcaatagctaatcctcaagaaaacttccttaaatgcttctcggaatatattcctaacaa |
| | | tccagcaaatccaaaattcatatacactcaacacgaccaattgtatatgtctgtcctgaatt |
| | | cgacaatacaaaatcttagattcacctctgatacaaccccaaaaccactcgttattgtcact |
| | | ccttcaaatgtctcccatatccaggccagtattctctgctccaagaaagttggtttgcagat |
| | | tcgaactcgaagcggtggccatgatgctgagggtttgtcctacatatctcaagtcccattt |
| | | gctatagtagacttgagaaacatgcatacggtcaaagtagatattcatagccaaactgcg |
| | | tgggttgaagccggagctacccttggagaagtttattattggatcaatgagatgaatgag |
| | | aattttagttttcctggtgggtattgccctactgttggcgtaggtggacactttagtggagg |
| | | aggctatggagcattgatgcgaaattatggccttgcggctgataatatcattgatgcacac |
| | | ttagtcaatgttgatggaaaagttctagatcgaaaatccatgggagaagatctattttggg |
| | | ctatacgtggtggaggaggagaaaactttggaatcattgcagcatggaaaatcaaactt |
| | | gttgttgtcccatcaaaggctactatattcagtgttaaaaagaacatggagatacatgggc |
| | | ttgtcaagttatttaacaaatggcaaaatattgcttacaagtatgacaaagatttaatgctca |
| | | cgactcacttcagaactaggaatattacagataatcatgggaagaataagactacagtac |
| | | atggttacttctcttccatttttcttggtggagtggatagtctagttgacttgatgaacaagag |
| | | ctttcctgagttgggtattaaaaaaactgattgcaaagaattgagctggattgatacaacc |
| | | atcttctacagtggtgttgtaaattacaacactgctaattttaaaaaggaaattttgcttgata |
| | | gatcagctgggaagaagacggctttctcaattaagttagactatgttaagaaactaatacc |
| | | tgaaactgcaatggtcaaaattttggaaaaattatatgaagaagaggtaggagttgggat |
| | | gtatgtgttgtacccttacggtggtataatggatgagatttcagaatcagcaattccattcc |
| | | ctcatcgagctggaataatgtatgaactaggtacactgctacctgggagaagcaagaa |
| | | gataacgaaaagcatataaactgggttcgaagtgtttataatttcacaactccttatgtgtc |
| | | ccaaaatccaagattggcgtatctcaattatagggaccttgatttaggaaaaactaatcct |
| | | gagagtcctaataattacacacaagcacgtatttggggtgaaaagtattttggtaaaaattt |
| | | taacaggttagttaaggtgaaaaccaaagctgatcccaataatttttttagaaacgaacaa |
| | | agtatcccacctcttccaccgcgtcatcattaaaatatattgatatttatatcattttacgtttct |
| | | cgttcagctttcttgtacaaagtggttcgatctagaggatccatggtgagcaagggcgag |
| | | gagctgttcaccggggtggtgcccatcctggtcgagctggacggcgacgtgaacggc |
| | | cacaagttcagcgtgtccggcgagggcgagggcgatgccacctacggcaagctgac |
| | | cctgaagttcatctgcaccaccggcaagctgcccgtgccctggcccaccctcgtgacc |
| | | accttcacctacggcgtgcagtgcttcagccgctaccccgaccacatgaagcagcacg |
| | | acttcttcaagtccgccatgcccgaaggctacgtccaggagcgcaccatcttcttcaag |
| | | gacgacggcaactacaagacccgcgccgaggtgaagttcgagggcgacaccctggt |
| | | gaaccgcatcgagctgaagggcatcgacttcaaggaggacggcaacatcctggggc |
| | | acaagctggagtacaactacaacagccacaacgtctatatcatggccgacaagcagaa |
| | | gaacggcatcaaggtgaacttcaagatccgccacaacatcgaggacggcagcgtgca |
| | | gctcgccgaccactaccagcagaacacccccatcggcgacggccccgtgctgctgcc |
| | | cgacaaccactacctgagcacccagtccgccctgagcaaagaccccaacgagaagc |
| | | gcgatcacatggtcctgctggagttcgtgaccgccgccgggatcactcacggcatgga |
| | | cgagctgtacaagtaaagcggcccgagctcgaatttccccgatcgttcaaacatttggc |
| | | aataaagtttcttaagattgaatcctgttgccggtcttgcgatgattatcatataatttctgttg |
| | | aattacgttaagcatgtaataattaacatgtaatgcatgacgttatttatgagatgggttttta |
| | | tgattagagtcccgcaattatacatttaatacgcgatagaaaacaaaatatagcgcgcaa |
| | | actaggataaattatcgcgcgcggtgtcatctatgttactagatcgggaattagcttcatca |
| | | acgcaagacatgcgcacgaccgtctgacaggagaggaataccgacgagcacagaa |
| | | aggacttgctcttggacgtaggcctatttctcaggcacatgtatcaagtgttcggacgtgg |
| | | gttttcgatggtgtatcagccgccgccaactgggagatgaggaggctttcttggggggc |
| | | agtcagcagttcatttcacaagacagaggaacttgtaaggagatgcactgatttatcttgg |
| | | cgcaaaccagcaggacgaattagtgggaatagcccgcgaatatctaagttatgcctgtc |
| | | ggcatgagcagaaacttccaattcgaaacagtttggagaggttgtttttgggcatacctttt |
| | | gttagtcagcctctcgattgctcatcgtcattacacagtaccgaagtagatcgatctagta |
| | | acatagatgacaccgcgcgcgataatttatcctagtttgcgcgctatattttgttttctatcg |
| | | cgtattaaatgtataattgcgggactctaatcataaaaacccatctcataaataacgtcatg |
| | | cattacatgttaattattacatgcttaacgtaattcaacagaaattatatgataatcatcgcaa |
| | | gaccggcaacaggattcaatcttaagaaactttattgccaaatgtttgaacgatctgcttc |
| | | gacgcactccttctttactccaccatctcgtccttattgaaaacgtgggtagcaccaaaac |
| | | gaatcaagtcgctggaactgaagttaccaatcacgctggatgatttgccagttggattaat |
| | | cttgcctttccccgcatgaataatattgatgaatgcatgcgtgaggggtatttcgattttgg |
| | | caatagctgcaattgccgcgacatcctccaacgagcataattcttcagaaaaatagcgat |
| | | gttccatgttgtcagggcatgcatgatgcacgttatgaggtgacggtgctaggcagtatt |
| | | ccctcaaagtttcatagtcagtatcatattcatcattgcattcctgcaagagagaattgaga |
| | | cgcaatccacacgctgcggcaaccttccggcgttcgtggtctatttgctcttggacgttgc |
| | | aaacgtaagtgttggatcccggtcggcatctactctattcctagccctcggacgagtgct |
| | | ggggcgtcggtaccactatcggcgagtacttctacacagccatcggtccagacggcc |
| | | gcgcttctgcgggcgatttgtgtacgcccgacagtcccggctccggatcggacgattgc |
| | | gtcgcatcgaccctgcgcccaagctgcatcatcgaaattgccgtcaaccaagctctgat |
| | | agagttggtcaagaccaatgcggagcatatacgcccggagccgcggcgatcctgcaa |
| | | gctccggatgcctccgctcgaagtagcgcgtctgctgctccatacaagccaaccacgg |
| | | cctccagaagaagatgttggcgacctcgtattgggaatccccgaacatcgcctcgctcc |
| | | agtcaatgaccgctgttatgcggccattgtccgtcaggacattgttggagccgaaatccg |
| | | cgtgcacgaggtgccggacttcggggcagtcctcggcccaaagcatcagctcatcga |
| | | gagcctgcgcgacggacgcactgacggtgtcgtccatcacagtttgccagtgatacac |
| | | atggggatcagcaatcgcgcatatgaaatcacgccatgtagtgtattgaccgattccttg |
| | | cggtccgaatgggccgaacccgctcgtctggctaagatcggccgcagcgatcgcatc |
| | | catggcctccgcgaccggctgcagaacagcgggcagttcggtttcaggcaggtcttgc |
| | | aacgtgacaccctgtgcacggcgggagatgcaataggtcaggctctcgctgaattccc |
| | | caatgtcaagcacttccggaatcgggagcgcggccgatgcaaagtgccgataaacata |
| | | acgatctttgtagaaaccatcggcgcagctatttacccgcaggacatatccacgccctcc |
| | | tacatcgaagctgaaagcacgagattcttcgccctccgagagctgcatcaggtcggag |
| | | acgctgtcgaacttttcgatcagaaacttctcgacagacgtcgcggtgagttcaggctttt |
| | | tcatatcggggtcgtcctctccaaatgaaatgaacttccttatatagaggaagggtcttgc |
| | | gaaggatagtgggattgtgcgtcatcccttacgtcagtggagatatcacatcaatccactt |
| | | gctttgaagacgtggaggaacgtcttctttttccacgatgctcctcgtgggtgggggtcc |
| | | atctttgggaccactgtcggcagaggcatcttgaacgatagcctttcctttatcgcaatgat |
| | | ggcatttgtaggtgccaccttccttttctactgtccttttgatgaagtgacagatagctggg |
| | | caatggaatccgaggaggtttcccgatattaccctagttgaaaagtctcaatagccctttg |
| | | gtcttctgagactgtatctttgatattcttggagtagacgagagtgtcgtgctccaccatgtt |
| | | gacggatctctaggacgcgtcctagaagctaattcactggccgtcgttttacaacgtcgt |
| | | gactgggaaaaccctggcgttacccaacttaatc gccttgcagcacatccccctttcgcc |
| | | agctggcgtaatagcgaagaggcccgcaccgatcgcccttcccaacagttgcgcagc |
| | | ctgaatggcgcccgctcctttcgctacttcccttcctactcgccacgttcgccggctttcc |
| | | ccgtcaagctctaaatcgggggctccctttagggttccgatttagtgctttacggcacctc |
| | | gaccccaaaaaacttgatagggtgatggttcacgtagtgggccatcgccctgatagac |
| | | ggtttttcgccctttgacgttggagtccacgttctttaatagtggactcttgttccaaactgg |
| | | aacaacactcaaccctatctcgggctattcttttgatttataagggattttgccgatttcgga |
| | | accaccatcaaacaggattttcgcctgctggggcaaaccagcgtggaccgcttgctgc |
| | | aactctctcagggccaggcggtgaagggcaatcagctgttgcccgtctcactggtgaa |
| | | aagaaaaaccaccccagtacattaaaaacgtccgcaatgtgttattaagagtctaagcgt |
| | | caatttgtttacaccacaatatatcctgccaccagccagccaacagctccccgaccggc |
| | | agctcggcacaaaatcaccactcgatacaggcagcccatcagtccgggacggcgtca |
| | | gcgggagagccgttgtaaggcggcagactttgctcatgttaccgatgctattcggaaga |
| | | acggcaactaagctgccgggtttgaaacacggatgatctcgcggagggtagcatgttg |
| | | attgtaacgatgacagagcgttgctgcctgtgatcaaatatcatctccctcgcagagatcc |
| | | gaattatcagccttcttattcatttctcgcttaaccgtgacaggctgtcgatcttgagaacta |
| | | tgccgacataataggaaatcgctggataaagccgctgaggaagctgagtggcgctattt |
| | | ctttagaagtgaacgttgacgatatcaactcccctatccattgctcaccgaatggtacagg |
| | | tcggggacccgaagttccgactgtcggcctgatgcatccccggctgatcgaccccaga |
| | | tctggggctgagaaagcccagtaaggaaacaactgtaggttcgagtcgcgagatcccc |
| | | cggaaccaaaggaagtaggttaaacccgctccgatcaggccgagccacgccaggcc |
| | | gagaacattggttcctgtaggcatcgggattggcggatcaaacactaaagctactggaa |
| | | cgagcagaagtcctccggccgccagttgccaggcggtaaaggtgagcagaggcacg |
| | | ggaggttgccacttgcgggtcagcacggttccgaacgccatggaaaccgcccccgcc |
| | | aggcccgctgcgacgccgacaggatctagcgctgcgtaggtgtcaacaccaacagc |
| | | gccacgcccgcagttccgcaaatagcccccaggaccgccatcaatcgtatcgggctac |
| | | ctagcagagcggcagagatgaacacgaccatcagcggctgcacagcgcctaccgtc |
| | | gccgcgaccccgcccggcaggcggtagaccgaaataaacaacaagctccagaatag |
| | | cgaaatattaagtgcgccgaggatgaagatgcgcatccaccagattcccgttggaatct |
| | | gtcggacgatcatcacgagcaataaacccgccggcaacgcccgcagcagcataccg |
| | | gcgacccctcggcctcgctgttcgggctccacgaaaacgccggacagatgcgccttgt |
| | | gagcgtccttggggccgtcctcctgtttgaagaccgacagcccaatgatctcgccgtcg |
| | | atgtaggcgccgaatgccacggcatctcgcaaccgttcagcgaacgcctccatgggct |
| | | ttttctcctcgtgctcgtaaacggacccgaacatctctggagctttcttcagggccgacaa |
| | | tcggatctcgcggaaatcctgcacgtcggccgctccaagccgtcgaatctgagccttaa |
| | | tcacaattgtcaattaaatcctctgatatcggcagttcgtagagcgcgccgtgcgtcccg |
| | | agcgatactgagcgaagcaagtgcgtcgagcagtgcccgcttgttcctgaaatgccag |
| | | taaagcgctggctgctgaacccccagccggaactgaccccacaaggccctagcgtttg |
| | | caatgcaccaggtcatcattgacccaggcgtgttccaccaggccgctgcctcgcaactc |
| | | ttcgcaggcttcgccgacctgctcgcgccacttcttcacgcgggtggaatccgatccgc |
| | | acatgaggcggaaggtttccagcttgagcgggtacggctcccggtgcgagctgaaata |
| | | gtcgaacatccgtcgggccgtcggcgacagcttgcggtacttctcccatatgaatacgt |
| | | gtagtggtcgccagcaaacagcacgacgatttcctcgtcgatcaggacctggcaacgg |
| | | gacgttacttgccacggtccaggacgcggaagcggtgcagcagcgacaccgattcca |
| | | ggtgcccaacgcggtcggacgtgaagcccatcgccgtcgcctgtaggcgcgacagg |
| | | cattcctcggccttcgtgtaataccggccattgatcgaccagcccaggtcctggcaaag |
| | | ctcgtagaacgtgaaggtgatcggctcgccgataggggtgcgcttcgcgtactccaac |
| | | acctgctgccacaccagttcgtcatcgtcggcccgcagctcgacgccggtgtaggtgat |
| | | cttcacgtccttgttgacgtggaaaatgaccttgttttgcagcgcctcgcgcgggattttct |
| | | tgttgcgcgtggtgaacagggcagagcgggccgtgtcgtttggcatcgctcgcatcgt |
| | | gtccggccacggcgcaatatcgaacaaggaaagctgcataccttgatctgctgcttcgt |
| | | gtgtttcagcaacgcggcctgcttggcctcgctgacctgttttgccaggtcctcgccggc |
| | | ggtattcgcttcaggtcgtcatagacctcgcgtgtcgatggtcatcgacttcgccaaacc |
| | | tgccgcctcctgttcgagacgacgcgaacgctccacggcggccgatggcgcgggca |
| | | gggcagggggagccagttgcacgctgtcgcgctcgatcttggccgtagcttgctggac |
| | | catcgagccgacggactggaaggtttcgcggggcgcacgcatgacggtgcggcttgc |
| | | gatggtacggcatcctcggcggaaaaccccgcgtcgatcagacttgcctgtatgccttc |
| | | cggtcaaacgtccgattcattcaccctccttgcgggattgccccgactcacgccggggc |
| | | aatgtgcccttattcctgatttgacccgcctggtgccttggtgtccagataatccaccttatc |
| | | ggcaatgaagtcggtcccgtagaccgtctggccgtccttctcgtacttggtattccgaatc |
| | | ttgccctgcacgaataccagcgaccccttgcccaaatacttgccgtgggcctcggcctg |
| | | agagccaaaacacttgatgcggaagaagtcggtgcgctcctgcttgtcgccggcatcg |
| | | ttgcgccacatctaggtactaaaacaattcatccagtaaaatataatattttattttctcccaa |
| | | tcaggcttgatccccagtaagtcaaaaaatagctcgacatactgacaccccgatatcct |
| | | ccctgatcgaccggacgcagaaggcaatgtcataccacttgtccgccctgccgcttctc |
| | | ccaagatcaataaagccacttactagccatctacacaaagatgttgctgtctcccaggtc |
| | | gccgtgggaaaagacaagttcctcttcgggcttaccgtctttaaaaaatcatacagctcg |
| | | cgcggatctttaaatggagtgtcttcttcccagttttcgcaatccacatcggccagatcgtt |
| | | attcagtaagtaatccaattcggctaagcggctgtctaagctattcgtatagggacaatcc |
| | | gatatgtcgatggagtgaaagagcctgatgcactccgcatacagctcgataatcttttca |
| | | gggctttgttcatcttcatactcttccgagcaaaggacgccatcggcctcactcatgagca |
| | | gattgctccagccatcatgccgttcaaagtgcaggacctttggaacaggcagctttcctt |
| | | ccagccatagcatcatgtccttttcccgttccacatcataggtggtccctttataccggctg |
| | | tccgtcattataaatataggttacattactcccaccagcttatataccttagcaggagacat |
| | | tccttccgtatatttacgcagcggtattatcgatcagtatttcaattccggtgatattctcatt |
| | | ttagccatttattatttccttcctcttttctacagtatttaaagataccccaagaagctaattata |
| | | acaagacgaactccaattcactgttccttgcattctaaaaccttaaataccagaaaacagc |
| | | tattcaaagttgttacaaagaggcgtataacatagtatcgacggagccgattagaaacc |
| | | acaattatgggtgatgctgccaacttactgatttagtgtatgatggtgtattgaggtgctcc |
| | | agtggcttctgtgtctatcagctgtccctcctgacagctactgacggggtggtgcgtaac |
| | | ggcaaaagcaccgccggacatcagcgctatctctgctctcactgccgtaaaacatggc |
| | | aactgcagttcacttacaccgcttctcaacccggtacgcaccagaaaatcattgatatgg |
| | | ccatgaatggcgttggatgccgggcaacagcccgcattatgggcgttggcctcaacac |
| | | gattttacgtcacttaaaaaactcaggccgcagtcggtaacctcgcgcatacagccggg |
| | | cagtgacgtcatcgtctgcgcggaaatggacgaacagtggggctatgtcggggctaaa |
| | | tcgcgccagcgctggctgttttacgcgtatgacagtctccggaagacggagttgcgca |
| | | cgtattcggtgaacgcactatggcgacgctggggcgtcttatgagcctgctgtcaccat |
| | | tgacgtggtgatatggatgacggatggctggccgctgtatgaatcccgcctgaaggga |
| | | aagctgcacgtaatcagcaagcgatatacgcagcgaattgagcggcataacctgaatct |
| | | gaggcagcacctggcacggctgggacggaagtcgctgtcgttctcaaaatcggtgga |
| | | gctgcatgacaaagtcatcgggcattatctgaacataaaacactatcaataagttggagt |
| | | cattacccaattatgatagaatttacaagctataaggttattgtcctgggtacaagcattag |
| | | tccatgcaagtattatgctagcccattctatagatatattgataagcgcgctgcctatgcct |
| | | tgccccctgaaatccttacatacggcgatatcttctatataaaagatatattatcttatcagta |
| | | ttgtcaatatattcaaggcaatctgcctcctcatcctcttcatcctcttcgtcttggtagctatt |
| | | aaatatggcgcttcatagagtaattctgtaaaggtccaattctcgttacatacctcggtata |
| | | atcttacctatcacctcaaatggttcgctgggtttatcgcacccccgaacacgagcacgg |
| | | cacccgcgaccactatgccaagaatgcccaaggtaaaaattgccggccccgccatga |
| | | agtccgtgaatgccccgacggccgaagtgaagggcaggccgccacccaggccgcc |
| | | gccctcactgcccggcacctggtcgctgaatgtcgatgccagcacctgcggcacgtca |
| | | atgcttccgggcgtcgcgctcgggctgatcgcccatcccgttactgccccgatcccggc |
| | | aatggcaaggactgccagcgctgccatttaggggtgaggccgttcgcggccgaggg |
| | | gcgcagcccctggggggatgggaggcccgcgttagcgggccgggagggttcgaga |
| | | agggggggcaccccccttcggcgtgcgcggtcacgcgcacagggcgcagccctggt |
| | | taaaaacaaggtttataaatattggtttaaaagcaggttaaaagacaggttagcggtggc |
| | | cgaaaaacgggcggaaacccttgcaaatgctggattttctgcctgtggacagcccctca |
| | | aatgtcaataggtgcgcccctcatctgtcagcactctgcccctcaagtgtcaaggatcgc |
| | | gcccctcatctgtcagtagtcgcgcccctcaagtgtcaataccgcagggcacttatcccc |
| | | aggcttgtccacatcatctgtgggaaactcgcgtaaaatcaggcgttttcgccgatttgcg |
| | | aggctggccagctccacgtcgccggccgaaatcgagcctgcccctcatctgtcaacgc |
| | | cgcgccgggtgagtcggcccctcaagtgtcaacgtccgcccctcatctgtcagtgagg |
| | | gccaagttttccgcgaggtatccacaacgccggcggccgcggtgtctcgcacacggct |
| | | tcgacggcgtttctggcgcgtttgcagggccatagacggccgccagcccagcggcga |
| | | gggcaaccagcccgg |
| 13 | pGWB5:35S:CBDAScds:Stop | tgagcgtcgcaaaggcgctcggtcttgccttgctcgtcggtgatgtacttcaccagctcc |
| | | gcgaagtcgctcttcttgatggagcgcatggggacgtgcttggcaatcacgcgcaccc |
| | | cccggccgttttagcggctaaaaaagtcatggctctgccctcgggcggaccacgccca |
| | | tcatgaccttgccaagctcgtcctgcttctcttcgatcttcgccagcagggcgaggatcgt |
| | | ggcatcaccgaaccgcgccgtgcgcgggtcgtcggtgagccagagtacagcaggcc |
| | | gcccaggcggcccaggtcgccattgatgcgggccagctcgcggacgtgctcatagtc |
| | | cacgacgcccgtgattttgtagccctggccgacggccagcaggtaggccgacaggct |
| | | catgccggccgccgccgccttttcctcaatcgctcttcgttcgtctggaaggcagtacac |
| | | cttgataggtgggctgcccttcctggttggcttggtttcatcagccatccgcttgccctcat |
| | | ctgttacgccggcggtagccggccagcctcgcagagcaggattcccgttgagcaccg |
| | | ccaggtgcgaataagggacagtgaagaaggaacacccgctcgcgggtgggcctactt |
| | | cacctatcctgcccggctgacgccgttggatacaccaaggaaagtctacacgaaccctt |
| | | tggcaaaatcctgtatatcgtgcgaaaaaggatggatataccgaaaaaatcgctataatg |
| | | accccgaagcagggttatgcagcggaaaagcgccacgcttcccgaagggagaaagg |
| | | cggacaggtatccggtaagcggcagggtcggaacaggagagcgcacgagggagct |
| | | tccagggggaaacgcctggtatctttatagtcctgtcgggtttcgccacctctgacttgag |
| | | cgtcgatttttgtgatgctcgtcaggggggcggagcctatggaaaaacgccagcaacg |
| | | cggcctttttacggacctggccttagctggccttagctcacatgttctacctgcgttatcc |
| | | cctgattctgtggataaccgtattaccgcctttgagtgagctgataccgctcgccgcagc |
| | | cgaacgaccgagcgcagcgagtcagtgagcgaggaagcggaagagcgccagaag |
| | | gccgccagagaggccgagcgcggccgtgaggcttggacgctagggcagggcatga |
| | | aaaagcccgtagcgggctgctacgggcgtctgacgcggtggaaagggggaggggat |
| | | gttgtctacatggctctgctgtagtgagtgggttgcgctccggcagcggtcctgatcaat |
| | | cgtcaccctttctcggtccttcaacgttcctgacaacgagcctccttttcgccaatccatcg |
| | | acaatcaccgcgagtccctgctc gaacgctgcgtcc ggaccggcttcgtcgaaggcgt |
| | | ctatcgcggcccgcaacagcggcgagagcggagcctgttcaacggtgccgccgcgc |
| | | tcgccggcatcgctgtcgccggcctgctcctcaagcacggccccaacagtgaagtagc |
| | | tgattgtcatcagcgcattgacggcgtccccggccgaaaaacccgcctcgcagaggaa |
| | | gcgaagctgcgcgtcggccgtttccatctgcggtgcgcccggtcgcgtgccggcatg |
| | | gatgcgcgcgccatcgcggtaggcgagcagcgcctgcctgaagctgcgggcattccc |
| | | gatcagaaatgagcgccagtcgtcgtcggctctcggcaccgaatgcgtatgattctccg |
| | | ccagcatggcttcggccagtgcgtcgagcagcgcccgcttgttcctgaagtgccagta |
| | | aagcgccggctgctgaacccccaaccgttccgccagtttgcgtgtcgtcagaccgtcta |
| | | cgccgacctcgttcaacaggtccagggcggcacggatcactgtattcggctgcaacttt |
| | | gtcatgcttgacactttatcactgataaacataatatgtccaccaacttatcagtgataaag |
| | | aatccgcgcgttcaatcggaccagcggaggctggtccggaggccagacgtgaaacc |
| | | caacatacccctgatcgtaattctgagcactgtcgcgctcgacgctgtcggcatcggcct |
| | | gattatgccggtgctgccgggcctcctgcgcgatctggttcactcgaacgacgtcaccg |
| | | cccactatggcattctgctggcgctgtatgcgaggtgcaatttgcctgcgcacctgtgct |
| | | gggcgcgctgtcggatcgtttcgggcggcggccaatcttgctcgtctcgctggccggc |
| | | gccagatctggggaaccctgtggttggcatgcacatacaaatggacgaacggataaac |
| | | cttttcacgcccttttaaatatccgattattctaataaacgctcttttctcttaggtttacccgcc |
| | | aatatatcctgtcaaacactgatagtttaaactgaaggcgggaaacgacaatctgatcat |
| | | gagcggagaattaagggagtcacgttatgacccccgccgatgacgcgggacaagcc |
| | | gttttacgtttggaactgacagaaccgcaacgttgaaggagccactcagccgcgggttt |
| | | ctggagtttaatgagctaagcacatacgtcagaaaccattattgcgcgttcaaaagtcgc |
| | | ctaaggtcactatcagctagcaaatatttcttgtcaaaaatgctccactgacgttccataaa |
| | | ttcccctcggtatccaattagagtctcatattcactctcaatccaaataatctgcaccggat |
| | | ctggatcgtttcgcatgattgaacaagatggattgcacgcaggttctccggccgcttggg |
| | | tggagaggctattc ggctatgactgggcacaacagacaatcggctgctctgatgccgc |
| | | cgtgttccggctgtcagcgcaggggcgcccggttctttttgtcaagaccgacctgtccg |
| | | gtgccctgaatgaactgcaggacgaggcagcgcggctatcgtggctggccacgacg |
| | | ggcgttccttgcgcagctgtgctcgacgttgtcactgaagcgggaagggactggctgct |
| | | attgggcgaagtgccggggcaggatctcctgtcatctcaccttgctcctgccgagaaag |
| | | tatccatcatggctgatgcaatgcggcggctgcatacgcttgatccggctacctgcccat |
| | | tcgaccaccaagcgaaacatcgcatcgagcgagcacgtactcggatggaagccggtc |
| | | ttgtcgatcaggatgatctggacgaagagcatcaggggctcgcgccagccgaactgtt |
| | | cgccaggctcaaggcgcgcatgcccgacggcgatgatctcgtcgtgacccatggcga |
| | | tgcctgcttgccgaatatcatggtggaaaatggccgcttttctggattcatcgactgtggc |
| | | cggctgggtgtggcggaccgctatcaggacatagcgttggctacccgtgatattgctga |
| | | agagcttggcggcgaatgggctgaccgcttcctcgtgctttacggtatcgccgctcccg |
| | | attcgcagcgcatcgccttctatcgccttcttgacgagttcttctgagcgggactctgggg |
| | | ttcgaaatgaccgaccaagcgacgcccaacctgccatcacgagatttcgattccaccgc |
| | | cgccttctatgaaaggttgggcttcggaatcgttttccgggacgccggctggatgatcct |
| | | ccagcgcggggatctcatgctggagttcttcgcccacgggatctctgcggaacaggcg |
| | | gtcgaaggtgccgatatcattacgacagcaacggccgacaagcacaacgccacgatc |
| | | ctgagcgacaatatgatcgggcccggcgtccacatcaacggcgtcggcggcgactgc |
| | | ccaggcaagaccgagatgcaccgcgatatcttgctgcgttcggatattttcgtggagttc |
| | | ccgccacagacccggatgatccccgatcgttcaaacatttggcaataaagtttcttaaga |
| | | ttgaatcctgttgccggtcttgcgatgattatcatataatttctgttgaattacgttaagcatgt |
| | | aataattaacatgtaatgcatgacgttatttatgagatgggtattatgattagagtcccgca |
| | | attatacatttaatacgcgatagaaaacaaaatatagcgcgcaaactaggataaattatcg |
| | | cgcgcggtgtcatctatgttactagatcgggcctcctgtcaatgctggcggcggctctgg |
| | | tggtggttctggtggcggctctgagggtggtggctctgagggtggcggttctgagggtg |
| | | gcggctctgagggaggcggttccggtggtggctctggttccggtgattttgattatgaaa |
| | | agatggcaaacgctaataagggggctatgaccgaaaatgccgatgaaaacgcgctac |
| | | agtctgacgctaaaggcaaacttgattctgtcgctactgattacggtgctgctatcgatgg |
| | | tttcattggtgacgtttccggccttgctaatggtaatggtgctactggtgattttgctggctct |
| | | aattcccaaatggctcaagtcggtgacggtgataattcacctttaatgaataataccgtca |
| | | atatttaccttccctccctcaatcggttgaatgtcgcccttttgtctttggcccaatacgcaa |
| | | accgcctctccccgcgcgttggccgattcattaatgcagctggcacgacaggtttcccg |
| | | actggaaagcgggcagtgagcgcaacgcaattaatgtgagttagctcactcattaggca |
| | | ccccaggctttacactttatgcttccggctcgtatgagtgtggaattgtgagcggataaca |
| | | atttcacacaggaaacagctatgaccatgattacgccaagcttgcatgcctgcaggtccc |
| | | cagattagccttttcaatttcagaaagaatgctaacccacagatggttagagaggcttacg |
| | | cagcaggtctcatcaagacgatctacccgagcaataatctccaggaaatcaaataccttc |
| | | ccaagaaggttaaagatgcagtcaaaagattcaggactaactgcatcaagaacacaga |
| | | gaaagatatatttctcaagatcagaagtactattccagtatggacgattcaaggcttgcttc |
| | | acaaaccaaggcaagtaatagagattggagtctctaaaaaggtagttcccactgaatca |
| | | aaggccatggagtcaaagattcaaatagaggacctaacagaactcgccgtaaagactg |
| | | gcgaacagttcatacagagtctcttacgactcaatgacaagaagaaaatcttcgtcaaca |
| | | tggtggagcacgacacacttgtctactccaaaaatatcaaagatacagtctcagaagac |
| | | caaagggcaattgagacttttcaacaaagggtaatatccggaaacctcctcggattccat |
| | | tgcccagctatctgtcactttattgtgaagatagtggaaaaggaaggtggctcctacaaat |
| | | gccatcattgcgataaaggaaaggccatcgttgaagatgcctctgccgacagtggtccc |
| | | aaagatggacccccacccacgaggagcatcgtggaaaaagaagacgttccaaccac |
| | | gtcttcaaagcaagtggattgatgtgatatctccactgacgtaagggatgacgcacaatc |
| | | ccactatccttcgcaagacccttcctctatataaggaagttcatttcatttggagagaacac |
| | | gggggactctaatcaaacaagtttgtacaaaaaagctgaacgagaaacgtaaaatgA |
| | | TGAAGTACTCAACATTCTCCTTTTGGTTTGTTTGCAA |
| | | GATAATATTTTTCTTTTTCTCATTCAATATCCAAACTT |
| | | CCATTGCTAATCCTCGAGAAAACTTCCTTAAATGCTT |
| | | CTCGCAATATATTCCCAATAATGCAACAAATCTAAA |
| | | ACTCGTATACACTCAAAACAACCCATTGTATATGTCT |
| | | GTCCTAAATTCGACAATACACAATCTTAGATTCAGCT |
| | | CTGACACAACCCCAAAACCACTTGTTATCGTCACTCC |
| | | TTCACATGTCTCTCATATCCAAGGCACTATTCTATGC |
| | | TCCAAGAAAGTTGGCTTGCAGATTCGAACTCGAAGT |
| | | GGTGGTCATGATTCTGAGGGCATGTCCTACATATCTC |
| | | AAGTCCCATTTGTTATAGTAGACTTGAGAAACATGCG |
| | | TTCAATCAAAATAGATGTTCATAGCCAAACTGCATG |
| | | GGTTGAAGCCGGAGCTACCCTTGGAGAAGTTTATTAT |
| | | TGGGTTAATGAGAAAAATGAGAGTCTTAGTTTGGCT |
| | | GCTGGGTATTGCCCTACTGTTTGCGCAGGTGGACACT |
| | | TTGGTGGAGGAGGCTATGGACCATTGATGAGAAGCT |
| | | ATGGCCTCGCGGCTGATAATATCATTGATGCACACTT |
| | | AGTCAACGTTCATGGAAAAGTGCTAGATCGAAAATC |
| | | TATGGGGGAAGATCTCTTTTGGGCTTTACGTGGTGGT |
| | | GGAGCAGAAAGCTTCGGAATCATTGTAGCATGGAAA |
| | | ATTAGACTGGTTGCTGTCCCAAAGTCTACTATGTTTA |
| | | GTGTTAAAAAGATCATGGAGATACATGAGCTTGTCA |
| | | AGTTAGTTAACAAATGGCAAAATATTGCTTACAAGT |
| | | ATGACAAAGATTTATTACTCATGACTCACTTCATAAC |
| | | TAGGAACATTACAGATAATCAAGGGAAGAATAAGAC |
| | | AGCAATACACACTTACTTCTCTTCAGTTTTCCTTGGT |
| | | GGAGTGGATAGTCTAGTCGACTTGATGAACAAGAGT |
| | | TTTCCTGAGTTGGGTATTAAAAAAACGGATTGCAGA |
| | | CAATTGAGCTGGATTGATACTATCATCTTCTATAGTG |
| | | GTGTTGTAAATTACGACACTGATAATTTTAACAAGGA |
| | | AATTTTGCTTGATAGATCCGCTGGGCAGAACGGTGCT |
| | | TTCAAGATTAAGTTAGACTACGTTAAGAAACCAATTC |
| | | CAGAATCTGTATTTGTCCAAATTTTGGAAAAATTATA |
| | | TGAAGAAGATATAGGAGCTGGGATGTATGCGTTGTA |
| | | CCCTTACGGTGGTATAATGGATGAGATTTCTGAATCA |
| | | GCAATTCCATTCCCTCATCGAGCTGGAATCTTGTATG |
| | | AGTTATGGTACATATGTAGCTGGGAGAAGCAAGAAG |
| | | ATAACGAAAAGCATCTAAACTGGATTAGAAATATTT |
| | | ATAACTTCATGACTCCTTATGTGTCCCAAAATCCAAG |
| | | ATTGGCATATCTCAATTATAGAGACCTTGATATAGGA |
| | | ATAAATGATCCCAAGAATCCAAATAATTACACACAA |
| | | GCACGTATTTGGGGTGAGAAGTATTTTGGTAAAAATT |
| | | TTGACAGGCTAGTAAAAGTGAAAACCCTGGTTGATC |
| | | CCAATAATTTTTTTAGAAACGAACAAAGCATCCCACC |
| | | TCTTCCACGGCATCATCATTAAaatatattgatatttatatcattttacg |
| | | tttctcgttcagctttcttgtacaaagtggttcgatctagaggatccatggtgagcaagggc |
| | | gaggagctgttcaccggggtggtgcccatcctggtcgagctggacggcgacgtgaac |
| | | ggccacaagttcagcgtgtccggcgagggcgagggcgatgccacctacggcaagct |
| | | gaccctgaagttcatctgcaccaccggcaagctgcccgtgccctggcccaccctcgtg |
| | | accaccttcacctacggcgtgcagtgcttcagccgctacccc gaccacatgaagcagc |
| | | acgacttcttcaagtccgccatgcccgaaggctacgtccaggagcgcaccatcttcttca |
| | | aggacgacggcaactacaagacccgcgccgaggtgaagttcgagggcgacaccctg |
| | | gtgaaccgcatc gagctgaagggcatcgacttcaaggaggacggcaacatcctgggg |
| | | cacaagctggagtacaactacaacagccacaacgtctatatcatggccgacaagcaga |
| | | agaacggcatcaaggtgaacttcaagatccgccacaacatcgaggacggcagcgtgc |
| | | agctcgccgaccactaccagcagaacacccccatcggcgacggccccgtgctgctgc |
| | | ccgacaaccactacctgagcacccagtccgccctgagcaaagaccccaacgagaag |
| | | cgcgatcacatggtcctgctggagttcgtgaccgccgccgggatcactcacggcatgg |
| | | acgagctgtacaagtaaagcggcccgagctcgaataccccgatcgttcaaacatagg |
| | | caataaagtttcttaagattgaatcctgttgccggtcttgcgatgattatcatataatttctgtt |
| | | gaattacgttaagcatgtaataattaacatgtaatgcatgacgttatttatgagatgggtatt |
| | | atgattagagtcccgcaattatacatttaatacgcgatagaaaacaaaatatagcgcgca |
| | | aactaggataaattatcgc gcgcggtgtcatctatgttactagatcgggaattagcttcatc |
| | | aacgcaagacatgcgcacgaccgtctgacaggagaggaatttccgacgagcacaga |
| | | aaggacttgctcttggacgtaggcctatttctcaggcacatgtatcaagtgttcggacgtg |
| | | ggttttcgatggtgtatcagccgccgccaactgggagatgaggaggctttcttgggggg |
| | | cagtcagcagttcatttcacaagacagaggaacttgtaaggagatgcactgatttatcttg |
| | | gcgcaaaccagcaggacgaattagtgggaatagcccgcgaatatctaagttatgcctgt |
| | | cggcatgagcagaaacttccaattcgaaacagtttggagaggttgtttttgggcatacctt |
| | | ttgttagtcagcctctcgattgctcatcgtcattacacagtaccgaagtttgatcgatctagt |
| | | aacatagatgacaccgcgcgcgataatttatcctagtttgcgcgctatattttgttttctatc |
| | | gcgtattaaatgtataattgcgggactctaatcataaaaacccatctcataaataacgtcat |
| | | gcattacatgttaattattacatgcttaacgtaattcaacagaaattatatgataatcatcgca |
| | | agaccggcaacaggattcaatcttaagaaactttattgccaaatgtttgaacgatctgcttc |
| | | gacgcactccttctttactccaccatctcgtccttattgaaaacgtgggtagcaccaaaac |
| | | gaatcaagtcgctggaactgaagttaccaatcacgctggatgatttgccagttggattaat |
| | | cttgcctttccccgcatgaataatattgatgaatgcatgcgtgaggggtatttcgattttgg |
| | | caatagctgcaattgccgcgacatcctccaacgagcataattcttcagaaaaatagcgat |
| | | gttccatgttgtcagggcatgcatgatgcacgttatgaggtgacggtgctaggcagtatt |
| | | ccctcaaagtttcatagtcagtatcatattcatcattgcattcctgcaagagagaattgaga |
| | | cgcaatccacacgctgcggcaaccttccggcgttcgtggtctatttgctcttggacgttgc |
| | | aaacgtaagtgttggatcccggtcggcatctactctattcctagccctcggacgagtgct |
| | | ggggcgtcggtaccactatcggcgagtacttctacacagccatcggtccagacggcc |
| | | gcgcttctgcgggcgatttgtgtacgcccgacagtcccggctccggatcggacgattgc |
| | | gtcgcatcgaccctgcgcccaagctgcatcatcgaaattgccgtcaaccaagctctgat |
| | | agagttggtcaagaccaatgcggagcatatacgcccggagccgcggcgatcctgcaa |
| | | gctccggatgcctccgctcgaagtagcgcgtctgctgctccatacaagccaaccacgg |
| | | cctccagaagaagatgttggcgacctcgtattgggaatccccgaacatcgcctcgctcc |
| | | agtcaatgaccgctgttatgcggccattgtccgtcaggacattgttggagccgaaatccg |
| | | cgtgcacgaggtgccggacttcggggcagtcctcggcccaaagcatcagctcatcga |
| | | gagcctgcgcgacggacgcactgacggtgtcgtccatcacagtttgccagtgatacac |
| | | atggggatcagcaatcgcgcatatgaaatcacgccatgtagtgtattgaccgattccttg |
| | | cggtccgaatgggccgaacccgctcgtctggctaagatcggccgcagcgatcgcatc |
| | | catggcctccgcgaccggctgcagaacagcgggcagttcggtttcaggcaggtcttgc |
| | | aacgtgacaccctgtgcacggcgggagatgcaataggtcaggctctcgctgaattccc |
| | | caatgtcaagcacttccggaatcgggagcgcggccgatgcaaagtgccgataaacata |
| | | acgatctttgtagaaaccatcggcgcagctatttacccgcaggacatatccacgccctcc |
| | | tacatcgaagctgaaagcacgagattcttcgccctccgagagctgcatcaggtcggag |
| | | acgctgtcgaacttttcgatcagaaacttctcgacagacgtcgcggtgagttcaggctttt |
| | | tcatatcggggtcgtcctctccaaatgaaatgaacttccttatatagaggaagggtcttgc |
| | | gaaggatagtgggattgtgcgtcatcccttacgtcagtggagatatcacatcaatccactt |
| | | gctttgaagacgtggaggaacgtcttctttttccacgatgctcctcgtgggtgggggtcc |
| | | atctttgggaccactgtcggcagaggcatcttgaacgatagcctttcctttatcgcaatgat |
| | | ggcatttgtaggtgccaccttccttttctactgtccttttgatgaagtgacagatagctggg |
| | | caatggaatccgaggaggtttcccgatattaccctagttgaaaagtctcaatagc cctttg |
| | | gtcttctgagactgtatctttgatattcttggagtagacgagagtgtcgtgctccaccatgtt |
| | | gacggatctctaggacgcgtcctagaagctaattcactggccgtcgttttacaacgtcgt |
| | | gactgggaaaaccctggcgttacccaacttaatcgccttgcagcacatccccctttcgcc |
| | | agctggcgtaatagcgaagaggcccgcaccgatcgcccttcccaacagttgcgcagc |
| | | ctgaatggcgcccgctcctttcgctacttcccttcctactcgccacgttcgccggctttcc |
| | | ccgtcaagctctaaatcgggggctccctttagggttccgatttagtgctttacggcacctc |
| | | gaccccaaaaaacttgatagggtgatggttcacgtagtgggccatcgccctgatagac |
| | | ggtttttcgccctttgacgttggagtccacgttctttaatagtggactcttgttccaaactgg |
| | | aacaacactcaaccctatctcgggctattcttttgatttataagggattttgccgatttcgga |
| | | accaccatcaaacaggattttcgcctgctggggcaaaccagcgtggaccgcttgctgc |
| | | aactctctcagggccaggcggtgaagggcaatcagctgttgcccgtctcactggtgaa |
| | | aagaaaaaccaccccagtacattaaaaacgtccgcaatgtgttattaagagtctaagcgt |
| | | caatttgtttacaccacaatatatcctgccaccagccagccaacagctccccgaccggc |
| | | agctcggcacaaaatcaccactcgatacaggcagcccatcagtccgggacggcgtca |
| | | gcgggagagccgttgtaaggcggcagactttgctcatgttaccgatgctattcggaaga |
| | | acggcaactaagctgccgggtttgaaacacggatgatctcgcggagggtagcatgttg |
| | | attgtaacgatgacagagcgttgctgcctgtgatcaaatatcatctccctcgcagagatcc |
| | | gaattatcagccttcttattcatttctcgcttaaccgtgacaggctgtcgatcttgagaacta |
| | | tgccgacataataggaaatcgctggataaagccgctgaggaagctgagtggcgctattt |
| | | ctttagaagtgaacgttgacgatatcaactcccctatccattgctcaccgaatggtacagg |
| | | tcggggacccgaagttccgactgtcggcctgatgcatccccggctgatcgaccccaga |
| | | tctggggctgagaaagcccagtaaggaaacaactgtaggttcgagtcgcgagatcccc |
| | | cggaaccaaaggaagtaggttaaacccgctccgatcaggccgagccacgccaggcc |
| | | gagaacattggttcctgtaggcatcgggattggcggatcaaacactaaagctactggaa |
| | | cgagcagaagtcctccggccgccagttgccaggcggtaaaggtgagcagaggcacg |
| | | ggaggttgccacttgcgggtcagcacggttccgaacgccatggaaaccgcccccgcc |
| | | aggcccgctgcgacgccgacaggatctagcgctgcgtttggtgtcaacaccaacagc |
| | | gccacgcccgcagttccgcaaatagcccccaggaccgccatcaatcgtatcgggctac |
| | | ctagcagagcggcagagatgaacacgaccatcagcggctgcacagcgcctaccgtc |
| | | gccgcgaccccgcccggcaggcggtagaccgaaataaacaacaagctccagaatag |
| | | cgaaatattaagtgcgccgaggatgaagatgcgcatccaccagattcccgttggaatct |
| | | gtcggacgatcatcacgagcaataaacccgccggcaacgcccgcagcagcataccg |
| | | gcgacccctcggcctcgctgttcgggctccacgaaaacgccggacagatgcgccttgt |
| | | gagcgtccttggggccgtcctcctgtttgaagaccgacagcccaatgatctcgccgtcg |
| | | atgtaggcgccgaatgccacggcatctcgcaaccgttcagcgaacgcctccatgggct |
| | | ttttctcctcgtgctcgtaaacggacccgaacatctctggagctttcttcagggccgacaa |
| | | tcggatctcgcggaaatcctgcacgtcggccgctccaagccgtcgaatctgagccttaa |
| | | tcacaattgtcaattaaatcctctgatatcggcagttcgtagagcgcgccgtgcgtcccg |
| | | agcgatactgagcgaagcaagtgcgtc gagcagtgccc gcttgttc ctgaaatgccag |
| | | taaagcgctggctgctgaacccccagccggaactgaccccacaaggccctagcgtttg |
| | | caatgcaccaggtcatcattgacccaggcgtgttccaccaggccgctgcctcgcaactc |
| | | ttcgcaggcttcgccgacctgctcgcgccacttcttcacgcgggtggaatccgatccgc |
| | | acatgaggcggaaggtttccagcttgagcgggtacggctcccggtgcgagctgaaata |
| | | gtcgaacatcc gtcgggccgtcggcgacagcttgcggtacttctcccatatgaatacgt |
| | | gtagtggtcgccagcaaacagcacgacgatttcctcgtcgatcaggacctggcaacgg |
| | | gacgttacttgccacggtccaggacgcggaagcggtgcagcagcgacaccgattcca |
| | | ggtgcccaacgcggtcggacgtgaagcccatcgccgtcgcctgtaggcgcgacagg |
| | | cattcctcggccttcgtgtaataccggccattgatcgaccagcccaggtcctggcaaag |
| | | ctcgtagaacgtgaaggtgatcggctcgccgataggggtgcgcttcgcgtactccaac |
| | | acctgctgccacaccagttcgtcatcgtc ggcccgcagctcgacgccggtgtaggtgat |
| | | cttcacgtccttgttgacgtggaaaatgaccttgttttgcagcgcctcgcgcgggattttct |
| | | tgttgcgcgtggtgaacagggcagagcgggccgtgtcgtttggcatcgctcgcatcgt |
| | | gtccggccacggcgcaatatcgaacaaggaaagctgcataccttgatctgctgcttcgt |
| | | gtgtttcagcaacgcggcctgcttggcctcgctgacctgttttgccaggtcctcgccggc |
| | | ggtattcgcttcaggtcgtcatagacctcgcgtgtcgatggtcatcgacttcgccaaacc |
| | | tgccgcctcctgttcgagacgacgcgaacgctccacggcggccgatggcgcgggca |
| | | gggcagggggagccagttgcacgctgtcgcgctcgatcttggccgtagcttgctggac |
| | | catcgagccgacggactggaaggtttcgcggggcgcacgcatgacggtgcggcttgc |
| | | gatggtacggcatcctcggcggaaaaccccgcgtcgatcagacttgcctgtatgccttc |
| | | cggtcaaacgtccgattcattcaccctccttgcgggattgccccgactcacgccggggc |
| | | aatgtgcccttattcctgatttgacccgcctggtgccttggtgtccagataatccaccttatc |
| | | ggcaatgaagtcggtcccgtagaccgtctggccgtccttctcgtacttggtattccgaatc |
| | | ttgccctgcacgaataccagcgaccccttgcccaaatacttgccgtgggcctcggcctg |
| | | agagccaaaacacttgatgcggaagaagtcggtgcgctcctgcttgtcgccggcatcg |
| | | ttgcgccacatctaggtactaaaacaattcatccagtaaaatataatattttattttctcccaa |
| | | tcaggcttgatccccagtaagtcaaaaaatagctcgacatactgacaccccgatatcct |
| | | ccctgatcgaccggacgcagaaggcaatgtcataccacttgtccgccctgccgcttctc |
| | | ccaagatcaataaagccacttactttgccatctttcacaaagatgttgctgtctcccaggtc |
| | | gccgtgggaaaagacaagacctcacgggcttaccgtcataaaaaatcatacagctcg |
| | | cgcggatctttaaatggagtgtcttcttcccagttttcgcaatccacatcggccagatcgtt |
| | | attcagtaagtaatccaattcggctaagcggctgtctaagctattcgtatagggacaatcc |
| | | gatatgtcgatggagtgaaagagcctgatgcactccgcatacagctcgataatcattca |
| | | gggctttgttcatcttcatactcttccgagcaaaggacgccatcggcctcactcatgagca |
| | | gattgctccagccatcatgccgttcaaagtgcaggacctttggaacaggcagctttcctt |
| | | ccagccatagcatcatgtccttttcccgttccacatcataggtggtccctttataccggctg |
| | | tccgtcatttttaaatataggttacattactcccaccagcttatataccttagcaggagacat |
| | | tccttccgtatcttttacgcagcggtatttttcgatcagttttttcaattccggtgatattctcatt |
| | | ttagccatttattatttccttcctcttttctacagtatttaaagataccccaagaagctaattata |
| | | acaagacgaactccaattcactgttccttgcattctaaaaccttaaataccagaaaacagc |
| | | tttttcaaagttgttacaaagaggcgtataacatagtatcgacggagccgattagaaacc |
| | | acaattatgggtgatgctgccaacttactgatttagtgtatgatggtgtttttgaggtgctcc |
| | | agtggcttctgtgtctatcagctgtccctcctgacagctactgacggggtggtgcgtaac |
| | | ggcaaaagcaccgccggacatcagcgctatctctgctctcactgccgtaaaacatggc |
| | | aactgcagttcacttacaccgcttctcaacccggtacgcaccagaaaatcattgatatgg |
| | | ccatgaatggcgaggatgccgggcaacagcccgcattatgggcgttggcctcaacac |
| | | gattttacgtcacttaaaaaactcaggccgcagtcggtaacctcgcgcatacagccggg |
| | | cagtgacgtcatcgtctgcgcggaaatggacgaacagtggggctatgtcggggctaaa |
| | | tcgcgccagcgctggctgttttacgcgtatgacagtctccggaagacggagttgcgca |
| | | cgtattcggtgaacgcactatggcgacgctggggcgtcttatgagcctgctgtcaccat |
| | | tgacgtggtgatatggatgacggatggctggccgctgtatgaatcccgcctgaaggga |
| | | aagctgcacgtaatcagcaagcgatatacgcagcgaattgagcggcataacctgaatct |
| | | gaggcagcacctggcacggctgggacggaagtcgctgtcgttctcaaaatcggtgga |
| | | gctgcatgacaaagtcatcgggcattatctgaacataaaacactatcaataagttggagt |
| | | cattacccaattatgatagaatttacaagctataaggttattgtcctgggtacaagcattag |
| | | tccatgcaagtttttatgctagcccattctatagatatattgataagcgcgctgcctatgcct |
| | | tgccccctgaaatccttacatacggcgatatcttctatataaaagatatattatcttatcagta |
| | | ttgtcaatatattcaaggcaatctgcctcctcatcctcttcatcctcttcgtcttggtagcttttt |
| | | aaatatggcgcttcatagagtaattctgtaaaggtccaattctcgttacatacctcggtata |
| | | atcttacctatcacctcaaatggttcgctgggtttatcgcacccccgaacacgagcacgg |
| | | cacccgcgaccactatgccaagaatgcccaaggtaaaaattgccggccccgccatga |
| | | agtccgtgaatgccccgacggccgaagtgaagggcaggccgccacccaggccgcc |
| | | gccctcactgcccggcacctggtcgctgaatgtcgatgccagcacctgcggcacgtca |
| | | atgcttccgggcgtcgcgctcgggctgatcgcccatcccgttactgccccgatcccggc |
| | | aatggcaaggactgccagcgctgccatttttggggtgaggccgttcgcggccgaggg |
| | | gcgcagcccctggggggatgggaggcccgcgttagcgggccgggagggttcgaga |
| | | agggggggcaccccccttcggcgtgcgcggtcacgcgcacagggcgcagccctggt |
| | | taaaaacaaggtttataaatattggtttaaaagcaggttaaaagacaggttagcggtggc |
| | | cgaaaaacgggcggaaacccttgcaaatgctggattttctgcctgtggacagcccctca |
| | | aatgtcaataggtgcgcccctcatctgtcagcactctgcccctcaagtgtcaaggatcgc |
| | | gcccctcatctgtcagtagtcgcgcccctcaagtgtcaataccgcagggcacttatcccc |
| | | aggcttgtccacatcatctgtgggaaactcgcgtaaaatcaggcgttttcgccgatttgcg |
| | | aggctggccagctccacgtcgccggccgaaatcgagcctgcccctcatctgtcaacgc |
| | | cgcgccgggtgagtcggcccctcaagtgtcaacgtccgcccctcatctgtcagtgagg |
| | | gccaagttttccgcgaggtatccacaacgccggcggccgcggtgtctcgcacacggct |
| | | tcgacggcgtttctggcgcgtttgcagggccatagacggccgccagcccagcggcga |
| | | gggcaaccagcccgg |

### Example 6

### Analysis of Metabolic Gene Disruption

After regeneration of multiple transformed cannabis and/or hemp plants, polynucleotide analysis is performed to confirm gene integration and to determine RNA expression levels. In addition, mRNA and protein levels of the disrupted gene are determined. The content of one or more bioactive metabolites, such as terpenes or cannabinoids in plant tissues can also be determined. For example, the content of one or more of THC, CBD, and/or Cannabichromene can be determined with well-established procedures, such as the methods described in US Patent Publication 20160139055, which is hereby incorporated in its entirety. Plants in which gene activity is disrupted and which have reduced THC and/or increased CBD content are selected.

1. A transgenic plant that comprises at least one genetic modification, wherein said genetic modification results in an increased level of the following compounds
(Divarinic acid (DA), Formula I) or a derivative or analog thereof; and 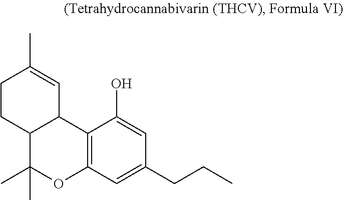 [SMILES: CCCc1cc(O)c2c(c1)OC(C)(C)C1CCC(C)=CC21]
or a derivative or analog thereof;
compared to a level of said compounds in a comparable plant lacking said genetic modification.

2. The transgenic plant of claim 1, wherein said at least one genetic modification further results in an increased level of a compound of:
(Olivetolic acid (OA), Formula II), 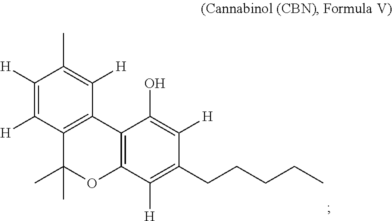 [SMILES: CCCCCc1cc(O)c2c(c1)OC(C)(C)c1ccc(C)cc1-2]
or a derivative or analog thereof, compared to a level of said compound in a comparable plant lacking said genetic modification.

3. The transgenic plant of claim 2, wherein said at least one genetic modification results in an increased level of a compound of Formula V, or a derivative or analog thereof, compared to a level of said compound in a comparable plant lacking said genetic modification.

4. (canceled)

5. (canceled)

6. (canceled)

7. (canceled)

8. The transgenic plant of claim2, wherein said at least one genetic modification results in an increased level of a compound of Formula II, or a derivative or analog thereof, compared to a level of said compound in a comparable plant lacking said genetic modification.

9.-21. (canceled)

22. The transgenic plant of claim 1, wherein said at least one genetic modification is in a gene sequence that encodes a protein.

23. The transgenic plant of claim 22, wherein said at least one genetic modification disrupts expression of said protein.

24. The transgenic plant of claim 22, wherein said at least one genetic modification decreases expression of said protein compared to a comparable plant lacking said genetic modification.

25. The transgenic plant of claim 24, wherein said at least one genetic modification decreases expression of said protein by at least 30%, 40%, 50%, 60%, 70%, 80%, 90%, or 100%, compared to a comparable plant lacking said genetic modification.

26. The transgenic plant of claim 22, wherein said at least one genetic modification is in a gene sequence that encodes a tetrahydrocannabinolic acid synthase, a cannabidiolic acid synthase, or a cannabichromenic acid synthase.

27. The transgenic plant of claim 22, wherein said transgenic plant comprises at least two genetic modifications each in a gene sequence that encodes a protein.

28. The transgenic plant of claim 22, wherein said transgenic plant comprises at least two genetic modifications each in a different gene sequence that encode different proteins.

29. The transgenic plant of claim 22, wherein said at least two genetic modifications disrupts expression of said proteins.

30. (canceled)

31. (canceled)

32. (canceled)

33.-72. (canceled)

73. The transgenic plant of claim 23, wherein said at least one genetic modification is in a gene sequence that encodes a cannabidiolic acid synthase or a cannabichromenic acid synthase.

74. The transgenic plant of claim 73, wherein said at least one genetic modification disrupts expression of said protein.

75. The transgenic plant of claim 74, wherein said at least one genetic modification decreases expression of said protein compared to a comparable plant lacking said genetic modification.

76. (canceled)

77. (canceled)

78.-101. (cancelled)

102. The transgenic plant of claim 1, wherein said transgenic plant further comprises an increased amount of cannabigerol (CBG), derivative or analog thereof, compared to an amount of the same compound in a comparable control plant without said genetic modification.

103. (canceled)

104. The transgenic plant of claim 102, wherein said at least said at least one genetic modification comprises modification of a first group of genes that comprise olivetolic acid cyclase (OAC) and olivetolic acid synthase (OLS).

105. (canceled)

106. (canceled)

107. (canceled)

108. The transgenic plant of claim 1, wherein said genetic modification comprises a disruption of a group of genes encoding CBCA synthase, CBDA synthase, and THCA synthase.

109.-150. (canceled)

151. A pharmaceutical composition comprising an extract of said transgenic plant of claim 2.

152. The pharmaceutical composition of claim 151, further comprising a pharmaceutically acceptable excipient, diluent, or carrier.

153. The pharmaceutical composition of claim 152, comprising said pharmaceutically acceptable excipient, wherein said pharmaceutically acceptable excipient is a lipid.

154. A transgenic plant that comprises at least one genetic modification, wherein said genetic modification results in at least about 50% more of a compound that is 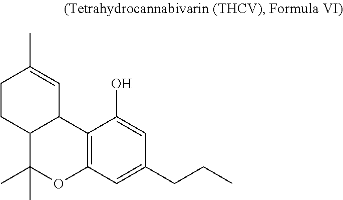 [SMILES: CCCc1cc(O)c2c(c1)OC(C)(C)C1CCC(C)=CC21]
or a derivative or analog thereof compared to a level of said compound in a comparable plant lacking said genetic modification.

155. A method of suppressing appetite in a subject, the method comprising administering an effective amount of the transgenic plant of claim 154 to the subject, thereby suppressing appetite in the subject.

156. The pharmaceutical composition of claim 152 in an oral form, a transdermal form, an oil formulation, an edible food, a food substrate, an aqueous dispersion, an emulsion, a solution, a suspension, an elixir, a gel, a syrup, an aerosol, a mist, a powder, a tablet, a lozenge, a gel, a lotion, a paste, a formulated stick, a balm, a cream, or an ointment.

157. A method of treating a disease or condition in a subject, the method comprising administering pharmaceutical composition of claim 152 to a subject, thereby treating the disease or condition.

158. The method of claim 157, wherein said disease or condition is selected from the group consisting of anorexia, emesis, pain, inflammation, multiple sclerosis, Parkinson's disease, Huntington's disease, Tourette's syndrome, Alzheimer's disease, epilepsy, glaucoma, osteoporosis, schizophrenia, cardiovascular disorders, cancer, and obesity.

159.-163. (canceled)


SEQUENCE LISTING (13 sequences)

SEQ ID NO: 1 - DNA, 1635 bp, Cannabis sativa
atgaattgctcaacattctccttttggtttgtttgcaaaataatatttttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatgtaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttccaccgcgtcatcat

SEQ ID NO: 2 - DNA, 485 bp, Cannabis sativa
aaaaaagaagaagaagaagaaagttgagaaagagaatggcagtgaagcatttgattgtattgaagttcaaagatgaaatcacagaagcccaaaaggaagaatttttcaagacgtatgtgaatcttgtgaatatcatcccagccatgaaagatgtatactggggtaaagatgtgactcaaaagaataaggaagaagggtacactcacatagttgaggtaacatttgagagtgtggagactattcaggactacattattcatcctgcccatgttggatttggagatgtctatcgttctttctgggaaaaacttctcatttttgactacacaccacgaaagtagactatatatagtagccgaccaagctgccttcatcttcatcttctcaaataatatatctaatatctaattatataataataactacttaataaaagactgtgtttataacattaaataataataataataaagtcttttgtagct

SEQ ID NO: 3 - DNA, 1158 bp, Cannabis sativa
atgaatcatcttcgtgctgagggtccggcctccgttctcgccattggcaccgccaatccggagaacattttattacaagatgagtttcctgactactattttcgcgtcaccaaaagtgaacacatgactcaactcaaagaaaagtttcgaaaaatatgtgacaaaagtatgataaggaaacgtaactgtttcttaaatgaagaacacctaaagcaaaacccaagattggtggagcacgagatgcaaactctggatgcacgtcaagacatgttggtagttgaggttccaaaacttgggaaggatgcttgtgcaaaggccatcaaagaatggggtcaacccaagtctaaaatcactcatttaatcttcactagcgcatcaaccactgacatgcccggtgcagactaccattgcgctaagcttctcggactgagtccctcagtgaagcgtgtgatgatgtatcaactaggctgttatggtggtggaaccgttctacgcattgccaaggacatagcagagaataacaaaggcgcacgagttctcgccgtgtgttgtgacataatggcttgcttgtttcgtgggccttcagagtctgacctcgaattactagtgggacaagctatctttggtgatggggctgctgcggtgattgttggagctgaacccgatgagtcagttggggaaaggccgatatttgagttggtgtcaactgggcaaacaatcttaccaaactcggaaggaactattgggggacatataagggaagcaggactgatatttgatttacataaggatgtgcctatgttgatctctaataatattgagaaatgtttgattgaggcatttactcctattgggattagtgattggaactccatattttggattacacacccaggtgggaaagctattttggacaaagtggaggagaagttgcatctaaagagtgataagtttgtggattcacgtcatgtgctgagtgagcatgggaatatgtctagctcaactgtcttgtttgttatggatgagttgaggaagaggtcgttggaggaagggaagtctaccactggagatggatttgagtggggtgttctttttgggtttggaccaggtttgactgtcgaaagagtggtcgtgcgtagtgttcccatcaaatattaa

SEQ ID NO: 4 - protein, 101 aa, Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 5 - DNA, 1188 bp, Cannabis sativa
atgggactctcatcagtttgtaccttttcatttcaaactaattaccatactttattaaatcctcacaataataatcccaaaacctcattattatgttatcgacaccccaaaacaccaattaaatactcttacaataattttccctctaaacattgctccaccaagagttttcatctacaaaacaaatgctcagaatcattatcaatcgcaaaaaattccattagggcagctactacaaatcaaactgagcctccagaatctgataatcattcagtagcaactaaaattttaaactttgggaaggcatgttggaaacttcaaagaccatatacaatcatagcatttacttcatgcgcttgtggattgtttgggaaagagttgttgcataacacaaatttaataagttggtctctgatgttcaaggcattcttttttttggtggctatattatgcattgcttcttttacaactaccatcaatcagatttacgatcttcacattgacagaataaacaagcctgatctaccactagcttcaggggaaatatcagtaaacacagcttggattatgagcataattgtggcactgtttggattgataataactataaaaatgaagggtggaccactctatatatttggctactgttttggtatttttggtgggattgtctattctgttccaccatttagatggaagcaaaatccttccactgcatttcttctcaatttcctggcccatattattacaaatttcacattttattatgccagcagagcagctcttggcctaccatttgagttgaggccttcttttactttcctgctagcatttatgaaatcaatgggttcagctttggctttaatcaaagatgcttcagacgttgaaggcgacactaaatttggcatatcaaccttggcaagtaaatatggttccagaaacttgacattattttgttctggaattgttctcctatcctatgtggctgctatacttgctgggattatctggccccaggctttcaacagtaacgtaatgttactttctcatgcaatcttagcattttggttaatcctccagactcgagattttgcgttaacaaattacgacccggaagcaggcagaagattttacgagttcatgtggaagctttattatgctgaatatttagtatatgttttcatataa

SEQ ID NO: 6 - DNA, 1635 bp, Cannabis sativa; identical to SEQ ID NO: 1

SEQ ID NO: 7 - DNA, 1635 bp, Cannabis sativa
atgaagtgctcaacattctccttttggtttgtttgcaagataatatttttctttttctcattcaatatccaaacttccattgctaatcctcgagaaaacttccttaaatgcttctcgcaatatattcccaataatgcaacaaatctaaaactcgtatacactcaaaacaacccattgtatatgtctgtcctaaattcgacaatacacaatcttagattcacctctgacacaaccccaaaaccacttgttatcgtcactccttcacatgtctctcatatccaaggcactattctatgctccaagaaagttggcttgcagattcgaactcgaagtggtggtcatgattctgagggcatgtcctacatatctcaagtcccatttgttatagtagacttgagaaacatgcgttcaatcaaaatagatgttcatagccaaactgcatgggttgaagccggagctacccttggagaagtttattattgggttaatgagaaaaatgagaatcttagtttggcggctgggtattgccctactgtttgcgcaggtggacactttggtggaggaggctatggaccattgatgagaaactatggcctcgcggctgataatatcattgatgcacacttagtcaacgttcatggaaaagtgctagatcgaaaatctatgggggaagatctcttttgggctttacgtggtggtggagcagaaagcttcggaatcattgtagcatggaaaattagactggttgctgtcccaaagtctactatgtttagtgttaaaaagatcatggagatacatgagcttgtcaagttagttaacaaatggcaaaatattgcttacaagtatgacaaagatttattactcatgactcacttcataactaggaacattacagataatcaagggaagaataagacagcaatacacacttacttctcttcagttttccttggtggagtggatagtctagtcgacttgatgaacaagagttttcctgagttgggtattaaaaaaacggattgcagacaattgagctggattgatactatcatcttctatagtggtgttgtaaattacgacactgataattttaacaaggaaattttgcttgatagatccgctgggcagaacggtgctttcaagattaagttagactacgttaagaaaccaattccagaatctgtatttgtccaaattttggaaaaattatatgaagaagatataggagctgggatgtatgcgttgtacccttacggtggtataatggatgagatttcagaatcagcaattccattccctcatcgagctggaatcttgtatgagttatggtacatatgtagttgggagaagcaagaagataacgaaaagcatctaaactggattagaaatatttataacttcatgactccttatgtgtccaaaaatccaagattggcatatctcaattatagagaccttgatataggaataaatgatcccaagaatccaaataattacacacaagcacgtatttggggtgagaagtattttggtaaaaattttgacaggctagtaaaagtgaaaaccctggttgatcccaataacttttttagaaacgaacaaagcatcccacctcttccacggcatcgtcattaa

SEQ ID NO: 8 - DNA, 1885 bp, Cannabis sativa
aaaaaaatcattaggactgaagaaaaatgaattgctcagcattttccttttggtttgtttgcaaaataatatttttctttctctcattccatatccaaatttcaatagctaatcctcgagaaaacttccttaaatgcttctcaaaacatattcccaacaatgtagcaaatccaaaactcgtatacactcaacacgaccaattgtatatgtctatcctgaattcgacaatacaaaatcttagattcatctctgatacaaccccaaaaccactcgttattgtcactccttcaaataactcccatatccaagcaactattttatgctctaagaaagttggcttgcagattcgaactcgaagcggtggccatgatgctgagggtatgtcctacatatctcaagtcccatttgttgtagtagacttgagaaacatgcattcgatcaaaatagatgttcatagccaaactgcgtgggttgaagccggagctacccttggagaagtttattattggatcaatgagaagaatgagaatcttagttttcctggtgggtattgccctactgttggcgtaggtggacactttagtggaggaggctatggagcattgatgcgaaattatggccttgcggctgataatattattgatgcacacttagtcaatgttgatggaaaagttctagatcgaaaatccatgggagaagatctgttttgggctatacgtggtggtggaggagaaaactttggaatcattgcagcatggaaaatcaaactggttgctgtcccatcaaagtctactatattcagtgttaaaaagaacatggagatacatgggcttgtcaagttatttaacaaatggcaaaatattgcttacaagtatgacaaagatttagtactcatgactcacttcataacaaagaatattacagataatcatgggaagaataagactacagtacatggttacttctcttcaatttttcatggtggagtggatagtctagtcgacttgatgaacaagagctttcctgagttgggtattaaaaaaactgattgcaaagaatttagctggattgatacaaccatcttctacagtggtgttgtaaattttaacactgctaattttaaaaaggaaattttgcttgatagatcagctgggaagaagacggctttctcaattaagttagactatgttaagaaaccaattccagaaactgcaatggtcaaaattttggaaaaattatatgaagaagatgtaggagctgggatgtatgtgttgtacccttacggtggtataatggaggagatttcagaatcagcaattccattccctcatcgagctggaataatgtatgaactttggtacactgcttcctgggagaagcaagaagataatgaaaagcatataaactgggttcgaagtgtttataattttacgactccttatgtgtcccaaaatccaagattggcgtatctcaattatagggaccttgatttaggaaaaactaatcatgcgagtcctaataattacacacaagcacgtatttggggtgaaaagtattttggtaaaaattttaacaggttagttaaggtgaaaactaaagttgatcccaataatttttttagaaacgaacaaagtatcccacctcttccaccgcatcatcattaattatctttaaatagatatatttcccttatcaattagttaatcattataccatacatacatttattgtatatagtttatctactcatattatgtatgctcccaagtatgaaaatctacattagaactgtgtagacaatcataagatatatttaataaaataaattgtctttcttatttcaatagcaaataaaataatattattttaaaaaaaaaaaaaaaaa

SEQ ID NO: 9 - DNA, 13490 bp, Artificial Sequence
  Description of Artificial Sequence Synthetic polynucleotide
agcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcaggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgataactgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctgcaagcaagaattcaagcttggagccagaaggtaattatccaagatgtagcatcaagaatccaatgtttacgggaaaaactatggaagtattatgtaagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgatgacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtaatggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagactatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagattatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtctccacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtttcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaaggatctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtgagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctactaggagcatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgcttgcctctgttccccagagggcagttttagagctagaaatagcaagttaaaataaggctagtccgttatcaacttgaaaaagtggcaccgagtcggtgctttttttctagacccagctttcttgtacaaagttggcattacgctttacgaattcccatggggagcatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgcttgctgttccccagagggcaggggttttagagctagaaatagcaagttaaaataaggctagtccgttatcaacttgaaaaagtggcaccgagtcggtgctttttttctagacccagctttcttgtacaaagttggcattacgctcagagaattcgcatgcggagcatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgcttgaacctcaagcacgagaacttgttttagagctagaaatagcaagttaaaataaggctagtccgttatcaacttgaaaaagtggcaccgagtcggtgctttttttctagacccagctttcttgtacaaagttggcattacgcttgtgtgagaccgaggatgcacatgtgaccgagggacacgaagtgatccgtttaaactatcagtgtttgacaggatatattggcgggtaaacctaagagaaaagagcgtttattagaataatcggatatttaaaagggcgtgaaaaggtttatccgttcgtccatttgtatgtgccagccgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggccaattcccgaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagcgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccattacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcagatcctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtcttgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagcccctgctcggatctgttggaccggacagtagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaacactctgtgggtctcatgccgaattcggatccggaggaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaagtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtcaagactggcgaacagttcatacagagtcttttacgactcaatgacaagaagaaaatcttcgtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgatacaatgaaaa

SEQ ID NO: 10 - DNA, 13377 bp, Artificial Sequence
  Description of Artificial Sequence Synthetic polynucleotide
ctcgagcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttcttgactcttcgcgatgtacgggccagatatgtcgaccgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcgcgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctcgcggcctggccggcgagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggcggcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggcgagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagttagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcacctctagatggcaggatatattgtggtgtaaacagtttaaacagtgttttactcctcatattaacttcggtcattagaggccacgatttgacacatttttactcaaaacaaaatgtttgcatatctcttataatttcaaattcaacacacaacaaataagagaaaaaacaaataatattaatttgagaatgaacaaaaggaccatatcattcattaactcttctccatccatttccatttcacagttcgatagcgaaaaccgaataaaaaacacagtaaattacaagcacaacaaatggtacaagaaaaacagttttcccaatgccataatactcgaacgtccggagttatcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccaagctcccattgttggtacccagcttgggtctagtcgtattaagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccattacataggcccatcggagctaacgcagtgaattcagaaatctcaaaattccggcagaacaattttgaatctcgatccgtagaaacgagacggtcattgttttagttccaccacgattatatttgaaatttacgtgagtgtgagtgagacttgcataagaaaataaaatctttagttgggaaaaaattcaataatataaatgggcttgagaaggaagcgagggataggcctttttctaaaataggcccatttaagctattaacaatcttcaaaagtaccacagcgcttaggtaaagaaagcagctgagtttatatatggttagagacgaagtagtgattggatggcaggtggaagaatggacacctgcgagagttttagagctagaaatagcaagttaaaataaggctagtccgttatcaacttgaaaaagtggcaccgagtcggtgctttttttacagtgaaagcttactgcgttagctccgatgggcctatgtaatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctcttaatacgactcactatagggagacccaagctggctagcaacaatggataagaagtactctatcggactcgatatcggaactaactctgttggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagattttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagggtgaacaccgagatcaccaaggctccactttctgcttctatgatcaagagatacgatgagcaccaccaggatctcacacttctcaaggctcttgttagacagcagctcccagagaagtacaaagaaatcttcttcgatcagtctaagaacggatacgctggttacatcgatggtggtgcatctcaagaagagttctacaagttcatcaagccaatcttggagaagatggatggaaccgaggaactcctcgtgaagctcaatagagaggatctccttaggaagcagaggaccttcgataacggatctatccctcatcagatccacctcggagagttgcacgctatccttagaaggcaagaggatttctacccattcctcaaggataacagagagaagattgagaagatcctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttcctaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagtggaaatctctggtgttgaggataggttcaacgcatctctcggaacctaccacgatctcctcaagatcattaaggataaggatttcttggataacgaggaaaacgaggatatcttggaggatatcgttcttaccctcaccctcttcgaggatagagagatgatagaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatcccctgcaatcaagaagggaatcctccagactgtgaagattgtggatgagttggtgaaggtgatgggacacaagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagctccagaacgagaagctctacctctactacttgcagaacggaagggatatgtatgtggatcaagagcttgatattaacaggctctctgattacgatgttgatcatatcgtgccacagtcttttatcaaagatgattctatcgataacaaggtgctcactaggtctgataagaacaggggtaagagtgataacgtgccaagtgaagaggttgtgaagaaaatgaagaactattggaggcagctcctcaacgctaagctcatcactcagagaaagttcgataacttgaccaaggctgagaggggaggactctctgaattggataaggcaggattcatcaagagacagctcgtggaaaccaggcagatcaccaaacatgtggcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatccaaagttggagtctgagttcgtgtacggtgattataaggtgtacgatgtgaggaagatgatcgctaagtctgagcaagagatcggaaaggctaccgctaagtatttcttctactctaacatcatgaatttcttcaagaccgagatcactctcgctaacggtgagatcagaaagaggccactcatcgagacaaacggtgaaacaggtgagatcgtgtgggataagggaagggatttcgctaccgttagaaaggtgctctctatgcctcaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttccttgaggctaagggatacaaagaggttaagaaggatctcatcatcaagctcccaaagtactcacttttcgagttggagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatcagtgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagcttggaggtgatcctaagaagaaaagaaaggttagatcttgatgacccgggtctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaaggccttgacaggatatattggcgggtaaacctaagagaaaagagcgtttattagaataacggatatttaaaactcgag

SEQ ID NO: 11 - DNA, 11850 bp, Artificial Sequence
  Description of Artificial Sequence Synthetic polynucleotide
gatctgagggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgatagttacagaaccgacgactcgtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactcgaagtcacagccaaaagccagacagagtctgatatctacccgcttcgcgtcggcatccggtcagtggcagtgaagggccaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttcagctgtctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaagctagccaccaccaccaccaccacgtgtgaattacaggtgaccagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttagcttcatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcttgaccatggta

SEQ ID NO: 12 - DNA, 17981 bp, Artificial Sequence
  Description of Artificial Sequence Synthetic polynucleotide
tgagcgtcgcaaaggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccagatctggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgggatctctgcggaacaggcggtcgaaggtgccgatatcattacgacagcaacggccgacaagcacaacgccacgatcctgagcgacaatatgatcgggcccggcgtccacatcaacggcgtcggcggcgactgcccaggcaagaccgagatgcaccgcgatatcttgctgcgttcggatattttcgtggagttcccgccacagacccggatgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggcctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctaatcaaacaagtttgtacaaaaaagctgaacgagaaacgtaaaatgatgaattgctcaacattctccttttggtttgtttgcaaaataatatttttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttccaccgcgtcatcattaaaatatattgatatttatatcattttacgtttctcgttcagctttcttgtacaaagtggttcgatctagaggatccatggtgagcaagggcgaggagctgttcaccggggtggtgcccatcctggtcgagctggacggcgacgtgaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaaagcggcccgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattagcttcatcaacgcaagacatgcgcacgaccgtctgacaggagaggaatttccgacgagcacagaaaggacttgctcttggacgtaggcctatttctcaggcacatgtatcaagtgttcggacgtgggttttcgatggtgtatcagccgccgccaactgggagatgaggaggctttcttggggggcagtcagcagttcatttcacaagacagaggaacttgtaaggagatgcactgatttatcttggcgcaaaccagcaggacgaattagtgggaatagcccgcgaatatctaagttatgcctgtcggcatgagcagaaacttccaattcgaaacagtttggagaggttgtttttgggcataccttttgttagtcagcctctcgattgctcatcgtcattacacagtaccgaagtttgatcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttcgacgcactccttctttactccaccatctcgtccttattgaaaacgtgggtagcaccaaaacgaatcaagtcgctggaactgaagttaccaatcacgctggatgatttgccagttggattaatcttgcctttccccgcatgaataatattgatgaatgcatgcgtgaggggtatttcgattttggcaatagctgcaattgccgcgacatcctccaacgagcataattcttcagaaaaatagcgatgttccatgttgtcagggcatgcatgatgcacgttatgaggtgacggtgctaggcagtattccctcaaagtttcatagtcagtatcatattcatcattgcattcctgcaagagagaattgagacgcaatccacacgctgcggcaaccttccggcgttcgtggtctatttgctcttggacgttgcaaacgtaagtgttggatcccggtcggcatctactctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatcggggtcgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttgacggatctctaggacgcgtcctagaagctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatatcaactcccctatccattgctcaccgaatggtacaggtcggggacccgaagttccgactgtcggcctgatgcatccccggctgatcgaccccagatctggggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaacttactgatttagtgtatgatggtgtttttgaggtgctccagtggcttctgtgtctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaattatgatagaatttacaagctataaggttattgtcctgggtttcaagcattagtccatgcaagtttttatgctttgcccattctatagatatattgataagcgcgctgcctatgccttgccccctgaaatccttacatacggcgatatcttctatataaaagatatattatcttatcagtattgtcaatatattcaaggcaatctgcctcctcatcctcttcatcctcttcgtcttggtagctttttaaatatggcgcttcatagagtaattctgtaaaggtccaattctcgttttcatacctcggtataatcttacctatcacctcaaatggttcgctgggtttatcgcacccccgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccgg

SEQ ID NO: 13 - DNA, 17978 bp, Artificial Sequence
  Description of Artificial Sequence Synthetic polynucleotide
tgagcgtcgcaaaggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccagatctggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgggatctctgcggaacaggcggtcgaaggtgccgatatcattacgacagcaacggccgacaagcacaacgccacgatcctgagcgacaatatgatcgggcccggcgtccacatcaacggcgtcggcggcgactgcccaggcaagaccgagatgcaccgcgatatcttgctgcgttcggatattttcgtggagttcccgccacagacccggatgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggcctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctaatcaaacaagtttgtacaaaaaagctgaacgagaaacgtaaaatgatgaagtactcaacattctccttttggtttgtttgcaagataatatttttctttttctcattcaatatccaaacttccattgctaatcctcgagaaaacttccttaaatgcttctcgcaatatattcccaataatgcaacaaatctaaaactcgtatacactcaaaacaacccattgtatatgtctgtcctaaattcgacaatacacaatcttagattcagctctgacacaaccccaaaaccacttgttatcgtcactccttcacatgtctctcatatccaaggcactattctatgctccaagaaagttggcttgcagattcgaactcgaagtggtggtcatgattctgagggcatgtcctacatatctcaagtcccatttgttatagtagacttgagaaacatgcgttcaatcaaaatagatgttcatagccaaactgcatgggttgaagccggagctacccttggagaagtttattattgggttaatgagaaaaatgagagtcttagtttggctgctgggtattgccctactgtttgcgcaggtggacactttggtggaggaggctatggaccattgatgagaagctatggcctcgcggctgataatatcattgatgcacacttagtcaacgttcatggaaaagtgctagatcgaaaatctatgggggaagatctcttttgggctttacgtggtggtggagcagaaagcttcggaatcattgtagcatggaaaattagactggttgctgtcccaaagtctactatgtttagtgttaaaaagatcatggagatacatgagcttgtcaagttagttaacaaatggcaaaatattgcttacaagtatgacaaagatttattactcatgactcacttcataactaggaacattacagataatcaagggaagaataagacagcaatacacacttacttctcttcagttttccttggtggagtggatagtctagtcgacttgatgaacaagagttttcctgagttgggtattaaaaaaacggattgcagacaattgagctggattgatactatcatcttctatagtggtgttgtaaattacgacactgataattttaacaaggaaattttgcttgatagatccgctgggcagaacggtgctttcaagattaagttagactacgttaagaaaccaattccagaatctgtatttgtccaaattttggaaaaattatatgaagaagatataggagctgggatgtatgcgttgtacccttacggtggtataatggatgagatttctgaatcagcaattccattccctcatcgagctggaatcttgtatgagttatggtacatatgtagctgggagaagcaagaagataacgaaaagcatctaaactggattagaaatatttataacttcatgactccttatgtgtcccaaaatccaagattggcatatctcaattatagagaccttgatataggaataaatgatcccaagaatccaaataattacacacaagcacgtatttggggtgagaagtattttggtaaaaattttgacaggctagtaaaagtgaaaaccctggttgatcccaataatttttttagaaacgaacaaagcatcccacctcttccacggcatcatcattaaaatatattgatatttatatcattttacgtttctcgttcagctttcttgtacaaagtggttcgatctagaggatccatggtgagcaagggcgaggagctgttcaccggggtggtgcccatcctggtcgagctggacggcgacgtgaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaaagcggcccgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattagcttcatcaacgcaagacatgcgcacgaccgtctgacaggagaggaatttccgacgagcacagaaaggacttgctcttggacgtaggcctatttctcaggcacatgtatcaagtgttcggacgtgggttttcgatggtgtatcagccgccgccaactgggagatgaggaggctttcttggggggcagtcagcagttcatttcacaagacagaggaacttgtaaggagatgcactgatttatcttggcgcaaaccagcaggacgaattagtgggaatagcccgcgaatatctaagttatgcctgtcggcatgagcagaaacttccaattcgaaacagtttggagaggttgtttttgggcataccttttgttagtcagcctctcgattgctcatcgtcattacacagtaccgaagtttgatcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttcgacgcactccttctttactccaccatctcgtccttattgaaaacgtgggtagcaccaaaacgaatcaagtcgctggaactgaagttaccaatcacgctggatgatttgccagttggattaatcttgcctttccccgcatgaataatattgatgaatgcatgcgtgaggggtatttcgattttggcaatagctgcaattgccgcgacatcctccaacgagcataattcttcagaaaaatagcgatgttccatgttgtcagggcatgcatgatgcacgttatgaggtgacggtgctaggcagtattccctcaaagtttcatagtcagtatcatattcatcattgcattcctgcaagagagaattgagacgcaatccacacgctgcggcaaccttccggcgttcgtggtctatttgctcttggacgttgcaaacgtaagtgttggatcccggtcggcatctactctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatcggggtcgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttgacggatctctaggacgcgtcctagaagctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatatcaactcccctatccattgctcaccgaatggtacaggtcggggacccgaagttccgactgtcggcctgatgcatccccggctgatcgaccccagatctggggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaacttactgatttagtgtatgatggtgtttttgaggtgctccagtggcttctgtgtctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaattatgatagaatttacaagctataaggttattgtcctgggtttcaagcattagtccatgcaagtttttatgctttgcccattctatagatatattgataagcgcgctgcctatgccttgccccctgaaatccttacatacggcgatatcttctatataaaagatatattatcttatcagtattgtcaatatattcaaggcaatctgcctcctcatcctcttcatcctcttcgtcttggtagctttttaaatatggcgcttcatagagtaattctgtaaaggtccaattctcgttttcatacctcggtataatcttacctatcacctcaaatggttcgctgggtttatcgcacccccgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccgg